SEQUENCE LISTING (176 sequences)

SEQ ID NO: 1 - protein, 1087 aa, Geobacillus stearothermophilus
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 2 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 3 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGTMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENTRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 4 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSKIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 5 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 6 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 7 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHRETLRRYVGIDERSGKIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 8 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDEHSGKIQTVVKTKLSEIKLDASGHFPMYGKESDPRTYEAIRQRLLEHNNDPKKAFQEPLYRPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKAINSANGGLELISHDHRFSLRGVGSRTLKRFEKYQVDVLGNIYKVRGEKRVGLASSAHSKPGKTIRPLQSTRD

SEQ ID NO: 9 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIGSANGGLELISHDHRFSLRGVGSRTLKRFEKYQVDVLGNIYKVRGEKRVGLASSAHSKPGKTIRPLQSTRD

SEQ ID NO: 10 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKAINSANGGLELISHDHRFSLRGVGSRTLKRFEKYQVDVLGNIYKVRGEKRVGLASSAHSKPGKTIRPLQSTRD

SEQ ID NO: 11 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHRETLRRYVGIDERSGKIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIGSANGGLELISHDHRFSLRGVGSRTLKRFEKYQVDVLGNIYKVRGEKRVGLASSAHSKPGKTIRPLQSTRD

SEQ ID NO: 12 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVATRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGRIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 13 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVPTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGRIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 14 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGGNYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 15 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGGNYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHRETLRRYVGIDERSGKIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 16 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGRNYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 17 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVATRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGRIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEVDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 18 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVTTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGRIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFESDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 19 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVPTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGRIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEDDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 20 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGLIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFESDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 21 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVRTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACATPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGLIQTVVKTKLSKIKLDASGHFPMYGKESDPRTYEAIRQRLLEHNNDPKKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 22 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDPEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYNQIRKAVDKVYGKGKSSSFLPIDFDTFGYALTLFKDDADTHSYLRNEYEQNGKRMPNLANKVYDNELIEELLNLSFTKFGHLSLKALRSILPYMEQGEVYSSACERAGYTFTGPKRKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHRETLRRYVGIDERSGKIQTVVKTKLSKIKLDASGHFPMYGKESDPRTYEAIRQRLLEHNNDPRKAFQGPLYKPKKNGEPGPVIRTVKIIDTRNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRA

SEQ ID NO: 23 - protein, 1087 aa, synthetic construct
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEGNRAILSSYRTVGEMIVKDPKFALHKRNKGENYINTIARDDLEREIRLIFSKQREFGDMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIQPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHRETLRRYVGIDERSGKIQTVVKTKLSKIKLDASGHFPMYGKESDPRTYEAIRQRLLEHNNDPKKAFQGPLYKPKKNGEPGPVIRTVKIIDTRNQVIPLNDGKTVAYNPNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHNKPGKTIRPLQSTRD

SEQ ID NO: 24 - protein, 1368 aa, Streptococcus pyogenes [sequence omitted]

SEQ ID NO: 25 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 26 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 27 - protein, 1306 aa, Acidaminococcus sp.
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 28 - protein, 1228 aa, unidentified
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 29 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 30 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 31 - protein, 1601 aa, Desulfonema ishimotonii
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 32 - protein, 966 aa, Ruminococcus flavefaciens
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 33 - protein, 1082 aa, Prevotella sp.
NQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMK

SEQ ID NO: 34 - protein, 1320 aa, Prevotella sp.
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSKFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKFKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSNSGTQFGGYLFRKKNEIGEYDYFLGISSKTQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIGNLLKTISCLKNKEEFHDLIKKDYQIFTEVQAVIDEICKQKTFIYFPVSNVELEKEMGDKDKPLCLFQISNKDLSFAKTFSANLRKKRGAENLHTMLFKALMSGNQDNLDLGSGAIFYRAKSLDGNKPTHPANEAIKCRNVANKDKVSLFTYDIYKNRRYMENKFLFHLSIVQNYKAAKDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGEMFVTRGQKIEKAVYQQFEKSLVDKLSYLVDKKRPYNELGGILKAYQLASSITKSNSDKQNGFLFYVPAWNTSKIDPVTGFTDLLRPKAMTIKEAQDFFGAFDNISYNDKGYFEFETNYDKFKIRMKGAQTRWTICTFGNRIKHKKDKNYWNYEEVELTEEFKKLFDKYDIDYRDGNLKEEILKIDNRKFFDALIKLLQLTLQLRNSDDKGNDYIVSPIANADGKFFNSNDGCKELPLDADANGAYNIARKGLWVVRQIKDKKDKISKLSNQEWLKFAQEKPYLK

SEQ ID NO: 35 - protein, 1323 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 36 - protein, 671 aa, Moloney murine leukemia virus
LNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 37 - RNA, 98 nt, synthetic construct
  source 1..98: mol_type = other RNA
tcagggttactatgataagggctttctgcctaaggcagactgacccgcggcgttggggatcgcctgtcgcccgcttttggcgggcattccccatcctt

SEQ ID NO: 38 - RNA, 82 nt, synthetic construct
  source 1..82: mol_type = other RNA
aagggctttctgcctaaggcagactgacccgcggcgttggggatcgcctgtcgcccgcttttggcgggcattccccatcctt

SEQ ID NO: 39 - RNA, 118 nt, synthetic construct
  source 1..118: mol_type = other RNA
gtcatagttcccctgagaaatcagggttactatgataagggctttctgcctaaggcagactgacccgcggcgttggggatcgcctgtcgcccgcttttggcgggcattccccatcctt

SEQ ID NO: 40 - protein, 93 aa, synthetic construct
IIKTAGGEEIKIKDLFAYYKTIHSGTAGLELVSHDCSFSLSGVGSRTLKRFEKYQVDVLGNIYKVRGEKRVGLASSAHSKTGETIRPLQSTRD

SEQ ID NO: 41 - protein, 93 aa, synthetic construct
TVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 42 - protein, 167 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 43 - protein, 178 aa, synthetic construct
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 44 - protein, 160 aa, synthetic construct
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 45 - protein, 161 aa, synthetic construct
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 46 - protein, 183 aa, synthetic construct
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 47 - protein, 164 aa, synthetic construct
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 48 - protein, 173 aa, synthetic construct
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 49 - protein, 161 aa, synthetic construct
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 50 - protein, 179 aa, synthetic construct
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 51 - protein, 198 aa, synthetic construct [sequence omitted]

SEQ ID NO: 52 - protein, 188 aa, synthetic construct
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 53 - protein, 198 aa, synthetic construct [sequence omitted]

SEQ ID NO: 54 - DNA, 21 bp, synthetic construct
  source 1..21: mol_type = other DNA
tgtttattgcagcttataatg

SEQ ID NO: 55 - DNA, 21 bp, synthetic construct
  source 1..21: mol_type = other DNA
taaagcaatagcatcacaaat

SEQ ID NO: 56 - DNA, 21 bp, synthetic construct
  source 1..21: mol_type = other DNA
cactgcattctagttgtggtt

SEQ ID NO: 57 - DNA, 21 bp, synthetic construct
  source 1..21: mol_type = other DNA
ttttttaaagcaagtaaaacc

SEQ ID NO: 58 - DNA, 21 bp, synthetic construct
  source 1..21: mol_type = other DNA
ggttacaaataaagcaatagc

SEQ ID NO: 59 - DNA, 21 bp, synthetic construct
  source 1..21: mol_type = other DNA
gataagatacattgatgagtt

SEQ ID NO: 60 - DNA, 21 bp, synthetic construct
  source 1..21: mol_type = other DNA
acctcccacacctccccctga

SEQ ID NO: 61 - DNA, 21 bp, synthetic construct
  source 1..21: mol_type = other DNA
ggacaaaccacaactagaatg

SEQ ID NO: 62 - DNA, 21 bp, synthetic construct
  source 1..21: mol_type = other DNA
ataacttcgtatagcatacat

SEQ ID NO: 63 - DNA, 21 bp, synthetic construct
  source 1..21: mol_type = other DNA
ataacttcgtataatgtatgc

SEQ ID NO: 64 - DNA, 21 bp, synthetic construct
  source 1..21: mol_type = other DNA
tctagaggatcataatcagcc

SEQ ID NO: 65 - DNA, 21 bp, synthetic construct
  source 1..21: mol_type = other DNA
gttttacttgctttaaaaaac

SEQ ID NO: 66 - DNA, 21 bp, synthetic construct
  source 1..21: mol_type = other DNA
aataaagcaatagcatcacaa

SEQ ID NO: 67 - DNA, 21 bp, synthetic construct
  source 1..21: mol_type = other DNA
gatacattgatgagtttggac

SEQ ID NO: 68 - DNA, 21 bp, synthetic construct
  source 1..21: mol_type = other DNA
ggctgattatgatcctctaga

SEQ ID NO: 69 - DNA, 21 bp, synthetic construct
  source 1..21: mol_type = other DNA
cctgaacctgaaacataaaat

SEQ ID NO: 70 - DNA, 21 bp, synthetic construct
  source 1..21: mol_type = other DNA
gctttatttgtaaccattata

SEQ ID NO: 71 - DNA, 21 bp, synthetic construct
  source 1..21: mol_type = other DNA
tagaggatctgcgactctaga

SEQ ID NO: 72 - DNA, 21 bp, synthetic construct
  source 1..21: mol_type = other DNA
gcgactctagaggatcataat

SEQ ID NO: 73 - DNA, 21 bp, synthetic construct
  source 1..21: mol_type = other DNA
cccacacctccccctgaacct

SEQ ID NO: 74 - DNA, 21 bp, synthetic construct
  source 1..21: mol_type = other DNA
tattgcagcttataatggtta

SEQ ID NO: 75 - DNA, 21 bp, synthetic construct
  source 1..21: mol_type = other DNA
gcaatagcatcacaaatttca

SEQ ID NO: 76 - DNA, 21 bp, synthetic construct
  source 1..21: mol_type = other DNA
gcattctagttgtggtttgtc

SEQ ID NO: 77 - DNA, 198 bp, synthetic construct
  source 1..198: mol_type = other DNA
gaagttcatctgcaccaccggcaagctgcccgtgccctggcccaccctcgtgaccaccctgagccatggcgtgcagtgcttcagccgctaccccgaccacatgaagcagcacgacttcttcaagtcagctatgccggaaggctacgtccaggagcgcaccatcttcttcaaggacgacggcaactacaagacccgcgc

SEQ ID NO: 78 - DNA, 198 bp, synthetic construct
  source 1..198: mol_type = other DNA
cttcaagtagacgtggtggccgttcgacgggcacgggaccgggtgggagcactggtgggactcggtaccgcacgtcacgaagtcggcgatggggctggtgtacttcgtcgtgctgaagaagttcagtcgatacggccttccgatgcaggtcctcgcgtggtagaagaagttcctgctgccgttgatgttctgggcgcg

SEQ ID NO: 79 - DNA, 259 bp, synthetic construct
  source 1..259: mol_type = other DNA
ccctgaagttcatctgcaccacggcaagctgcccgtgccctggcccaccctcgtgaccaccctgacctatggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgac

SEQ ID NO: 80 - DNA, 163 bp, synthetic construct
  source 1..163: mol_type = other DNA
acggcaagctgaccctgaagttcatctgcaccaccggcaagctgcccgtgccctggcccaccctcgtgaccacactgagccacggcgtgcagtgcttcagccgctaccccgaccacatgaagcagcacgacttcttcaagtccgccatgcccgaaggctacgt

SEQ ID NO: 81 - DNA, 163 bp, synthetic construct
  source 1..163: mol_type = other DNA
tgccgttcgactgggacttcaagtagacgtggtggccgttcgacgggcacgggaccgggtgggagcactggtgtgactcggtgccgcacgtcacgaagtcggcgatggggctggtgtacttcgtcgtgctgaagaagttcaggcggtacgggcttccgatgca

SEQ ID NO: 82 - DNA, 280 bp, synthetic construct
  source 1..280: mol_type = other DNA
gccacctacggcaagctgaccctgaagttcatctgcaccaccggcaagctgcccgtgccctggcccaccctcgtgaccaccctgacctatggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgact

SEQ ID NO: 83 - DNA, 13 bp, synthetic construct
  source 1..13: mol_type = other DNA
cgccatgcccgaa

SEQ ID NO: 84 - DNA, 13 bp, synthetic construct
  source 1..13: mol_type = other DNA
agctatgccggaa

SEQ ID NO: 85 - protein, 87 aa, synthetic construct
TLKFICTTGKLPVPWPTLVTTLTYGVQCFSRYPDHMKQHDFFKSAMPEGYVQERTIFFKDDGNYKTRAEVKFEGDTLVNRIELKGID

SEQ ID NO: 86 - protein, 93 aa, synthetic construct
ATYGKLTLKFICTTGKLPVPWPTLVTTLTYGVQCFSRYPDHMKQHDFFKSAMPEGYVQERTIFFKDDGNYKTRAEVKFEGDTLVNRIELKGID

SEQ ID NO: 87 - DNA, 21 bp, synthetic construct
  source 1..21: mol_type = other DNA
acgacttcttcaagtccgcca

SEQ ID NO: 88 - DNA, 21 bp, synthetic construct
  source 1..21: mol_type = other DNA
acctgagcacccagtccaagc

SEQ ID NO: 89 - DNA, 21 bp, synthetic construct
  source 1..21: mol_type = other DNA
caagctggagtacaactacaa

SEQ ID NO: 90 - DNA, 21 bp, synthetic construct
  source 1..21: mol_type = other DNA
gggcacgggcagcttgccggt

SEQ ID NO: 91 - DNA, 21 bp, synthetic construct
  source 1..21: mol_type = other DNA
ttgaagaagatggtgcgctcc

SEQ ID NO: 92 - DNA, 21 bp, synthetic construct
  source 1..21: mol_type = other DNA
atgcggttcaccagggtgtcg

SEQ ID NO: 93 - DNA, 10 bp, synthetic construct
  source 1..10: mol_type = other DNA
aagccacgga

SEQ ID NO: 94 - DNA, 10 bp, synthetic construct
  source 1..10: mol_type = other DNA
aactcacgga

SEQ ID NO: 95 - DNA, 68 bp, synthetic construct
  source 1..68: mol_type = other DNA
ccgtgccctggcccactttggtgaccactttgacatatggcgtacaatgcttttcgcgctaccccgac

SEQ ID NO: 96 - DNA, 10 bp, synthetic construct
  source 1..10: mol_type = other DNA
ttcggtgcct

SEQ ID NO: 97 - DNA, 10 bp, synthetic construct
  source 1..10: mol_type = other DNA
ttgagtgcct

SEQ ID NO: 98 - DNA, 22 bp, synthetic construct
  source 1..22: mol_type = other DNA
cgcgaaaagcattgtacgccat

SEQ ID NO: 99 - DNA, 22 bp, synthetic construct
  source 1..22: mol_type = other DNA
tgtacgccatatgtcaaagtgg

SEQ ID NO: 100 - DNA, 23 bp, synthetic construct
  source 1..23: mol_type = other DNA
cactttggtgaccactttgacat

SEQ ID NO: 101 - DNA, 23 bp, synthetic construct
  source 1..23: mol_type = other DNA
ttggtgaccactttgacatatgg

SEQ ID NO: 102 - DNA, 15 bp, synthetic construct
  source 1..15: mol_type = other DNA
ggagaaggaggaatt

SEQ ID NO: 103 - protein, 16 aa, synthetic construct
KDNIVLEGGITLSGGQ

SEQ ID NO: 104 - DNA, 51 bp, synthetic construct
  source 1..51: mol_type = other DNA
aaagacaatatagttctttgagaaggtggaatcacactgagtggaggtcaa

SEQ ID NO: 105 - DNA, 15 bp, synthetic construct
  source 1..15: mol_type = other DNA
cctcttcctccttaa

SEQ ID NO: 106 - DNA, 10 bp, synthetic construct
  source 1..10: mol_type = other DNA
acaacaatgg

SEQ ID NO: 107 - protein, 16 aa, synthetic construct
DSITLQQRKAFGVIPQ

SEQ ID NO: 108 - DNA, 51 bp, synthetic construct
  source 1..51: mol_type = other DNA
gattcaataactttgcaacagtgaaggaaagcctttggagtgataccacag

SEQ ID NO: 109 - DNA, 10 bp, synthetic construct
  source 1..10: mol_type = other DNA
tgttgttacc

SEQ ID NO: 110 - DNA, 24 bp, synthetic construct
  source 1..24: mol_type = other DNA
gggaagaagtggctgatctcagcc

SEQ ID NO: 111 - DNA, 24 bp, synthetic construct
  source 1..24: mol_type = other DNA
ctataaaagagagagccgttatcg

SEQ ID NO: 112 - DNA, 24 bp, synthetic construct
  source 1..24: mol_type = other DNA
ctgatctcagccaccgcgaaaatg

SEQ ID NO: 113 - DNA, 24 bp, synthetic construct
  source 1..24: mol_type = other DNA
acagagtgatattattgacacgcc

SEQ ID NO: 114 - DNA, 24 bp, synthetic construct
  source 1..24: mol_type = other DNA
ccacttcttccccgataacggaga

SEQ ID NO: 115 - DNA, 24 bp, synthetic construct
  source 1..24: mol_type = other DNA
actttacccggtggtgcatatcgg

SEQ ID NO: 116 - DNA, 24 bp, synthetic construct
  source 1..24: mol_type = other DNA
ggccagtgtgccggtctccgttat

SEQ ID NO: 117 - DNA, 24 bp, synthetic construct
  source 1..24: mol_type = other DNA
agagtccgagcagaaagagaaggg

SEQ ID NO: 118 - DNA, 24 bp, synthetic construct
  source 1..24: mol_type = other DNA
atccccctggccagtgcacgtctg

SEQ ID NO: 119 - DNA, 24 bp, synthetic construct
  source 1..24: mol_type = other DNA
gagcgtgatattaccctgttatcc

SEQ ID NO: 120 - DNA, 24 bp, synthetic construct
  source 1..24: mol_type = other DNA
ggggatgaaagctggcgcatgatg

SEQ ID NO: 121 - DNA, 24 bp, synthetic construct
  source 1..24: mol_type = other DNA
cacgtctgctgtcagataaagtct

SEQ ID NO: 122 - DNA, 24 bp, synthetic construct
  source 1..24: mol_type = other DNA
ccctggccagtgcacgtctgctgt

SEQ ID NO: 123 - DNA, 24 bp, synthetic construct
  source 1..24: mol_type = other DNA
gatctcagccaccgcgaaaatgac

SEQ ID NO: 124 - DNA, 24 bp, synthetic construct
  source 1..24: mol_type = other DNA
tggccagtgtgccggtctccgtta

SEQ ID NO: 125 - DNA, 24 bp, synthetic construct
  source 1..24: mol_type = other DNA
agctttcatccccgatatgcacca

SEQ ID NO: 126 - DNA, 24 bp, synthetic construct
  source 1..24: mol_type = other DNA
aacggctctctcttttataggtgt

SEQ ID NO: 127 - DNA, 24 bp, synthetic construct
  source 1..24: mol_type = other DNA
tgcacgtctgctgtcagataaagt

SEQ ID NO: 128 - DNA, 24 bp, synthetic construct
  source 1..24: mol_type = other DNA
agtctcccgtgaactttacccggt

SEQ ID NO: 129 - DNA, 24 bp, synthetic construct
  source 1..24: mol_type = other DNA
tggtgcatatcggggatgaaagct

SEQ ID NO: 130 - protein, 7 aa, unidentified
PKKKRKV

SEQ ID NO: 131 - protein, 16 aa, unidentified
KRPAATKKAGQAKKKK

SEQ ID NO: 132 - protein, 9 aa, unidentified
PAAKRVKLD

SEQ ID NO: 133 - protein, 11 aa, unidentified
RQRRNELKRSP

SEQ ID NO: 134 - protein, 38 aa, unidentified
NQSSNFGPMKGGNFGGRSSGPYGGGGQYFAKPRNQGGY

SEQ ID NO: 135 - protein, 42 aa, unidentified
RMRIZFKNKGKDTAELRRRRVEVSVELRKAKKDEQILKRRNV

SEQ ID NO: 136 - protein, 8 aa, unidentified
VSRKRPRP

SEQ ID NO: 137 - protein, 8 aa, unidentified
PPKKARED

SEQ ID NO: 138 - protein, 8 aa, Homo sapiens
PQPKKKPL

SEQ ID NO: 139 - protein, 12 aa, Mus musculus
SALIKKKKKMAP

SEQ ID NO: 140 - protein, 5 aa, Influenza virus
DRLRR

SEQ ID NO: 141 - protein, 7 aa, Influenza virus
PKQKKRK

SEQ ID NO: 142 - protein, 10 aa, Hepatitus virus
RKLKKKIKKL

SEQ ID NO: 143 - protein, 10 aa, Mus musculus
REKKKFLKRR

SEQ ID NO: 144 - protein, 20 aa, Homo sapiens
KRKGDEVDGVDEVAKKKSKK

SEQ ID NO: 145 - protein, 17 aa, Homo sapiens
RKCLQAGMNLEARKTKK

SEQ ID NO: 146 - protein, 11 aa, Human immunodeficiency virus
YGRKKRRQRRR

SEQ ID NO: 147 - protein, 12 aa, unidentified
RRQRRTSKLMKR

SEQ ID NO: 148 - protein, 27 aa, unidentified
GWTLNSAGYLLGKINLKALAALAKKIL

SEQ ID NO: 149 - protein, 33 aa, unidentified
KALAWEAKLAKALAKALAKHLAKALAKALKCEA

SEQ ID NO: 150 - protein, 16 aa, unidentified
RQIKIWFQNRRMKWKK

SEQ ID NO: 151 - protein, 9 aa, unidentified
RKKRRQRRR

SEQ ID NO: 152 - protein, 8 aa, unidentified
RKKRRQRR

SEQ ID NO: 153 - protein, 11 aa, unidentified
YARAAARQARA

SEQ ID NO: 154 - protein, 11 aa, unidentified
THRLPRRRRRR

SEQ ID NO: 155 - protein, 11 aa, unidentified
GGRRARRRRRR

SEQ ID NO: 156 - protein, 5 aa, synthetic construct
  REGION >5: note = The entire sequence of amino acids 1-5 can be repeated one or more times
GSGGS

SEQ ID NO: 157 - protein, 6 aa, synthetic construct
  REGION >6: note = The entire sequence of amino acids 1-6 can be repeated one or more times
GGSGGS

SEQ ID NO: 158 - protein, 5 aa, synthetic construct
  REGION >5: note = The entire sequence of amino acids 1-5 can be repeated one or more times
GGGGS

SEQ ID NO: 159 - protein, 4 aa, synthetic construct
GGSG

SEQ ID NO: 160 - protein, 5 aa, synthetic construct
GGSGG

SEQ ID NO: 161 - protein, 5 aa, synthetic construct
GSGSG

SEQ ID NO: 162 - protein, 5 aa, synthetic construct
GSGGG

SEQ ID NO: 163 - protein, 5 aa, synthetic construct
GGGSG

SEQ ID NO: 164 - protein, 5 aa, synthetic construct
GSSSG

SEQ ID NO: 165 - protein, 1081 aa, Geobacillus thermodenitrificans
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRP

SEQ ID NO: 166 - protein, 1082 aa, Geobacillus sp.
MKYKIGLDIGITSIGWAVINLDIPRIEDLGVRIFDRAENPKTGESLALPRRLARSARRRLRRRKHRLERIRRLFIREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNSFSLRSIGSRTLKRFEKYQVDVLGNIYKVRGEKRVGVASSSHSKAGETIRPL

SEQ ID NO: 167 - protein, 1087 aa, Geobacillus sp.
MRYKIGLDIGITSVGWAVINLDIPRIEDLGVRIFDRAENPQTGESLALPRRLARSARRRLRRRKHRLERIRRLLIREGVLTKEELDKLFEEKHEIGVWQLRVEALDRKLNNDELARVLLHLAKRRGFKSNRKSERSNKENSTMLKHIEENRVILSGYRTVGEMIVKDPKFTLHKRNKGENYTNTIARDDLEREIRLIFSKQREFGNMSCTEEFENEYTALWASQRPFASKDDIEKKVGLCIFEPKEKRAPKATYTFQSFIAWEHINKLRLVSPSGARGLTDEERHLLYKQAFLKNKITYHDIRTLLNLPDDTYFKGIVYDRNMPLKQNETIRFLELDAYHQIRKAIDKVYGKGKSSSFLPIDFDTFGYALTLFKDDADIRSYLRNEYEQNGKRMPNLADKAYDNELIEELLNLSFTKFGHLSLKALRNILPYMEQGEVYSSACEQAGYTFTGPKKKQKTALLPTIPPIANPVVMRALTQARKVVNAIIKKYGSPVSIHIELARDLSQTFDERRKTKREQDENRKKNETAIRQLIEYGLTLNPTGLDIVKFKLWSEQNGRCAYSLQPIEIERLLEPGYTEVDHVIPYSRSLDDSYTNKVLVLTKENREKGNRIPAEYLGVGTERWQQFETFVLTNKQFSKKKRDRLLRLHYDESEETEFKNRNLNDTRYISRFFANFIREHLKFADSDNKQKVYTVNGRITAHLRSRWDFNKNREESDLHHAVDAAIVACTTPSDIARVTAFYQRREQNKELAQKTEPQFPQPWPHFADELRARLSKHPQESIKALNLGNYDNEKLESLQPVFVSRMPKRSVTGAAHQETLRRYIGIDERSGKIQTVVKTKLSEIKLDASGHFPMYGKESDPRTYEAIRQRLLEHNNDPKKAFQKPLYKPKKNGEPGPIIRTVKVIDTKNQVIPLNDGRTVAYNSNIIRVDVFEKDGKYYCVPIYTMDMMKGILPNKAIEPNKPYSEWKEMTEDYTFRFSLYPNDLIRIELPREKTVKTAAGEEINIKDMFVYYKTIDSANGGLELISHDNSFSLRGIGSRTLKRFEKYQVDVLGNIYKVRGEKRVGLASSSHPKAGETIRPLQSTRD

SEQ ID NO: 168 - protein, 1087 aa, Geobacillus sp.
MRYKIGLDIGITSVGWAVINLDIPRIEDLGVRIFDRAENPQTGESLALPRRLARSARRRLRRRKHRLERIRRLIIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYIAIWASQRPVASKDDIEKKVGFCTFEPKEKRAPKATYTFQSFIAWEHINKLRLISPSGTRGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIRSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYTEVDHVIPYSRSLDDSYTNKVLVLTKENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKIIKTAGGEEIKIKDLFAYYKTIHSGTAGLELVSHDCSFSLSGVGSRTLKRFEKYQVDVLGNIYKVRGEKRVGLASSAHSKTGETIRPLQSTRD

SEQ ID NO: 169 - protein, 1087 aa, Geobacillus jurassicus
MRHKIGLDIGITSVGWAVINLDIPRIEDLGVRIFDRAENPQTGESLALPRRLARSARRRLRRRKHRLERIRRLLIREGILTKEELDKLFEEKHEIDVWQLRVEALDRKLNNDELARVLLHLAKRRGFKSNRKSERNNKENSTMLKHIEENRAILSGYRTVAEMIVKDPKFAFHKRNKGENYTNTIARDDLEREIKLIFTKQREYGNIVCTETFENEYIMIWASQRPFASKNDIEKKVGFCTFEPKEKRAPKATYTFQSFVAWEHINKLRLVSPTGTRGLTDEERRLLYEQAFHKNKITYHDIRTLLHLPDDTYFKGIVYDRGAPLKQSETIRFLELDAYHQIRKAVDKVYGKGKSSSFLPIDFDTFGYALTLFKDDADIRSYLRNEYEQNGKRMPNLANKVYDNELIEELLNLSFTKFGHLSLKALRNILPYMEQGEVYSVACERAGYTFTGPKKKQKTLLLPNIPPIANPVVMRALTQARKVVNAIIKKYGSPVSIHIELARDLSQTFDERRKTKREQDENRKKNETAIRQLMEYGLTINPTGHDIVKFKLWSEQNGRCAYSLQPIEIERLLEPGYVEVDHVIPYSRSLDDSYTNKVLVLTKENREKGNRIPAEYLGVGTERWQQFKTFVLTNKQFSKKKRDRLLRLHYDENEEAEWKNRNLNDTRYISRFFANFIREHLKFAESDDKQKVYTVNGRVTAHLRSRWDFNKNREESDLHHAVDAAIVACTSPSDIARVTAFYQRREQCKESAKKAEPHFPQPWPHFADELRARLSKNPKESIKALNLGNYDDQKLESLQPVFVSRMPKRSVTGAAHQETLRRYIGIDERSGKIQTVVKTKLSEIKLDETGHFPMYGKESDPRTYEAIRQRLLEHNNDPKKAFQEPLYKPKKNGEPGPIIRTVKIIDTKNQVIPLNDGKTVAYNSNIVRVDVFEKDGKYYCVPVYTMDIMKGVLPNKSIEPNKPYSEWKEMTEDYTFRFSLYPNDLIRIELPREKTIKTAGGEEIKIKDLFAYYKTIHSGTAGLELVSHDCSFSLSGVGSRTLKRFEKYQVDVLGNIYKVRGEKRVGLASSAHSKTGETVRPLQSTRD

SEQ ID NO: 170 - protein, 1087 aa, Geobacillus sp.
MRYKIGLDIGITSVGWAVINLDIPRIEDLGVRIFDRAENPQTGESLALPRRLARSARRRLRRRKHRLERIRRLLIREGVLTKEELDKLFEEKHEIDVWQLRVEALDRKLNNDELVRVLLHLAKRRGFKSNRQSERSNKENSTMLKHIEENRAILSGYRTVGEMIVKDPKFALHKRNKGENYTNTIARDDLEREIRLIFSKQREFGNMSCTEEFENEYIAIWASQRPVASKDDIEKKVGFCTFEPKEKRAPKATYTFQSFIAWEHINKLRLISPSGTRGLTDEERRLLYEQAFQKNKITYHDIRTLLHLPDDTYFKGIVYDWGESLKQNENIRFLELDAYHQIRKAIDKVYGKGKSSSFLPIDFDTFGYALTLFKDDADIRSYLRNEYEQNGKRIANLANKVYDNELIEELLNLSFTKFGHLSLKALRNILPYMEQGEVYSSACEKAGYTFTGPKKKQKTVLLPTIPPIVNPVVMRALTQARKVVNAIIKKYGSPVSIHIELARDLSQTFDERRKTKREQDENRKRNETAIRQLMEYGLTLNPTGLDIVKFKLWSEQNGRCAYSLQPIEIERLLEPGYVEVDHVIPYSRSLDDSYTNKVLVLTKENREKGNRIPAEYLGVGTERWQQFETFVLTNKQFSKKKRDRLLRLHYDENEEMEFKSRNLNDTRYISRFFANFIREHLKFAESDDKQKVYTVNGRVTSHLRSRWDFNKNREESDLHHAVDAAIVACTTPSDIAKVTAFYQRREQNKELAKKTEPQFPQPWPHFADELRARLSKHPKESIKALNLGNYDDQKLESLQPVFVSRMPKRSVTGAAHQETLRRYIGIDERSGKIQTVVKTKLSEIKLDASGHFPMYGKESDPRTYEAIRQRLLEHNNDPKKAFQEPLYKPKKNGEPGPIIRTVKIIDTKNQVIPLNDGKTVAYNSNIVRIDVFEKDGKYYCVPVYTMDIMKGVLPNKAIEPNKPYSEWKEMTEDYTFRFSLYPNDLIRIELPREKTVKTAAGEEINIKDMFVYYKTIDSANGGLELISHDNSFSLRGVGSRTLKRFEKYQVDVLGNIYKVRGEKRVGLASSAHSKTGETIRPLQSTRD

SEQ ID NO: 171 - protein, 1087 aa, Geobacillus stearothermophilus
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYIAIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLQLHYDENEETEFKNRNLNDTRYISRFFANFIREHLKFAESDDKQKVYTVNGRVTAHLRSRWEFNKNREESDLHHAVDAVIVACTTPSDIAKITAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSATGGLELISHDHSFSLRGVGSRTLKRFEKYQVDVLGNIYKVRGEKRVGLASSAHSKTGETIRPLQSTRD

SEQ ID NO: 172 - protein, 1087 aa, Geobacillus stearothermophilus
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYIAIWASQRPVASKDDIEKKVGFCAFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKEKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRSAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 173 - protein, 1086 aa, Geobacillus stearothermophilus
MRYKIGLDIGITSVGWAVMNLDIPRIEDLGVRIFERAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYIAIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKREQDENRKRNETAIRQLMEYGLTLNPTGLDIVKFKLWSEQNGRCAYSLQPIEIERLLEPGYVEVDHVIPYSRSLDDSYTNKVLVLTKENREKGNRIPAEYLGVGTERWQQFETFVLTNKQFSKKKRDRLLRLHYDENEETEFKNRNLNDTRYISRFFANFIREHLKFAESDDKQKVYTVNGRVTSHLRSRWDFNKNREESDLHHAVDAAIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKIIKTAGGEEIKIKDLFAYYKTIHSGTAGLELVSHDCSFSLSGVGSRTLKRFEKYQVDVLGNIYKVRGEKRVGLASSAHSKTGETIRPVTINS

SEQ ID NO: 174 - protein, 1087 aa, Geobacillus stearothermophilus
MRYKIGLDIGITSVGWAVINLDIPRIEDLGVRIFERAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSGYRTVGEMIVKDPKFALHKRNKGENYTNTIARDDLEHEIRLIFSKQREFGNMSCTEKFENEYITIWASQRPVASKDDIEKKVGFCTFEPKEKRAPKATYTFQSFIAWEHINKLRLISPSGARGLTDEERRLLYEQAFQKNKITYHDIRTLLHLPDDIYFKGIVYDWGESLKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLQLHYDENEETEFKNRNLNDTRYISRFFANFIREHLKFAESDDKQKVYTVNGRVTAHLRSRWEFNKNREESDLHHAVDAVIVACTTPSDIAKITAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPIIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTTTGEEINVKDVFVYYKTIDSATGGLELISHDNRFSLRGVGSRTLKRFEKYQVDVLGNIYKVRGEKRVGLASSAHSKTGETIRPLQSTRD

SEQ ID NO: 175 - protein, 1087 aa, Geobacillus sp.
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKKEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 176 - protein, 1082 aa, synthetic construct
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEGNQSILSSYRTVAEMVVKDPKFSLHKRNKEDNYINTVARDDLEREIKLIFAKQREYGDIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIQPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHRETLRRYIGIDERSGKIQTVVKKKLSKIQLDKTGHFPMYGKESDPRTYEAIRQRLLEHNNDPKKAFQGPLYKPKKNGELGPIIRTIKIIDTRNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

(19) United States
(12) Patent Application Publication
(10) Pub. No.: US 2026/0185070 A1
(43) Pub. Date: Jul. 2, 2026

(54) CRISPR-Cas Effector Polypeptides, Lipid Nanoparticles, and Methods of Use Thereof

(71) Applicant: The Regents of the University of California, Oakland, CA (US)

(72) Inventors: Jennifer A. Doudna, Berkeley, CA (US); Niren Murthy, El Cerrito, CA (US); Kai Chen, Berkeley, CA (US); Hesong Han, Berkeley, CA (US); Sheng Zhao, El Cerrito, CA (US)

(21) Appl. No.: 19/130,012

(22) PCT Filed: Oct. 25, 2023
(86) PCT No.: PCT/US2023/077805

§ 371 Date: May 14, 2025

Related U.S. Application Data
(60) Provisional application No. 63/427,276, filed on Nov. 22, 2022.

(51) Int. Cl. C12N 9/22; C12N 15/11; C12N 15/88; C12N 15/90

(52) U.S. Cl. CPC C12N 9/226; C12N 15/11; C12N 15/88; C12N 15/907; C12N 2310/20

(57) ABSTRACT

The present disclosure provides variant CRISPR-Cas effector polypeptides, nucleic acids encoding same, and compositions comprising the variant CRISPR-Cas effector polypeptides. The present disclosure provides methods of modifying a target nucleic acid, using a variant CRISPR-Cas effector polypeptide of the present disclosure. The present disclosure provides lipid nanoparticles comprising a CRISPR-Cas effector polypeptide and a guide nucleic acid.

### CROSS-REFERENCE

This application claims the benefit of U.S. Provisional Patent Application No. 63/427,276 filed Nov. 22, 2022, which application is incorporated herein by reference in its entirety.

### INCORPORATION BY REFERENCE OF SEQUENCE LISTING PROVIDED AS AN XML FILE

A Sequence Listing is provided herewith as a Sequence Listing XML, “BERK-479WO_SEQ_LIST” created on Oct. 20, 2023 and having a size of 213,287 bytes. The contents of the Sequence Listing XML are incorporated by reference herein in their entirety.

### INTRODUCTION

Clustered Regularly Interspaced Short Palindromic Repeats (CRISPR)-Cas systems comprise a CRISPR-associated (Cas) effector polypeptide and a guide nucleic acid. Such CRISPR-Cas systems can bind to and modify a targeted nucleic acid. The programmable nature of these CRISPR-Cas effector systems has facilitated their use as a versatile technology for use in, e.g., gene editing.

Viral-based delivery of CRISPR genome editors is currently the most effective method for editing cells in vivo. However, viral-based delivery of genome editors is also problematic because of their immunogenicity, the risk of genome integration of the viral vector, and the risk of off-target DNA damage caused by the continuous expression of virally delivered genome editors. Non-viral strategies for delivering CRISPR editors have the potential to address the limitations of viral-based delivery.

Lipid-nanoparticle (LNP)/mRNA complexes are currently used for delivering genome editors in vivo and have been remarkably successful at genome editing in the liver. However, developing LNP/mRNA complexes that can efficiently edit non-liver tissues remains a challenge. Achieving highly efficient LNP-mediated delivery of CRISPR mRNAs and guide RNAs is challenging because of the metabolic instability of the sgRNA, the activation of Toll-like receptors (TLRs) by mRNA, and the low translational efficiency of the large mRNAs encoding genome editors. These challenges are inherent to the mRNA format, and consequently, alternatives to LNP/mRNA-mediated delivery of CRISPR enzymes are desirable.

Direct delivery of genome editors in the form of ribonucleoprotein (RNP) complexes has potential to address several of the limitations associated with mRNA and viral-based delivery of CRISPR enzymes. In particular, RNPs should elicit lower levels of TLR activation than mRNA and have lower levels of off-target DNA damage due to their shorter intracellular half-life. In addition, RNPs have the potential to edit cells in vivo with higher efficiency than mRNA-based delivery methods because the sgRNA is stabilized by complexation with the Cas protein and it avoids the problems associated with translating large mRNAs.

Methods and compositions for more efficient CRISPR-based DNA targeting are needed, and such methods and compositions are provided herein.

### SUMMARY

The present disclosure provides variant CRISPR-Cas effector polypeptides, nucleic acids encoding same, and compositions comprising the variant CRISPR-Cas effector polypeptides. The present disclosure provides methods of modifying a target nucleic acid, using a variant CRISPR-Cas effector polypeptide of the present disclosure. The present disclosure provides lipid nanoparticles comprising a CRISPR-Cas effector polypeptide and a guide nucleic acid.

### BRIEF DESCRIPTION OF THE DRAWINGS

FIG. 1 provides the amino acid sequence of a Geobacillus stearothermophilus CRISPR-Cas effector polypeptide, also referred to herein as “GeoCas9.” (SEQ ID NO: 1)

FIG. 2-23 provide amino acid sequences of variant CRISPR-Cas effector polypeptides. (SEQ ID NOs: 2-23, respectively).

FIG. 24 provides examples of combinations of amino acid substitutions.

FIG. 25A-25L provide amino acid sequences of various CRISPR-Cas effector polypeptides. (SEQ ID NOs.: 24-35, respectively)

FIG. 26 provides the amino acid sequence of a reverse transcriptase. (SEQ ID NO: 36)

FIG. 27 provides a schematic depiction of the domains of a GeoCas9 polypeptide. I, II, and III are the split RuvC domains.

FIG. 28A-28C depict the effect of direct delivery of ribonucleoprotein (RNP) (100 μmol) on gene editing in neural progenitor cells (NPCs), 48 hours after direct delivery of the RNP, where the RNP comprises Streptococcus pyogenes Cas9 (SpyCas9), GeoCas9, or no CRISPR-Cas effector polypeptide (negative control).

FIG. 29 schematically depicts the screening method used to generate variants of GeoCas9 using directed evolution.

FIG. 30 schematically depicts the iterative approach to generating variants of GeoCas9 using directed evolution.

FIG. 31 depicts the editing scope test used to assess GeoCas9 variants (g1-g23 spacers SEQ ID NO:54-76, respectively; corresponding PAM sequences in parentheses).

FIG. 32A-32C depict the results of the editing scope test, using various protospacer adjacent motif (PAM) sequences.

FIG. 33A-33C depict the results of genome editing, using GeoCas9 variants, in NPCs. FIG. 33B presents g7, g8, g16 and g17 spacers which are SEQ ID NOs:60-61, 69-70, respectively. The respective PAM sequences are in parentheses. The figures depict the editing scope test used to assess further engineered GeoCas9 variants and the results of the editing scope test, using various protospacer adjacent motif (PAM) sequences.

FIG. 34A-34D depict a comparison of nucleofection, direct delivery, and lipid nanoparticle (LNP) delivery of SpyCas9, LbCpf1, and GeoCas9(R1-W1) on gene editing in NPCs.

FIG. 35A-35B depict LNPs for RNP delivery.

FIG. 36 depict the effect of LNPs of various lipid compositions on gene editing in NPCs. The LNPs comprised RNP comprising GeoCas9. The figure depicts the screening of different LNP formulations to deliver GeoCas9 RNP for genome editing in NPCs. The figure depicts the gene editing ability of GeoCas9 and SpyCas9 RNPs for GFP knockdown in HEK293 cells, using various protospacer and PAM sequences, when delivered using LNPs.

FIG. 37A-37B depict green fluorescent protein (GFP) knockdown by RNP comprising GeoCas9 variants, delivered using an LNP.

FIG. 38 depicts a system for assessing homology-directed repair (HDR) via co-delivery of an RNP and a single-stranded (ss) DNA template, in which the RNP is delivered in an LNP. PAM-spacer deactivation sequences: from left to right SEQ ID NOs:83-84. HDR-Donor1: SEQ ID NO:77. HDR-Donor2: SEQ ID NO:78. mEGFP DNA sequence: SEQ ID NO:79. mEGFP amino acid sequence: SEQ ID NO:85.

FIG. 39 depicts a system for assessing HDR via co-delivery of an RNP and a ssDNA template, in which the RNP is delivered in an LNP. ssDNA-template1: SEQ ID NO:80. ssDNA-template2: SEQ ID NO:81. mEGFP DNA sequence: SEQ ID NO:82. mEGFP amino acid sequence: SEQ ID NO:86.

FIG. 40A-40F. GeoCas9 engineering for improved editing efficiency and broadened PAM compatibility. FIG. 40A. Schematic of the direct evolution system based on bacterial selection to engineer GeoCas9. FIG. 40B. Evolutionary lineage of GeoCas9 mutants. FIG. 40C. Melting temperature measurement of the WT and engineered GeoCas9 proteins. FIG. 40D. Schematic of GeoCas9-mediated genome editing of Ai9 tdTomato NPCs to turn on fluorescent signals, and spacer and PAM sequences chosen for GeoCas9 (g1-g22 spacers SEQ ID NOs:54-59, 62-68, 71-73, 76, 60-61, 70, 69, 74, respectively). FIG. 40E. Genome editing efficiencies quantified based on tdTom⁺ signals with the whole lineage of GeoCas9 mutants paired with different sgRNAs. FIG. 40F. GeoCa9 engineering with altered PAM specificity. n=4 for each group, mean±s.e.m

FIG. 41A-41B. Using lipid nanoparticles to encapsulate and deliver GeoCas9 RNP. FIG. 41A. Comparison of three different genome editors for Ai9 NPC editing using two delivery methods based on nucleofection and LNPs. n=4 for each group, mean±s.e.m. FIG. 41B. Schematic of lipid structures used in this study, and two formulations optimized for GeoCas9 RNP delivery. Dynamic light scattering (DLS) assay used for LNP particle size measurement.

FIG. 42A-42C. LNP strategy to deliver GeoCas9 RNP for genome editing in different cell lines. FIG. 42A. Comparison of the genome editing levels in Ai9 NPCs based on nucleofection and LNP-assisted delivery of GeoCas9 RNP. FIG. 42B. sgRNA engineering for improved LNP delivery. FIG. 42C. Comparison of the genome editing levels in HEK293T cells based on nucleofection and LNP-assisted delivery of GeoCas9 RNP (spacers from top to bottom SEQ ID NOs:87-92). (Left) Schematic of GeoCas9-mediated genome editing to knock down EGFP in HEK293 T cell. (Right) Genome editing efficiencies quantified based on EGFP(−) signals using the engineered GeoCas9 paired with different sgRNAs. n=4 for each group, mean±s.e.m.

FIG. 43A-43B. Co-delivery of GeoCas9 RNPs and ssDNA templates for HDR. FIG. 43A. Characterization of lipid nanoparticles encapsulating GeoCas9 RNPs and ssDNA templates. FIG. 43B. Co-delivery of GeoCas9 RNPs and ssDNA HDR templates to edit the chromophore of EGFP to BFP in HEK293T cells (from top to bottom SEQ ID NOs:93-101). (Upper) Target and donor designs for GeoCas9-mediated chromophore editing. (Lower) Genome editing efficiencies quantified based on EGFP/BFP signals using the engineered GeoCas9 (in the form of NLS-GeoCas9(RJW 1)-2NLS) paired with different sgRNAs±ssDNA templates. n=4 for each group, mean±s.e.m.

FIG. 44A-44B. LNP-based delivery of RNPs and ssDNA templates for HDR targeting endogenous sites. FIG. 44A. Genome editing efficiencies (indels and HDR) by the engineered GeoCas9 paired with different sgRNAs±ssDNA templates, as quantified by NGS. FIG. 44B. Editing of pathogenic mutations in the CFTR gene through HDR (from top to bottom SEQ ID NOs:102-109). (Left) Target and donor designs for GeoCas9-mediated editing of pathogenic mutations. (Right) Genome editing efficiencies quantified by NGS. n=4 for each group, mean±s.e.m.

FIG. 45A-45D. In vivo genome editing by LNP-based delivery of GeoCas9 RNP. FIG. 45A. Schematic of the procedure for in vivo genome editing with Ai9 mouse models. FIG. 45B. LNP formulations used for in vivo genome editing. FIG. 45C. In vivo genome editing levels in different tissues based on GeoCas9 RNP delivery by different LNP formulations, as quantified by tdTomato signals using flow cytometer. n=6 for each group, mean±s.e.m. FIG. 45D. Nuclei staining with DAPI (blue) and imaging of tdTomato (red) in the edited and non-edited tissues.

FIG. 46A-46D. Directed evolution of GeoCas9. FIG. 46A. Modelled GeoCas9 structure with mutations highlighted. FIG. 46B. Two rounds of selection to identify improved GcoCas9 mutants. FIG. 46C. Mutants and beneficial mutations identified in each round of selection. FIG. 46D. Target cleavage activities of WT-GeoCas9 and R1W1 mutant in the bacterial assay using different spacer (Nos. 1-20 SEQ ID NOs:110-129, respectively) and PAM sequences, as reflected by the bacterial survival rates.

FIG. 47A-47C. FIG. 47A. RNP stability comparison of WT-GeoCas9, GeoCas9(R1W1), and SpyCas9 using DLS assay. FIG. 47B. Comparison of WT-GeoCas9 and GeoCas9(R1W1) for their genome editing activities in HEK293T cells to knock down EGFP using different spacer (from top to bottom SEQ ID NOs:87-92) and PAM sequences. FIG. 47C. Mutant ThermoCas9 with improved genome editing activities through mutation transfer from GeoCas9 to ThermoCas9. n=4 for each group, mean±s.e.m.

FIG. 48A-48C. Optimization of LNP formulation for GeoCas9 RNP delivery. FIG. 48A. Comparison of three different genome editors for Ai9 NPC editing based on RNP delivery by LNPs. FIG. 48B. Optimization of the percentage of pegylated lipid ADP-2k in LNP formulations. FIG. 48C. Comparison of different pegylated lipids for their GeoCas9 RNP delivery efficiency and cytotoxicity with NPCs. n=4 for each group, mean±s.e.m.

FIG. 49A-49B. FIG. 49A. pH-sensitive acetyl linker used in synthetic lipid design. FIG. 49B. Endocytosis pathway in LNP-based delivery promoted by the pH-sensitive acetyl linker in the lipids.

FIG. 50A-50B. FIG. 50A. Schematic of the whole procedure for LNP-based RNP delivery in cell culture. FIG. 50b. Effect of volume ratio (aqueous/organic) and salt concentration on the packaging efficiency of GeoCas9 RNP in LNP. n=4 for each group, mean±s.e.m.

FIG. 51. Co-delivery of GeoCas9 RNPs and ssDNA HDR templates to edit the chromophore of EGFP to BFP in HEK293T cells.

FIG. 52. Effect of different anionic polymer additives on the packaging efficiency of RNPs in LNPs.

FIG. 53. Sequence alignment of GeoCas9 (SEQ ID NO: 1) with ThermoCas9 (SEQ ID NO: 165).

FIG. 54. Sequence of ThermoCas9 (SEQ ID NO: 165) and ThermoCas9 (R1W1) (SEQ ID NO: 176).

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Temperature, wash solution salt concentration, and other conditions may be adjusted as necessary according to factors such as length of the region of complementation and the degree of complementation.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a variant GeoCas protein/guide RNA complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{D}) of less than 10⁻⁶ M. less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{D}.

By “binding domain” it is meant a protein domain that is able to bind non-covalently to another molecule. A binding domain can bind to, for example, a DNA molecule (a DNA-binding domain), an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

Polypeptides as described herein also include polypeptides having various amino acid additions, deletions, or substitutions relative to the amino acid sequence of a polypeptide of the present disclosure. In some embodiments, polypeptides that are homologs of a polypeptide of the present disclosure contain non-conservative changes of certain amino acids relative to the native sequence of a polypeptide of the present disclosure. In some embodiments, polypeptides that are homologs of a polypeptide of the present disclosure contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D). Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A modification of an amino acid to produce a chemically similar amino acid may be referred to as an analogous amino acid.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Biol. 215:403-10.

A DNA sequence that “encodes” a particular RNA is a DNA nucleotide sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.).

A “protein coding sequence” or a sequence that encodes a particular protein or polypeptide, is a nucleotide sequence that is transcribed into mRNA (in the case of DNA) and is translated (in the case of mRNA) into a polypeptide in vitro or in vivo when placed under the control of appropriate regulatory sequences.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., variant CRISPR-Cas effector polypeptide fusion polypeptide, and the like) and/or regulate translation of an encoded polypeptide.

As used herein, a “promoter” or a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase.

“Heterologous,” as used herein, means a nucleotide sequence or an amino acid sequence that is not found in the native nucleic acid or protein, respectively. For example, relative to a subject variant CRISPR-Cas effector polypeptide, a heterologous polypeptide comprises an amino acid sequence from a protein other than the variant CRISPR-Cas effector polypeptide. As another example, a variant CRISPR-Cas effector polypeptide can be fused to a polypeptide (also referred to as a “fusion partner”) other than the variant CRISPR-Cas effector polypeptide; the sequence of the fusion partner can be considered a heterologous polypeptide (it is heterologous to the variant CRISPR-Cas effector polypeptide). As another example, in a guide nucleic acid, a heterologous guide nucleotide sequence (present in a targeting segment) that can hybridize with a target nucleotide sequence (target region) of a target nucleic acid is a nucleotide sequence that is not found in nature in a guide nucleic acid together with a binding segment that can bind to a variant CRISPR-Cas effector polypeptide of the present disclosure. For example, in some cases, a heterologous target nucleotide sequence (present in a heterologous targeting segment) is from a different source than a binding nucleotide sequence (present in a binding segment) that can bind to a variant CRISPR-Cas effector polypeptide of the present disclosure. For example, a guide nucleic acid may comprise a guide nucleotide sequence (present in a targeting segment) that can hybridize with a target nucleotide sequence present in a eukaryotic target nucleic acid. A guide nucleic acid of the present disclosure can be generated by human intervention and can comprise a nucleotide sequence not found in a naturally-occurring guide nucleic acid.

The term “naturally-occurring” as used herein as applied to a nucleic acid, a protein, a cell, or an organism, refers to a nucleic acid, cell, protein, or organism that is found in nature.

Thus, the term “recombinant” polypeptide does not necessarily refer to a polypeptide whose amino acid sequence does not naturally occur. Instead, a “recombinant” polypeptide is encoded by a recombinant non-naturally occurring DNA sequence, but the amino acid sequence of the polypeptide can be naturally occurring (“wild type”) or non-naturally occurring (e.g., a variant, a mutant, etc.). Thus, a “recombinant” polypeptide is the result of human intervention, but may have a naturally occurring amino acid sequence.

A “vector” or “expression vector” is a replicon, such as plasmid, phage, virus, artificial chromosome, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence (or the coding sequence can also be said to be operably linked to the promoter) if the promoter affects its transcription or expression.

The terms “recombinant expression vector,” or “DNA construct” are used interchangeably herein to refer to a DNA molecule comprising a vector and an insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

A cell has been “genetically modified” or “transformed” or “transfected” by exogenous DNA or exogenous RNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023), and the like.

The choice of method of genetic modification is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

A “target nucleic acid” as used herein is a polynucleotide (e.g., DNA such as genomic DNA) that includes a site (“target site” or “target sequence”) targeted by a variant CRISPR-Cas polypeptide of the present disclosure. The target sequence is the sequence to which the guide sequence of a guide RNA (e.g., a dual guide RNA or a single-molecule guide RNA) will hybridize. For example, the target site (or target sequence) 5′-GAGCAUAUC-3′ within a target nucleic acid is targeted by (or is bound by, or hybridizes with, or is complementary to) the sequence 5′-GAUAUGCUC-3′. Suitable hybridization conditions include physiological conditions normally present in a cell. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand.”

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events.

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

By “cleavage domain” or “active domain” or “nuclease domain” of a nuclease it is meant the polypeptide sequence or domain within the nuclease which possesses the catalytic activity for nucleic acid cleavage. A cleavage domain can be contained in a single polypeptide chain or cleavage activity can result from the association of two (or more) polypeptides. A single nuclease domain may consist of more than one isolated stretch of amino acids within a given polypeptide.

The term “stem cell” is used herein to refer to a cell (e.g., plant stem cell, vertebrate stem cell) that has the ability both to self-renew and to generate a differentiated cell type (see Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and may or may not retain the capacity to proliferate further. Stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to multiple differentiated progeny.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282(5391):1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be the target cells of the methods described herein.

By “embryonic stem cell” (ESC) is meant a PSC that was isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells may be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. Nos. 7,029,913, 5,843,780, and 6,200,806, the disclosures of which are incorporated herein by reference. Methods for proliferating hESCs in the undifferentiated form are described in WO 99/20741, WO 01/51616, and WO 03/020920.

By “embryonic germ stem cell” (EGSC) or “embryonic germ cell” or “EG cell” is meant a PSC that is derived from germ cells and/or germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e. ectoderm, mesoderm and endoderm. For example, somatic cells would include both neurons and neural progenitors, the latter of which may be able to naturally give rise to all or some cell types of the central nervous system but cannot give rise to cells of the mesoderm or endoderm lineages.

By “mitotic cell” it is meant a cell undergoing mitosis. Mitosis is the process by which a eukaryotic cell separates the chromosomes in its nucleus into two identical sets in two separate nuclei. It is generally followed immediately by cytokinesis, which divides the nuclei, cytoplasm, organelles and cell membrane into two cells containing roughly equal shares of these cellular components.

By “post-mitotic cell” it is meant a cell that has exited from mitosis, i.e., it is “quiescent”, i.e. it is no longer undergoing divisions. This quiescent state may be temporary, i.e. reversible, or it may be permanent.

By “meiotic cell” it is meant a cell that is undergoing meiosis. Meiosis is the process by which a cell divides its nuclear material for the purpose of producing gametes or spores. Unlike mitosis, in meiosis, the chromosomes undergo a recombination step which shuffles genetic material between chromosomes. Additionally, the outcome of meiosis is four (genetically unique) haploid cells, as compared with the two (genetically identical) diploid cells produced from mitosis.

In some instances, a component (e.g., a nucleic acid component (e.g., a guide RNA); a protein component (e.g., a variant CRISPR-Cas polypeptide); and the like) includes a label moiety. The terms “label”, “detectable label”, or “label moiety” as used herein refer to any moiety that provides for signal detection and may vary widely depending on the particular nature of the assay. Label moieties of interest include both directly detectable labels (direct labels; e.g., a fluorescent label) and indirectly detectable labels (indirect labels; e.g., a binding pair member). A fluorescent label can be any fluorescent label (e.g., a fluorescent dye (e.g., fluorescein, Texas red, rhodamine, ALEXAFLUOR® labels, and the like), a fluorescent protein (e.g., green fluorescent protein (GFP), enhanced GFP (EGFP), yellow fluorescent protein (YFP), red fluorescent protein (RFP), cyan fluorescent protein (CFP), cherry, tomato, tangerine, and any fluorescent derivative thereof), etc.). Suitable detectable (directly or indirectly) label moieties for use in the methods include any moiety that is detectable by spectroscopic, photochemical, biochemical, immunochemical, electrical, optical, chemical, or other means. For example, suitable indirect labels include biotin (a binding pair member), which can be bound by streptavidin (which can itself be directly or indirectly labeled). Labels can also include: a radiolabel (a direct label)(e.g., ³H, ¹²⁵I, ³⁵S, ¹⁴C, or ³²P) an enzyme (an indirect label)(e.g., peroxidase, alkaline phosphatase, galactosidase, luciferase, glucose oxidase, and the like); a fluorescent protein (a direct label)(e.g., green fluorescent protein, red fluorescent protein, yellow fluorescent protein, and any convenient derivatives thereof); a metal label (a direct label); a colorimetric label; a binding pair member; and the like. By “partner of a binding pair” or “binding pair member” is meant one of a first and a second moiety, wherein the first and the second moiety have a specific binding affinity for each other. Suitable binding pairs include, but are not limited to: antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Any binding pair member can be suitable for use as an indirectly detectable label moiety.

Any given component, or combination of components can be unlabeled, or can be detectably labeled with a label moiety. In some cases, when two or more components are labeled, they can be labeled with label moieties that are distinguishable from one another.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a CRISPR-Cas effector polypeptide” includes a plurality of such polypeptides and reference to “the guide nucleic acid” includes reference to one or more guide nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B. and C; A. B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides variant CRISPR-Cas effector polypeptides, nucleic acids encoding same, and compositions comprising the variant CRISPR-Cas effector polypeptides. The present disclosure provides methods of modifying a target nucleic acid, using a variant CRISPR-Cas effector polypeptide of the present disclosure. The present disclosure provides lipid nanoparticles comprising a CRISPR-Cas effector polypeptide and a guide nucleic acid.

### Variant Crispr-Cas Polypeptides

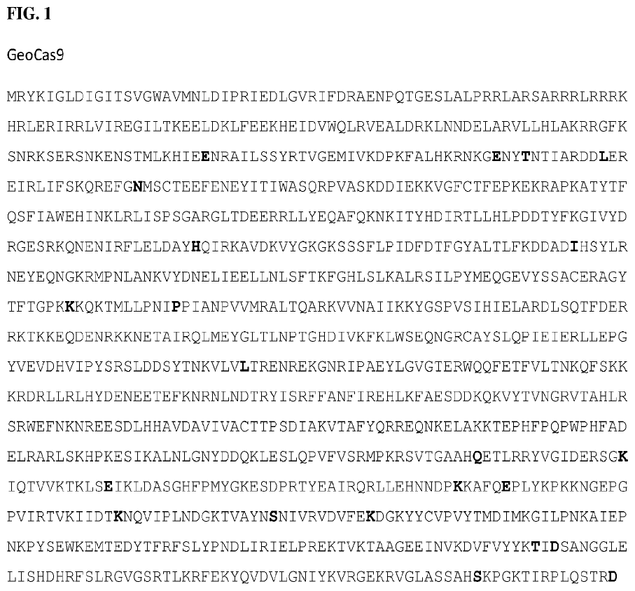

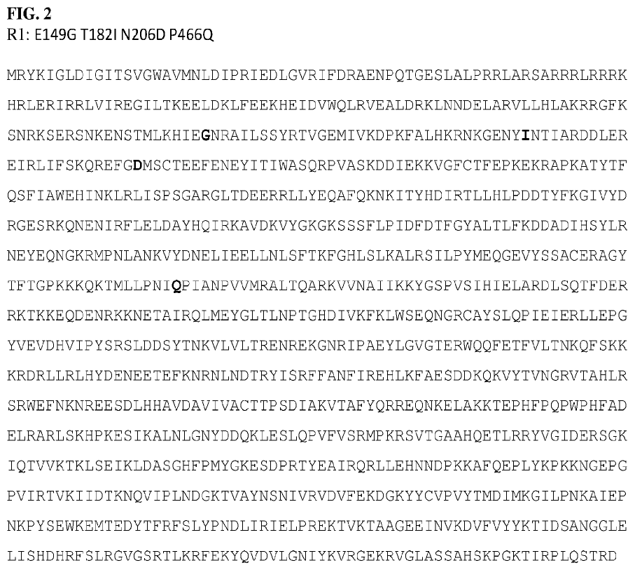

The present disclosure provides variant CRISPR-Cas polypeptides (also referred to herein as “GeoCas9 variants”). In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, or at least 99%, amino acid sequence identity to the amino acid sequence depicted in FIG. 1 (SEQ ID NO: 1), where the variant CRISPR-Cas polypeptide has at least one amino acid substitution compared to the amino acid sequence depicted in FIG. 1 (e.g., where the variant CRISPR-Cas polypeptide has: i) a single amino acid substitution; ii) from 1 to 4 amino acid substitutions; iii) 2 amino acid substitutions; iv) 5 amino acid substitutions; v) 6 amino acid substitutions; vi) 7 amino acid substitutions; vii) 8 amino acid substitutions; viii) 9 amino acid substitutions; ix) 10 amino acid substitutions; or x) from 10 amino acid substitutions to 14 amino acid substitutions). Examples of substitutions are depicted in FIG. 24.

In some cases, a variant CRISPR-Cas polypeptide of the present disclosure exhibits gene editing activity that is at least 10%, at least 15%, at least 20%, at least 25%, at least 50%, at least 75%, at least 100% (or 2-fold), at least 2.5-fold, at least 3-fold, at least 4-fold, at least 5-fold, at least 10-fold, or more than 10-fold, greater than the gene editing activity of a CRISPR-Cas polypeptide comprising the amino acid sequence depicted in FIG. 1 (SEQ ID NO: 1). Whether a variant CRISPR-Cas polypeptide exhibits greater gene activity than the gene editing activity of a CRISPR-Cas polypeptide comprising the amino acid sequence depicted in FIG. 1 can be determined using any of a variety of assays. One such method is a green fluorescent protein (GFP) to blue fluorescent protein (BFP) conversion assay, as described in Glaser et al. (2016) Mol. Ther. Nucl. Acids 5:e334.

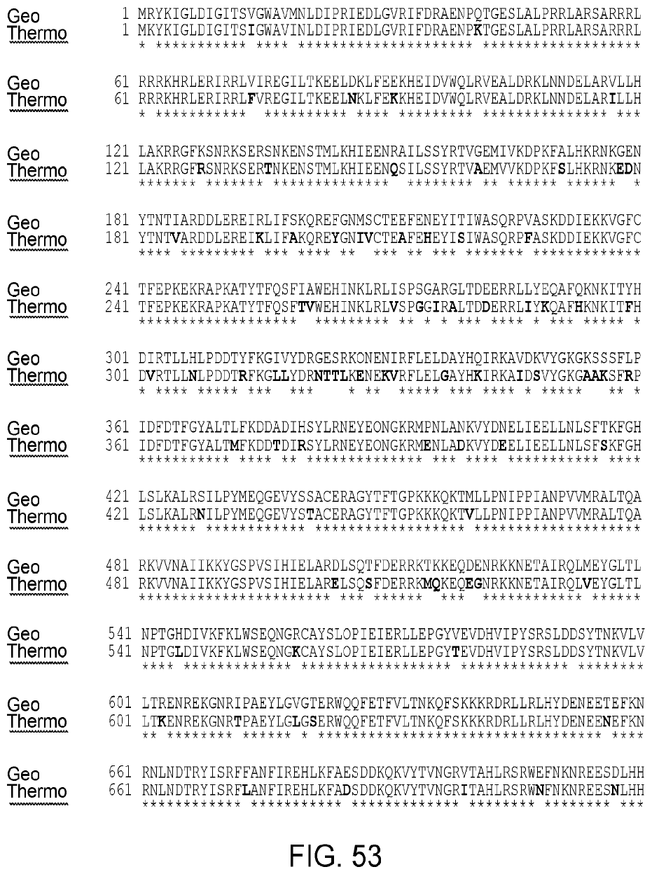

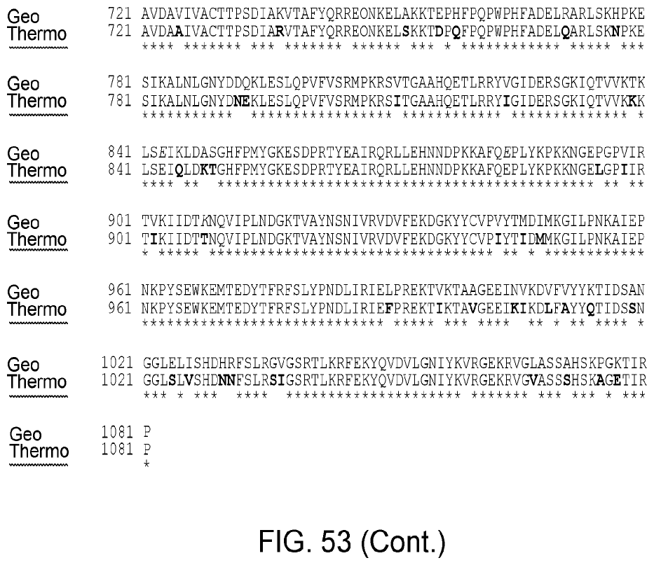

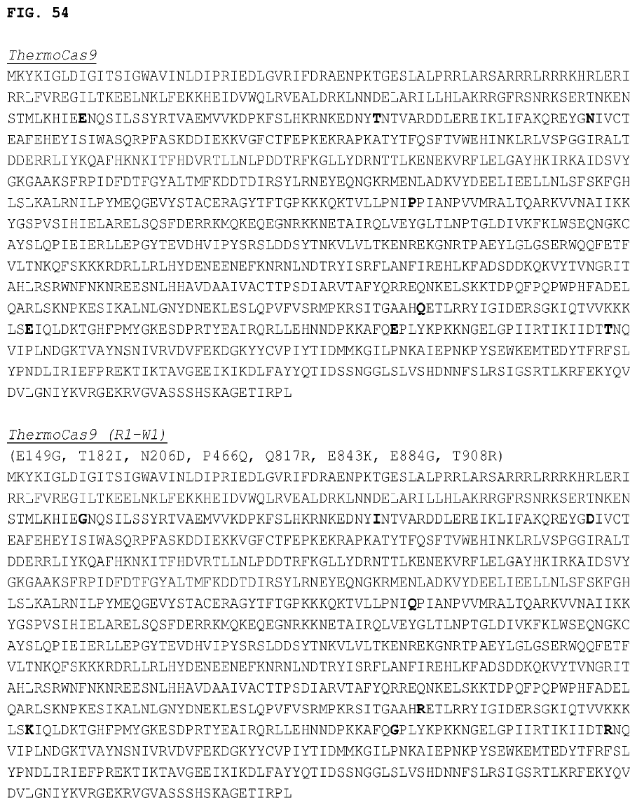

Any of the mutations/substitutions described herein can be used to guide mutation/substitution of other Cas9 proteins (e.g., in addition to the GeoCas9 of FIG. 1), e.g., by making substitutions at the corresponding positions (e.g., via a sequence alignment). See, e.g., FIG. 53 and FIG. 54. For example, the present disclosure thus also provides CRISPR-Cas polypeptides that can be referred to herein as “ThermoCas9 variants”. For all of the embodiments provided herein that refer to variant CRISPR-Cas polypeptides and variant CRISPR-Cas fusion polypeptides (e.g., compositions, methods, nucleic acids, cells, systems, organisms, LNPs, etc.)—the same is provided for CRISPR-Cas polypeptides (e.g., ThermoCas9 variants) and CRISPR-Cas (e.g., ThermoCas9 variant) fusion polypeptides—even when not expressly mentioned. As an illustrative example, it is intended and is to be understood that disclosure of a nucleic acid encoding a variant CRISPR-Cas polypeptide is equally applicable to CRISPR-Cas polypeptides (e.g., ThermoCas9 variants) and should therefore be considered to be disclosure of such.

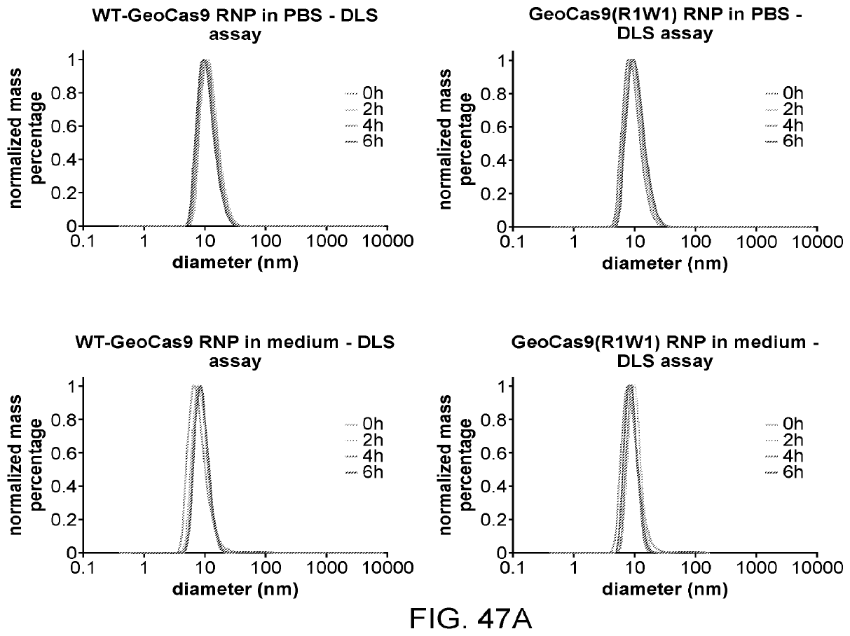

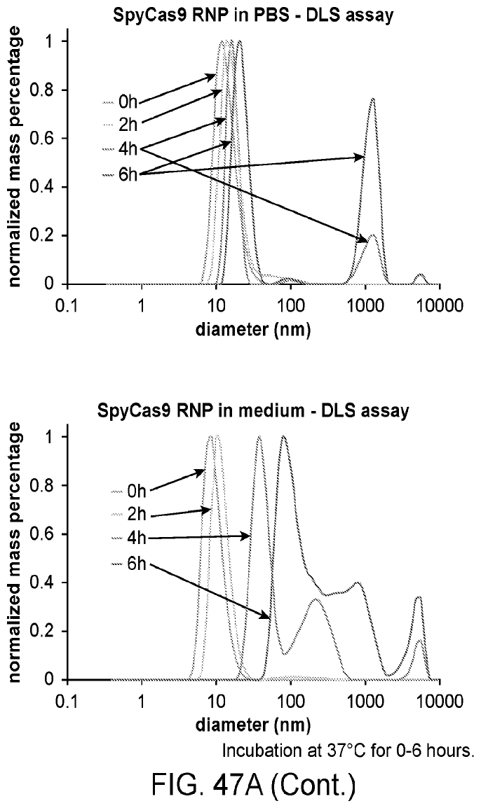

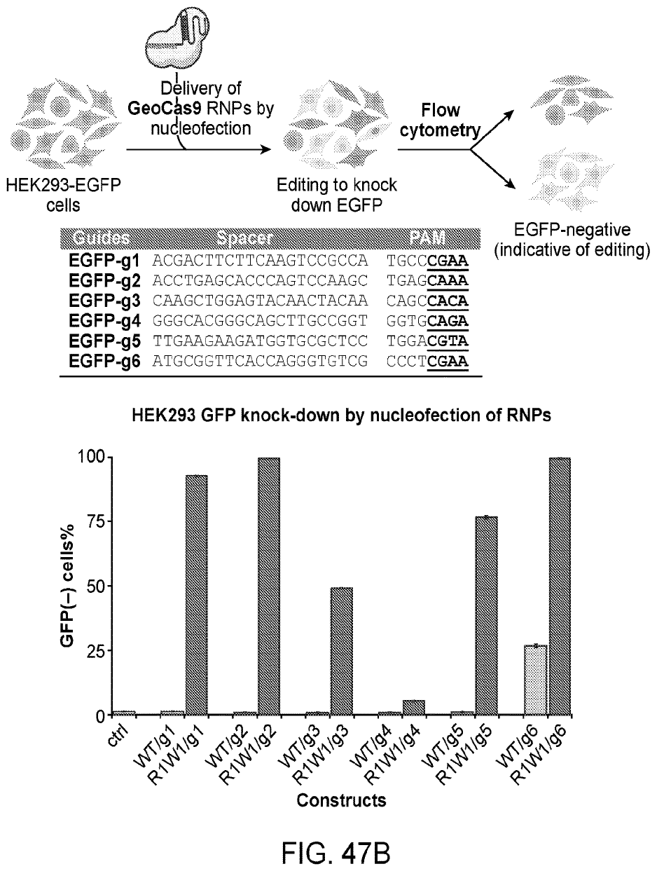

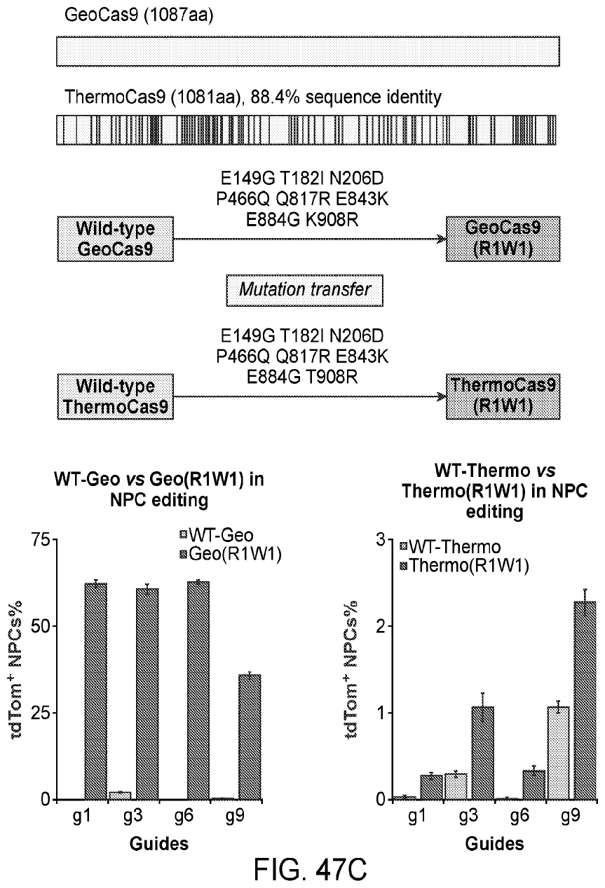

In some cases, a CRISPR-Cas polypeptide (e.g., ThermoCas9 variant) of the present disclosure comprises an amino acid sequence having at least 50%, at least 55%, at least 60%, at least 65%, at least 70%, at least 75%, at least 80%, at least 85%, at least 90%, at least 95%, or at least 99%, amino acid sequence identity to the ThermoCas9 amino acid sequence of SEQ ID NO: 165, where the CRISPR-Cas polypeptide (e.g., ThermoCas9 variant) has at least one amino acid substitution compared to the amino acid sequence of SEQ ID NO: 165 (e.g., where the variant CRISPR-Cas polypeptide has: i) a single amino acid substitution; ii) from 1 to 4 amino acid substitutions; iii) 2 amino acid substitutions; iv) 5 amino acid substitutions; v) 6 amino acid substitutions; vi) 7 amino acid substitutions; vii) 8 amino acid substitutions; viii) 9 amino acid substitutions; ix) 10 amino acid substitutions; or x) from 10 amino acid substitutions to 14 amino acid substitutions). Examples of substitutions are depicted in FIGS. 47C and 54. In some cases, a CRISPR-Cas polypeptide (e.g., ThermoCas9 variant) of the present disclosure comprises an amino acid sequence having at least 50%, at least 55%, at least 60%, at least 65%, at least 70%, at least 75%, at least 80%, at least 85%, at least 90%, at least 95%, or at least 99%, amino acid sequence identity to the amino acid sequence of any one of SEQ ID NOs: 165-175 (see, e.g., Mougiakos et al., Nat Commun. 2017 Nov. 21; 8(1):1647, including Supplementary Table 1), where the CRISPR-Cas polypeptide (e.g., ThermoCas9 variant) has at least one amino acid substitution (e.g., where the variant CRISPR-Cas polypeptide has: i) a single amino acid substitution; ii) from 1 to 4 amino acid substitutions; iii) 2 amino acid substitutions; iv) 5 amino acid substitutions; v) 6 amino acid substitutions; vi) 7 amino acid substitutions; vii) 8 amino acid substitutions; viii) 9 amino acid substitutions; ix) 10 amino acid substitutions; or x) from 10 amino acid substitutions to 14 amino acid substitutions).

In some cases, a CRISPR-Cas polypeptide (e.g., ThermoCas9 variant) of the present disclosure exhibits gene editing activity that is at least 10%, at least 15%, at least 20%, at least 25%, at least 50%, at least 75%, at least 100% (or 2-fold), at least 2.5-fold, at least 3-fold, at least 4-fold, at least 5-fold, at least 10-fold, or more than 10-fold, greater than the gene editing activity of a CRISPR-Cas polypeptide comprising the amino acid sequence of SEQ ID NO: 165. In some cases, a CRISPR-Cas polypeptide (e.g., ThermoCas9 variant) of the present disclosure exhibits gene editing activity that is at least 10%, at least 15%, at least 20%, at least 25%, at least 50%, at least 75%, at least 100% (or 2-fold), at least 2.5-fold, at least 3-fold, at least 4-fold, at least 5-fold, at least 10-fold, or more than 10-fold, greater than the gene editing activity of a corresponding wild type CRISPR-Cas polypeptide (e.g., one comprising the amino acid sequence of any one of SEQ ID NOs: 165-175). Whether a CRISPR-Cas polypeptide (e.g., ThermoCas9 variant) exhibits greater gene editing activity can be determined using any of a variety of assays. One such method is a green fluorescent protein (GFP) to blue fluorescent protein (BFP) conversion assay, as described in Glaser et al. (2016) Mol. Ther. Nucl. Acids 5:e334.

### Variants Comprising Substitutions of E149, T182, N206, and P466

In some cases, a variant CRISPR-Cas polypeptide of the present disclosure comprises a substitution of E149, T182, N206, and P466, based on the amino acid numbering of the GeoCas9 amino acid sequence depicted in FIG. 1 (SEQ ID NO: 1). For example, in some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to the amino acid sequence depicted in FIG. 1, where the variant CRISPR-Cas polypeptide comprises a substitution of E149, T182, N206, and P466, based on the amino acid numbering of the GeoCas9 amino acid sequence depicted in FIG. 1. E149 is substituted with any amino acid other than Glu. In some cases, E149 is substituted with Gly. T182 is substituted with any amino acid other than Thr. In some cases, T182 is substituted with Ile. N206 is substituted with any amino acid other than Asn. In some cases, N206 is substituted with Asp. P466 is substituted with any amino acid other than Pro. In some cases, P466 is substituted with Gln. In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 2, where amino acid 149 is Gly, amino acid 182 is Ile, amino acid 206 is Asp, and amino acid 466 is Gln.

In some cases, the variant CRISPR-Cas effector polypeptide further comprises a substitution of one or more of E179, L190, H340, 1379, K455, L601, Q817, K832, E843, K879, E884, K908, S924, K934, T1015, D1017, 51073, and D1087, based on the amino acid numbering of the GeoCas9 amino acid sequence depicted in FIG. 1 (SEQ ID NO: 1). For example, in some cases, a variant CRISPR-Cas polypeptide comprises: a) a substitution of E149, T182, N206, P466, and E843, based on the amino acid numbering of the GeoCas9 amino acid sequence depicted in FIG. 1; or b) a substitution of E149, T182, N206, P466, E884, and K908, based on the amino acid numbering of the GeoCas9 amino acid sequence depicted in FIG. 1; or c) a substitution of E149, T182, N206, P466, E843, E884, and K908, based on the amino acid numbering of the GeoCas9 amino acid sequence depicted in FIG. 1; or d) a substitution of E149, T182, N206, P466, E843, E884, K908, and Q817, based on the amino acid numbering of the GeoCas9 amino acid sequence depicted in FIG. 1; or e) a substitution of E149, T182, N206, P466, E843, E884, K908, T1015, and D1017, based on the amino acid numbering of the GeoCas9 amino acid sequence depicted in FIG. 1; or f) a substitution of E149, T182, N206, P466, E843, E884, K908, and D1017, based on the amino acid numbering of the GeoCas9 amino acid sequence depicted in FIG. 1; or g) a substitution of E149, T182, N206, P466, E843, E884, K908, L601, and K832, based on the amino acid numbering of the GeoCas9 amino acid sequence depicted in FIG. 1; or h) a substitution of E149, T182, N206, P466, E843, E884, K908, and E179, based on the amino acid numbering of the GeoCas9 amino acid sequence depicted in FIG. 1; or i) a substitution of E149, T182, N206, P466, E843, E884, K908, E179, and Q817, based on the amino acid numbering of the GeoCas9 amino acid sequence depicted in FIG. 1; or j) a substitution of E149, T182, N206, P466, E843, E884, K908, K934, K832, and L601, based on the amino acid numbering of the GeoCas9 amino acid sequence depicted in FIG. 1; or k) a substitution of E149, T182, N206, P466, E843, E884, K908, K934, and K832, based on the amino acid numbering of the GeoCas9 amino acid sequence depicted in FIG. 1; or l) a substitution of E149, T182, N206, P466, E843, E884, K908, K832, L601, and T730, based on the amino acid numbering of the GeoCas9 amino acid sequence depicted in FIG. 1; or m) a substitution of E149, T182, N206, P466. E843, E884, K908, Q817, L190, H340, 1379, K455, K879, and D1087, based on the amino acid numbering of the GeoCas9 amino acid sequence depicted in FIG. 1; or n) a substitution of E149, T182, N206, P466, E843, E884, K908, Q817, S924, and S1073, based on the amino acid numbering of the GeoCas9 amino acid sequence depicted in FIG. 1.

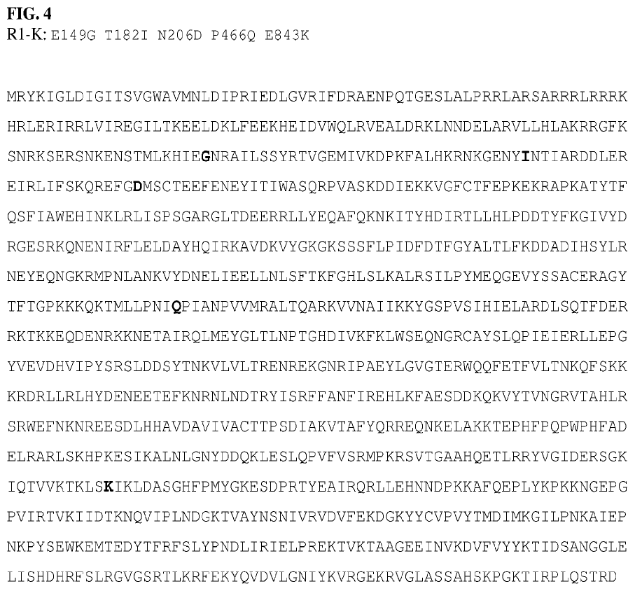

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 4, where amino acid 149 is Gly, amino acid 182 is Ile, amino acid 206 is Asp, amino acid 466 is Gln, and amino acid 843 is Lys.

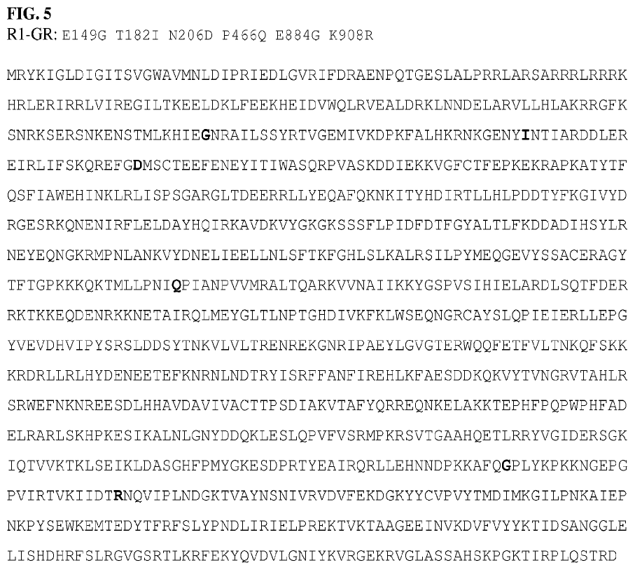

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 5, where amino acid 149 is Gly, amino acid 182 is Ile, amino acid 206 is Asp, amino acid 466 is Gln, amino acid 884 is Gly, and amino acid 908 is Arg.

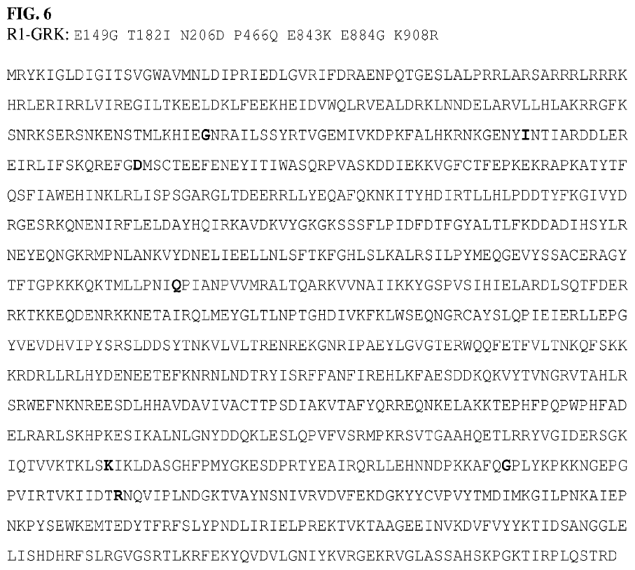

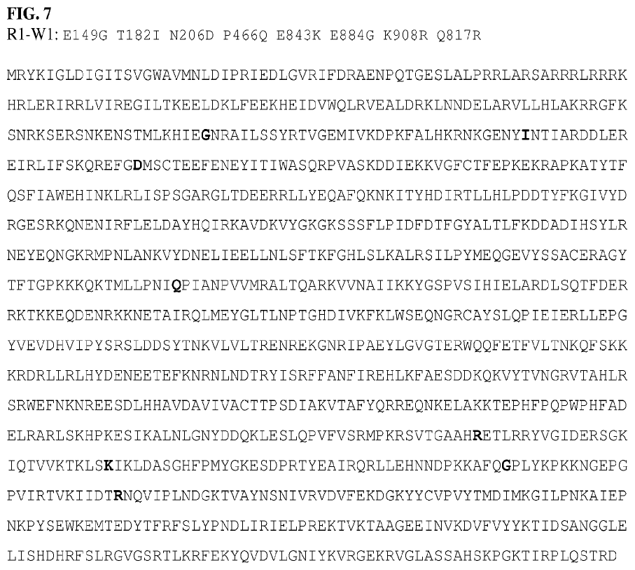

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 6, where amino acid 149 is Gly, amino acid 182 is Ile, amino acid 206 is Asp, amino acid 466 is Gln, amino acid 843 is Lys, amino acid 884 is Gly, and amino acid 908 is Arg.

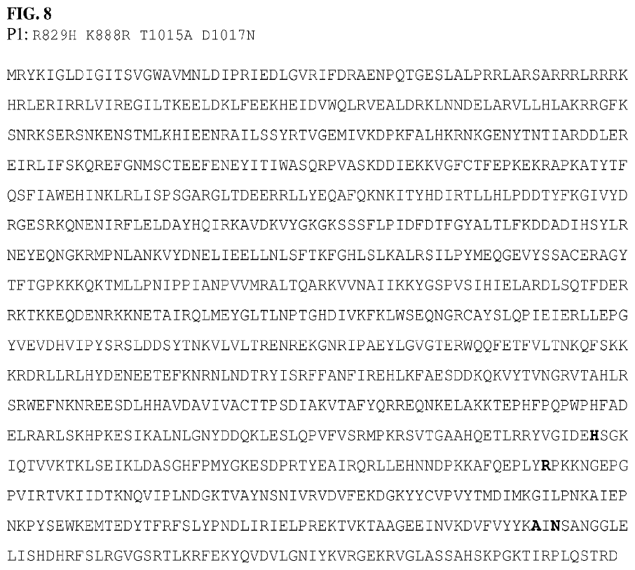

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 7, where amino acid 149 is Gly, amino acid 182 is Ile, amino acid 206 is Asp, amino acid 466 is Gln, amino acid 843 is Lys, amino acid 884 is Gly, amino acid 908 is Arg, and amino acid 817 is Arg.

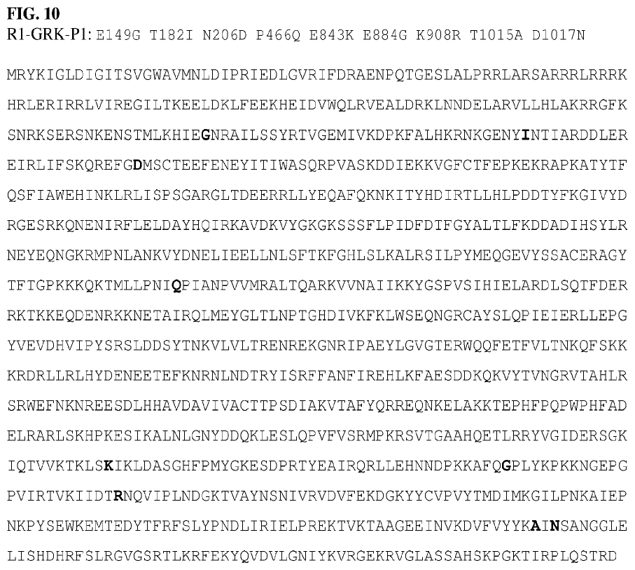

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 10, where amino acid 149 is Gly, amino acid 182 is Ile, amino acid 206 is Asp, amino acid 466 is Gln, amino acid 843 is Lys, amino acid 884 is Gly, amino acid 908 is Arg, amino acid 1015 is Ala, and amino acid 1017 is Asn.

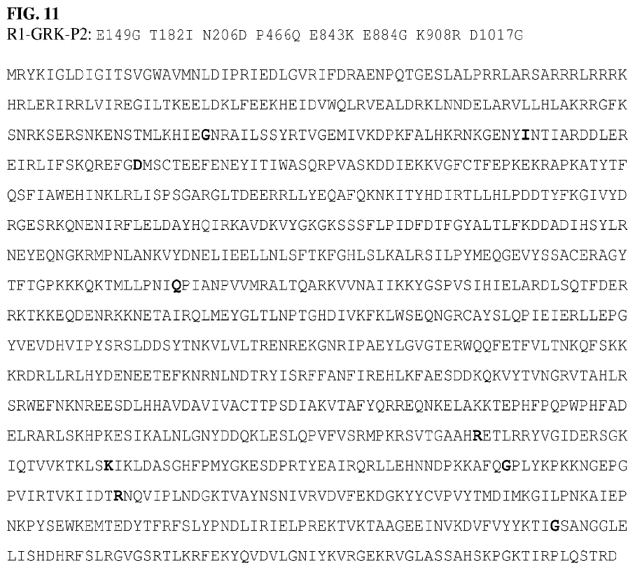

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 11, where amino acid 149 is Gly, amino acid 182 is Ile, amino acid 206 is Asp, amino acid 466 is Gin, amino acid 843 is Lys, amino acid 884 is Gly, amino acid 908 is Arg, and amino acid 1017 is Gly.

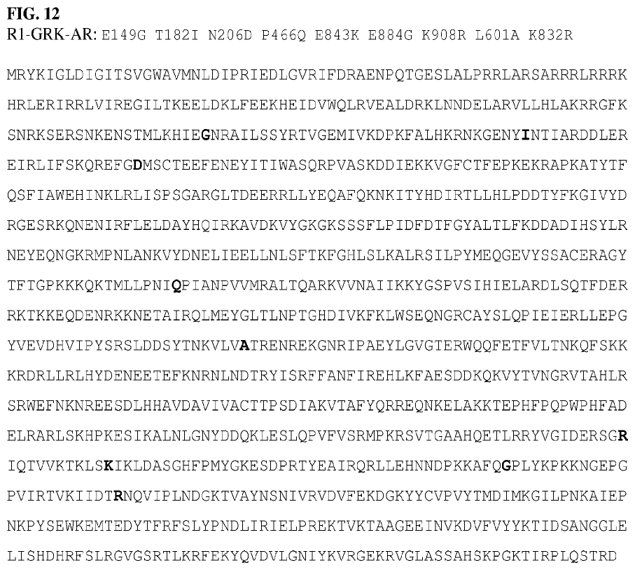

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 12, where amino acid 149 is Gly, amino acid 182 is Ile, amino acid 206 is Asp, amino acid 466 is Gln, amino acid 843 is Lys, amino acid 884 is Gly, amino acid 908 is Arg, amino acid 601 is Ala, and amino acid 832 is Arg.

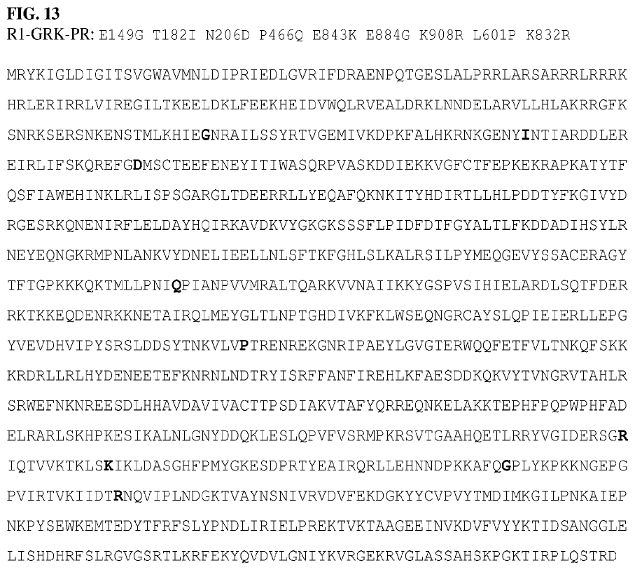

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 13, where amino acid 149 is Gly, amino acid 182 is Ile, amino acid 206 is Asp, amino acid 466 is Gln, amino acid 843 is Lys, amino acid 884 is Gly, amino acid 908 is Arg, amino acid 601 is Pro, and amino acid 832 is Arg.

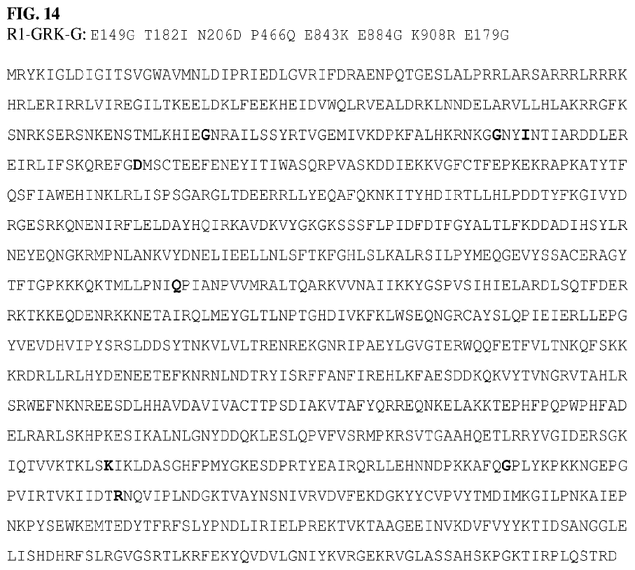

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 14, where amino acid 149 is Gly, amino acid 182 is Ile, amino acid 206 is Asp, amino acid 466 is Gln, amino acid 843 is Lys, amino acid 884 is Gly, amino acid 908 is Arg, and amino acid 179 is Gly.

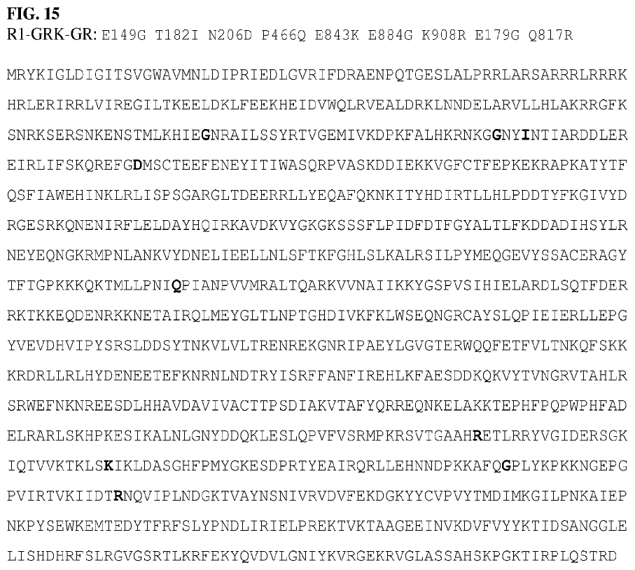

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 15, where amino acid 149 is Gly, amino acid 182 is Ile, amino acid 206 is Asp, amino acid 466 is Gln, amino acid 843 is Lys, amino acid 884 is Gly, amino acid 908 is Arg, amino acid 179 is Gly, and amino acid 817 is Gln.

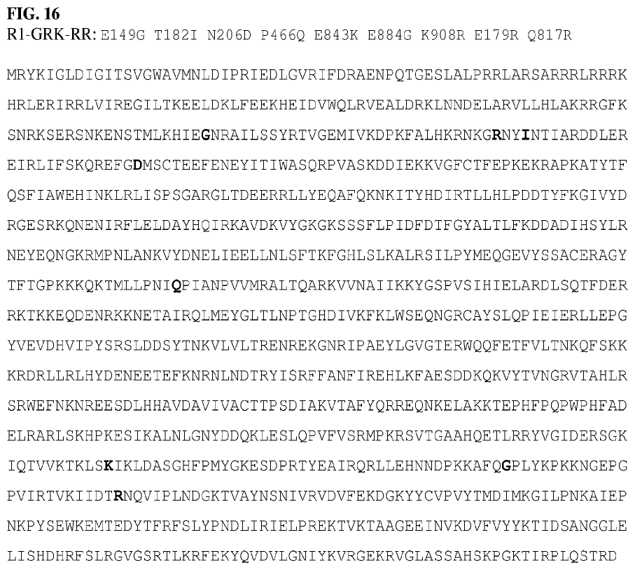

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 16, where amino acid 149 is Gly, amino acid 182 is Ile, amino acid 206 is Asp, amino acid 466 is Gln, amino acid 843 is Lys, amino acid 884 is Gly, amino acid 908 is Arg, amino acid 179 is Arg, and amino acid 817 is Gln.

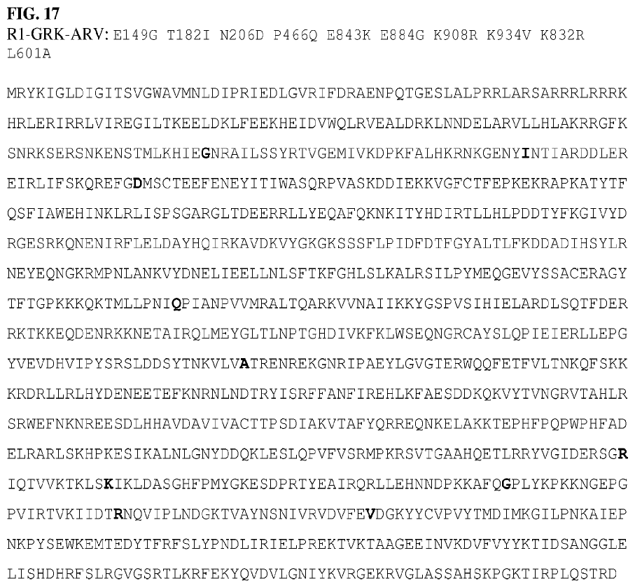

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 17, where amino acid 149 is Gly, amino acid 182 is Ile, amino acid 206 is Asp, amino acid 466 is Gin, amino acid 843 is Lys, amino acid 884 is Gly, amino acid 908 is Arg, amino acid 934 is Val, amino acid 832 is Arg, and amino acid 601 is Ala.

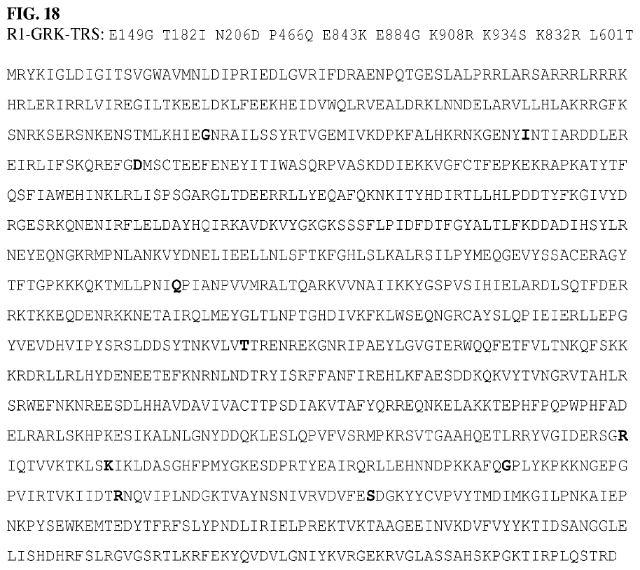

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 18, where amino acid 149 is Gly, amino acid 182 is Ile, amino acid 206 is Asp, amino acid 466 is Gln, amino acid 843 is Lys, amino acid 884 is Gly, amino acid 908 is Arg, amino acid 934 is Scr, amino acid 832 is Arg, and amino acid 601 is Thr.

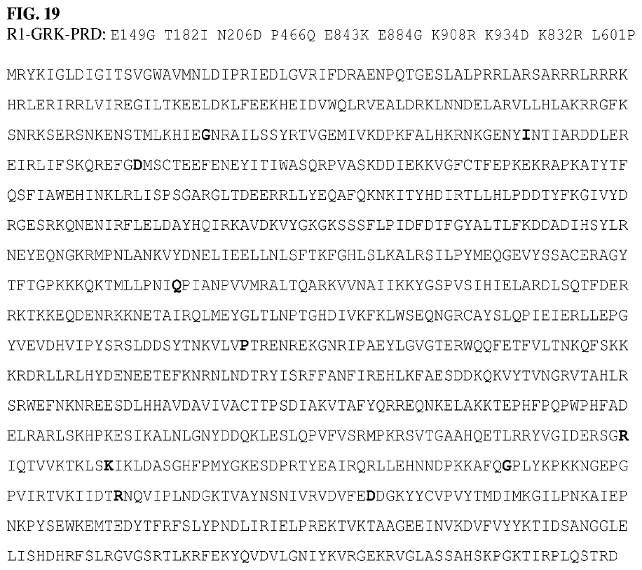

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 19, where amino acid 149 is Gly, amino acid 182 is Ile, amino acid 206 is Asp, amino acid 466 is Gin, amino acid 843 is Lys, amino acid 884 is Gly, amino acid 908 is Arg, amino acid 934 is Asp, amino acid 832 is Arg, and amino acid 601 is Pro.

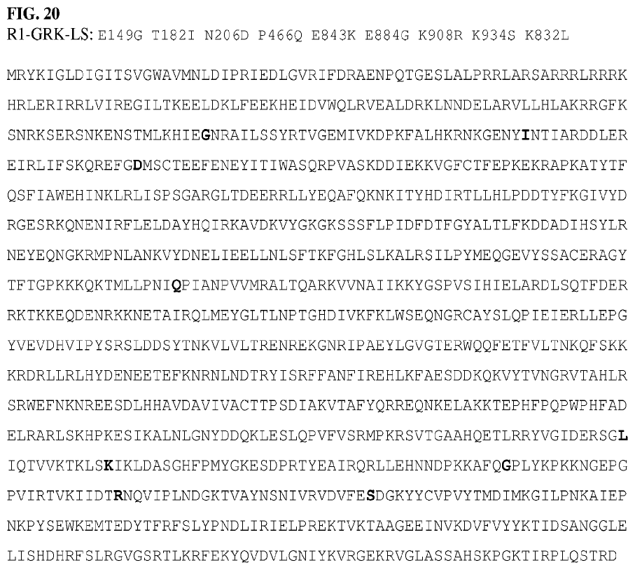

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 20, where amino acid 149 is Gly, amino acid 182 is Ile, amino acid 206 is Asp, amino acid 466 is Gln, amino acid 843 is Lys, amino acid 884 is Gly, amino acid 908 is Arg, amino acid 934 is Ser, and amino acid 832 is Leu.

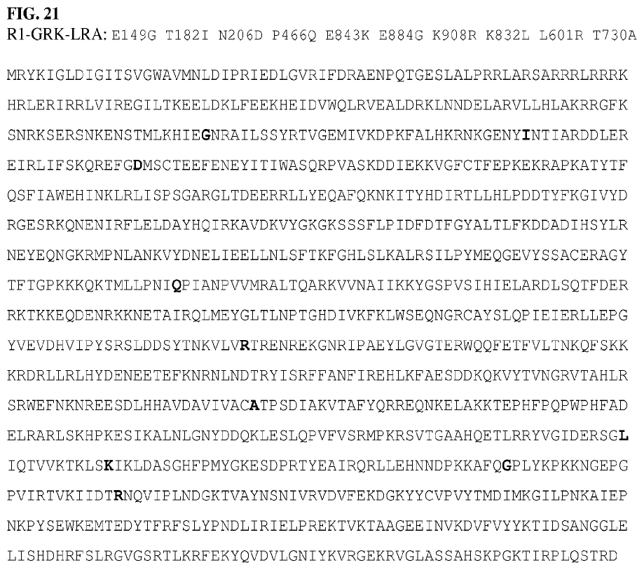

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 21, where amino acid 149 is Gly, amino acid 182 is Ile, amino acid 206 is Asp, amino acid 466 is Gln, amino acid 843 is Lys, amino acid 884 is Gly, amino acid 908 is Arg, amino acid 832 is Leu, amino acid 601 is Arg, and amino acid 730 is Ala.

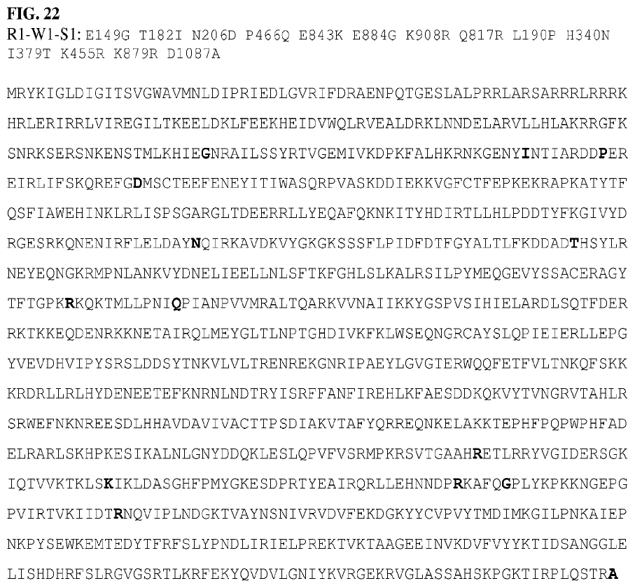

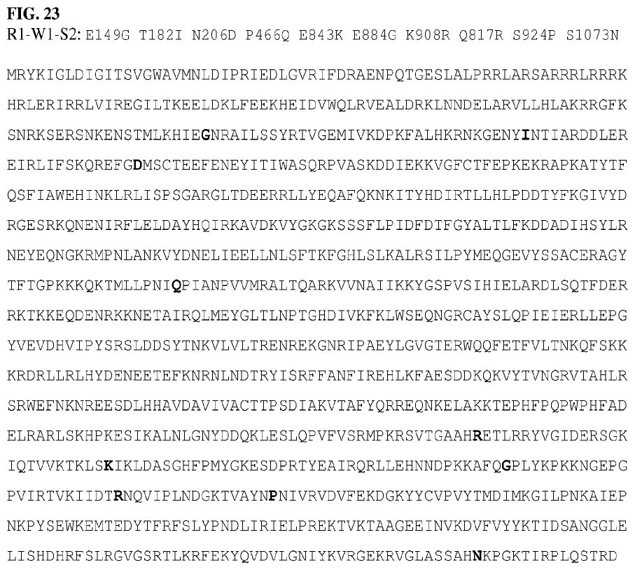

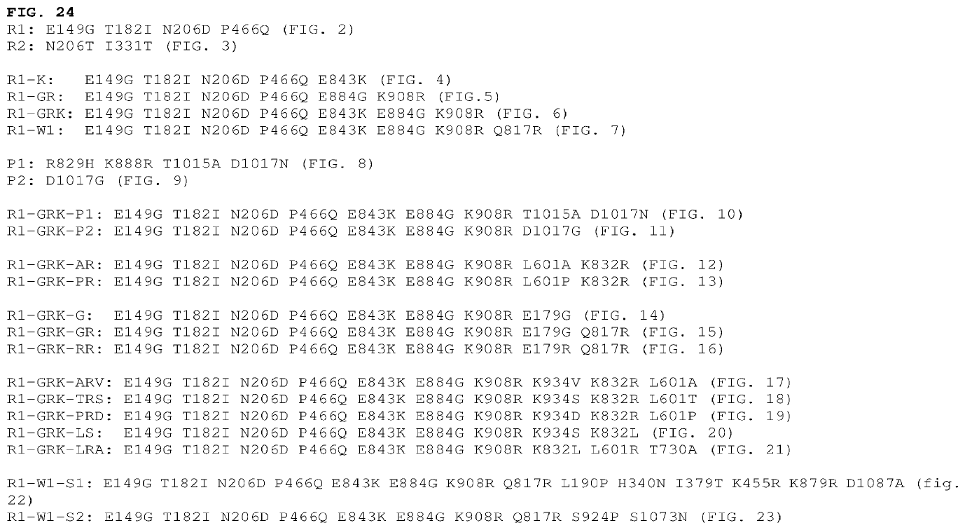

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 22, where amino acid 149 is Gly, amino acid 182 is Ile, amino acid 206 is Asp, amino acid 466 is Gin, amino acid 843 is Lys, amino acid 884 is Gly, amino acid 908 is Arg, amino acid 817 is Arg, amino acid 190 is Pro, amino acid 340 is Asn, amino acid 379 is Thr, amino acid 455 is Arg, amino acid 879 is Arg, and amino acid 1087 is Ala.

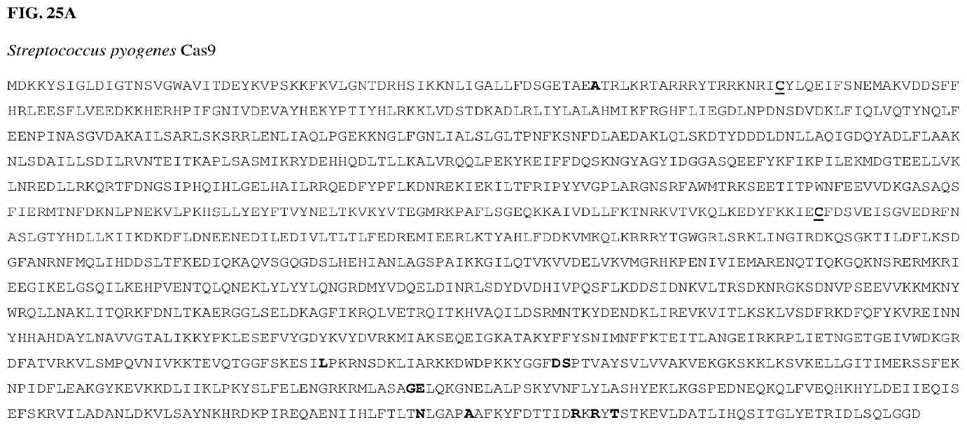

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 23, where amino acid 149 is Gly, amino acid 182 is Ile, amino acid 206 is Asp, amino acid 466 is Gln, amino acid 843 is Lys, amino acid 884 is Gly, amino acid 908 is Arg, amino acid 817 is Arg, amino acid 924 is Pro, and amino acid 1073 is Asn.

As noted above, corresponding substitutions/mutations can be made in Cas9 proteins other than the GeoCas9 of FIG. 1, e.g., in ThermoCas9 and/or its homologs (see, e.g., SEQ ID NOs: 165-175). For example, in some cases, a CRISPR-Cas polypeptide (e.g., ThermoCas9 variant) of the present disclosure comprises a substitution at a position corresponding to E149, T182, N206, and P466 of SEQ ID NO: 1. As such, in some cases, a CRISPR-Cas polypeptide (e.g., ThermoCas9 variant) of the present disclosure comprises a substitution at a position corresponding to E149, T182, N206, and P466 of SEQ ID NO: 165. In some cases, a CRISPR-Cas polypeptide (e.g., ThermoCas9 variant) of the present disclosure comprises a substitution of E149, T182, N206, and P466 based on the amino acid numbering of the ThermoCas9 amino acid sequence of SEQ ID NO: 165.

For example, in some cases, a CRISPR-Cas polypeptide (e.g., ThermoCas9 variant) of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to the amino acid sequence of SEQ ID NO: 165, where the CRISPR-Cas polypeptide (e.g., ThermoCas9 variant) comprises a substitution of E149, T182, N206, and P466, based on the amino acid numbering of the ThermoCas9 amino acid sequence of SEQ ID NO: 165. In some cases, a CRISPR-Cas polypeptide (e.g., ThermoCas9 variant) of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to the amino acid sequence of any one of SEQ ID NOs: 165-175, where the CRISPR-Cas polypeptide (e.g., ThermoCas9 variant) comprises a substitution at positions corresponding to E149, T182, N206, and P466 of SEQ ID NO: 165. E149 is substituted with any amino acid other than Glu. In some cases, E149 is substituted with Gly. T182 is substituted with any amino acid other than Thr. In some cases, T182 is substituted with Ile. N206 is substituted with any amino acid other than Asn. In some cases, N206 is substituted with Asp. P466 is substituted with any amino acid other than Pro. In some cases, P466 is substituted with Gln.

In some cases, a CRISPR-Cas polypeptide (e.g., ThermoCas9 variant) of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence of SEQ ID NO: 176, where amino acid 149 is Gly, amino acid 182 is Ile, amino acid 206 is Asp, amino acid 466 is Gln, amino acid 843 is Lys, amino acid 884 is Gly, amino acid 908 is Arg, and amino acid 817 is Arg.

In some cases, the CRISPR-Cas polypeptide (e.g., ThermoCas9 variant) further comprises a substitution of one or more of E179, L190, H340, 1379, K455, L601, Q817, K832, E843, K879, E884, K908, S924, K934, T1015, D1017, S1073, and D1087, based on the amino acid numbering of the GeoCas9 amino acid sequence depicted in FIG. 1. For example, in some cases, the CRISPR-Cas polypeptide (e.g., ThermoCas9 variant) further comprises a substitution of one or more positions corresponding to E179, L190, H340, 1379, K455, L601, Q817, K832, E843, K879, E884, K908, S924, K934, T1015, D1017, S1073, and D1087 of the amino acid numbering of the GeoCas9 amino acid sequence depicted in FIG. 1. For example, in some cases, a CRISPR-Cas polypeptide (e.g., ThermoCas9 variant) comprises: a) a substitution of E149, T182, N206, P466, and E843, based on the amino acid numbering of SEQ ID NO: 165; or b) a substitution of E149, T182, N206, P466, E884, and T908, based on the amino acid numbering of SEQ ID NO: 165; or c) a substitution of E149, T182, N206, P466, E843. E884, and T908, based on the amino acid numbering of SEQ ID NO: 165; or d) a substitution of E149, T182, N206, P466, E843, E884, T908, and Q817, based on the amino acid numbering of SEQ ID NO: 165. In some cases, a CRISPR-Cas polypeptide (e.g., ThermoCas9 variant) of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to the amino acid sequence of any one of SEQ ID NOs: 165-175, where the CRISPR-Cas polypeptide (e.g., ThermoCas9 variant) comprises: a) a substitution at positions corresponding to E149, T182, N206, P466, and E843 of SEQ ID NO: 165; or b) a substitution at positions corresponding to E149, T182, N206, P466, E884, and T908 of SEQ ID NO: 165; or c) a substitution at positions corresponding to E149, T182, N206, P466, E843, E884, and T908 of SEQ ID NO: 165; or d) a substitution at positions corresponding to E149, T182, N206, P466, E843, E884, T908, and Q817 of SEQ ID NO: 165. E149 is substituted with any amino acid other than Glu. In some cases, E149 is substituted with Gly. T182 is substituted with any amino acid other than Thr. In some cases, T182 is substituted with Ile. N206 is substituted with any amino acid other than Asn. In some cases, N206 is substituted with Asp. P466 is substituted with any amino acid other than Pro. In some cases, P466 is substituted with Gln. E843 is substituted with any amino acid other than E. In some cases, E843 is substituted with K. E884 is substituted with any amino acid other than E. In some cases, E884 is substituted with G. T908 is substituted with any amino acid other than T. In some cases, T908 is substituted with R. Q817 is substituted with any amino acid other than Q. In some cases, Q817 is substituted with R.

### Variants Comprising a Substitution of D1017

In some cases, a variant CRISPR-Cas polypeptide of the present disclosure comprises a substitution of D1017, based on the amino acid numbering of the GeoCas9 amino acid sequence depicted in FIG. 1 (SEQ ID NO: 1). For example, in some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to the amino acid sequence depicted in FIG. 1, where the variant CRISPR-Cas polypeptide comprises a substitution of D1017, based on the amino acid numbering of the GeoCas9 amino acid sequence depicted in FIG. 1. D1017 is substituted with an amino acid other than Asp. In some cases, D1017 is substituted with Gly. In some cases, D1017 is substituted with Asn. In some cases, a variant CRISPR-Cas polypeptide of the present disclosure comprises a substitution of D1017, based on the amino acid numbering of the GeoCas9 amino acid sequence depicted in FIG. 1. For example, in some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to the amino acid sequence depicted in FIG. 1, where the variant CRISPR-Cas polypeptide comprises a substitution of D1017, based on the amino acid numbering of the GeoCas9 amino acid sequence depicted in FIG. 1, and where the variant CRISPR-Cas polypeptide further includes a substitution of one or more of R829, K888, and T1015, based on the amino acid numbering of the GeoCas9 amino acid sequence depicted in FIG. 1.

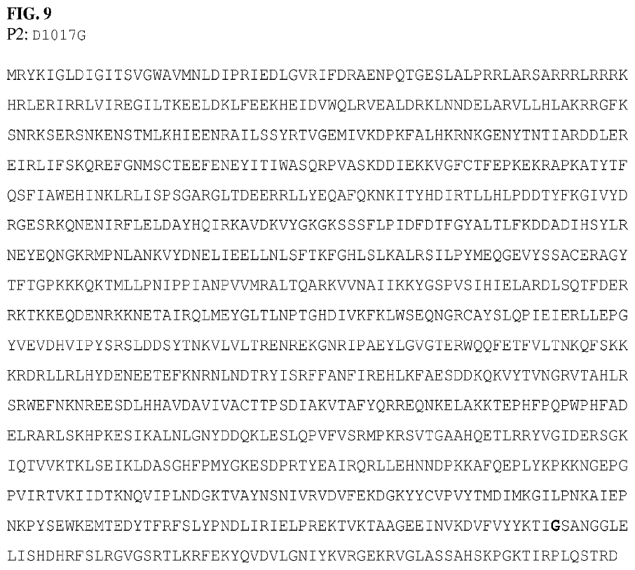

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 9, where amino acid 1017 is other than Asp. In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 9, where amino acid 1017 is Gly.

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 10, where amino acid 1017 is Asn, amino acid 829 is His, amino acid 888 is Arg, and amino acid 1015 is Ala.

In some cases, a CRISPR-Cas polypeptide (e.g., ThermoCas9 variant) of the present disclosure comprises a substitution of D1017, based on the amino acid numbering of the GeoCas9 amino acid sequence depicted in FIG. 1. For example, in some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to SEQ ID NO: 165, where the CRISPR-Cas polypeptide comprises a substitution at a position corresponding to D1017 of SEQ ID NO: 1. The amino acid at this position is substituted with an amino acid other than Asp. In some cases, the amino acid at this position is substituted with Gly. In some cases, the amino acid at this position is substituted with Asn. For example, in some cases, a CRISPR-Cas polypeptide (e.g., ThermoCas9 variant) of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to SEQ ID NO: 165, where the CRISPR-Cas polypeptide comprises a substitution at a position corresponding to D1017 of SEQ ID NO: 1, and where the CRISPR-Cas polypeptide further includes a substitution of one or more positions corresponding to R829, K888, and T1015 of SEQ ID NO: 1.

In some cases, a CRISPR-Cas polypeptide (e.g., ThermoCas9 variant) of the present disclosure comprises a substitution of D1017, based on the amino acid numbering of the GeoCas9 amino acid sequence depicted in FIG. 1. For example, in some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%. amino acid sequence identity to any one of SEQ ID NOs: 165-175, where the CRISPR-Cas polypeptide comprises a substitution at a position corresponding to D1017 of SEQ ID NO: 1. The amino acid at this position is substituted with an amino acid other than Asp. In some cases, the amino acid at this position is substituted with Gly. In some cases, the amino acid at this position is substituted with Asn. For example, in some cases, a CRISPR-Cas polypeptide (e.g., ThermoCas9 variant) of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to any one of SEQ ID NOs: 165-175, where the CRISPR-Cas polypeptide comprises a substitution at a position corresponding to D1017 of SEQ ID NO: 1, and where the CRISPR-Cas polypeptide further includes a substitution of one or more positions corresponding to R829, K888, and T1015 of SEQ ID NO: 1.

### Variants Comprising Substitutions of N206 and 1331

In some cases, a variant CRISPR-Cas polypeptide of the present disclosure comprises a substitution of N206 and a substitution of 1331, based on the amino acid numbering of the GeoCas9 amino acid sequence depicted in FIG. 1 (SEQ ID NO: 1). For example, in some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to the amino acid sequence depicted in FIG. 1, where the variant CRISPR-Cas polypeptide comprises a substitution of N206 and a substitution of 1331, based on the amino acid numbering of the GeoCas9 amino acid sequence depicted in FIG. 1. N206 is substituted with an amino acid other than Asn; and 1331 is substituted with an amino acid other than Ile. In some cases, N206 is substituted with Thr; and in some cases, 1331 is substituted with Thr.

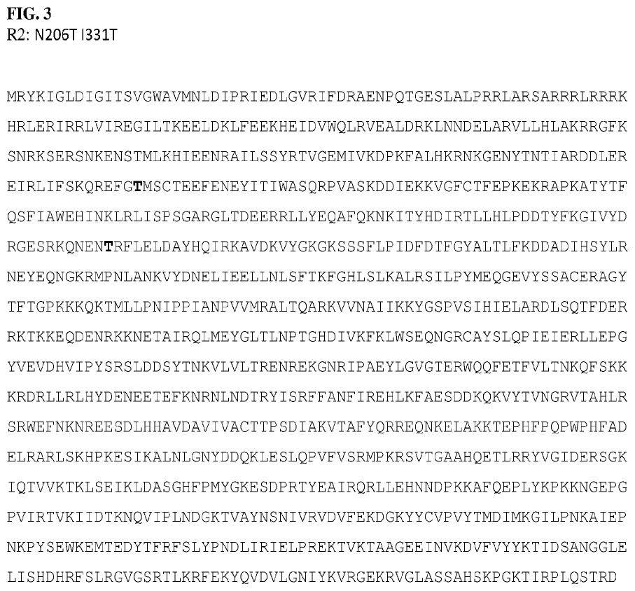

In some cases, a variant CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to the amino acid sequence depicted in FIG. 3, where amino acid 206 is Thr and amino acid 331 is Thr.

In some cases, a CRISPR-Cas polypeptide (e.g., ThermoCas9 variant) of the present disclosure comprises a substitution of N206 and a substitution of 1331, based on the amino acid numbering of the GeoCas9 amino acid sequence depicted in FIG. 1. For example, in some cases, a CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to the amino acid sequence of SEQ ID NO: 165, where the CRISPR-Cas polypeptide comprises substitutions at positions corresponding to N206 and 1331 of SEQ ID NO: 1. In some cases, a CRISPR-Cas polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, or at least 99%, amino acid sequence identity to the amino acid sequence of any one of SEQ ID NOs: 165-175, where the CRISPR-Cas polypeptide comprises substitutions at positions corresponding to N206 and 1331 of SEQ ID NO: 1. The amino acid at the position corresponding to N206 is substituted with an amino acid other than Asn; and the amino acid at the position corresponding to 1331 is substituted with an amino acid other than Ile. In some cases, the amino acid at the position corresponding to N206 is substituted with Thr; and in some cases, the amino acid at the position corresponding to 1331 is substituted with Thr.

### Length

A variant CRISPR-Cas effector polypeptide of the present disclosure can have a length of from 1050 amino acids to 1120 amino acids. For example, in some cases, a variant CRISPR-Cas effector polypeptide has a length of from 1050 amino acids to 1055 amino acids, from 1055 amino acids to 1060 amino acids, from 1060 amino acids to 1065 amino acids, from 1065 amino acids to 1070 amino acids, from 1070 amino acids to 1075 amino acids, from 1075 amino acids to 1080 amino acids, from 1080 amino acids to 1085 amino acids, from 1085 amino acids to 1090 amino acids, from 1090 amino acids to 1095 amino acids, from 1095 amino acids to 1100 amino acids, from 1100 amino acids to 1105 amino acids, from 1105 amino acids to 1110 amino acids, from 1110 amino acids to 1115 amino acids, or from 1115 amino acids to 1120 amino acids. In some cases, a variant CRISPR-Cas effector polypeptide has a length of from 1080 amino acids to 1095 amino acids. In some cases, a variant CRISPR-Cas effector polypeptide has a length of from 1080 to 1090 amino acids. In some cases, a variant CRISPR-Cas effector polypeptide has a length of 1087 amino acids.

A CRISPR-Cas polypeptide (e.g., ThermoCas9 variant) of the present disclosure can have a length of from 1050 amino acids to 1120 amino acids. For example, in some cases, a CRISPR-Cas polypeptide (e.g., ThermoCas9 variant) has a length of from 1050 amino acids to 1055 amino acids, from 1055 amino acids to 1060 amino acids, from 1060 amino acids to 1065 amino acids, from 1065 amino acids to 1070 amino acids, from 1070 amino acids to 1075 amino acids, from 1075 amino acids to 1080 amino acids, from 1080 amino acids to 1085 amino acids, from 1085 amino acids to 1090 amino acids, from 1090 amino acids to 1095 amino acids, from 1095 amino acids to 1100 amino acids, from 1100 amino acids to 1105 amino acids, from 1105 amino acids to 1110 amino acids, from 1110 amino acids to 1115 amino acids, or from 1115 amino acids to 1120 amino acids. In some cases, a CRISPR-Cas polypeptide (e.g., ThermoCas9 variant) has a length of from 1080 amino acids to 1095 amino acids. In some cases, a CRISPR-Cas polypeptide (e.g., ThermoCas9 variant) has a length of from 1080 to 1090 amino acids. In some cases, a CRISPR-Cas polypeptide (e.g., ThermoCas9 variant) has a length of 1087 amino acids.

### Thermostability

A variant CRISPR-Cas effector polypeptide of the present disclosure is enzymatically active in a temperature range of from 15° C. to 75° C. In some case, a variant CRISPR-Cas effector polypeptide of the present disclosure is enzymatically active in a temperature range of from 20° C. to 75° C. In some case, a variant CRISPR-Cas effector polypeptide of the present disclosure is enzymatically active in a temperature range of from 25° C. to 75° C. In some cases, a variant CRISPR-Cas effector polypeptide of the present disclosure exhibits maximal enzymatic activity (as defined by cleavage rate of a target nucleic acid when complexed with a guide RNA) in a temperature range of from 50° C. to 70° C. In some cases, a variant CRISPR-Cas effector polypeptide of the present disclosure, at a temperature of from 40° C. to 49° C., cleaves a target nucleic acid at a cleavage rate that is at least 30%, at least 35%, at least 40%, at least 45%, at least 50%, at least 60%, at least 70%, at least 80%, or at least 90%, of the maximal cleavage rate that is obtained in a temperature range of from 50° C. to 70° C. In some cases, a variant CRISPR-Cas effector polypeptide of the present disclosure, at a temperature of from 30° C. to 40° C., cleaves a target nucleic acid at a cleavage rate that is at least at least 10%, at least 15%, at least 20%, at least 25%, at least 30%, at least 35%, at least 40%, at least 45%, at least 50%, at least 60%, at least 70%, at least 80%, or at least 90%, of the maximal cleavage rate that is obtained in a temperature range of from 50° C. to 70° C.

A CRISPR-Cas polypeptide (e.g., ThermoCas9 variant) of the present disclosure is enzymatically active in a temperature range of from 15° C. to 75° C. In some case, a CRISPR-Cas polypeptide (e.g., ThermoCas9 variant) of the present disclosure is enzymatically active in a temperature range of from 20° C. to 75° C. In some case, a CRISPR-Cas polypeptide (e.g., ThermoCas9 variant) of the present disclosure is enzymatically active in a temperature range of from 25° C. to 75° C. In some cases, a CRISPR-Cas polypeptide (e.g., ThermoCas9 variant) of the present disclosure exhibits maximal enzymatic activity (as defined by cleavage rate of a target nucleic acid when complexed with a guide RNA) in a temperature range of from 50° C. to 70° C. In some cases, a CRISPR-Cas polypeptide (e.g., ThermoCas9 variant) of the present disclosure, at a temperature of from 40° C. to 49° C., cleaves a target nucleic acid at a cleavage rate that is at least 30%, at least 35%, at least 40%, at least 45%, at least 50%, at least 60%, at least 70%, at least 80%, or at least 90%, of the maximal cleavage rate that is obtained in a temperature range of from 50° C. to 70° C. In some cases, a CRISPR-Cas polypeptide (e.g., ThermoCas9 variant) of the present disclosure, at a temperature of from 30° C. to 40° C., cleaves a target nucleic acid at a cleavage rate that is at least at least 10%, at least 15%, at least 20%, at least 25%, at least 30%, at least 35%, at least 40%, at least 45%, at least 50%, at least 60%, at least 70%, at least 80%, or at least 90%, of the maximal cleavage rate that is obtained in a temperature range of from 50° C. to 70° C.

The terms “enzymatic activity” and “enzymatically active,” (e.g., as used herein with reference to a variant CRISPR-Cas effector polypeptide of the present disclosure), refers to cleavage of one or both strands of a target nucleic acid by the variant CRISPR-Cas effector polypeptide when the variant CRISPR-Cas effector polypeptide is complexed with a guide RNA.

In some cases, a variant CRISPR-Cas effector polypeptide (or a CRISPR-Cas polypeptide (e.g., ThermoCas9 variant) of the present disclosure) of the present disclosure is stable, and retains enzymatic activity, in serum. For example, in some cases, a variant CRISPR-Cas effector polypeptide (or a CRISPR-Cas polypeptide (e.g., ThermoCas9 variant) of the present disclosure) of the present disclosure, when present in undiluted serum (e.g., undiluted human serum) in vitro at 37° C., retains at least 50%, at least 60%, at least 70%, at least 80%, at least 90%, at least 95%, at least 98%, or 100%, of the starting enzymatic activity (e.g., the enzymatic activity of the RNA-guided endonuclease before being added to the serum), for a period of time of at least 30 minutes, at least 1 hour, at least 2 hours, at least 4 hours, at least 8 hours, at least 12 hours, at least 24 hours, at least 36 hours, at least 3 days, at least 4 days, at least 5 days, at least 6 days, at least 7 days, at least 2 weeks, at least 4 weeks, at least 2 months, at least 4 months, at least 6 months, or more than 6 months. For example, in some cases, a variant CRISPR-Cas effector polypeptide (or a CRISPR-Cas polypeptide (e.g., ThermoCas9 variant) of the present disclosure) of the present disclosure, when present in undiluted serum (e.g., undiluted human serum) in vitro at 37° C. retains at least 50%, at least 60%, at least 70%, at least 80%, at least 90%, at least 95%, at least 98%, or 100%, of the starting enzymatic activity (e.g., the enzymatic activity of the variant CRISPR-Cas effector polypeptide (or a CRISPR-Cas polypeptide (e.g., ThermoCas9 variant) of the present disclosure) before being added to the serum), for a period of time of from about 30 minutes to longer than 6 months, e.g., from about 30 minutes to about 1 hour, from about 1 hour to about 2 hours, from about 2 hours to about 4 hours, from about 4 hours to about 8 hours, from about 8 hours to about 12 hours, from about 12 hours to about 24 hours, from about 24 hours to about 36 hours, from about 36 hours to about 2 days, from about 2 days to about 7 days, from about 1 week to about 4 weeks, from about 1 month to about 6 months, or more than 6 months.

In some cases, a variant CRISPR-Cas effector polypeptide (or a CRISPR-Cas polypeptide (e.g., ThermoCas9 variant) of the present disclosure) of the present disclosure, when present in serum (e.g., human serum) in vivo at 37° C. retains at least 50%, at least 60%, at least 70%, at least 80%, at least 90%, at least 95%, at least 98%, or 100%, of the starting enzymatic activity (e.g., the enzymatic activity of the variant CRISPR-Cas effector polypeptide(or a CRISPR-Cas polypeptide (e.g., ThermoCas9 variant) of the present disclosure) before being added to the serum), for a period of time of at least 30 minutes, at least 1 hour, at least 2 hours, at least 4 hours, at least 8 hours, at least 12 hours, at least 24 hours, at least 36 hours, at least 3 days, at least 4 days, at least 5 days, at least 6 days, at least 7 days, at least 2 weeks, at least 4 weeks, at least 2 months, at least 4 months, at least 6 months, or more than 6 months. In some cases, a variant CRISPR-Cas effector polypeptide (or a CRISPR-Cas polypeptide (e.g., ThermoCas9 variant) of the present disclosure) of the present disclosure, when present in serum (e.g., human serum) in vivo at 37° C., retains at least 50%, at least 60%, at least 70%, at least 80%, at least 90%, at least 95%, at least 98%, or 100%, of the starting enzymatic activity (e.g., the enzymatic activity of the v before being added to the serum), for a period of time of from about 30 minutes to longer than 6 months, e.g., from about 30 minutes to about 1 hour, from about 1 hour to about 2 hours, from about 2 hours to about 4 hours, from about 4 hours to about 8 hours, from about 8 hours to about 12 hours, from about 12 hours to about 24 hours, from about 24 hours to about 36 hours, from about 36 hours to about 2 days, from about 2 days to about 7 days, from about 1 week to about 4 weeks, from about 1 month to about 6 months, or more than 6 months.

### Protospacer Adjacent Motif (PAM)

A variant CRISPR-Cas effector polypeptide (or a CRISPR-Cas polypeptide (e.g., ThermoCas9 variant) of the present disclosure) of the present disclosure binds to target DNA at a target sequence defined by the region of complementarity between the DNA-targeting RNA (guide RNA) and the target DNA. As is the case for many CRISPR-Cas effector polypeptides, site-specific binding (and/or cleavage) of a double stranded target DNA occurs at locations determined by both (i) base-pairing complementarity between the guide RNA and the target DNA; and (ii) a short motif [referred to as the protospacer adjacent motif (PAM)] in the target DNA.

In some embodiments, the PAM for a variant CRISPR-Cas effector polypeptide (or a CRISPR-Cas polypeptide (e.g., ThermoCas9 variant)) of the present disclosure is immediately 5′ of the target sequence of the non-complementary strand of the target DNA (the complementary strand hybridizes to the guide sequence of the guide RNA while the non-complementary strand does not directly hybridize with the guide RNA and is the reverse complement of the non-complementary strand). In some cases, a variant CRISPR-Cas effector polypeptide (or a CRISPR-Cas polypeptide (e.g., ThermoCas9 variant)) of the present disclosure binds to a target nucleic acid comprising a PAM comprising a GMAA sequence, where M is A or C (amino). In some cases, variant CRISPR-Cas effector polypeptide (or a CRISPR-Cas polypeptide (e.g., ThermoCas9 variant)) binds to a target nucleic acid comprising a PAM comprising a CRAA sequence, where R is G or A (purine). In some cases, a suitable PAM comprises a CNNA sequence, where N is any nucleotide. In some cases, a suitable PAM comprises the nucleotide sequence GAAA. In some cases, a suitable PAM comprises the nucleotide sequence GCAA. In some cases, a suitable PAM comprises a NNNNCRAA sequence, where N is any nucleotide. In some cases, a suitable PAM comprises a NNNNCNNA sequence, where N is any nucleotide.

### Guide RNAs

A nucleic acid that binds to a variant CRISPR-Cas effector polypeptide (or a CRISPR-Cas polypeptide (e.g., ThermoCas9 variant) of the present disclosure) of the present disclosure, forming a ribonucleoprotein complex (RNP), and targets the complex to a specific location within a target nucleic acid (e.g., a target DNA) is referred to herein as a simply as a “guide RNA.” It is to be understood that in some cases, a hybrid DNA/RNA can be made such that guide RNA suitable for use in a complex with a variant CRISPR-Cas effector polypeptide (or a CRISPR-Cas polypeptide (e.g., ThermoCas9 variant) of the present disclosure) of the present disclosure includes DNA bases in addition to RNA bases, but the term “guide RNA” is still used to encompass such a molecule herein.

A guide RNA can be said to include two segments, a targeting segment and a protein-binding segment. The targeting segment of a guide RNA includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a variant CRISPR-Cas effector polypeptide (or a CRISPR-Cas polypeptide (e.g., ThermoCas9 variant) of the present disclosure). The protein-binding segment of a subject guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the guide RNA (the guide sequence of the guide RNA) and the target nucleic acid.

A guide RNA and a variant CRISPR-Cas effector polypeptide (or a CRISPR-Cas polypeptide (e.g., ThermoCas9 variant) of the present disclosure) of the present disclosure (e.g., a variant CRISPR-Cas effector polypeptide; or a fusion polypeptide comprising a variant CRISPR-Cas effector polypeptide and a heterologous fusion partner), form a complex (e.g., bind via non-covalent interactions). The guide RNA provides target specificity to the complex by including a targeting segment, which includes a guide sequence (a nucleotide sequence that is complementary to a sequence of a target nucleic acid). The variant CRISPR-Cas effector polypeptide (or fusion protein) (or a CRISPR-Cas polypeptide (e.g., ThermoCas9 variant) of the present disclosure) of the complex provides the site-specific activity (e.g., cleavage activity provided by the variant CRISPR-Cas effector polypeptide and/or an activity provided by the fusion partner in the case of a fusion polypeptide comprising a variant CRISPR-Cas effector polypeptide (or a CRISPR-Cas polypeptide (e.g., ThermoCas9 variant) of the present disclosure). In other words, the variant CRISPR-Cas effector polypeptide (or a CRISPR-Cas polypeptide (e.g., ThermoCas9 variant) of the present disclosure) is guided to a target nucleic acid sequence (e.g. a target sequence) by virtue of its association with the guide RNA.

The “guide sequence” also referred to as the “targeting sequence” of a guide RNA can be modified so that the guide RNA can target a variant CRISPR-Cas effector polypeptide (e.g., a variant CRISPR-Cas effector polypeptide, or a fusion polypeptide comprising a variant CRISPR-Cas effector polypeptide) (or a CRISPR-Cas polypeptide (e.g., ThermoCas9 variant) of the present disclosure) to any desired sequence of any desired target nucleic acid, with the exception (e.g., as described herein) that the PAM sequence can be taken into account. Thus, for example, a guide RNA can have a guide sequence with complementarity to (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

A subject guide RNA can also be said to include an “activator” and a “targeter” (e.g., an “activator-RNA” and a “targeter-RNA,” respectively). When the “activator” and a “targeter” are two separate molecules the guide RNA is referred to herein as a “dual guide RNA”, a “dgRNA,” a “double-molecule guide RNA”, or a “two-molecule guide RNA.” In some embodiments, the activator and targeter are covalently linked to one another (e.g., via intervening nucleotides) and the guide RNA is referred to herein as a “single guide RNA”, an “sgRNA,” a “single-molecule guide RNA,” or a “one-molecule guide RNA”. Thus, a subject single guide RNA comprises a targeter (e.g., targeter-RNA) and an activator (e.g., activator-RNA) that are linked to one another (e.g., by intervening nucleotides), and hybridize to one another to form the double stranded RNA duplex (dsRNA duplex) of the protein-binding segment of the guide RNA, thus resulting in a stem-loop structure. Thus, the targeter and the activator each have a duplex-forming segment, where the duplex forming segment of the targeter and the duplex-forming segment of the activator have complementarity with one another and hybridize to one another.

In some embodiments, the linker of a single guide RNA is a stretch of nucleotides. In some cases, the targeter and activator of a single guide RNA are linked to one another by intervening nucleotides and the linker can have a length of from 3 to 20 nucleotides (nt) (e.g., from 3 to 15, 3 to 12, 3 to 10, 3 to 8, 3 to 6, 3 to 5, 3 to 4, 4 to 20, 4 to 15, 4 to 12, 4 to 10, 4 to 8, 4 to 6, or 4 to 5 nt). In some embodiments, the linker of a single guide RNA can have a length of from 3 to 100 nucleotides (nt) (e.g., from 3 to 80, 3 to 50, 3 to 30, 3 to 25, 3 to 20, 3 to 15, 3 to 12, 3 to 10, 3 to 8, 3 to 6, 3 to 5, 3 to 4, 4 to 100, 4 to 80, 4 to 50, 4 to 30, 4 to 25, 4 to 20, 4 to 15, 4 to 12, 4 to 10, 4 to 8, 4 to 6, or 4 to 5 nt). In some embodiments, the linker of a single guide RNA can have a length of from 3 to 10 nucleotides (nt) (e.g., from 3 to 9, 3 to 8, 3 to 7, 3 to 6, 3 to 5, 3 to 4, 4 to 10, 4 to 9, 4 to 8, 4 to 7, 4 to 6, or 4 to 5 nt).

### Guide Sequence of a GeoCas Guide RNA

The targeting segment of a subject guide RNA includes a guide sequence (i.e., a targeting sequence), which is a nucleotide sequence that is complementary to a sequence (a target site) in a target nucleic acid. In other words, the targeting segment of a guide RNA can interact with a target nucleic acid (e.g., double stranded DNA (dsDNA), single stranded DNA (ssDNA), single stranded RNA (ssRNA), or double stranded RNA (dsRNA)) in a sequence-specific manner via hybridization (i.e., base pairing). The guide sequence of a guide RNA can be modified (e.g., by genetic engineering)/designed to hybridize to any desired target sequence (e.g., while taking the PAM into account, e.g., when targeting a dsDNA target) within a target nucleic acid (e.g., a eukaryotic target nucleic acid such as genomic DNA).

In some cases, the percent complementarity between the guide sequence and the target site of the target nucleic acid is 60% or more (e.g., 65% or more, 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid is 80% or more (e.g., 85% or more, 90% or more, 95% or more, 97% or more, 98% or more, 99% or more, or 100%). In some cases, the percent complementarity between the guide sequence and the target site of the target nucleic acid is 90% or more (e.g., 95% or more, 97% or more, 98% or more, 99% or more, or 100%). In some cases, the percent complementarity between the guide sequence and the target site of the target nucleic acid is 100%.

In some cases, the percent complementarity between the guide sequence and the target site of the target nucleic acid is 100% over the seven contiguous 3′-most nucleotides of the target site of the target nucleic acid.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target site of the target nucleic acid is 90% or more (e.g., 95% or more, 97% or more, 98% or more, 99% or more, or 100%) over 19 or more (e.g., 20 or more, 21 or more, 22 or more) contiguous nucleotides. In some cases, the percent complementarity between the guide sequence and the target site of the target nucleic acid is 100% over 19 or more (e.g., 20 or more, 21 or more, 22 or more) contiguous nucleotides.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 19-25 contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 19-25 contiguous nucleotides. In some cases, the percent complementarity between the guide sequence and the target site of the target nucleic acid is 90% or more (e.g., 95% or more, 97% or more, 98% or more, 99% or more, or 100%) over 19-25 contiguous nucleotides. In some cases, the percent complementarity between the guide sequence and the target site of the target nucleic acid is 100% over 19-25 contiguous nucleotides.

In some cases, the guide sequence has a length in a range of from 19-30 nucleotides (nt) (e.g., from 19-25, 19-22, 19-20, 20-30, 20-25, or 20-22 nt). In some cases, the guide sequence has a length in a range of from 19-25 nucleotides (nt) (e.g., from 19-22, 19-20, 20-25, 20-25, or 20-22 nt). In some cases, the guide sequence has a length of 19 or more nt (e.g., 20 or more, 21 or more, or 22 or more nt; 19 nt, 20 nt, 21 nt, 22 nt, 23 nt, 24 nt, 25 nt, etc.). In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt.

The same description above applies to the guide sequence of any guide RNA used herein, e.g., a ThermoCas9 guide RNA.

### Protein-Binding Segment of a Guide RNA

The protein-binding segment of a subject guide RNA interacts with a variant CRISPR-Cas effector polypeptide (or a CRISPR-Cas polypeptide (e.g., ThermoCas9 variant) of the present disclosure). The guide RNA guides the bound variant CRISPR-Cas effector polypeptide (or a CRISPR-Cas polypeptide (e.g., ThermoCas9 variant) of the present disclosure) to a specific nucleotide sequence within target nucleic acid via the above mentioned guide sequence. The protein-binding segment of a guide RNA comprises two stretches of nucleotides (the duplex-forming segment of the activator and the duplex-forming segment of the targeter) that are complementary to one another and hybridize to form a double stranded RNA duplex (dsRNA duplex). Thus, the protein-binding segment includes a dsRNA duplex.

In some cases, the dsRNA duplex region formed between the activator and targeter (i.e., the activator/targeter dsRNA duplex) (e.g., in dual or single guide RNA format) includes a range of from 8-25 base pairs (bp) (e.g., from 8-22, 8-18, 8-15, 8-12, 12-25, 12-22, 12-18, 12-15, 13-25, 13-22, 13-18, 13-15, 14-25, 14-22, 14-18, 14-15, 15-25, 15-22, 15-18, 17-25, 17-22, or 17-18 bp, e.g., 15 bp, 16 bp, 17 bp, 18 bp, 19 bp, 20 bp, 21 bp, etc.). In some cases, the duplex region (e.g., in dual or single guide RNA format) includes 8 or more bp (e.g., 10 or more, 12 or more, 15 or more, or 17 or more bp). In some cases, not all nucleotides of the duplex region are paired, and therefore the duplex forming region can include a bulge. The term “bulge” herein is used to mean a stretch of nucleotides (which can be one nucleotide) that do not contribute to a double stranded duplex, but which are surround 5′ and 3′ by nucleotides that do contribute, and as such a bulge is considered part of the duplex region. In some cases, the dsRNA duplex formed between the activator and targeter (i.e., the activator/targeter dsRNA duplex) includes 1 or more bulges (e.g., 2 or more, 3 or more, 4 or more bulges). In some cases, the dsRNA duplex formed between the activator and targeter (i.e., the activator/targeter dsRNA duplex) includes 2 or more bulges (e.g., 3 or more, 4 or more bulges). In some cases, the dsRNA duplex formed between the activator and targeter (i.e., the activator/targeter dsRNA duplex) includes 1-5 bulges (e.g., 1-4, 1-3, 2-5, 2-4, or 2-3 bulges).

Thus, in some cases, the duplex-forming segments of the activator and targeter have 70%-100% complementarity (e.g., 75%-100%, 80%-10%, 85%-100%, 90%-100%, 95%-100% complementarity) with one another. In some cases, the duplex-forming segments of the activator and targeter have 70%-100% complementarity (e.g., 75%-100%, 80%-10%, 85%-100%, 90%-100%, 95%-100% complementarity) with one another. In some cases, the duplex-forming segments of the activator and targeter have 85%-100% complementarity (e.g., 90%-100%, 95%-100% complementarity) with one another. In some cases, the duplex-forming segments of the activator and targeter have 70%-95% complementarity (e.g., 75%-95%, 80%-95%, 85%-95%, 90%-95% complementarity) with one another.

In other words, in some cases, the dsRNA duplex formed between the activator and targeter (i.e., the activator/targeter dsRNA duplex) includes two stretches of nucleotides that have 70%-100% complementarity (e.g., 75%-100%, 80%-10%, 85%-100%, 90%-100%, 95%-100% complementarity) with one another. In some cases, the activator/targeter dsRNA duplex includes two stretches of nucleotides that have 85%-100% complementarity (e.g., 90%-100%, 95%-100% complementarity) with one another. In some cases, the activator/targeter dsRNA duplex includes two stretches of nucleotides that have 70%-95% complementarity (e.g., 75%-95%, 80%-95%. 85%-95%, 90%-95% complementarity) with one another.

The duplex region of a subject guide RNA (in dual guide or single guide RNA format) can include one or more (1, 2, 3, 4, 5, etc.) mutations relative to a naturally occurring duplex region. For example, in some cases a base pair can be maintained while the nucleotides contributing to the base pair from each segment (targeter and activator) can be different. In some cases, the duplex region of a subject guide RNA includes more paired bases, less paired bases, a smaller bulge, a larger bulge, fewer bulges, more bulges, or any convenient combination thereof, as compared to a naturally occurring duplex region (of a naturally occurring guide RNA).

In some cases, the activator (e.g., activator-RNA) of a subject guide RNA (in dual or single guide RNA format) includes at least two internal RNA duplexes (i.e., two internal hairpins in addition to the activator/targeter dsRNA). The internal RNA duplexes (hairpins) of the activator can be positioned 5′ of the activator/targeter dsRNA duplex. In some cases, the activator includes one hairpin positioned 5′ of the activator/targeter dsRNA duplex. In some cases, the activator includes two hairpins positioned 5′ of the activator/targeter dsRNA duplex. In some cases, the activator includes three hairpins positioned 5′ of the activator/targeter dsRNA duplex. In some cases, the activator includes two or more hairpins (e.g., 3 or more or 4 or more hairpins) positioned 5′ of the activator/targeter dsRNA duplex. In some cases, the activator includes 2 to 5 hairpins (e.g., 2 to 4, or 2 to 3 hairpins) positioned 5′ of the activator/targeter dsRNA duplex.

In some cases, the activator-RNA (e.g., in dual or single guide RNA format) comprises at least 2 nucleotides (nt) (e.g., at least 3 or at least 4 nt) 5′ of the 5′-most hairpin stem. In some cases, the activator-RNA (e.g., in dual or single guide RNA format) comprises at least 4 nt 5′ of the 5′-most hairpin stem.

In some cases, the activator-RNA (e.g., in dual or single guide format) has a length of 65 nucleotides (nt) or more (e.g., 66 or more, 67 or more, 68 or more, 69 or more, 70 or more, or 75 or more nt). In some cases, the activator-RNA (e.g., in dual or single guide format) has a length of 66 nt or more (e.g., 67 or more, 68 or more, 69 or more, 70 or more, or 75 or more nt). In some cases, the activator-RNA (e.g., in dual or single guide format) has a length of 67 nt or more (e.g., 68 or more, 69 or more, 70 or more, or 75 or more nt). In some cases, the activator-RNA has a length of from 80 nt to 100 nt. In some cases, the activator-RNA has a length of 80 nt, 81 nt, 82 nt, 83 nt, 84 nt, 85 nt, 86 nt, 87 nt, 88 nt, 89 nt, 90 nt, 91 nt, 92 nt, 93 nt, 94 nt, 95 nt, 96 nt, 97 nt, 98 nt, 99 nt, or 100 nt (or more than 100 nt).

In some cases, the activator-RNA (e.g., in dual or single guide format) includes 45 or more nucleotides (nt) (e.g., 46 or more, 47 or more, 48 or more, 49 or more, 50 or more, 51 or more, 52 or more, 53 or more, 54 or more, or 55 or more nt) 5′ of the dsRNA duplex formed between the activator and the targeter (the activator/targeter dsRNA duplex). In some cases, the activator is truncated at the 5′ end relative to a naturally occurring activator. In some cases, the activator is extended at the 5′ end relative to a naturally occurring activator.

In some cases, the tracrRNA (e.g., the portion of the activator-RNA that does not include the duplex-forming segments) has a length of at least 75 nucleotides (nt). In some cases, the tracrRNA has a length of from 75 nt to 100 nt. In some cases, the tracrRNA has a length of 75 nt. In some cases, the tracrRNA has a length of 75 nt, 76 nt, 77 nt, 78 nt, 79 nt, 80 nt, 81 nt, 82 nt, 83 nt, 84 nt, or 85 nt (or more than 85 nt). In a single-molecule guide RNA (sgRNA), the tracrRNA can be considered the nucleotide sequence that is 3′ of the duplex-forming segment. For example, in some cases, a sgRNA of the present disclosure comprises, in order from 5′ to 3′: i) a “spacer” nucleotide sequence (having a length of from 15 to 25 nt) that hybridizes with (has complementarity to) a target nucleotide sequence in a target nucleic acid); ii) a first duplex forming segment comprising a first stretch of complementary nucleotides (where the first duplex-forming segment has a length of from 10 nucleotides to 25 nucleotides, or more than 25 nucleotides); iii) a linker; iv) a second duplex-forming segment comprising a second stretch of complementary nucleotides (where the second duplex-forming segment has a length that is the same, or nearly the same, as the first duplex-forming segment), where the first duplex-forming segment and the second duplex-forming segment hybridize to one another via the first and second stretches of complementary nucleotides to form a double stranded RNA (dsRNA) duplex, where the two complementary stretches of nucleotides are covalently linked to one another via the linker; and v) a tracrRNA, where the tracrRNA has a length of from about 75 nt to 100 nt, or more than 100 nt.

The term “activator” or “activator RNA” is used herein to mean a tracrRNA-like molecule (tracrRNA: “trans-acting CRISPR RNA”) of a dual guide RNA (and therefore of a single guide RNA when the “activator” and the “targeter” are linked together by, e.g., intervening nucleotides). Thus, for example, a guide RNA (dgRNA or sgRNA) comprises an activator sequence (e.g., a tracrRNA sequence). A tracr molecule (a tracrRNA) is a naturally existing molecule that hybridizes with a CRISPR RNA molecule (a crRNA) to form a dual guide RNA. The term “activator” is used herein to encompass naturally existing tracrRNAs, but also to encompass tracrRNAs with modifications (e.g., truncations, extensions, sequence variations, base modifications, backbone modifications, linkage modifications, etc.) where the activator retains at least one function of a tracrRNA (e.g., contributes to the dsRNA duplex to which protein binds). In some cases, the activator provides one or more stem loops that can interact with protein. An activator can be referred to as having a tracr sequence (tracrRNA sequence) and in some cases is a tracrRNA, but the term “activator” is not limited to naturally existing tracrRNAs.

The term “targeter” or “targeter RNA” is used herein to refer to a crRNA-like molecule (crRNA: “CRISPR RNA”) of a dual guide RNA (and therefore of a single guide RNA when the “activator” and the “targeter” are linked together, e.g., by intervening nucleotides). Thus, for example, a guide RNA (dgRNA or sgRNA) comprises a guide sequences and a duplex-forming segment (e.g., a duplex forming segment of a crRNA, which can also be referred to as a crRNA repeat). Because the sequence of a targeting segment (the segment that hybridizes with a target sequence of a target nucleic acid) of a targeter is modified by a user to hybridize with a desired target nucleic acid, the sequence of a targeter will often be a non-naturally occurring sequence. However, the duplex-forming segment of a targeter (described in more detail herein), which hybridizes with the duplex-forming segment of an activator, can include a naturally existing sequence (e.g., can include the sequence of a duplex-forming segment of a naturally existing crRNA, which can also be referred to as a crRNA repeat). Thus, the term targeter is used herein to distinguish from naturally occurring crRNAs, despite the fact that part of a targeter (e.g., the duplex-forming segment) often includes a naturally occurring sequence from a crRNA. However, the term “targeter” encompasses naturally occurring crRNAs.

As noted above, a targeter comprises both the guide sequence of the guide RNA and a stretch (a “duplex-forming segment”) of nucleotides that forms one half of the dsRNA duplex of the protein-binding segment of the guide RNA. A corresponding tracrRNA-like molecule (activator) comprises a stretch of nucleotides (a duplex-forming segment) that forms the other half of the dsRNA duplex of the protein-binding segment of the guide RNA. In other words, a stretch of nucleotides of the targeter is complementary to and hybridizes with a stretch of nucleotides of the activator to form the dsRNA duplex of the protein-binding segment of a guide RNA. As such, each targeter can be said to have a corresponding activator (which has a region that hybridizes with the targeter). The targeter molecule additionally provides the guide sequence. Thus, a targeter and an activator (as a corresponding pair) hybridize to form a guide RNA. The particular sequence of a given naturally existing crRNA or tracrRNA molecule can be characteristic of the species in which the RNA molecules are found..

### Example Guide RNA Sequences

In some cases, a single-guide RNA comprises, in order from 5′ to 3′: i) a spacer (e.g., a nucleotide sequence that hybridizes to (binds to) a target nucleotide sequence in a target nucleic acid) having a length of from 18 nt to 25 nt (e.g., 18 nt. 19 nt, 20 nt, 21 nt, 22 tt, 23 nt, 24 nt, or 25 nt); ii) a crRNA (duplex-forming RNA segment) comprising two complementary stretches of nucleotides that hybridize to form a double-stranded RNA duplex, where each of the two complementary stretches of nucleotides has a length of from about 19 nt to 25 nt (e.g., 19 nt, 20 nt, 21 nt, 22, nt, 23 nt, 24 nt, or 25 nt); and iii) a tracrRNA (e.g., RNA segment not including the duplex-forming crRNA) having a length of at least 75 nt, e.g., having a length of from 75 nt to 100 nt, e.g., 75 nt, 76 nt, 77 nt, 78 nt, 79 nt, 80 nt, 81 nt, 82 nt, 83 nt, 84 nt, 85 nt. 86 nt, 87 nt, 88 nt, 89 nt, 90 nt. 91 nt, 92 nt, 93 nt, 94 nt, 95 nt, 96 nt. 97 nt, 98 nt. 99 nt, or 100 nt. The crRNA can comprise two complementary stretches of nucleotides that hybridize to form a double-stranded RNA duplex, where the two complementary stretches of nucleotides are covalently linked by intervening nucleotides, e.g., are covalently linked by a linker having a length of from 4 nt to 50 nt (or more than 50 nt), e.g., having a length of 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, from 20 nt to 25 nt, from 25 nt to 30 nt, from 30 nt to 40 nt, from 40 nt to 50 nt, or more than 50 nt). In some cases, the linker has a length of 4 nt.

In some cases, a single-guide RNA comprises a tracrRNA comprising a nucleotide sequence having at least 80%, at least 90%, at least 95%, at least 98%, at least 99%, or 100%, nucleotide sequence identity to the following nucleotide sequence:

(SEQ ID NO: 37)
UCAGGGUUACUAUGAUAAGGGCUUUCUGCCUAAGGCAGACUGAC
CCGCGGCGUUGGGGAUCGCCUGUCGCCCGCUUUUGGCGGGCAUU
CCCCAUCCUU.

In some cases, a single-guide RNA comprises a tracrRNA comprising a nucleotide sequence having at least 80%, at least 90%, at least 95%, at least 98%, at least 99%, or 100%, nucleotide sequence identity to the following nucleotide sequence:

(SEQ ID NO: 38)
AAGGGCUUUCUGCCUAAGGCAGACUGACCCGCGGCGUUGGGGAUC
GCCUGUCGCCCGCUUUUGGCGGGCAUUCCCCAUCCUU.

In some cases, a single-guide RNA comprises a tracrRNA comprising a nucleotide sequence having no more than 1 nucleotide (nt), no more than 2 nt, no more than 3 nt, no more than 4 nt, no more than 5 nt, no more than 6 nt, no more than 7 nt, no more than 8 nt, no more than 9 nt, no more than 10 nt, no more than 11 nt, no more than 12 nt, no more than 13 nt, no more than 14 nt, no more than 15 nt, no more than 16 nt, no more than 17 nt, no more than 18 nt, no more than 19 nt, or no more than 20 nt, differences from the following nucleotide sequence:

(SEQ ID NO: 37)
UCAGGGUUACUAUGAUAAGGGCUUUCUGCCUAAGGCAGACUGACC
CGCGGCGUUGGGGAUCGCCUGUCGCCCGCUUUUGGCGGGCAUUCC
CCAUCCUU.

In some cases, a single-guide RNA comprises a tracrRNA comprising a nucleotide sequence having no more than 1 nucleotide (nt), no more than 2 nt, no more than 3 nt, no more than 4 nt, no more than 5 nt, no more than 6 nt, no more than 7 nt, no more than 8 nt, no more than 9 nt, no more than 10 nt, no more than 11 nt, no more than 12 nt, no more than 13 nt, no more than 14 nt, no more than 15 nt, no more than 16 nt, no more than 17 nt, no more than 18 nt, no more than 19 nt, or no more than 20 nt, differences from the following nucleotide sequence: nucleotide sequence identity to the following nucleotide sequence:

(SEQ ID NO: 38)
AAGGGCUUUCUGCCUAAGGCAGACUGACCCGCGGCGUUGGGGAUC
GCCUGUCGCCCGCUUUUGGCGGGCAUUCCCCAUCCUU.

As one non-limiting example, a single-guide RNA comprises the following nucleotide sequence:

(SEQ ID NO: 39)
AGACUGACCCGCGGCGUUGGGGAUCGCCUGUCGCCCGCUUUU
GGCGGGCAUUCCCCAUCC(spacer)GUCAUAGUUCCCCUGA
gaaaUCAGGGUUACUAUGAUAAGGGCUUUCUGCCUAAGGCUU

where: i) the “spacer” comprises a nucleotide sequence that hybridizes to a target nucleotide sequence in a target nucleic acid; ii) the double-underlined regions are the duplex-forming crRNA; iii) the underlined and bolded “gaaa” sequence is the linker (or “tetraloop” in this example); and iv) the bolded sequence (without underlining) is the tracrRNA.

### Heterologous PI

In some cases, a variant CRISPR-Cas effector polypeptide (or a CRISPR-Cas polypeptide (e.g., ThermoCas9 variant) of the present disclosure) comprises a heterologous protospacer adjacent motif (PAM) interacting domain. In some cases, the heterologous PI domain comprises an amino acid sequence having at least 50% amino acid sequence identity to the following amino acid sequence: IIKTAGGEEIKIKDLFAYYKTIHSGTAGLELVSHDCSFSLSGVGSRTLKRFEKYQVDVLGNIYKVR GEKRVGLASSAHSKTGETIRPLQSTRD (SEQ ID NO:40). In some cases, the heterologous PI domain comprises an amino acid sequence having less than 83% amino acid sequence identity to the following amino acid sequence:

(SEQ ID NO: 41)
TVKTAAGEEINVKDVFVYYKTIDSANGGLELISHDHRFSLRGVGS
RTLKRFEKYQVDVLGNIYKVRGEKRVGLASSAHSKPGKTIRPLQS
TRD.

### CRISPR-Cas Effector Fusion Polypeptides

As noted above, in some cases, a variant CRISPR-Cas effector polypeptide (or a CRISPR-Cas polypeptide (e.g., ThermoCas9 variant) of the present disclosure) of the present disclosure is a fusion polypeptide (also referred to herein as “a variant CRISPR-Cas effector fusion polypeptide” or, simply a “CRISPR-Cas effector fusion polypeptide”) that comprises: i) a variant CRISPR-Cas effector polypeptide of the present disclosure (or a CRISPR-Cas polypeptide (e.g., ThermoCas9 variant) of the present disclosure); and ii) one or more heterologous polypeptides. The term “heterologous polypeptide” is used interchangeably herein with “fusion partner.”

In some cases, the fusion partner (heterologous polypeptide) is a reverse transcriptase. In some cases, the fusion partner (heterologous polypeptide) is a deaminase. In some cases, the fusion partner is a nuclear localization signal (NLS).

### Reverse Transcriptases

In some cases, a variant CRISPR-Cas effector fusion polypeptide (or a CRISPR-Cas polypeptide (e.g., ThermoCas9 variant) of the present disclosure) comprises: i) a variant CRISPR-Cas effector polypeptide (or a CRISPR-Cas polypeptide (e.g., ThermoCas9 variant) of the present disclosure); and ii) one or more heterologous polypeptides (one or more “fusion partners”), where at least one of the one or more heterologous polypeptides is a reverse transcriptase. Such a variant CRISPR-Cas effector fusion polypeptide (or a CRISPR-Cas polypeptide (e.g., ThermoCas9 variant) of the present disclosure) optionally also includes one or more NLSs. In some cases, the CRISPR-Cas effector polypeptide is catalytically inactive. In some cases, the CRISPR-Cas effector polypeptide is a nickase (e.g., cleaves only one strand of a double-stranded target DNA). Reverse transcriptases are known in the art; see, e.g., Cot6 and Roth (2008) Virus Res. 134:186. Suitable reverse transcriptases include, e.g., a murine leukemia virus reverse transcriptase; a Rous sarcoma virus reverse transcriptase; a human immunodeficiency virus type I reverse transcriptase; a Moloney murine leukemia virus reverse transcriptase; a transcription xenopolymerase (RTX); avian mycloblastosis virus reverse transcriptase (AMV-RT); a Eubacteriun rectale maturase reverse transcriptase (Marathon®; and the like. The reverse transcriptase fusion partner can include one or more mutations. For example, in some cases, the reverse transcriptase is a M-MLV reverse transcriptase polypeptide that comprises one or more mutations selected from the group consisting of D200N, T306K, W313F, T330P and L603W.

A CRISPR-Cas effector fusion polypeptide comprising: i) a CRISPR-Cas effector polypeptide, where the CRISPR-Cas effector polypeptide is a nickase; and ii) a reverse transcriptase, is referred as a “prime editor” (“PE”). In some cases, the CRISPR-Cas effector polypeptide is a Cas9 polypeptide comprising an H840A substitution. In some cases, the CRISPR-Cas effector polypeptide is a Cas12a/b nickase. In some cases, the reverse transcriptase is a pentamutant of M-MLV RT (e.g., comprising the following substitutions: D200N/L603W/T330P/T306K/W313F) (where D200, L603, T330, T306, and W313 correspond to D199, L602, T329, T305, and W312 of the M-MLV RT amino acid sequence depicted in FIG. 26).

In some cases, a suitable reverse transcriptase comprises an amino acid sequence having at least 50%, at least 60%, at least 70%, at least 80%, at least 85%, at least 90%, at least 95%, at least 98%, at least 99%, or 100%, amino acid sequence identity to the reverse transcriptase amino acid sequence depicted in FIG. 26.

### Base Editors

In some cases, a CRISPR-Cas effector fusion polypeptide comprises: i) a CRISPR-Cas effector polypeptide; and ii) one or more heterologous polypeptides (one or more “fusion partners”), where at least one of the one or more heterologous polypeptides is a deaminase. Such a CRISPR-Cas effector fusion polypeptide optionally also includes one or more NLSs. In some cases, the CRISPR-Cas effector polypeptide is catalytically inactive. In some cases, the CRISPR-Cas effector polypeptide is a nickase (e.g., cleaves only one strand of a double-stranded target DNA). Suitable base editors include, e.g., an adenosine deaminase; a cytidine deaminase (e.g., an activation-induced cytidine deaminase (AID)); APOBEC3G; and the like); and the like. A suitable adenosine deaminase is any enzyme that is capable of deaminating adenosine in DNA. In some cases, the deaminase is a TadA deaminase.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 42)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTD

In some cases, a suitable adenosine deaminase comprises an amino acid sequence having PGP28,DNA at least 80%, at least 85%, at least 90%, at least 95%, at least 98%, at least 99%, or 100%, amino acid sequence identity to the following amino acid sequence:

(SEQ ID NO: 43)
MRRAFITGVFFLSEVEFSHEYWMRHALTLAKRAWDEREVPVGAVL
VHNNRVIGEGWNRPIGRHDPTAHAEIMALRQGGLVMQNYRLIDAT
LYVTLEPCVMCAGAMIHSRIGRVVFGARDAKTGAAGSLMDVLHHP
GMNHRVEITEGILADECAALLSDFFRMRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following Staphylococcus aureus TadA amino acid sequence:

(SEQ ID NO: 44)
MGSHMTNDIYFMTLAIEEAKKAAQLGEVPIGAIITKDDEVIARAH
NLRETLQQPTAHAEHIAIERAAKVLGSWRLEGCTLYVTLEPCVMC
AGTIVMSRIPRVVYGADDPKGGCSGSLMNLLQQSNFNHRAIVDKG
VLKEACSTLLTTFFKNLRANKKSTN:

In some cases, a suitable adenosine deaminase comprises an amino acid sequence having at least 80%, at least 85%, at least 90%, at least 95%, at least 98%, at least 99%, or 100%, amino acid sequence identity to the following Bacillus subtilis TadA amino acid sequence:

(SEQ ID NO: 45)
MTQDELYMKEAIKEAKKAEEKGEVPIGAVLVINGEIIARAHNLRE
TEQRSIAHAEMLVIDEACKALGTWRLEGATLYVTLEPCPMCAGAV
VLSRVEKVVFGAFDPKGGCSGTLMNLLQEERFNHQAEVVSGVLEE
ECGGMLSAFFRELRKKKKAARKNLSE

In some cases, a suitable adenosine deaminase comprises an amino acid sequence having at least 80%, at least 85%, at least 90%, at least 95%, at least 98%, at least 99%, or 100%, amino acid sequence identity to the following Salmonella typhimurium TadA:

(SEQ ID NO: 46)
MPPAFITGVTSLSDVELDHEYWMRHALTLAKRAWDEREVPVGAVL
VHNHRVIGEGWNRPIGRHDPTAHAEIMALRQGGLVLQNYRLLDTT
LYVTLEPCVMCAGAMVHSRIGRVVFGARDAKTGAAGSLIDVLHHP
GMNHRVEIIEGVLRDECATLLSDFFRMRRQEIKALKKADRAEGAG
PAV

In some cases, a suitable adenosine deaminase comprises an amino acid sequence having at least 80%, at least 85%, at least 90%, at least 95%, at least 98%, at least 99%, or 100%, amino acid sequence identity to the following Shewanella putrefaciens TadA amino acid sequence:

(SEQ ID NO: 47)
MDEYWMQVAMQMAEKAEAAGEVPVGAVLVKDGQQIATGYNLSISQ
HDPTAHAEILCLRSAGKKLENYRLLDATLYITLEPCAMCAGAMVH
SRIARVVYGARDEKTGAAGTVVNLLQHPAFNHQVEVTSGVLAEAC
SAQLSRFFKRRRDEKKALKLAQRAQQGIE

In some cases, a suitable adenosine deaminase comprises an amino acid sequence having at least 80%, at least 85%, at least 90%, at least 95%, at least 98%, at least 99%, or 100%, amino acid sequence identity to the following Haemophilus influenzae F3031 TadA amino acid sequence:

(SEQ ID NO: 48)
MDAAKVRSEFDEKMMRYALELADKAEALGEIPVGAVLVDDARNII
GEGWNLSIVQSDPTAHAEIIALRNGAKNIQNYRLLNSTLYVTLEP
CTMCAGAILHSRIKRLVFGASDYKTGAIGSRFHFFDDYKMNHTLE
ITSGVLAEECSQKLSTFFQKRREEKKIEKALLKSLSDK

In some cases, a suitable adenosine deaminase comprises an amino acid sequence having at least 80%, at least 85%, at least 90%, at least 95%, at least 98%, at least 99%, or 100%, amino acid sequence identity to the following Caulobacter crescentus TadA amino acid sequence:

(SEQ ID NO: 49)
MRTDESEDQDHRMMRLALDAARAAAEAGETPVGAVILDPSTGEVI
ATAGNGPIAAHDPTAHAEIAAMRAAAAKLGNYRLTDLTLVVTLEP
CAMCAGAISHARIGRVVFGADDPKGGAVVHGPKFFAQPTCHWRPE
VTGGVLADESADLLRGFFRARRKAKI

In some cases, a suitable adenosine deaminase comprises an amino acid sequence having at least 80%, at least 85%, at least 90%, at least 95%, at least 98%, at least 99%, or 100%, amino acid sequence identity to the following Geobacter sulfurreducens TadA amino acid sequence:

(SEQ ID NO: 50)
MSSLKKTPIRDDAYWMGKAIREAAKAAARDEVPIGAVIVRDGAVI
GRGHNLREGSNDPSAHAEMIAIRQAARRSANWRLTGATLYVTLEP
CLMCMGAIILARLERVVFGCYDPKGGAAGSLYDLSADPRLNHQVR
LSPGVCQEECGTMLSDFFRDLRRRKKAKATPALFIDERKVPPEP

Cytidine deaminases suitable for inclusion in a Cas12L fusion polypeptide include any enzyme that is capable of deaminating cytidine in DNA.

In some cases, the cytidine deaminase is a deaminase from the apolipoprotein B mRNA-editing complex (APOBEC) family of deaminases. In some cases, the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In some cases, the cytidine deaminase is an activation induced deaminase (AID).

In some cases, a suitable cytidine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 51)
MDSLLMNRRKFLYQFKNVRWAKGRRETYLCYVVKRRDSATSFSLD
FGYLRNKNGCHVELLFLRYISDWDLDPGRCYRVTWFTSWSPCYDC
ARHVADFLRGNPNLSLRIFTARLYFCEDRKAEPEGLRRLHRAGVQ
IAIMTFKDYFYCWNTFVENHERTFKAWEGLHENSVRLSRQLRRIL
LPLYEVDDLRDAFRTLGL

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKENHERTFK AWEGLHENSV RLSRQLRRIL LPLYEVDDLR DAFRTLGL (SEQ ID NO:52).

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKDYFYCWNT FVENHERTFK AWEGLHENSV RLSRQLRRIL LPLYEVDDLR DAFRTLGL (SEQ ID NO:53).

### NLSs and CPPs

In some cases, a heterologous polypeptide (a fusion partner) provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some cases, a CRISPR-Cas effector fusion polypeptide does not include an NLS so that the protein is not targeted to the nucleus (which can be advantageous, e.g., when the target nucleic acid is an RNA that is present in the cytosol). In some cases, the heterologous polypeptide can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases, a variant CRISPR-Cas effector protein (or a CRISPR-Cas polypeptide (e.g., ThermoCas9 variant) of the present disclosure) includes (is fused to) a nuclear localization signal (NLS) (e.g., in some cases 2 or more, 3 or more, 4 or more, or 5 or more NLSs). Thus, in some cases, a CRISPR-Cas effector polypeptide includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

In some cases, a variant CRISPR-Cas effector polypeptide (or a CRISPR-Cas polypeptide (e.g., ThermoCas9 variant) of the present disclosure) includes (is fused to) between 1 and 10 NLSs (e.g., 1-9, 1-8, 1-7, 1-6, 1-5, 2-10, 2-9, 2-8, 2-7, 2-6, or 2-5 NLSs). In some cases, a CRISPR-Cas effector protein includes (is fused to) between 2 and 5 NLSs (e.g., 2-4, or 2-3 NLSs).

Non-limiting examples of NLSs include an NLS sequence derived from: the NLS of the SV40 virus large T-antigen, having the amino acid sequence PKKKRKV (SEQ ID NO:130); the NLS from nucleoplasmin (e.g., the nucleoplasmin bipartite NLS with the sequence KRPAATKKAGQAKKKK (SEQ ID NO:131)); the c-myc NLS having the amino acid sequence PAAKRVKLD (SEQ ID NO:132) or RQRRNELKRSP (SEQ ID NO:133); the hRNPA1 M9 NLS having the sequence NQSSNFGPMKGGNFGGRSSGPYGGGGQYFAKPRNQGGY (SEQ ID NO:134); the sequence RMRIZFKNKGKDTAELRRRRVEVSVELRKAKKDEQILKRRNV (SEQ ID NO:135) of the IBB domain from importin-alpha; the sequences VSRKRPRP (SEQ ID NO: 136) and PPKKARED (SEQ ID NO:137) of the myoma T protein; the sequence PQPKKKPL (SEQ ID NO:138) of human p53; the sequence SALIKKKKKMAP (SEQ ID NO:139) of mouse c-abl IV; the sequences DRLRR (SEQ ID NO:140) and PKQKKRK (SEQ ID NO:141) of the influenza virus NS1; the sequence RKLKKKIKKL (SEQ ID NO:142) of the Hepatitis virus delta antigen; the sequence REKKKFLKRR (SEQ ID NO:143) of the mouse Mx1 protein; the sequence KRKGDEVDGVDEVAKKKSKK (SEQ ID NO:144) of the human poly(ADP-ribose) polymerase; and the sequence RKCLQAGMNLEARKTKK (SEQ ID NO:145) of the steroid hormone receptors (human) glucocorticoid. In general, NLS (or multiple NLSs) are of sufficient strength to drive accumulation of the CRISPR-Cas effector protein in a detectable amount in the nucleus of a eukaryotic cell. Detection of accumulation in the nucleus may be performed by any suitable technique. For example, a detectable marker may be fused to the CRISPR-Cas effector protein such that location within a cell may be visualized. Cell nuclei may also be isolated from cells, the contents of which may then be analyzed by any suitable process for detecting protein, such as immunohistochemistry. Western blot, or enzyme activity assay. Accumulation in the nucleus may also be determined indirectly.

In some cases, a variant CRISPR-Cas effector fusion polypeptide (or a CRISPR-Cas polypeptide (e.g., ThermoCas9 variant) of the present disclosure) includes a “Protein Transduction Domain” or PTD (also known as a CPP—cell penetrating peptide), which refers to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus a polypeptide (e.g., linked to a variant CRISPR-Cas effector protein (or a CRISPR-Cas polypeptide (e.g., ThermoCas9 variant) of the present disclosure) such as a dCRISPR-Cas effector, a nickase CRISPR-Cas effector, or a fusion CRISPR-Cas effector protein, to generate a fusion protein). In some embodiments, a PTD is covalently linked to the carboxyl terminus of a polypeptide (e.g., linked to a variant CRISPR-Cas effector protein (or a CRISPR-Cas polypeptide (e.g., ThermoCas9 variant) of the present disclosure) such as a dCRISPR-Cas effector protein, a nickase CRISPR-Cas effector, or a fusion CRISPR-Cas effector protein to generate a fusion protein). In some cases, the PTD is inserted internally in the CRISPR-Cas effector fusion polypeptide (i.e., is not at the N- or C-terminus of the CRISPR-Cas effector fusion polypeptide) at a suitable insertion site. In some cases, a subject CRISPR-Cas effector fusion polypeptide includes (is conjugated to, is fused to) one or more PTDs (e.g., two or more, three or more, four or more PTDs). In some cases, a PTD includes a nuclear localization signal (NLS) (e.g., in some cases 2 or more, 3 or more, 4 or more, or 5 or more NLSs). Thus, in some cases, a CRISPR-Cas effector fusion polypeptide includes one or more NLSs (e.g., 2 or more, 3 or more, 4 or more, or 5 or more NLSs). In some embodiments, a PTD is covalently linked to a nucleic acid (e.g., a CRISPR-Cas effector guide nucleic acid, a polynucleotide encoding a CRISPR-Cas effector guide nucleic acid, a polynucleotide encoding a CRISPR-Cas effector fusion polypeptide, a donor polynucleotide, etc.). Examples of PTDs include but are not limited to a minimal undecapeptide protein transduction domain (corresponding to residues 47-57 of HIV-1 TAT comprising YGRKKRRQRRR; SEQ ID NO:146); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:147); Transportan GWTLNSAGYLLGKINLKALAALAKKIL (SEQ ID NO:148); KALAWEAKLAKALAKALAKHLAKALAKALKCEA (SEQ ID NO:149); and RQIKIWFQNRRMKWKK (SEQ ID NO:150). Exemplary PTDs include but are not limited to, YGRKKRRQRRR (SEQ ID NO:146), RKKRRQRRR (SEQ ID NO:151); an arginine homopolymer of from 3 arginine residues to 50 arginine residues; Exemplary PTD domain amino acid sequences include, but are not limited to, any of the following: YGRKKRRQRRR (SEQ ID NO:146); RKKRRQRR (SEQ ID NO:152); YARAAARQARA (SEQ ID NO:153); THRLPRRRRRR (SEQ ID NO:154); and GGRRARRRRRR (SEQ ID NO:155). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### Linkers (e.g., for Fusion Partners)

In some embodiments, a variant CRISPR-Cas effector polypeptide (or a CRISPR-Cas polypeptide (e.g., ThermoCas9 variant) of the present disclosure) can be fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)ₙ, glycine-serine polymers (including, for example, (GS)ₙ, (GSGGS)ₙ (SEQ ID NO:156), (GGSGGS)ₙ (SEQ ID NO:157), and (GGGGS)ₙ (SEQ ID NO:158), where n is an integer of at least one, e.g., where n is 1, 2, 3, 4, 5, 6, 7, 8, 9, or 10), glycine-alanine polymers, alanine-serine polymers. Exemplary linkers can comprise amino acid sequences including, but not limited to, GGSG (SEQ ID NO:159), GGSGG (SEQ ID NO:160). GSGSG (SEQ ID NO:161), GSGGG (SEQ ID NO:162), GGGSG (SEQ ID NO:163), GSSSG (SEQ ID NO:164), and the like. The ordinarily skilled artisan will recognize that design of a peptide conjugated to any desired element can include linkers that are all or partially flexible, such that the linker can include a flexible linker as well as one or more portions that confer less flexible structure.

### Nucleic Acids and Expression Vectors

The present disclosure provides a nucleic acid comprising a nucleotide sequence encoding a variant CRISPR-Cas effector polypeptide of the present disclosure. The present disclosure provides a nucleic acid comprising a nucleotide sequence encoding a variant CRISPR-Cas effector fusion polypeptide of the present disclosure. The present disclosure provides a nucleic acid comprising a nucleotide sequence encoding a variant CRISPR-Cas effector polypeptide. In some cases, the nucleic acid comprises a nucleotide sequence encoding one or more guide RNAs. In some cases, the nucleic acid comprises a nucleotide sequence encoding a donor template nucleic acid. The present disclosure provides a plurality of nucleic acids, comprising: i) a first nucleic acid comprising a nucleotide sequence encoding a variant CRISPR-Cas effector polypeptide of the present disclosure; and ii) a second nucleic acid, where the second nucleic acid is a guide nucleic acid. The present disclosure provides a plurality of nucleic acids, comprising: i) a first nucleic acid comprising a nucleotide sequence encoding a variant CRISPR-Cas effector polypeptide of the present disclosure; ii) a second nucleic acid, where the second nucleic acid is a guide nucleic acid; and iii) a third nucleic acid, where the third nucleic acid is a donor nucleic acid.

The present disclosure provides a nucleic acid comprising a nucleotide sequence encoding a CRISPR-Cas polypeptide (e.g., ThermoCas9 variant) of the present disclosure. The present disclosure provides a nucleic acid comprising a nucleotide sequence encoding a CRISPR-Cas polypeptide (e.g., ThermoCas9 variant) fusion polypeptide of the present disclosure. The present disclosure provides a nucleic acid comprising a nucleotide sequence encoding a CRISPR-Cas polypeptide (e.g., ThermoCas9 variant). In some cases, the nucleic acid comprises a nucleotide sequence encoding one or more guide RNAs. In some cases, the nucleic acid comprises a nucleotide sequence encoding a donor template nucleic acid. The present disclosure provides a plurality of nucleic acids, comprising: i) a first nucleic acid comprising a nucleotide sequence encoding a CRISPR-Cas polypeptide (e.g., ThermoCas9 variant) of the present disclosure; and ii) a second nucleic acid, where the second nucleic acid is a guide nucleic acid. The present disclosure provides a plurality of nucleic acids, comprising: i) a first nucleic acid comprising a nucleotide sequence encoding a CRISPR-Cas polypeptide (e.g., ThermoCas9 variant) of the present disclosure; ii) a second nucleic acid, where the second nucleic acid is a guide nucleic acid; and iii) a third nucleic acid, where the third nucleic acid is a donor nucleic acid.

The present disclosure provides one or more nucleic acids comprising one or more of: a) a donor polynucleotide sequence; b) a nucleotide sequence encoding a variant CRISPR-Cas effector polypeptide, or a variant CRISPR-Cas effector fusion polypeptide; c) a guide RNA; and d) a nucleotide sequence encoding a guide RNA (which can include two separate nucleotide sequences in the case of dual guide RNA format or which can include a single nucleotide sequence in the case of single guide RNA format). The present disclosure provides a nucleic acid comprising a nucleotide sequence encoding a variant CRISPR-Cas effector fusion polypeptide. The present disclosure provides a recombinant expression vector that comprises a nucleotide sequence encoding a variant CRISPR-Cas effector polypeptide. The present disclosure provides a recombinant expression vector that comprises a nucleotide sequence encoding a variant CRISPR-Cas effector fusion polypeptide. The present disclosure provides a recombinant expression vector that comprises: a) a nucleotide sequence encoding a variant CRISPR-Cas effector polypeptide; and b) a nucleotide sequence encoding a guide RNA(s). The present disclosure provides a recombinant expression vector that comprises: a) a nucleotide sequence encoding a variant CRISPR-Cas effector fusion polypeptide; and b) a nucleotide sequence encoding a guide RNA(s). In some cases, the nucleotide sequence encoding the variant CRISPR-Cas effector polypeptide and/or the nucleotide sequence encoding the guide RNA is operably linked to a promoter that is operable in a cell type of choice (e.g., a prokaryotic cell, a eukaryotic cell, a plant cell, an animal cell, a mammalian cell, a primate cell, an invertebrate, a rodent cell, a human cell, etc.).

The present disclosure provides one or more nucleic acids comprising one or more of: a) a donor polynucleotide sequence; b) a nucleotide sequence encoding a CRISPR-Cas polypeptide (e.g., ThermoCas9 variant), or a CRISPR-Cas polypeptide (e.g., ThermoCas9 variant); c) a guide RNA; and d) a nucleotide sequence encoding a guide RNA (which can include two separate nucleotide sequences in the case of dual guide RNA format or which can include a single nucleotide sequence in the case of single guide RNA format). The present disclosure provides a nucleic acid comprising a nucleotide sequence encoding a CRISPR-Cas polypeptide (e.g., ThermoCas9 variant). The present disclosure provides a recombinant expression vector that comprises a nucleotide sequence encoding a CRISPR-Cas polypeptide (e.g., ThermoCas9 variant). The present disclosure provides a recombinant expression vector that comprises a nucleotide sequence encoding a CRISPR-Cas polypeptide (e.g., ThermoCas9 variant). The present disclosure provides a recombinant expression vector that comprises: a) a nucleotide sequence encoding a CRISPR-Cas polypeptide (e.g., ThermoCas9 variant); and b) a nucleotide sequence encoding a guide RNA(s). The present disclosure provides a recombinant expression vector that comprises: a) a nucleotide sequence encoding a CRISPR-Cas polypeptide (e.g., ThermoCas9 variant); and b) a nucleotide sequence encoding a guide RNA(s). In some cases, the nucleotide sequence encoding the CRISPR-Cas polypeptide (e.g., ThermoCas9 variant) and/or the nucleotide sequence encoding the guide RNA is operably linked to a promoter that is operable in a cell type of choice (e.g., a prokaryotic cell, a eukaryotic cell, a plant cell, an animal cell, a mammalian cell, a primate cell, an invertebrate, a rodent cell, a human cell, etc.).

In some cases, a nucleotide sequence encoding a variant CRISPR-Cas effector polypeptide or a variant CRISPR-Cas effector fusion polypeptide (or a CRISPR-Cas polypeptide, e.g., a ThermoCas9 variant) is codon optimized. This type of optimization can entail a mutation of a variant CRISPR-Cas effector polypeptide-encoding or variant CRISPR-Cas effector fusion polypeptide-encoding (or CRISPR-Cas polypeptide, e.g., ThermoCas9 variant) nucleotide sequence to mimic the codon preferences of the intended host organism or cell while encoding the same protein. Thus, the codons can be changed, but the encoded protein remains unchanged. For example, if the intended target cell was a human cell, a human codon-optimized variant CRISPR-Cas effector polypeptide-encoding or variant CRISPR-Cas effector fusion polypeptide-encoding nucleotide sequence could be used. As another non-limiting example, if the intended host cell were a mouse cell, then a mouse codon-optimized variant CRISPR-Cas effector polypeptide-encoding or variant CRISPR-Cas effector fusion polypeptide-encoding nucleotide sequence could be generated. As another non-limiting example, if the intended host cell were a plant cell, then a plant codon-optimized variant CRISPR-Cas effector polypeptide-encoding or variant CRISPR-Cas effector fusion polypeptide-encoding nucleotide sequence could be generated. As another non-limiting example, if the intended host cell were an insect cell, then an insect codon-optimized variant CRISPR-Cas effector polypeptide-encoding or variant CRISPR-Cas effector fusion polypeptide-encoding nucleotide sequence could be generated.

The present disclosure provides one or more recombinant expression vectors that include (in different recombinant expression vectors in some cases, and in the same recombinant expression vector in some cases): (i) a nucleotide sequence of a donor template nucleic acid (where the donor template comprises a nucleotide sequence having homology to a target sequence of a target nucleic acid (e.g., a target genome)); (ii) a nucleotide sequence that encodes a guide RNA that hybridizes to a target sequence of the target locus of the targeted genome (e.g., a single or dual guide RNA) (e.g., operably linked to a promoter that is operable in a target cell such as a eukaryotic cell); and (iii) a nucleotide sequence encoding a variant CRISPR-Cas effector polypeptide or variant CRISPR-Cas fusion effector polypeptide (e.g., operably linked to a promoter that is operable in a target cell such as a eukaryotic cell). The present disclosure provides one or more recombinant expression vectors that include (in different recombinant expression vectors in some cases, and in the same recombinant expression vector in some cases): (i) a nucleotide sequence of a donor template nucleic acid (where the donor template comprises a nucleotide sequence having homology to a target sequence of a target nucleic acid (e.g., a target genome)); and (ii) a nucleotide sequence that encodes a guide RNA that hybridizes to a target sequence of the target locus of the targeted genome (e.g., a single or dual guide RNA) (e.g., operably linked to a promoter that is operable in a target cell such as a eukaryotic cell). The present disclosure provides one or more recombinant expression vectors that include (in different recombinant expression vectors in some cases, and in the same recombinant expression vector in some cases): (i) a nucleotide sequence that encodes a guide RNA that hybridizes to a target sequence of the target locus of the targeted genome (e.g., a single or dual guide RNA) (e.g., operably linked to a promoter that is operable in a target cell such as a eukaryotic cell); and (ii) a nucleotide sequence encoding a variant CRISPR-Cas effector polypeptide or a variant CRISPR-Cas effector fusion polypeptide (e.g., operably linked to a promoter that is operable in a target cell such as a eukaryotic cell).

The present disclosure provides one or more recombinant expression vectors that include (in different recombinant expression vectors in some cases, and in the same recombinant expression vector in some cases): (i) a nucleotide sequence of a donor template nucleic acid (where the donor template comprises a nucleotide sequence having homology to a target sequence of a target nucleic acid (e.g., a target genome)); (ii) a nucleotide sequence that encodes a guide RNA that hybridizes to a target sequence of the target locus of the targeted genome (e.g., a single or dual guide RNA) (e.g., operably linked to a promoter that is operable in a target cell such as a eukaryotic cell); and (iii) a nucleotide sequence encoding a CRISPR-Cas polypeptide (e.g., ThermoCas9 variant) or CRISPR-Cas (e.g., ThermoCas9 variant) fusion effector polypeptide (e.g., operably linked to a promoter that is operable in a target cell such as a eukaryotic cell). The present disclosure provides one or more recombinant expression vectors that include (in different recombinant expression vectors in some cases, and in the same recombinant expression vector in some cases): (i) a nucleotide sequence of a donor template nucleic acid (where the donor template comprises a nucleotide sequence having homology to a target sequence of a target nucleic acid (e.g., a target genome)); and (ii) a nucleotide sequence that encodes a guide RNA that hybridizes to a target sequence of the target locus of the targeted genome (e.g., a single or dual guide RNA) (e.g., operably linked to a promoter that is operable in a target cell such as a eukaryotic cell). The present disclosure provides one or more recombinant expression vectors that include (in different recombinant expression vectors in some cases, and in the same recombinant expression vector in some cases): (i) a nucleotide sequence that encodes a guide RNA that hybridizes to a target sequence of the target locus of the targeted genome (e.g., a single or dual guide RNA) (e.g., operably linked to a promoter that is operable in a target cell such as a eukaryotic cell); and (ii) a nucleotide sequence encoding a CRISPR-Cas polypeptide (e.g., ThermoCas9 variant) or a CRISPR-Cas polypeptide (e.g., ThermoCas9 variant) fusion polypeptide (e.g., operably linked to a promoter that is operable in a target cell such as a eukaryotic cell).

Suitable expression vectors include viral expression vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Human Gene Ther 5:1088 1097, 1999; WO 94/12649, WO 93/03769; WO 93/19191; WO 94/28938; WO 95/11984 and WO 95/00655); adeno-associated virus (AAV) (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, a recombinant expression vector of the present disclosure is a recombinant adeno-associated virus (AAV) vector. In some cases, a recombinant expression vector of the present disclosure is a recombinant lentivirus vector. In some cases, a recombinant expression vector of the present disclosure is a recombinant retroviral vector.

Depending on the host/vector system utilized, any of a number of suitable transcription and translation control elements, including constitutive and inducible promoters, transcription enhancer elements, transcription terminators, etc. may be used in the expression vector.

In some embodiments, a nucleotide sequence encoding a guide RNA is operably linked to a control element, e.g., a transcriptional control element, such as a promoter. In some embodiments, a nucleotide sequence encoding a variant CRISPR-Cas effector polypeptide or a variant CRISPR-Cas effector fusion polypeptide is operably linked to a control element, e.g., a transcriptional control element, such as a promoter. In some embodiments, a nucleotide sequence encoding a CRISPR-Cas polypeptide (e.g., ThermoCas9 variant) or a CRISPR-Cas (e.g., ThermoCas9 variant) fusion polypeptide is operably linked to a control element, e.g., a transcriptional control element, such as a promoter.

The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells, e.g., hematopoietic stem cells (e.g., mobilized peripheral blood (mPB) CD34(+) cell, bone marrow (BM) CD34(+) cell, etc.).

Non-limiting examples of eukaryotic promoters (promoters functional in a eukaryotic cell) include EF1α, those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the GeoCas protein, thus resulting in a fusion GeoCas polypeptide.

In some cases, a nucleotide sequence encoding a guide RNA and/or a variant CRISPR-Cas effector polypeptide or a variant CRISPR-Cas fusion effector polypeptide is operably linked to an inducible promoter. In some cases, a nucleotide sequence encoding a guide RNA and/or variant CRISPR-Cas effector polypeptide or a variant CRISPR-Cas effector fusion polypeptide is operably linked to a constitutive promoter. In some cases, a nucleotide sequence encoding a guide RNA and/or a CRISPR-Cas polypeptide (e.g., ThermoCas9 variant) or a CRISPR-Cas (e.g., ThermoCas9 variant) fusion effector polypeptide is operably linked to an inducible promoter. In some cases, a nucleotide sequence encoding a guide RNA and/or CRISPR-Cas polypeptide (e.g., ThermoCas9 variant) or a CRISPR-Cas (e.g., ThermoCas9 variant) fusion effector polypeptide is operably linked to a constitutive promoter.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1), and the like.

In some cases, a nucleotide sequence encoding a guide RNA is operably linked to (under the control of) a promoter operable in a eukaryotic cell (e.g., a U6 promoter, an enhanced U6 promoter, an H1 promoter, and the like). As would be understood by one of ordinary skill in the art, when expressing an RNA (e.g., a guide RNA) from a nucleic acid (e.g., an expression vector) using a U6 promoter (e.g., in a eukaryotic cell), or another PolIII promoter, the RNA may need to be mutated if there are several Ts in a row (coding for Us in the RNA). This is because a string of Ts (e.g., 5 Ts) in DNA can act as a terminator for polymerase III (PolIII). Thus, in order to ensure transcription of a guide RNA (e.g., the activator portion and/or targeter portion, in dual guide or single guide format) in a eukaryotic cell it may sometimes be necessary to modify the sequence encoding the guide RNA to eliminate runs of Ts. In some cases, a nucleotide sequence encoding a variant CRISPR-Cas effector polypeptide or a variant CRISPR-Cas effector fusion polypeptide is operably linked to a promoter operable in a eukaryotic cell (e.g., a CMV promoter, an EF1α promoter, an estrogen receptor-regulated promoter, and the like). In some cases, a nucleotide sequence encoding a CRISPR-Cas polypeptide (e.g., ThermoCas9 variant) or a CRISPR-Cas (e.g., ThermoCas9 variant) fusion polypeptide is operably linked to a promoter operable in a eukaryotic cell (e.g., a CMV promoter, an EF1α promoter, an estrogen receptor-regulated promoter, and the like).

Examples of inducible promoters include, but are not limited to T7 RNA polymerase promoter, T3 RNA polymerase promoter, isopropyl-beta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; estrogen and/or an estrogen analog; IPTG; etc.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

The present disclosure provides a recombinant expression vector comprising: i) an insertion site for insertion of a nucleotide sequence encoding a targeting sequence (a nucleotide sequence that hybridizes to a target sequence in a target nucleic acid); and ii) a nucleotide sequence encoding a guide RNA as described above. For example, a nucleotide sequence encoding a guide RNA can encode the following nucleotide sequence:

GUCAUAGUUCCCCUGAgaaaUCAGGGUUACUAUGAUAAGGGCUUUCUGCC UAAGGCAGACUGACCCGCGGCGUUGGGGAUCGCCUGUCGCCCGCUUUUGGCGGGCAUUC CCCAUCCUU (SEQ ID NO: 39). In some cases, the recombinant expression vector further comprises a nucleotide sequence encoding a variant CRISPR-Cas effector polypeptide or a variant CRISPR-Cas effector fusion polypeptide of the present disclosure).

Methods of introducing a nucleic acid (e.g., a nucleic acid comprising a donor polynucleotide sequence, one or more nucleic acids encoding a variant CRISPR-Cas effector polypeptide or a variant CRISPR-Cas effector fusion polypeptide and/or a GeoCas guide RNA, and the like) into a host cell are known in the art, and any convenient method can be used to introduce a nucleic acid (e.g., an expression construct) into a cell. Likewise, methods of introducing a nucleic acid (e.g., a nucleic acid comprising a donor polynucleotide sequence, one or more nucleic acids encoding a CRISPR-Cas effector polypeptide (e.g., ThermoCas9 variant) or a CRISPR-Cas effector (e.g., ThermoCas9 variant) fusion polypeptide and/or a guide RNA, and the like) into a host cell are known in the art, and any convenient method can be used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

Introducing the recombinant expression vector into cells can occur in any culture media and under any culture conditions that promote the survival of the cells. Introducing the recombinant expression vector into a target cell can be carried out in vivo or ex vivo. Introducing the recombinant expression vector into a target cell can be carried out in vitro.

In some cases, a variant CRISPR-Cas effector polypeptide or a variant CRISPR-Cas effector fusion polypeptide can be provided as RNA. Likewise, in some cases, a CRISPR-Cas effector polypeptide (e.g., ThermoCas9 variant) or a CRISPR-Cas effector (e.g., ThermoCas9 variant) fusion polypeptide can be provided as RNA. The RNA can be provided by direct chemical synthesis or may be transcribed in vitro from a DNA (e.g., encoding the protein). Once synthesized, the RNA may be introduced into a cell by any of the well-known techniques for introducing nucleic acids into cells (e.g., microinjection, electroporation, transfection, etc.).

Nucleic acids may be provided to the cells using well-developed transfection techniques; see, e.g. Angel and Yanik (2010) PLoS ONE 5(7): el 1756, and the commercially available TransMessenger® reagents from Qiagen, StemfectTM RNA Transfection Kit from Stemgent, and TransIT®-mRNA Transfection Kit from Mirus Bio LLC. See also Beumer et al. (2008) PNAS 105(50):19821-19826.

Vectors may be provided directly to a target host cell. In other words, in some cases, a target host cell is with one or more recombinant expression vectors comprising the subject nucleic acids (e.g., recombinant expression vectors having the donor template sequence and/or encoding the guide RNA; recombinant expression vectors encoding the variant CRISPR-Cas effector polypeptide or variant CRISPR-Cas effector fusion polypeptide; etc.) such that the vectors are taken up by the cells. Methods for contacting cells with nucleic acid vectors that are plasmids, include electroporation, calcium chloride transfection, microinjection, and lipofection are well known in the art. For viral vector delivery, cells can be contacted with viral particles comprising the subject viral expression vectors.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

Vectors used for providing the nucleic acids encoding guide RNA and/or a variant CRISPR-Cas effector polypeptide or a variant CRISPR-Cas effector fusion polypeptide to a target host cell can include suitable promoters for driving the expression, that is, transcriptional activation, of the nucleic acid of interest. In other words, in some cases, the nucleic acid of interest will be operably linked to a promoter. This may include ubiquitously acting promoters, for example, the CMV-R-actin promoter, or inducible promoters, such as promoters that are active in particular cell populations or that respond to the presence of drugs such as tetracycline. By transcriptional activation, it is intended that transcription will be increased above basal levels in the target cell by 10 fold, by 100 fold, more usually by 1000 fold. In addition, vectors used for providing a nucleic acid encoding a guide RNA and/or a variant CRISPR-Cas effector polypeptide or a variant CRISPR-Cas effector fusion polypeptide to a cell may include nucleic acid sequences that encode for selectable markers in the target cells, so as to identify cells that have taken up the guide RNA and/or variant CRISPR-Cas effector polypeptide or a variant CRISPR-Cas effector fusion polypeptide.

A variant CRISPR-Cas effector polypeptide or a variant CRISPR-Cas effector fusion polypeptide can be isolated and purified in accordance with conventional methods of recombinant synthesis. A lysate may be prepared of the expression host and the lysate purified using high performance liquid chromatography (HPLC), exclusion chromatography, gel electrophoresis, affinity chromatography, or other purification technique. For the most part, the compositions which are used will comprise 20% or more by weight of the desired product, more usually 75% or more by weight, preferably 95% or more by weight, and for therapeutic purposes, usually 99.5% or more by weight, in relation to contaminants related to the method of preparation of the product and its purification. Usually, the percentages will be based upon total protein. Thus, in some cases, a variant CRISPR-Cas effector polypeptide or a variant CRISPR-Cas effector fusion polypeptide of the present disclosure is at least 80% pure, at least 85% pure, at least 90% pure, at least 95% pure, at least 98% pure, or at least 99% pure (e.g., free of contaminants, proteins (proteins other than a variant CRISPR-Cas effector polypeptide or a variant CRISPR-Cas effector fusion polypeptide) or other macromolecules, etc.).

To induce cleavage or any desired modification to a target nucleic acid (e.g., genomic DNA), or any desired modification to a polypeptide associated with target nucleic acid, a guide RNA and/or a variant CRISPR-Cas effector polypeptide or a variant CRISPR-Cas effector fusion polypeptide of the present disclosure and/or the donor template sequence, whether they be introduced as nucleic acids or polypeptides, are provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days.

In cases in which two or more different targeting complexes are provided to the cell (e.g., two different guide RNAs that are complementary to different sequences within the same or different target nucleic acid), the complexes may be provided simultaneously (e.g. as two polypeptides and/or nucleic acids), or delivered simultaneously. Alternatively, they may be provided consecutively, e.g. the targeting complex being provided first, followed by the second targeting complex, etc. or vice versa.

To improve the delivery of a DNA vector into a target cell, the DNA can be protected from damage and its entry into the cell facilitated, for example, by using lipoplexes and polyplexes. Thus, in some cases, a nucleic acid of the present disclosure (e.g., a recombinant expression vector of the present disclosure) can be covered with lipids in an organized structure like a micelle or a liposome. When the organized structure is complexed with DNA it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively charged DNA. Also as a result of their charge, they interact with the cell membrane. Endocytosis of the lipoplex then occurs, and the DNA is released into the cytoplasm. The cationic lipids also protect against degradation of the DNA by the cell.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

Dendrimers, a highly branched macromolecule with a spherical shape, may be also be used to genetically modify stem cells. The surface of the dendrimer particle may be functionalized to alter its properties. In particular, it is possible to construct a cationic dendrimer (i.e., one with a positive surface charge). When in the presence of genetic material such as a DNA plasmid, charge complementarity leads to a temporary association of the nucleic acid with the cationic dendrimer. On reaching its destination, the dendrimer-nucleic acid complex can be taken up into a cell by endocytosis.

In some cases, a nucleic acid of the disclosure (e.g., an expression vector) includes an insertion site for a guide sequence of interest. For example, a nucleic acid can include an insertion site for a guide sequence of interest, where the insertion site is immediately adjacent to a nucleotide sequence encoding the portion of a guide RNA that does not change when the guide sequence is changed to hybridized to a desired target sequence (e.g., sequences that contribute to the protein-binding portion of the guide RNA, e.g, the sequences that contribute to the dsRNA duplex(es) of the guide RNA—this portion of the guide RNA can also be referred to as the ‘scaffold’ or ‘constant region’ of the guide RNA). Thus, in some cases, a subject nucleic acid (e.g., an expression vector) includes a nucleotide sequence encoding a GeoCas9 guide RNA, except that the portion encoding the guide sequence portion of the guide RNA is an insertion sequence (an insertion site). An insertion site is any nucleotide sequence used for the insertion of a desired sequence. “Insertion sites” for use with various technologies are known to those of ordinary skill in the art and any convenient insertion site can be used. An insertion site can be for any method for manipulating nucleic acid sequences. For example, in some cases the insertion site is a multiple cloning site (MCS) (e.g., a site including one or more restriction enzyme recognition sequences), a site for ligation independent cloning, a site for recombination-based cloning (e.g., recombination based on att sites), a nucleotide sequence recognized by a CRISPR/Cas (e.g. Cas9) based technology, and the like.

An insertion site can be any desirable length, and can depend on the type of insertion site (e.g., can depend on whether (and how many) the site includes one or more restriction enzyme recognition sequences, whether the site includes a target site for a GeoCas9 protein, etc.). In some cases, an insertion site of a subject nucleic acid is 3 or more nucleotides (nt) in length (e.g., 5 or more, 8 or more, 10 or more, 15 or more, 17 or more, 18 or more, 19 or more, 20 or more or 25 or more, or 30 or more nt in length). In some cases, the length of an insertion site of a subject nucleic acid has a length in a range of from 2 to 50 nucleotides (nt) (e.g., from 2 to 40 nt, from 2 to 30 nt, from 2 to 25 nt, from 2 to 20 nt, from 5 to 50 nt, from 5 to 40 nt, from 5 to 30 nt, from 5 to 25 nt, from 5 to 20 nt, from 10 to 50 nt, from 10 to 40 nt, from 10 to 30 nt, from 10 to 25 nt, from 10 to 20 nt, from 17 to 50 nt, from 17 to 40 nt, from 17 to 30 nt, from 17 to 25 nt). In some cases, the length of an insertion site of a subject nucleic acid has a length in a range of from 5 to 40 nt.

### Nucleic Acid Modifications

In some embodiments, a guide RNA has one or more modifications, e.g., a base modification, a backbone modification, etc., to provide the nucleic acid with a new or enhanced feature (e.g., improved stability). A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′. the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound; however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Suitable nucleic acid modifications include, but are not limited to: 2′Omethyl modified nucleotides, 2′ fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Additional details and additional modifications are described below.

A 2′-O-Methyl modified nucleotide (also referred to as 2′-O-Methyl RNA) is a naturally occurring modification of RNA found in tRNA and other small RNAs that arises as a post-transcriptional modification. Oligonucleotides can be directly synthesized that contain 2′-O-Methyl RNA. This modification increases Tm of RNA:RNA duplexes but results in only small changes in RNA:DNA stability. It is stabile with respect to attack by single-stranded ribonucleases and is typically 5 to 10-fold less susceptible to DNases than DNA. It is commonly used in antisense oligos as a means to increase stability and binding affinity to the target message.

2′ Fluoro modified nucleotides (e.g., 2′ Fluoro bases) have a fluorine modified ribose which increases binding affinity (Tm) and also confers some relative nuclease resistance when compared to native RNA. These modifications are commonly employed in ribozymes and siRNAs to improve stability in serum or other biological fluids.

LNA bases have a modification to the ribose backbone that locks the base in the C3′-endo position, which favors RNA A-type helix duplex geometry. This modification significantly increases Tm and is also very nuclease resistant. Multiple LNA insertions can be placed in an oligo at any position except the 3′-end. Applications have been described ranging from antisense oligos to hybridization probes to SNP detection and allele specific PCR. Due to the large increase in Tm conferred by LNAs, they also can cause an increase in primer dimer formation as well as self-hairpin formation. In some cases, the number of LNAs incorporated into a single oligo is 10 bases or less.

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleotide linkage resistant to nuclease degradation. Phosphorothioate bonds can be introduced between the last 3-5 nucleotides at the 5′- or 3′-end of the oligo to inhibit exonuclease degradation. Including phosphorothioate bonds within the oligo (e.g., throughout the entire oligo) can help reduce attack by endonucleases as well.

In some cases, a guide nucleic acid has one or more nucleotides that are 2′-O-Methyl modified nucleotides. In some cases, a subject nucleic acid (e.g., a guide RNA etc.) has one or more 2′ Fluoro modified nucleotides. In some cases, a subject nucleic acid (e.g., guide RNA, etc.) has one or more LNA bases. In some cases, a subject nucleic acid (e.g., guide RNA, etc.) has one or more nucleotides that are linked by a phosphorothioate bond (i.e., the subject nucleic acid has one or more phosphorothioate linkages). In some cases, a subject nucleic acid (e.g., a guide RNA, etc.) has a 5′ cap (e.g., a 7-methylguanylate cap (m7G)). In some cases, a subject nucleic acid (e.g., a guide RNA, etc.) has a combination of modified nucleotides. For example, a subject nucleic acid (e.g., a guide RNA, etc.) can have a 5′ cap (e.g., a 7-methylguanylate cap (m7G)) in addition to having one or more nucleotides with other modifications (e.g., a 2′-O-Methyl nucleotide and/or a 2′ fluoro modified nucleotide and/or a LNA base and/or a phosphorothioate linkage).

### Modified Backbones and Modified Internucleoside Linkages

Examples of suitable nucleic acids (e.g., a guide RNA) containing modifications include nucleic acids containing modified backbones or non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some cases, a guide nucleic acid comprises one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(=O)(OH)—O—CH₂—). MMI type internucleoside linkages are disclosed in the above referenced U.S. Pat. No. 5,489,677, the disclosure of which is incorporated herein by reference in its entirety. Suitable amide internucleoside linkages are disclosed in U.S. Pat. No. 5,602,240, the disclosure of which is incorporated herein by reference in its entirety.

Also suitable are nucleic acids having morpholino backbone structures as described in, e.g., U.S. Pat. No. 5,034,506. For example, in some embodiments, a subject nucleic acid comprises a 6-membered morpholino ring in place of a ribose ring. In some of these embodiments, a phosphorodiamidate or other non-phosphodiester internucleoside linkage replaces a phosphodicster linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Minetics

A subject nucleic acid can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262, the disclosures of which are incorporated herein by reference in their entirety.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506, the disclosure of which is incorporated herein by reference in its entirety. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602, the disclosure of which is incorporated herein by reference in its entirety). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456, the disclosure of which is incorporated herein by reference in its entirety). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (e.g., Wahlestedt et al., Proc. Natl. Acad. Sci. U.S.A., 2000, 97, 5633-5638, the disclosure of which is incorporated herein by reference in its entirety).

The synthesis and preparation of the LNA monomers adenine, cytosine, guanine, 5-methyl-cytosine, thymine and uracil, along with their oligomerization, and nucleic acid recognition properties have been described (e.g., Koshkin et al., Tetrahedron, 1998, 54, 3607-3630, the disclosure of which is incorporated herein by reference in its entirety). LNAs and preparation thereof are also described in WO 98/39352 and WO 99/14226, as well as U.S. applications 20120165514, 20100216983, 20090041809, 20060117410, 20040014959, 20020094555, and 20020086998, the disclosures of which are incorporated herein by reference in their entirety.

### Modified Sugar Moieties

A subject nucleic acid (e.g., a guide nucleic acid, e.g., a guide RNA) can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂),O)ₘCH₃, O(CH₂)ₙOCH₃, O(CH₂)ₙNH₂, O(CH₂)ₙCH₃, O(CH₂)ₙONH₂, and O(CH₂)ₙON((CH₂)·CH₃)₂, where n and m are from 1 to about 10.

Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂ CH₂OCH₃, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A subject nucleic acid (e.g., a guide RNA) may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C). 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993; the disclosures of which are incorporated herein by reference in their entirety. Certain of these nucleobases are useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278; the disclosure of which is incorporated herein by reference in its entirety) and are suitable base substitutions, e.g., when combined with 2′-O-methoxyethyl sugar modifications.

### Conjugates

Another possible modification of a guide nucleic acid involves chemically linking to the polynucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a subject nucleic acid.

Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N. Y Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

A conjugate may include a “Protein Transduction Domain” or PTD (also known as a CPP—cell penetrating peptide), which may refer to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle (e.g., the nucleus). In some embodiments, a PTD is covalently linked to the 3′ end of an exogenous polynucleotide. In some embodiments, a PTD is covalently linked to the 5′ end of an exogenous polynucleotide.

Introducing Components into a Target Cell

A guide RNA (or a nucleic acid comprising a nucleotide sequence encoding same) and/or a variant CRISPR-Cas effector polypeptide of the present disclosure (or a nucleic acid comprising a nucleotide sequence encoding same) and/or a variant CRISPR-Cas effector fusion polypeptide of the present disclosure (or a nucleic acid that includes a nucleotide sequence encoding a variant CRISPR-Cas effector fusion polypeptide of the present disclosure) and/or a donor polynucleotide (donor template) can be introduced into a host cell by any of a variety of well-known methods.

Any of a variety of compounds and methods can be used to deliver to a target cell a system of the present disclosure (e.g., where a system of the present disclosure comprises: a) a variant CRISPR-Cas effector polypeptide and a guide RNA; b) a variant CRISPR-Cas effector polypeptide, a guide RNA, and a donor template nucleic acid; c) a variant CRISPR-Cas effector fusion polypeptide of the present disclosure and a guide RNA; d) a variant CRISPR-Cas effector fusion polypeptide of the present disclosure, a guide RNA, and a donor template nucleic acid; e) an mRNA encoding a variant CRISPR-Cas effector; and a guide RNA; f) an mRNA encoding a variant CRISPR-Cas effector; a guide RNA; and a donor template nucleic acid; g) an mRNA encoding a variant CRISPR-Cas effector fusion polypeptide of the present disclosure; and a guide RNA; h) an mRNA encoding a variant CRISPR-Cas effector fusion polypeptide of the present disclosure; a guide RNA and a donor template nucleic acid; i) a recombinant expression vector comprising: i) a nucleotide sequence encoding a variant CRISPR-Cas effector polypeptide; and ii) a nucleotide sequence encoding a guide RNA; j) a recombinant expression vector comprising: i) a nucleotide sequence encoding a variant CRISPR-Cas effector polypeptide; ii) a nucleotide sequence encoding a guide RNA; and iii) a nucleotide sequence encoding a donor template nucleic acid; k) a recombinant expression vector comprising: i) a nucleotide sequence encoding a variant CRISPR-Cas effector fusion polypeptide of the present disclosure; and ii) a nucleotide sequence encoding a guide RNA; l) a recombinant expression vector comprising: i) a nucleotide sequence encoding a variant CRISPR-Cas effector fusion polypeptide of the present disclosure; ii) a nucleotide sequence encoding a guide RNA; and a iii) nucleotide sequence encoding a donor template nucleic acid; m) a first recombinant expression vector comprising a nucleotide sequence encoding a variant CRISPR-Cas effector polypeptide, and a second recombinant expression vector comprising a nucleotide sequence encoding a guide RNA; n) a first recombinant expression vector comprising a nucleotide sequence encoding a variant CRISPR-Cas effector polypeptide of the present disclosure, and a second recombinant expression vector comprising a nucleotide sequence encoding a guide RNA; and a donor template nucleic acid; o) a first recombinant expression vector comprising a nucleotide sequence encoding a variant CRISPR-Cas effector fusion polypeptide of the present disclosure, and a second recombinant expression vector comprising a nucleotide sequence encoding a guide RNA; p) a first recombinant expression vector comprising a nucleotide sequence encoding a variant CRISPR-Cas effector fusion polypeptide of the present disclosure, and a second recombinant expression vector comprising a nucleotide sequence encoding a guide RNA; and a donor template nucleic acid; q) a recombinant expression vector comprising: i) a nucleotide sequence encoding a variant CRISPR-Cas effector polypeptide; ii) a nucleotide sequence encoding a first guide RNA; and iii) a nucleotide sequence encoding a second guide RNA; or r) a recombinant expression vector comprising: i) a nucleotide sequence encoding a variant CRISPR-Cas effector fusion polypeptide of the present disclosure; ii) a nucleotide sequence encoding a first guide RNA; and iii) a nucleotide sequence encoding a second guide RNA; or some variation of one of (a) through (r). As a non-limiting example, a system of the present disclosure can be combined with a lipid. As another non-limiting example, a system of the present disclosure can be combined with a particle, or formulated into a particle.

Methods of introducing a nucleic acid into a host cell are known in the art, and any convenient method can be used to introduce a subject nucleic acid (e.g., an expression construct/vector) into a target cell (e.g., prokaryotic cell, eukaryotic cell, plant cell, animal cell, mammalian cell, human cell, and the like). Suitable methods include, e.g., viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: S0169-409X(12)00283-9. doi: 10.1016/j.addr.2012.09.023). and the like.

### Compositions

The present disclosure provides compositions, including pharmaceutical compositions, comprising a variant CRISPR-Cas effector polypeptide of the present disclosure, a variant CRISPR-Cas effector fusion polypeptide of the present disclosure, a nucleic acid of the present disclosure, a recombinant expression vector of the present disclosure, an RNP of the present disclosure, or a system of the present disclosure.

Pharmaceutical compositions can include, depending on the formulation desired, pharmaceutically-acceptable, non-toxic carriers of diluents, which arc defined as vehicles commonly used to formulate pharmaceutical compositions for animal or human administration. The diluent is selected so as not to affect the biological activity of the combination. Examples of such diluents are distilled water, buffered water, physiological saline, phosphate buffered saline (PBS). Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are generally of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Cells

The present disclosure provides a modified cell comprising a variant CRISPR-Cas effector polypeptide or a variant CRISPR-Cas effector fusion polypeptide and/or a nucleic acid comprising a nucleotide sequence encoding a variant CRISPR-Cas effector polypeptide or a variant CRISPR-Cas effector fusion polypeptide. Likewise, the present disclosure provides a modified cell comprising a CRISPR-Cas polypeptide (e.g., ThermoCas9 variant) or a CRISPR-Cas (e.g., ThermoCas9 variant) fusion polypeptide and/or a nucleic acid comprising a nucleotide sequence encoding a CRISPR-Cas polypeptide (e.g., ThermoCas9 variant) or a CRISPR-Cas (e.g., ThermoCas9 variant) fusion polypeptide. The present disclosure provides a modified cell (e.g., a genetically modified cell) comprising nucleic acid comprising a nucleotide sequence encoding a variant CRISPR-Cas effector polypeptide or a variant CRISPR-Cas effector fusion polypeptide. The present disclosure provides a genetically modified cell that is genetically modified with an mRNA comprising a nucleotide sequence encoding a variant CRISPR-Cas effector polypeptide or a variant CRISPR-Cas effector fusion polypeptide. The present disclosure provides a genetically modified cell that is genetically modified with a recombinant expression vector comprising a nucleotide sequence encoding a variant CRISPR-Cas effector polypeptide or a variant CRISPR-Cas effector fusion polypeptide. The present disclosure provides a genetically modified cell that is genetically modified with a recombinant expression vector comprising: a) a nucleotide sequence encoding a variant CRISPR-Cas effector polypeptide or a variant CRISPR-Cas effector fusion polypeptide; and b) a nucleotide sequence encoding a guide RNA of the present disclosure. The present disclosure provides a genetically modified cell that is genetically modified with a recombinant expression vector comprising: a) a nucleotide sequence encoding a variant CRISPR-Cas effector polypeptide or a variant CRISPR-Cas effector fusion polypeptide; b) a nucleotide sequence encoding a guide RNA of the present disclosure; and c) a nucleotide sequence encoding a donor template. The present disclosure provides a cell that is modified to comprise a system of the present disclosure.

A cell that serves as a recipient for a variant CRISPR-Cas effector polypeptide or a variant CRISPR-Cas effector fusion polypeptide and/or a nucleic acid comprising a nucleotide sequence encoding a variant CRISPR-Cas effector polypeptide or a variant CRISPR-Cas effector fusion polypeptide and/or a guide RNA of the present disclosure, can be any of a variety of cells, including, e.g., in vitro cells; in vivo cells; ex vivo cells; primary cells; cancer cells; animal cells; plant cells; algal cells; fungal cells; etc. Likewise for a cell that serves as a recipient for a CRISPR-Cas polypeptide (e.g., ThermoCas9 variant) or a CRISPR-Cas (e.g., ThermoCas9 variant) fusion polypeptide. A cell that serves as a recipient for a variant CRISPR-Cas effector polypeptide or a variant CRISPR-Cas effector fusion polypeptide and/or a nucleic acid comprising a nucleotide sequence encoding a variant CRISPR-Cas effector polypeptide or a variant CRISPR-Cas effector fusion polypeptide and/or a guide RNA of the present disclosure is referred to as a “host cell” or a “target cell.” Likewise for a cell that serves as a recipient for a CRISPR-Cas polypeptide (e.g., ThermoCas9 variant) or a CRISPR-Cas (e.g., ThermoCas9 variant) fusion polypeptide.

A host cell or a target cell can be a recipient of a system of the present disclosure. A host cell or a target cell can be a recipient of an RNP of the present disclosure. A host cell or a target cell can be a recipient of a single component of a system of the present disclosure.

Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatos, rice, cassava, sugarcane, pumpkin, hay, potatos, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell from a mushroom), an animal cell, a cell from an invertebrate animal (e.g., an insect, an arachnid, a fruit fly, a enidarian, an echinoderm, a nematode, etc.), a cell from a vertebrate animal (e.g., fish, amphibian, reptile, bird, mammal), a cell from a mammal (e.g., an ungulate (e.g., a pig, a cow, a goat, a sheep, a camel); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be an in vivo cell (e.g., a cell in an individual). A cell can be an isolated cell. A cell can be a cell inside of an organism. A cell can be an organism. A cell can be a cell in a cell culture (e.g., in vitro cell culture). A cell can be one of a collection of cells. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a mammalian cell or derived from a mammalian cell. A cell can be a rodent cell or derived from a rodent cell. A cell can be a human cell or derived from a human cell. A cell can be a microbe cell or derived from a microbe cell. A cell can be a fungi cell or derived from a fungi cell. A cell can be an insect cell. A cell can be an arthropod cell. A cell can be a protozoan cell. A cell can be a helhninth cell. A cell can be an insect cell. A cell can be an arachnid cell.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte (liver cell), a pancreatic cell, a lung cell, etc. For example, in some cases, the cell is a liver cell or lung cell in vivo. In some cases, the cell is a liver cell in vivo. In some cases, the cell is a lung cell in vivo. In some cases, the cell is a liver cell in vivo and an LNP/RNP formulation is used to target the liver (e.g., an LNP formulation that includes an acid-degradable PEGylated lipid). In some cases, the cell is a lung cell in vivo and an LNP/RNP formulation is used to target the lung (e.g., an LNP formulation that includes ADC as a cationic lipid, e.g., one that also includes an acid-degradable PEGylated lipid).

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogeneic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19. KRT7, LGR5, CA9, FXYD2, CDH6, CLDN18, TSPAN8, BPIFB1, OLFM4, CDH17, and PPARGC1A.

In some embodiments, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes).

A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

A cell is in some cases a plant cell. A plant cell can be a cell of a monocotyledon. A cell can be a cell of a dicotyledon.

In some cases, the cell is a plant cell. For example, the cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf—green), lettuce (oak leaf—red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera. Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

In some cases, a suitable cell is a prokaryotic cell. In some cases, the cell is a thermophilic prokaryotic cell. In some cases, the cell is an obligate thermophile (e.g., an obligate thermophilic prokaryotic cell). In some cases, the cell is a microorganism selected from a Cupriavidus sp., Ralstonia sp, Xanthobacter sp., Rhodococcus sp., Hydrogenovibrio sp., Rhodopseudomonas sp., Rhodobacter sp., Hydrogenobacter sp., Arthrobacter sp., Paracoccus sp., Mycobacterium sp., Streptomyces sp., and Bacillus sp.

In some cases, the host cell is a microorganism. In some cases, the microorganism is Rhodococcus opacus or Rhodococcus jostii or Rhodococcus sp. In some cases, the microorganism is Hydrogenovibrio marinus. In some cases, the microorganism is Rhodopseudomonas capsulata, Rhodopseudomonas palustris, or Rhodobacter sphaeroides. In some cases, the microorganism is an oxyhydrogen or knallgas strain. In some cases, the microorganism is selected from: Aquifex pyrophilus and Aquifex aeolicus or other Aquifex sp.; Cupriavidus necator or Cupriavidus metallidurans or other Cupriavidus sp.; Corynebacterium autotrophicum or other Corynebacterium sp.; Nocardia autotrophica and Nocardia opaca and other Nocardia sp.; purple non-sulfur photosynthetic bacteria including but not limited to Rhodopseudomonas palustris, Rhodopseudomonas capsulata. Rhodopseudomonas viridis, Rhodopseudomonas sulfoviridis, Rhodopseudomonas blastica, Rhodopseudomonas spheroides, Rhodopseudomonas acidophila and other Rhodopseudomonas sp., Rhodospirillum rubrum, and other Rhodospirillum sp.; Rhodococcus opacus and other Rhodococcus sp.; Rhizobium japonicum and other Rhizobium sp.; Thiocapsa roseopersicina and other Thiocapsa sp.; Pseudomonas facilis and Pseudomonas flava and Pseudomonas putida and Pseudomonas hydrogenovora, Pseudomonas hydrogenothermophila, Pseudomonas palleronii and Pseudomonas pseudoflava and Pseudomonas saccharophila and Pseudomonas thermophila and other Pseudomonas sp.; Hydrogenomonas pantotropha, Hydrogenomonas eutropha, Hydrogenomonas facilis, and other Hydrogenomonas sp.; Hydrogenobacter thermophilus and Hydrogenobacter halophilus and Hydrogenobacter hydrogenophilus and other Hydrogenobacter sp.; Hydrogenophilus islandicus and other Hydrogenophilus sp.; Hydrogenovibrio marinus and other Hydrogenovibrio sp.; Hydrogenothermus marinus and other Hydrogenothermus sp.; Helicobacter pylori and other Helicobacter sp.; Xanthobacter autotrophicus and Xanthobacter flavus other Xanthobacter sp.; Hydrogenophaga flava and Hydrogenophaga palleronii and Hydrogenophaga pseudoflava and other Hydrogenophaga sp.; Bradyrhizobium japonicum and other Bradyrhizobium sp.; Ralstonia eutropha and other Ralstonia sp.; Alcaligenes eutrophus and Alcaligenes facilis and Alcaligenes hydrogenophilus and Alcaligenes latus and Alcaligenes paradoxus and Alcaligenes ruhlandii and other Alcaligenes sp.; Amycolata sp.; Aquaspirillum autotrophicum and other Aquaspirillum sp.; Arthrobacter strain 1/X and other Arthrobacter sp.; Azospirillum lipoferum and other Azospirillum sp.; Variovorax paradoxus, and other Variovorax sp.; Acidovorax facilis, and other Acidovorax sp.; Bacillus schlegelii and Bacillus tusciae and other Bacillus sp.; Calderobacterium hydrogenophilum and other Calderobacterium sp.; Derxia gummosa and other Derxia sp.; Flavobacterium autothermophilum and other Flavobacterium sp.; Microcyclus aquaticus and other Microcyclus; Mycobacterium gordoniae and other Mycobacterium sp.; Paracoccus denitrificans and other Paracoccus sp.; Persephonella marina and Persephonella guaymasensis and other Persephonella sp.; Renobacter vacuolatum and other Renobacter sp.; Thermocrinis ruber and other Thermocrinis sp.; Wautersia sp.; cyanobacteria including but not limited to Anabaena oscillarioides, Anabaena spiroides, Anabaena cylindrica, and other Anabaena sp.; green algae including but not limited to Scenedesmus obliquus and other Scenedesmus sp., Chlamydomonas reinhardtii and other Chlamydomonas sp., Ankistrodesmus sp., Rhaphidium polymorphium and other Rhaphidium sp; as well as a consortiums of microorganisms that include oxyhydrogen microorganisms.

In some cases, the host cell is an obligate and/or facultative chemoautotrophic microorganism selected from: Acetoanaerobium sp.; Acetobacterium sp.; Acetogenium sp.; Achromobacter sp.; Acidianus sp.; Acinetobacter sp.; Actinomadura sp.; Aeromonas sp.; Alcaligenes sp.; Alcaligenes sp.; Arcobacter sp.; Aureobacterium sp.; Bacillus sp.; Beggiatoa sp.; Butyribacterium sp.; Carboxydothermus sp.; Clostridium sp.; Comamonas sp.; Dehalobacter sp.; Dehalococcoide sp.; Dehalospirillum sp.; Desulfobacterium sp.; Desulfomonile sp.; Desulfotomaculum sp.; Desulfovibrio sp.; Desulfurosarcina sp.; Ectothiorhodospira sp.; Enterobacter sp.; Eubacterium sp.; Ferroplasma sp.; Halothibacillus sp.; Hydrogenobacter sp.; Hydrogenomonas sp.; Leptospirillum sp.; Metallosphaera sp.; Methanobacterium sp.; Methanobrevibacter sp.; Methanococcus sp.; Methanosarcina sp.; Micrococcus sp.; Nitrobacter sp.; Nitrosococcus sp.; Nitrosolobus sp.; Nitrosomonas sp.; Nitrosospira sp.; Nitrosovibrio sp.; Nitrospina sp.; Oleomonas sp.; Paracoccus sp.; Peptostreptococcus sp.; Planctomycetes sp.; Pseudomonas sp.; Ralstonia sp.; Rhodobacter sp.; Rhodococcus sp.; Rhodocyclus sp.; Rhodomicrobium sp.; Rhodopseudomonas sp.; Rhodospirillum sp.; Shewanella sp.; Streptomyces sp.; Sulfobacillus sp.; Sulfolobus sp.; Thiobacillus sp.; Thiomicrospira sp.; Thioploca sp.; Thiosphaera sp.; Thiothrix sp.; sulfur-oxidizers; hydrogen-oxidizers; iron-oxidizers; acetogens; and methanogens; consortiums of microorganisms that include chemoautotrophs; chemoautotrophs native to at least one of hydrothermal vents, geothermal vents, hot springs, cold seeps, underground aquifers, salt lakes, saline formations, mines, acid mine drainage, mine tailings, oil wells, refinery wastewater, coal seams, deep sub-surface; waste water and sewage treatment plants; geothermal power plants, sulfatara fields, and soils; and extremophiles selected from one or more of thermophiles, hyperthermophiles, acidophiles, halophiles, and psychrophiles.

### Systems and Ribonucleoprotein Complexes

The present disclosure provides systems and ribonucleoprotein (RNP) complexes comprising a variant CRISPR-Cas effector polypeptide or a variant CRISPR-Cas effector fusion polypeptide. An RNP can comprise a variant CRISPR-Cas effector polypeptide and a guide RNA. An RNP can comprise a variant CRISPR-Cas effector polypeptide, a guide RNA, and a donor nucleic acid. Likewise for CRISPR-Cas polypeptides (e.g., ThermoCas9 variants) and CRISPR-Cas (e.g., ThermoCas9 variant) fusion polypeptides.

A system of the present disclosure can comprise: a) a variant CRISPR-Cas effector polypeptide and a guide RNA; b) a variant CRISPR-Cas effector polypeptide, a guide RNA, and a donor template nucleic acid; c) a variant CRISPR-Cas effector fusion polypeptide of the present disclosure and a guide RNA; d) a variant CRISPR-Cas effector fusion polypeptide of the present disclosure, a guide RNA, and a donor template nucleic acid; e) an mRNA encoding a variant CRISPR-Cas effector; and a guide RNA; f) an mRNA encoding a variant CRISPR-Cas effector; a guide RNA; and a donor template nucleic acid; g) an mRNA encoding a variant CRISPR-Cas effector fusion polypeptide of the present disclosure; and a guide RNA; h) an mRNA encoding a variant CRISPR-Cas effector fusion polypeptide of the present disclosure; a guide RNA and a donor template nucleic acid; i) a recombinant expression vector comprising: i) a nucleotide sequence encoding a variant CRISPR-Cas effector polypeptide; and ii) a nucleotide sequence encoding a guide RNA; j) a recombinant expression vector comprising: i) a nucleotide sequence encoding a variant CRISPR-Cas effector polypeptide; ii) a nucleotide sequence encoding a guide RNA; and iii) a nucleotide sequence encoding a donor template nucleic acid; k) a recombinant expression vector comprising: i) a nucleotide sequence encoding a variant CRISPR-Cas effector fusion polypeptide of the present disclosure; and ii) a nucleotide sequence encoding a guide RNA; l) a recombinant expression vector comprising: i) a nucleotide sequence encoding a variant CRISPR-Cas effector fusion polypeptide of the present disclosure; ii) a nucleotide sequence encoding a guide RNA; and a iii) nucleotide sequence encoding a donor template nucleic acid; m) a first recombinant expression vector comprising a nucleotide sequence encoding a variant CRISPR-Cas effector polypeptide, and a second recombinant expression vector comprising a nucleotide sequence encoding a guide RNA; n) a first recombinant expression vector comprising a nucleotide sequence encoding a variant CRISPR-Cas effector polypeptide of the present disclosure, and a second recombinant expression vector comprising a nucleotide sequence encoding a guide RNA; and a donor template nucleic acid; o) a first recombinant expression vector comprising a nucleotide sequence encoding a variant CRISPR-Cas effector fusion polypeptide of the present disclosure, and a second recombinant expression vector comprising a nucleotide sequence encoding a guide RNA; p) a first recombinant expression vector comprising a nucleotide sequence encoding a variant CRISPR-Cas effector fusion polypeptide of the present disclosure, and a second recombinant expression vector comprising a nucleotide sequence encoding a guide RNA; and a donor template nucleic acid; q) a recombinant expression vector comprising: i) a nucleotide sequence encoding a variant CRISPR-Cas effector polypeptide; ii) a nucleotide sequence encoding a first guide RNA; and iii) a nucleotide sequence encoding a second guide RNA; or r) a recombinant expression vector comprising: i) a nucleotide sequence encoding a variant CRISPR-Cas effector fusion polypeptide of the present disclosure; ii) a nucleotide sequence encoding a first guide RNA; and iii) a nucleotide sequence encoding a second guide RNA; or some variation of one of (a) through (r). As a non-limiting example, a system of the present disclosure can be combined with a lipid. As another non-limiting example, a system of the present disclosure can be combined with a particle, or formulated into a particle. Likewise for CRISPR-Cas polypeptides (e.g., ThermoCas9 variants) and CRISPR-Cas (e.g., ThermoCas9 variant) fusion polypeptides.

### Multicellular Non-Human Organisms

The present disclosure provides a multicellular non-human organism comprising one or more of: a) a nucleic acid comprising a nucleotide sequence encoding a variant CRISPR-Cas effector polypeptide or a variant CRISPR-Cas effector fusion polypeptide; b) a guide RNA; c) a nucleic acid comprising a nucleotide sequence encoding a guide RNA; d) a donor template; and e) a nucleic acid comprising a nucleotide sequence encoding a donor template. Likewise for CRISPR-Cas polypeptides (e.g., ThermoCas9 variants) and CRISPR-Cas (e.g., ThermoCas9 variant) fusion polypeptides. The nucleic acid(s) can be integrated into the genome of the host organism. The nucleic acid(s) can be integrated into the genome of all cells of the host organism. The nucleic acid(s) can be integrated into the genome of a subset of the cells of the host organism. The nucleic acid(s) can be extrachromosomal. A multicellular, non-human organism can comprise a system of the present disclosure.

### Transgenic, Non-Human Animals

The present disclosure provides a transgenic non-human animal, which animal comprises a transgene comprising a nucleic acid comprising a nucleotide sequence encoding a variant CRISPR-Cas effector polypeptide or a variant CRISPR-Cas effector fusion polypeptide. Likewise for CRISPR-Cas polypeptides (e.g., ThermoCas9 variants) and CRISPR-Cas (e.g., ThermoCas9 variant) fusion polypeptides. In some cases, the genome of the transgenic non-human animal comprises a nucleotide sequence encoding a variant CRISPR-Cas effector polypeptide or a variant CRISPR-Cas effector fusion polypeptide. Likewise for CRISPR-Cas polypeptides (e.g., ThermoCas9 variants) and CRISPR-Cas (e.g., ThermoCas9 variant) fusion polypeptides. In some cases, the transgenic non-human animal is homozygous for the genetic modification. In some cases, the transgenic non-human animal is heterozygous for the genetic modification. In some embodiments, the transgenic non-human animal is a vertebrate, for example, a fish (e.g., salmon, trout, zebra fish, gold fish, puffer fish, cave fish, etc.), an amphibian (frog, newt, salamander, etc.), a bird (e.g., chicken, turkey, etc.), a reptile (e.g., snake, lizard, etc.), a non-human mammal (e.g., an ungulate, e.g., a pig, a cow, a goat, a sheep, etc.; a lagomorph (e.g., a rabbit); a rodent (e.g., a rat, a mouse); a non-human primate; etc.), etc. In some cases, the transgenic non-human animal is an invertebrate. In some cases, the transgenic non-human animal is an insect (e.g., a mosquito; an agricultural pest; etc.). In some cases, the transgenic non-human animal is an arachnid.

Nucleotide sequences encoding a variant CRISPR-Cas effector polypeptide or a variant CRISPR-Cas effector fusion polypeptide can be under the control of (i.e., operably linked to) an unknown promoter (e.g., when the nucleic acid randomly integrates into a host cell genome) or can be under the control of (i.e., operably linked to) a known promoter. Likewise for CRISPR-Cas polypeptides (e.g., ThermoCas9 variants) and CRISPR-Cas (e.g., ThermoCas9 variant) fusion polypeptides. Suitable known promoters can be any known promoter and include constitutively active promoters (e.g., cytomegalovirus promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc.

### Transgenic Plants

As described above, in some cases, a nucleic acid (e.g., a recombinant expression vector) of the present disclosure (e.g., a nucleic acid comprising a nucleotide sequence encoding a variant CRISPR-Cas effector polypeptide or a variant CRISPR-Cas effector fusion polypeptide) is used as a transgene to generate a transgenic plant that produces a variant CRISPR-Cas effector polypeptide or a variant CRISPR-Cas effector fusion polypeptide of the present disclosure. Likewise for CRISPR-Cas polypeptides (e.g., ThermoCas9 variants) and CRISPR-Cas (e.g., ThermoCas9 variant) fusion polypeptides. The present disclosure provides a transgenic plant comprising a nucleotide sequence encoding a variant CRISPR-Cas effector polypeptide or a variant CRISPR-Cas effector fusion polypeptide of the present disclosure. Likewise for CRISPR-Cas polypeptides (e.g., ThermoCas9 variants) and CRISPR-Cas (e.g., ThermoCas9 variant) fusion polypeptides. In some cases, the genome of the transgenic plant comprises a subject nucleic acid. In some cases, the transgenic plant is homozygous for the genetic modification. In some embodiments, the transgenic plant is heterozygous for the genetic modification.

Methods of introducing exogenous nucleic acids into plant cells are well known in the art. Such plant cells are considered “transformed,” as defined above. Suitable methods include viral infection (such as double stranded DNA viruses), transfection, conjugation, protoplast fusion, electroporation, particle gun technology, calcium phosphate precipitation, direct microinjection, silicon carbide whiskers technology, Agrobacterium-mediated transformation and the like. The choice of method is generally dependent on the type of cell being transformed and the circumstances under which the transformation is taking place (i.e. in vitro, ex vivo, or in vivo).

Transformation methods based upon the soil bacterium Agrobacterium tumefaciens are particularly useful for introducing an exogenous nucleic acid molecule into a vascular plant. The wild type form of Agrobacterium contains a Ti (tumor-inducing) plasmid that directs production of tumorigenic crown gall growth on host plants. Transfer of the tumor-inducing T-DNA region of the Ti plasmid to a plant genome requires the Ti plasmid-encoded virulence genes as well as T-DNA borders, which are a set of direct DNA repeats that delineate the region to be transferred. An Agrobacterium-based vector is a modified form of a Ti plasmid, in which the tumor inducing functions are replaced by the nucleic acid sequence of interest to be introduced into the plant host.

Agrobacterium-mediated transformation generally employs cointegrate vectors or binary vector systems, in which the components of the Ti plasmid are divided between a helper vector, which resides permanently in the Agrobacterium host and carries the virulence genes, and a shuttle vector, which contains the gene of interest bounded by T-DNA sequences. A variety of binary vectors is well known in the art and are commercially available, for example, from Clontech (Palo Alto, Calif.). Methods of coculturing Agrobacterium with cultured plant cells or wounded tissue such as leaf tissue, root explants, hypocotyledons, stem pieces or tubers, for example, also are well known in the art. See, e.g., Glick and Thompson, (eds.), Methods in Plant Molecular Biology and Biotechnology, Boca Raton, Fla.: CRC Press (1993).

Microprojectile-mediated transformation also can be used to produce a subject transgenic plant. This method, first described by Klein et al. (Nature 327:70-73 (1987)), relies on microprojectiles such as gold or tungsten that are coated with the desired nucleic acid molecule by precipitation with calcium chloride, spermidine or polyethylene glycol. The microprojectile particles are accelerated at high speed into an angiosperm tissue using a device such as the BIOLISTIC PD-1000 (Biorad; Hercules Calif.).

A nucleic acid of the present disclosure (e.g., a nucleic acid (e.g., a recombinant expression vector) comprising a nucleotide sequence encoding a variant CRISPR-Cas effector polypeptide or a variant CRISPR-Cas effector fusion polypeptide of the present disclosure)) may be introduced into a plant in a manner such that the nucleic acid is able to enter a plant cell(s), e.g., via an in vivo or ex vivo protocol. By “in vivo,” it is meant in the nucleic acid is administered to a living body of a plant e.g. infiltration. By “ex vivo” it is meant that cells or explants are modified outside of the plant, and then such cells or organs are regenerated to a plant. A number of vectors suitable for stable transformation of plant cells or for the establishment of transgenic plants have been described, including those described in Weissbach and Weissbach, (1989) Methods for Plant Molecular Biology Academic Press, and Gelvin et al., (1990) Plant Molecular Biology Manual, Kluwer Academic Publishers. Specific examples include those derived from a Ti plasmid of Agrobacterium tumefaciens, as well as those disclosed by Herrera-Estrella et al. (1983) Nature 303: 209, Bevan (1984) Nucl Acid Res. 12: 8711-8721, Klee (1985) Bio/Technolo 3: 637-642. Alternatively, non-Ti vectors can be used to transfer the DNA into plants and cells by using free DNA delivery techniques. By using these methods transgenic plants such as wheat, rice (Christou (1991) Bio/Technology 9:957-9 and 4462) and corn (Gordon-Kamm (1990) Plant Cell 2: 603-618) can be produced. An immature embryo can also be a good target tissue for monocots for direct DNA delivery techniques by using the particle gun (Weeks et al. (1993) Plant Physiol 102: 1077-1084; Vasil (1993) Bio/Technolo 10: 667-674; Wan and Lemeaux (1994) Plant Physiol 104: 37-48 and for Agrobacterium-mediated DNA transfer (Ishida et al. (1996) Nature Biotech 14: 745-750). Exemplary methods for introduction of DNA into chloroplasts are biolistic bombardment, polyethylene glycol transformation of protoplasts, and microinjection (Danieli et al Nat. Biotechnol 16:345-348, 1998; Staub et al Nat. Biotechnol 18: 333-338, 2000; O'Neill et al Plant J. 3:729-738, 1993; Knoblauch et al Nat. Biotechnol 17: 906-909; U.S. Pat. Nos. 5,451,513, 5,545,817, 5,545,818, and 5,576,198; in Intl. Application No. WO 95/16783; and in Boynton et al., Methods in Enzymology 217: 510-536 (1993), Svab et al., Proc. Natl. Acad. Sci. USA 90: 913-917 (1993), and McBride et al., Proc. Natl. Acad. Sci. USA 91: 7301-7305 (1994)). Any vector suitable for the methods of biolistic bombardment, polyethylene glycol transformation of protoplasts and microinjection will be suitable as a targeting vector for chloroplast transformation. Any double stranded DNA vector may be used as a transformation vector, especially when the method of introduction does not utilize Agrobacterium.

Plants which can be genetically modified include grains, forage crops, fruits, vegetables, oil seed crops, palms, forestry, and vines. Specific examples of plants which can be modified follow: maize, banana, peanut, field peas, sunflower, tomato, canola, tobacco, wheat, barley, oats, potato, soybeans, cotton, carnations, sorghum, lupin and rice.

The present disclosure provides transformed plant cells, tissues, plants and products that contain the transformed plant cells. A feature of the subject transformed cells, and tissues and products that include the same is the presence of a subject nucleic acid integrated into the genome, and production by plant cells of a variant CRISPR-Cas effector polypeptide or a variant CRISPR-Cas effector fusion polypeptide of the present disclosure. Recombinant plant cells of the present invention are useful as populations of recombinant cells, or as a tissue, seed, whole plant, stem, fruit, leaf, root, flower, stem, tuber, grain, animal feed, a field of plants, and the like.

Nucleotide sequences encoding a variant CRISPR-Cas effector polypeptide or a variant CRISPR-Cas effector fusion polypeptide can be under the control of (i.e., operably linked to) an unknown promoter (e.g., when the nucleic acid randomly integrates into a host cell genome) or can be under the control of (i.e., operably linked to) a known promoter. Suitable known promoters can be any known promoter and include constitutively active promoters, inducible promoters, spatially restricted and/or temporally restricted promoters, etc.

### Utility

The above-described variant CRISPR-Cas effector polypeptides, variant CRISPR-Cas effector fusion polypeptide fusion polypeptides, nucleic acids, RNPs, and expression vectors, find use in a variety of methods. For example, the above-described variant CRISPR-Cas effector polypeptides, variant CRISPR-Cas effector fusion polypeptides, nucleic acids, RNPs, and expression vectors, find use in modifying a target nucleic acid.

### Methods of Modifying a Target Nucleic Acid

A variant CRISPR-Cas effector polypeptide or a variant CRISPR-Cas effector fusion polypeptide, finds use in a variety of methods (e.g., in combination with a guide RNA and in some cases further in combination with a donor template). For example, a variant CRISPR-Cas effector polypeptide or a variant CRISPR-Cas effector fusion polypeptide of the present disclosure can be used to (i) modify (e.g., cleave, e.g., nick; methylate; etc.) target nucleic acid (DNA or RNA; single stranded or double stranded); (ii) modulate transcription of a target nucleic acid; (iii) label a target nucleic acid; (iv) bind a target nucleic acid (e.g., for purposes of isolation, labeling, imaging, tracking, etc.); (v) modify a polypeptide (e.g., a histone) associated with a target nucleic acid; and the like. Likewise for CRISPR-Cas polypeptides (e.g., ThermoCas9 variants) and CRISPR-Cas (e.g., ThermoCas9 variant) fusion polypeptides. Thus, the present disclosure provides a method of modifying a target nucleic acid. In some cases, a method of the present disclosure for modifying a target nucleic acid comprises contacting the target nucleic acid with: a) a variant CRISPR-Cas effector polypeptide or a variant CRISPR-Cas effector fusion polypeptide; and b) one or more (e.g., two) guide RNAs. Likewise for CRISPR-Cas polypeptides (e.g., ThermoCas9 variants) and CRISPR-Cas (e.g., ThermoCas9 variant) fusion polypeptides. In some cases, a method of the present disclosure for modifying a target nucleic acid comprises contacting the target nucleic acid with: a) a variant CRISPR-Cas effector polypeptide or a variant CRISPR-Cas effector fusion polypeptide; b) a guide RNA; and c) a donor nucleic acid (e.g., a donor template). Likewise for CRISPR-Cas polypeptides (e.g., ThermoCas9 variants) and CRISPR-Cas (e.g., ThermoCas9 variant) fusion polypeptides. In some cases, the contacting step is carried out in a cell in vitro. In some cases, the contacting step is carried out in a cell in vivo. In some such cases the cell is a lung cell or a liver cell. In some cases, the contacting step is carried out in a cell ex vivo. In some cases, a method of the present disclosure for modifying a target nucleic acid comprises contacting a cell that comprises the target nucleic acid with a system of the present disclosure.

Because a method that uses a variant CRISPR-Cas effector polypeptide or a variant CRISPR-Cas effector fusion polypeptide includes binding of the GeoCas9 polypeptide to a particular region in a target nucleic acid (by virtue of being targeted there by an associated guide RNA), the methods are generally referred to herein as methods of binding (e.g., a method of binding a target nucleic acid). However, it is to be understood that in some cases, while a method of binding may result in nothing more than binding of the target nucleic acid, in other cases, the method can have different final results (e.g., the method can result in modification of the target nucleic acid, e.g., cleavage/methylation/etc., modulation of transcription from the target nucleic acid; modulation of translation of the target nucleic acid; genome editing; modulation of a protein associated with the target nucleic acid; isolation of the target nucleic acid; etc.). Likewise for CRISPR-Cas polypeptides (e.g., ThermoCas9 variants) and CRISPR-Cas (e.g., ThermoCas9 variant) fusion polypeptides.

For example, the present disclosure provides (but is not limited to) methods of cleaving a target nucleic acid; methods of editing a target nucleic acid; methods of modulating transcription from a target nucleic acid; methods of isolating a target nucleic acid, methods of binding a target nucleic acid, methods of imaging a target nucleic acid, methods of modifying a target nucleic acid, and the like.

As used herein, the terms/phrases “contact a target nucleic acid” and “contacting a target nucleic acid”, for example, with a variant CRISPR-Cas effector polypeptide or a variant CRISPR-Cas effector fusion polypeptide, encompass all methods for contacting the target nucleic acid. For example, a variant CRISPR-Cas effector polypeptide or a variant CRISPR-Cas effector fusion polypeptide can be provided to a cell as protein, RNA (encoding the variant CRISPR-Cas effector polypeptide or variant CRISPR-Cas effector fusion polypeptide), or DNA (encoding the variant CRISPR-Cas effector polypeptide or the variant CRISPR-Cas effector fusion polypeptide); while a guide RNA can be provided as a guide RNA or as a nucleic acid encoding the guide RNA. As such, when, for example, performing a method in a cell (e.g., inside of a cell in vitro, inside of a cell in vivo, inside of a cell ex vivo), a method that includes contacting the target nucleic acid encompasses the introduction into the cell of any or all of the components in their active/final state (e.g., in the form of a protein(s) for a variant CRISPR-Cas effector polypeptide or a variant CRISPR-Cas effector fusion polypeptide; in the form of an RNA in some cases for a guide RNA), and also encompasses the introduction into the cell of one or more nucleic acids encoding one or more of the components (e.g., nucleic acid(s) comprising nucleotide sequence(s) encoding a variant CRISPR-Cas effector polypeptide or a variant CRISPR-Cas effector fusion polypeptide, nucleic acid(s) comprising nucleotide sequence(s) encoding guide RNA(s), nucleic acid comprising a nucleotide sequence encoding a donor template, and the like). Because the methods can also be performed in vitro outside of a cell, a method that includes contacting a target nucleic acid, (unless otherwise specified) encompasses contacting outside of a cell in vitro, inside of a cell in vitro, inside of a cell in vivo, inside of a cell ex vivo, etc.

In some cases, a method of the present disclosure for modifying a target nucleic acid comprises contacting a target nucleic acid with a variant CRISPR-Cas effector polypeptide or a variant CRISPR-Cas effector fusion polypeptide. In some cases, a method of the present disclosure for modifying a target nucleic acid comprises contacting a target nucleic acid with a variant CRISPR-Cas effector polypeptide or a variant CRISPR-Cas effector fusion polypeptide and a guide RNA. In some cases, a method of the present disclosure for modifying a target nucleic acid comprises contacting a target nucleic acid with a variant CRISPR-Cas effector polypeptide or a variant CRISPR-Cas effector fusion polypeptide, a first guide RNA, and a GeoCas9 guide RNA In some cases, a method of the present disclosure for modifying a target nucleic acid comprises contacting a target nucleic acid with a variant CRISPR-Cas effector polypeptide or a variant CRISPR-Cas effector fusion polypeptide, a guide RNA, and a donor DNA template.

### Target Nucleic Acids and Target Cells of Interest

A variant CRISPR-Cas effector polypeptide or a variant CRISPR-Cas effector fusion polypeptide, when bound to a guide RNA, can bind to a target nucleic acid, and in some cases, can bind to and modify a target nucleic acid. A target nucleic acid can be any nucleic acid (e.g., DNA, RNA), can be double stranded or single stranded, can be any type of nucleic acid (e.g., a chromosome (genomic DNA), derived from a chromosome, chromosomal DNA, plasmid, viral, extracellular, intracellular, mitochondrial, chloroplast, linear, circular, etc.) and can be from any organism (e.g., as long as the guide RNA comprises a nucleotide sequence that hybridizes to a target sequence in a target nucleic acid, such that the target nucleic acid can be targeted).

A target nucleic acid can be DNA or RNA. A target nucleic acid can be double stranded (e.g., dsDNA, dsRNA) or single stranded (e.g., ssRNA, ssDNA). In some cases, a target nucleic acid is single stranded. In some cases, a target nucleic acid is a single stranded RNA (ssRNA). In some cases, a target ssRNA (e.g., a target cell ssRNA, a viral ssRNA, etc.) is selected from: mRNA, rRNA, tRNA, non-coding RNA (ncRNA), long non-coding RNA (lncRNA), and microRNA (miRNA). In some cases, a target nucleic acid is a single stranded DNA (ssDNA) (e.g., a viral DNA). As noted above, in some cases, a target nucleic acid is single stranded.

A target nucleic acid can be located anywhere, for example, outside of a cell in vitro, inside of a cell in vitro, inside of a cell in vivo, inside of a cell ex vivo. Suitable target cells (which can comprise target nucleic acids such as genomic DNA) include, but are not limited to: a bacterial cell; an archaeal cell; a cell of a single-cell eukaryotic organism; a plant cell; an algal cell, e.g., Botryococcus braunii, Chlamydomonas reinhardtii, Nannochloropsis gaditana, Chiorella pyrenoidosa, Sargassum patens, C. agardh, and the like; a fungal cell (e.g., a yeast cell); an animal cell; a cell from an invertebrate animal (e.g. fruit fly, a enidarian, an echinoderm, a nematode, etc.); a cell of an insect (e.g., a mosquito; a bee; an agricultural pest; etc.); a cell of an arachnid (e.g., a spider; a tick; etc.); a cell from a vertebrate animal (e.g., a fish, an amphibian, a reptile, a bird, a mammal); a cell from a mammal (e.g., a cell from a rodent; a cell from a human; a cell of a non-human mammal; a cell of a rodent (e.g., a mouse, a rat); a cell of a lagomorph (e.g., a rabbit); a cell of an ungulate (e.g., a cow, a horse, a camel, a llama, a vicuia, a sheep, a goat, etc.); a cell of a marine mammal (e.g., a whale, a seal, an elephant seal, a dolphin, a sea lion; etc.) and the like. Any type of cell may be of interest (e.g. a stem cell, e.g. an embryonic stem (ES) cell, an induced pluripotent stem (iPS) cell, a germ cell (e.g., an oocyte, a sperm, an oogonia, a spermatogonia, etc.), an adult stem cell, a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.).

Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in vitro. Target cells can be unicellular organisms and/or can be grown in culture. If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. can be conveniently harvested by biopsy.

In some of the above applications, the subject methods may be employed to induce target nucleic acid cleavage, target nucleic acid modification, and/or to bind target nucleic acids (e.g., for visualization, for collecting and/or analyzing, etc.) in mitotic or post-mitotic cells in vivo and/or ex vivo and/or in vitro (e.g., to disrupt production of a protein encoded by a targeted mRNA, to cleave or otherwise modify target DNA, to genetically modify a target cell, and the like). Because the guide RNA provides specificity by hybridizing to target nucleic acid, a mitotic and/or post-mitotic cell of interest in the disclosed methods may include a cell from any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens, C. agardh, and the like, a fungal cell (e.g., a yeast cell), an animal cell, a cell from an invertebrate animal (e.g. fruit fly, cnidarian, echinoderm, nematode, etc.). a cell from a vertebrate animal (e.g., fish, amphibian, reptile, bird, mammal), a cell from a mammal, a cell from a rodent, a cell from a human, etc.). In some cases, a GeoCas9 protein (and/or nucleic acid encoding the protein such as DNA and/or RNA), and/or GeoCas9 guide RNA (and/or a DNA encoding the guide RNA), and/or donor template, and/or RNP can be introduced into an individual (i.e., the target cell can be in vivo) (e.g., a mammal, a rat, a mouse, a pig, a primate, a non-human primate, a human, etc.). In some case, such an administration can be for the purpose of treating and/or preventing a disease, e.g., by editing the genome of targeted cells.

Plant cells include cells of a monocotyledon, and cells of a dicotyledon. The cells can be root cells, leaf cells, cells of the xylem, cells of the phloem, cells of the cambium, apical meristem cells, parenchyma cells, collenchyma cells, sclerenchyma cells, and the like. Plant cells include cells of agricultural crops such as wheat, corn, rice, sorghum, millet, soybean, etc. Plant cells include cells of agricultural fruit and nut plants, e.g., plant that produce apricots, oranges, lemons, apples, plums, pears, almonds, etc.

Additional examples of target cells are listed above in the section titled “Modified cells.” Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatos, rice, cassava, sugarcane, pumpkin, hay, potatos, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomnonas reinhardtii, Nannochloropsis gaditana, Chlorella pyrenoidosa, Sargassun patens, C. agardh, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be and in vivo cell (e.g., a cell in an individual). A cell can be an isolated cell. A cell can be a cell inside of an organism. A cell can be an organism. A cell can be a cell in a cell culture (e.g., in vitro cell culture). A cell can be one of a collection of cells. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a mammalian cell or derived from a mammalian cell. A cell can be a rodent cell or derived from a rodent cell. A cell can be a human cell or derived from a human cell. A cell can be a microbe cell or derived from a microbe cell. A cell can be a fungi cell or derived from a fungi cell. A cell can be an insect cell. A cell can be an arthropod cell. A cell can be a protozoan cell. A cell can be a helminth cell.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogenic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some embodiments, the stem cell is a hematopoietic stem cell (HSC). HSCs are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

A cell is in some cases a plant cell. A plant cell can be a cell of a monocotyledon. A cell can be a cell of a dicotyledon.

In some cases, the cell is a plant cell. For example, the cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Rice, Sorghum, Soybeans, Sugarbeets, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf—green), lettuce (oak leaf—red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictvoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

In some cases, a suitable cell is a prokaryotic cell. In some cases, the cell is a thermophilic prokaryotic cell. In some cases, the cell is an obligate thermophile (e.g., an obligate thermophilic prokaryotic cell). In some cases, the cell is a microorganism selected from a Cupriavidus sp., Ralstonia sp, Xanthobacter sp., Rhodococcus sp., Hydrogenovibrio sp., Rhodopseudomonas sp., Rhodobacter sp., Hydrogenobacter sp., Arthrobacter sp., Paracoccus sp., Mycobacterium sp., Streptomyces sp., and Bacillus sp.

In some cases, the host cell is a microorganism. In some cases, the microorganism is Rhodococcus opacus or Rhodococcus jostii or Rhodococcus sp. In some cases, the microorganism is Hydrogenovibrio marinus. In some cases, the microorganism is Rhodopseudomonas capsulata, Rhodopseudomonas palustris, or Rhodobacter sphaeroides. In some cases, the microorganism is an oxyhydrogen or knallgas strain. In some cases, the microorganism is selected from: Aquifex pyrophilus and Aquifex aeolicus or other Aquifex sp.; Cupriavidus necator or Cupriavidus metallidurans or other Cupriavidus sp.; Corynebacterium autotrophicum or other Corynebacterium sp.; Nocardia autotrophica and Nocardia opaca and other Nocardia sp.; purple non-sulfur photosynthetic bacteria including but not limited to Rhodopseudomonas palustris, Rhodopseudomonas capsulata, Rhodopseudomonas viridis, Rhodopseudomonas sulfoviridis, Rhodopseudomonas blastica, Rhodopseudomonas spheroides. Rhodopseudomonas acidophila and other Rhodopseudomonas sp., Rhodospirillum rubrum, and other Rhodospirillum sp.; Rhodococcus opacus and other Rhodococcus sp.; Rhizobium japonicum and other Rhizobium sp.; Thiocapsa roseopersicina and other Thiocapsa sp.; Pseudomonasfacilis and Pseudomonas flava and Pseudomonas putida and Pseudomonas hydrogenovora, Pseudomonas hydrogenothermophila, Pseudomonas palleronii and Pseudomonas pseudoflava and Pseudomonas saccharophila and Pseudomonas thermophila and other Pseudomonas sp.; Hydrogenomonas pantotropha, Hydrogenomonas eutropha. Hydrogenomonas facilis. and other Hydrogenomonas sp.; Hydrogenobacter thermophilus and Hydrogenobacter halophilus and Hydrogenobacter hydrogenophilus and other Hydrogenobacter sp.; Hydrogenophilus islandicus and other Hydrogenophilus sp.; Hydrogenovibrio marinus and other Hydrogenovibrio sp.; Hydrogenothermus marinus and other Hydrogenothermus sp.; Helicobacter pylori and other Helicobacter sp.; Xanthobacter autotrophicus and Xanthobacter flavus other Xanthobacter sp.; Hydrogenophagaflava and Hydrogenophaga palleronii and Hydrogenophaga pseudoflava and other Hydrogenophaga sp.; Bradyrhizobium japonicum and other Bradyrhizobium sp.; Ralstonia eutropha and other Ralstonia sp.; Alcaligenes eutrophus and Alcaligenes facilis and Alcaligenes hydrogenophilus and Alcaligenes latus and Alcaligenes paradoxus and Alcaligenes ruhlandii and other Alcaligenes sp.; Amycolata sp.; Aquaspirillum autotrophicum and other Aquaspirillum sp.; Arthrobacter strain 11/X and other Arthrobacter sp.; Azospirillum lipoferum and other Azospirillum sp.; Variovorax paradoxus, and other Variovorax sp.; Acidovorax facilis, and other Acidovorax sp.; Bacillus schlegelii and Bacillus tusciae and other Bacillus sp.; Calderobacterium hydrogenophilum and other Calderobacterium sp.; Derxia gummosa and other Derxia sp.; Flavobacterium autothermophilum and other Flavobacterium sp.; Microcyclus aquaticus and other Microcyclus; Mycobacterium gordoniae and other Mycobacterium sp.; Paracoccus denitrificans and other Paracoccus sp.; Persephonella marina and Persephonella guaymasensis and other Persephonella sp.; Renobacter vacuolatum and other Renobacter sp.; Thermocrinis ruber and other Thermocrinis sp.; Wautersia sp.; cyanobacteria including but not limited to Anabaena oscillarioides, Anabaena spiroides, Anabaena cylindrica, and other Anabaena sp.; green algae including but not limited to Scenedesmus obliquus and other Scenedesmus sp., Chlamydomonas reinhardtii and other Chlamydomonas sp., Ankistrodesmus sp., Rhaphidium polymorphium and other Rhaphidium sp; as well as a consortiums of microorganisms that include oxyhydrogen microorganisms.

In some cases, the host cell is an obligate and/or facultative chemoautotrophic microorganism selected from: Acetoanaerobium sp.; Acetobacterium sp.; Acetogenium sp.; Achromobacter sp.; Acidianus sp.; Acinetobacter sp.; Actinomadura sp.; Aeromonas sp.; Alcaligenes sp.; Alcaligenes sp.; Arcobacter sp.; Aureobacterium sp.; Bacillus sp.; Beggiatoa sp.; Butyribacterium sp.; Carboxydothermus sp.; Clostridium sp.; Comamonas sp.; Dehalobacter sp.; Dehalococcoide sp.; Dehalospirillum sp.; Desulfobacterium sp.; Desulfomonile sp.; Desulfotomaculum sp.; Desulfovibrio sp.; Desulfurosarcina sp.; Ectothiorhodospira sp.; Enterobacter sp.; Eubacterium sp.; Ferroplasma sp.; Halothibacillus sp.; Hydrogenobacter sp.; Hydrogenomonas sp.; Leptospirillum sp.; Metallosphaera sp.; Methanobacterium sp.; Methanobrevibacter sp.; Methanococcus sp.; Methanosarcina sp.; Micrococcus sp.; Nitrobacter sp.; Nitrosococcus sp.; Nitrosolobus sp.; Nitrosomonas sp.; Nitrosospira sp.; Nitrosovibrio sp.; Nitrospina sp.; Oleomonas sp.; Paracoccus sp.; Peptostreptococcus sp.; Planctomycetes sp.; Pseudomonas sp.; Ralstonia sp.; Rhodobacter sp.; Rhodococcus sp.; Rhodocyclus sp.; Rhodomicrobium sp.; Rhodopseudomonas sp.; Rhodospirillum sp.; Shewanella sp.; Streptomyces sp.; Sulfobacillus sp.; Sulfolobus sp.; Thiobacillus sp.; Thiomicrospira sp.; Thioploca sp.; Thiosphaera sp.; Thiothrix sp.; sulfur-oxidizers; hydrogen-oxidizers; iron-oxidizers; acetogens; and methanogens; consortiums of microorganisms that include chemoautotrophs; chemoautotrophs native to at least one of hydrothermal vents, geothermal vents, hot springs, cold seeps, underground aquifers, salt lakes, saline formations, mines, acid mine drainage, mine tailings, oil wells, refinery wastewater, coal seams, deep sub-surface; waste water and sewage treatment plants; geothermal power plants, sulfatara fields, and soils; and extremophiles selected from one or more of thermophiles, hyperthermophiles, acidophiles, halophiles, and psychrophiles.

### Donor Polynucleotide (Donor Template)

Guided by a dual or single guide RNA, a variant CRISPR-Cas effector polypeptide or a variant CRISPR-Cas effector fusion polypeptide in some cases generates site-specific double strand breaks (DSBs) or single strand breaks (SSBs) (e.g., when the GeoCas9 protein is a nickase variant) within double-stranded DNA (dsDNA) target nucleic acids, which are repaired either by non-homologous end joining (NHEJ) or homology-directed recombination (HDR).

In some cases, contacting a target DNA (with a variant CRISPR-Cas effector polypeptide or a variant CRISPR-Cas effector fusion polypeptide and a guide RNA) occurs under conditions that are permissive for nonhomologous end joining or homology-directed repair. Thus, in some cases, a subject method includes contacting the target DNA with a donor polynucleotide (e.g., by introducing the donor polynucleotide into a cell), wherein the donor polynucleotide, a portion of the donor polynucleotide, a copy of the donor polynucleotide, or a portion of a copy of the donor polynucleotide integrates into the target DNA. In some cases, the method does not comprise contacting a cell with a donor polynucleotide, and the target DNA is modified such that nucleotides within the target DNA are deleted.

In some cases, guide RNA (or DNA encoding same) and a variant CRISPR-Cas effector polypeptide or a variant CRISPR-Cas effector fusion polypeptide (or a nucleic acid encoding same, such as an RNA or a DNA, e.g, one or more expression vectors) are coadministered (e.g., contacted with a target nucleic acid, administered to cells, etc.) with a donor polynucleotide sequence that includes at least a segment with homology to the target DNA sequence, the subject methods may be used to add, i.e. insert or replace, nucleic acid material to a target DNA sequence (e.g. to “knock in” a nucleic acid, e.g., one that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, etc.), to modify a nucleic acid sequence (e.g., introduce a mutation, remove a disease-causing mutation by introducing a correct sequence), and the like. As such, a complex comprising a GeoCas9 guide RNA and GeoCas9 protein is useful in any in vitro or in vivo application in which it is desirable to modify DNA in a site-specific, i.e. “targeted”, way, for example gene knock-out, gene knock-in, gene editing, gene tagging, etc., as used in, for example, gene therapy, e.g. to treat a disease or as an antiviral, antipathogenic, or anticancer therapeutic, the production of genetically modified organisms in agriculture, the large scale production of proteins by cells for therapeutic, diagnostic, or research purposes, the induction of iPS cells, biological research, the targeting of genes of pathogens for deletion or replacement, etc.

In applications in which it is desirable to insert a polynucleotide sequence into the genome where a target sequence is cleaved, a donor polynucleotide (a nucleic acid comprising a donor sequence) can also be provided to the cell. By a “donor sequence” or “donor polynucleotide” or “donor template” it is meant a nucleic acid sequence to be inserted at the site cleaved by the GeoCas9 protein (e.g., after dsDNA cleavage, after nicking a target DNA, after dual nicking a target DNA, and the like). The donor polynucleotide can contain sufficient homology to a genomic sequence at the target site, e.g. 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) can support homology-directed repair. Donor polynucleotides can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair (e.g., for gene correction, e.g., to convert a disease-causing base pair to a non disease-causing base pair). In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

In some cases, the donor sequence is provided to the cell as single-stranded DNA. In some cases, the donor sequence is provided to the cell as double-stranded DNA. It may be introduced into a cell in linear or circular form. If introduced in linear form, the ends of the donor sequence may be protected (e.g., from exonucleolytic degradation) by any convenient method and such methods are known to those of skill in the art. For example, one or more dideoxynucleotide residues can be added to the 3′ terminus of a linear molecule and/or self-complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described elsewhere herein for nucleic acids encoding a guide RNA and/or a variant CRISPR-Cas effector polypeptide or a variant CRISPR-Cas effector fusion polypeptide and/or donor polynucleotide.

### Lipid Nanoparticles (Lnps)

The present disclosure provides lipid nanoparticles (LNPs) comprising: i) a variant CRISPR-Cas effector polypeptide or a variant CRISPR-Cas effector fusion polypeptide of the present disclosure; and ii) a guide RNA. In some cases, an LNP comprises: i) a variant CRISPR-Cas effector polypeptide or a variant CRISPR-Cas effector fusion polypeptide of the present disclosure; ii) a guide RNA; and iii) a donor nucleic acid. In some cases, an LNP comprises a system of the present disclosure.

In some cases, an LNP of the present disclosure comprises: a) a first lipid, wherein the first lipid comprises a covalently attached poly(ethylene glycol) (PEG) moiety; b) at least a second and a third lipid, wherein the second lipid and the third lipid do not comprise a covalently attached PEG moiety; c) a CRISPR-Cas effector polypeptide; and d) a guide nucleic acid. A lipid that comprises a covalently linked PEG moiety is also referred to herein as a “PEGylated lipid.”

DOTAP has the following structure:

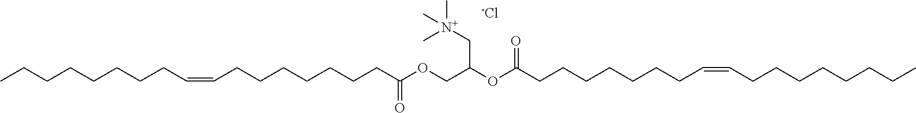

DOPE has the following structure:

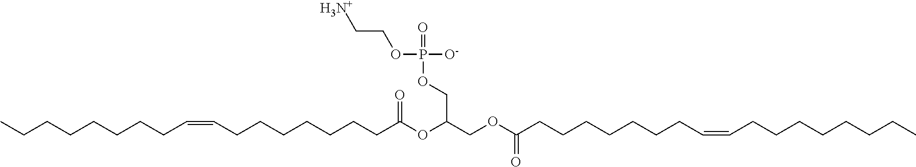 [SMILES: CCCCCCCC/C=C\CCCCCCCC(=O)OCC(COP(=O)([O-])OCC[NH3+])OC(=O)CCCCCCC/C=C\CCCCCCCC]

D-Lin has the following structure:

Cholesterol has the following structure:

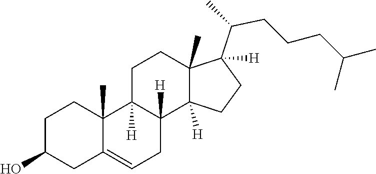

M-2k has the following structure:

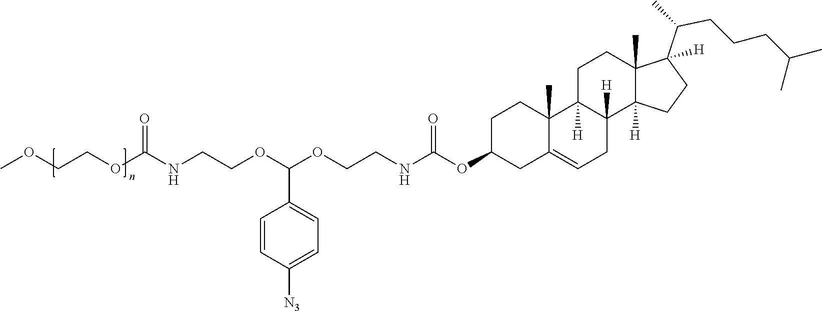

In some cases, the first lipid is PEGylated DOPE (1,2-dioleyl-sn-glycero-3-phosphoethanolamine). In some cases, the first lipid is PEGylated DMG (1,2-Dimyristoyl-rac-glycerol). In some cases, the first lipid is M-PEG (also referred to herein as “M-2k”). In some cases, the first lipid is selected from the group consisting of M-PEG, DMG-mPEG, DOPE-mPEG, DOPE-PEG-CO₂H, DOPE-PEG-NH₂. In some cases, the PEG has a molecular weight of from about 1.5 kD to about 3 kD. In some cases, the PEG has a molecular weight of about 2 kD.

In some cases, the second and third lipids are selected from the group consisting of DOPE (1,2-dioleyl-sn-glycero-3-phosphoethanolamine), DSPE (1,2-distearoyl-sn-glycero-3-phosphoethanolamine), DOTAP (1,2-dioleoyl-3-trimethylammonium-propane), DSTAP (1,2-stearoyl-3-trimethylammonium-propane), D-Lin, C12-300, and cholesterol.

In some cases, a LNP comprises DOTAP, D-Lin, DOPE, cholesterol, and M-PEG-2k. In some cases, the LNP comprises DOTAP, D-Lin, DOPE, cholesterol, and M-PEG-2k in a molar ratio of 18-20:36:12-14:40:1.5-2.5 DOTAP:D-Lin:DOPE:cholesterol:M-PEG-2k. In some cases, the LNP comprises DOTAP, D-Lin, DOPE, cholesterol, and M-PEG-2k in a molar ratio of20:26:12.5:40:1.5 DOTAP, D-Lin, DOPE, cholesterol, and M-PEG-2k.

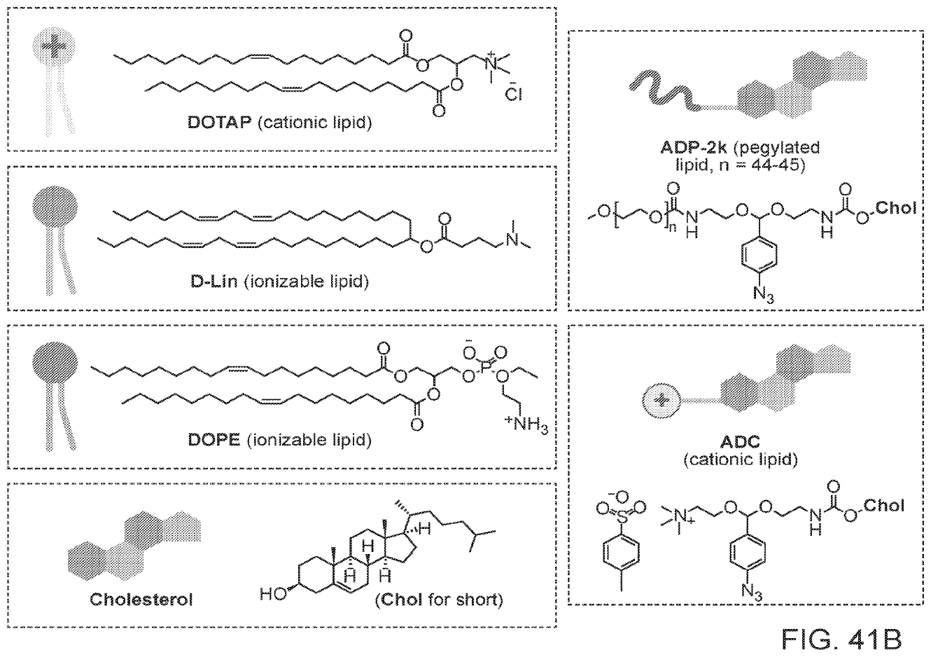

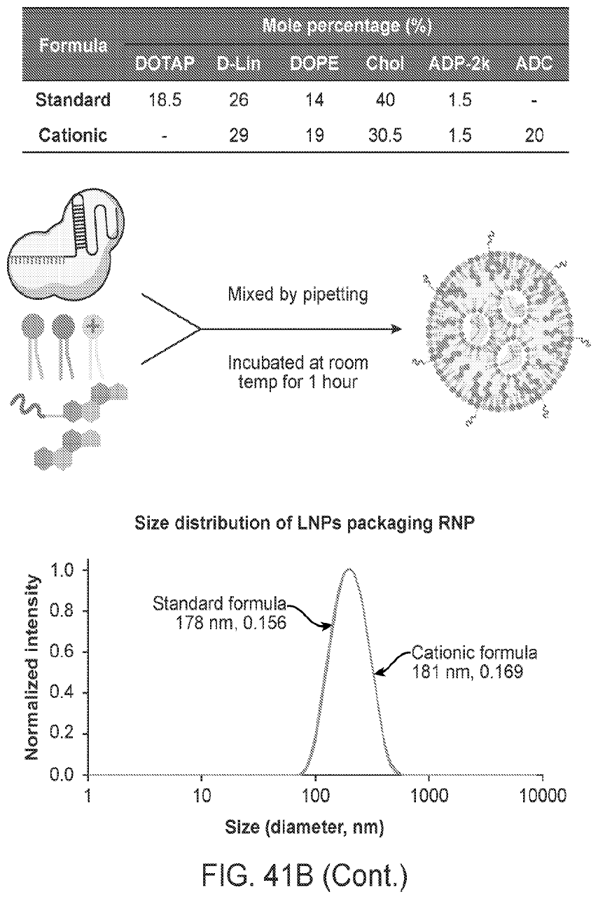

In some cases, ADC is used as the cationic lipid instead of DOTAP (see, e.g., FIG. 41B). This can, in some cases, lead to targeting of lung tissue.

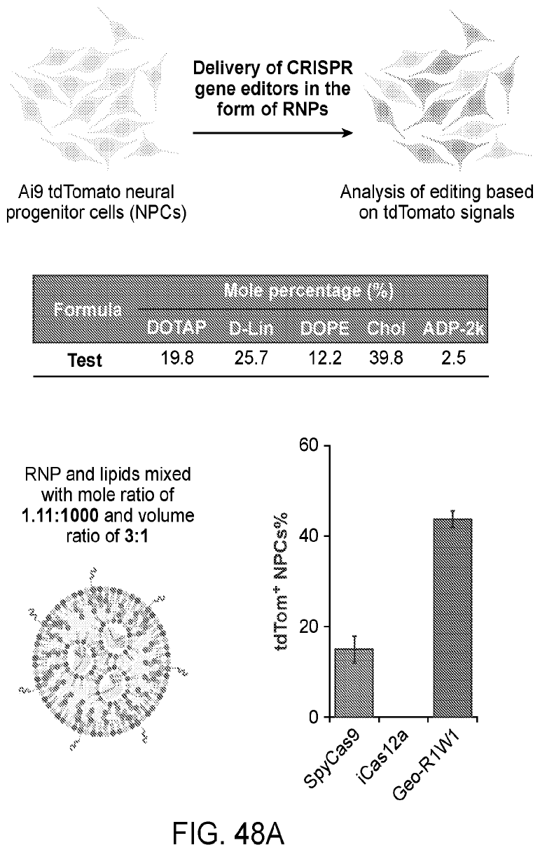

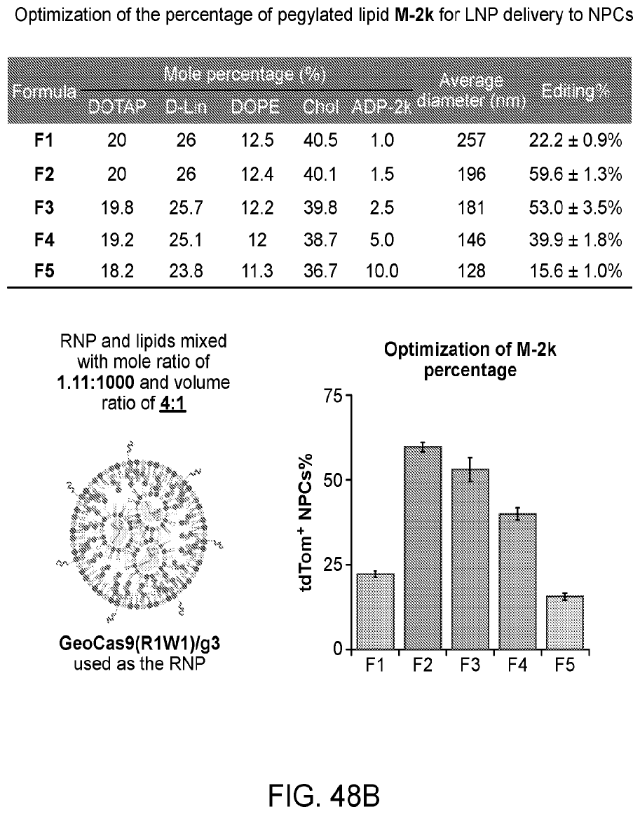

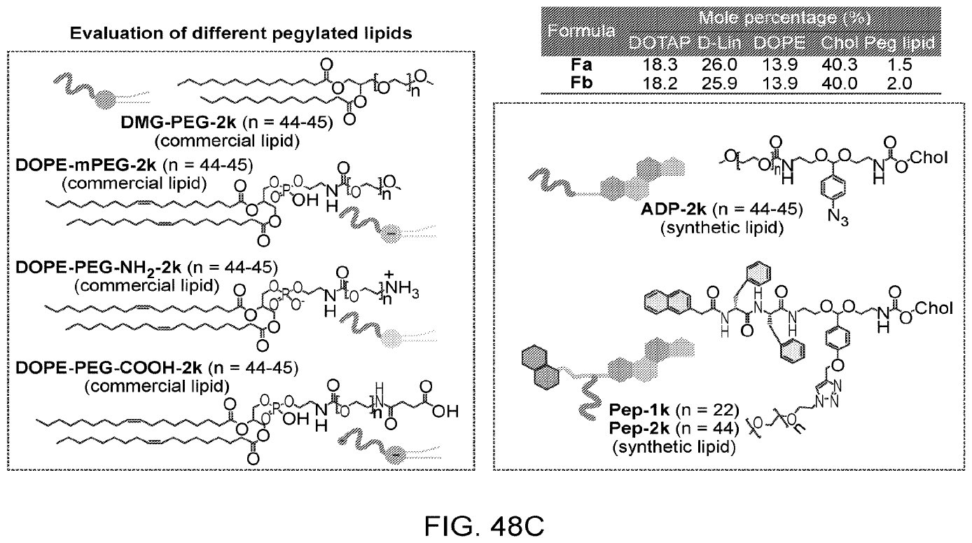

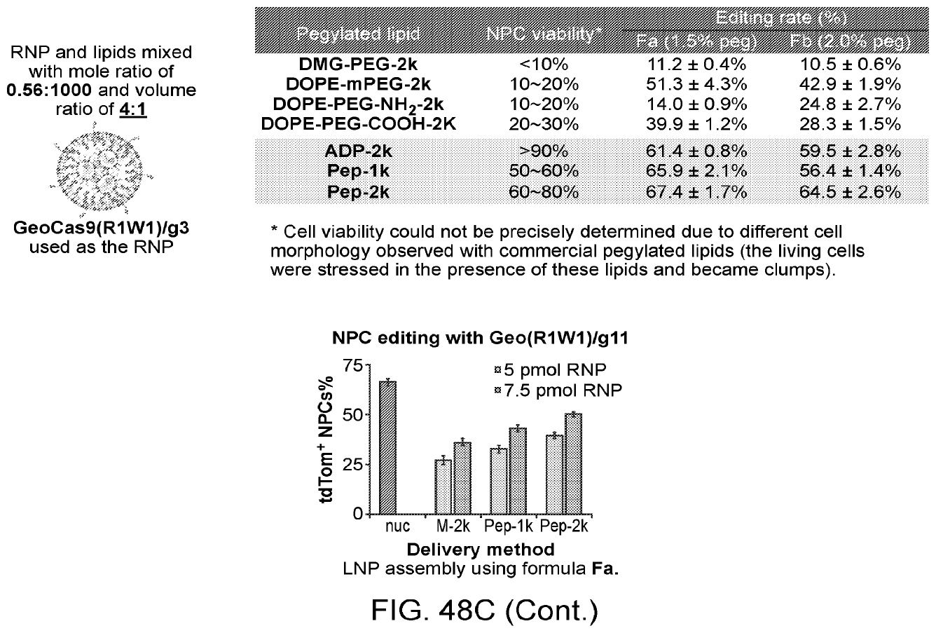

In some embodiments, an acid-degradable PEGylated lipid (a PEGylated lipid with an acid-degradable linker) is used. Examples of such include, but are not necessarily limited to: ADP-2k, Pep-1k, and Pep-2k (see, e.g., FIG. 48C). For structures and various lipid formulations, see, e.g., FIG. 41B and FIG. 48C.

As noted above, in some cases, an LNP of the present disclosure comprises a CRISPR-Cas effector polypeptide. Suitable CRISPR-Cas effector polypeptides include Type II CRISPR-Cas effector polypeptides, Type III CRISPR Cas effector polypeptides, Type V CRISPR Cas effector polypeptides, and Type VI CRISPR-Cas effector polypeptides. Suitable CRISPR-Cas effector polypeptides include fusion polypeptides that comprise: i) a CRISPR-Cas effector polypeptide; and ii) one or more heterologous polypeptides. In some cases, an LNP of the present disclosure comprises a variant CRISPR-Cas effector polypeptide, or a variant CRISPR-Cas effector fusion polypeptide, of the present disclosure.

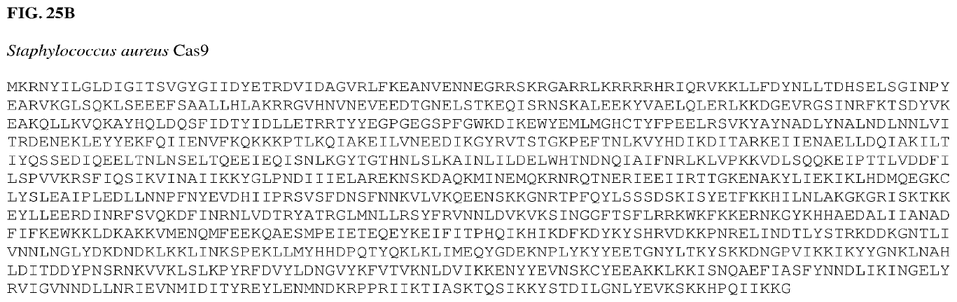

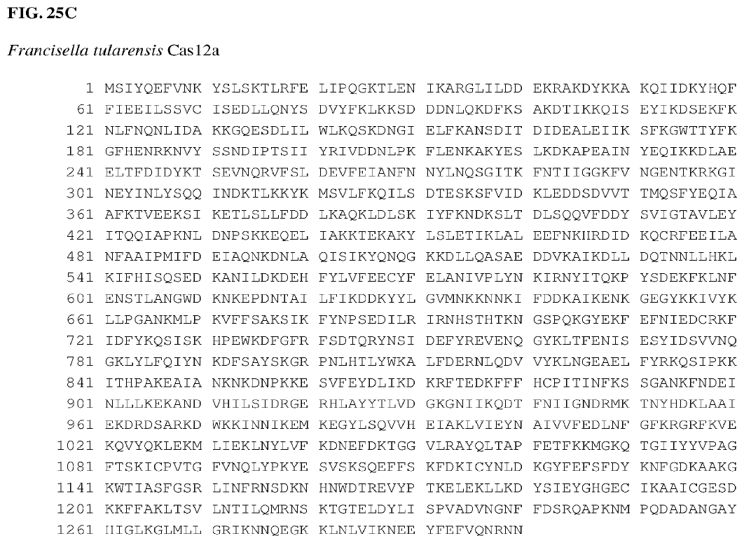

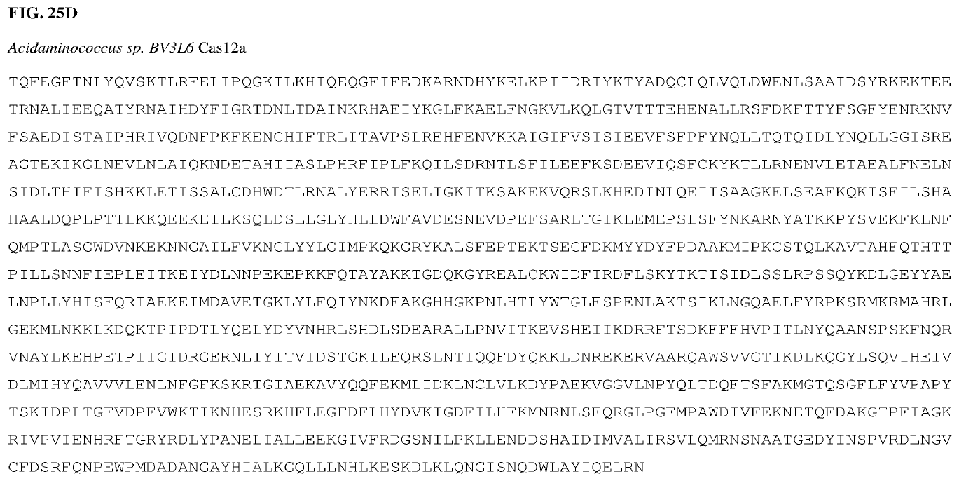

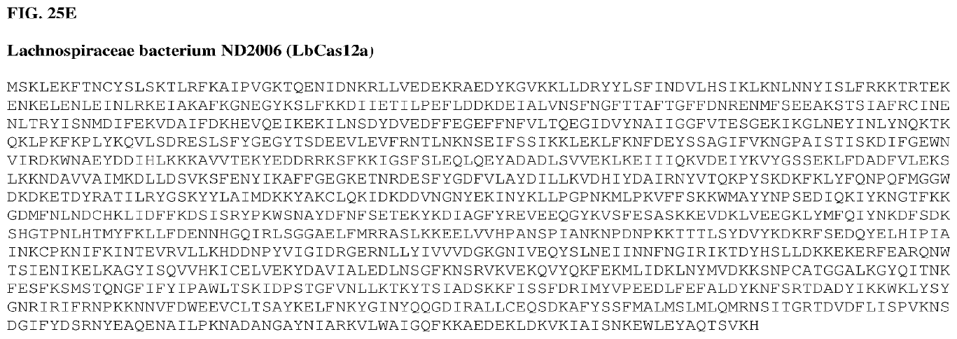

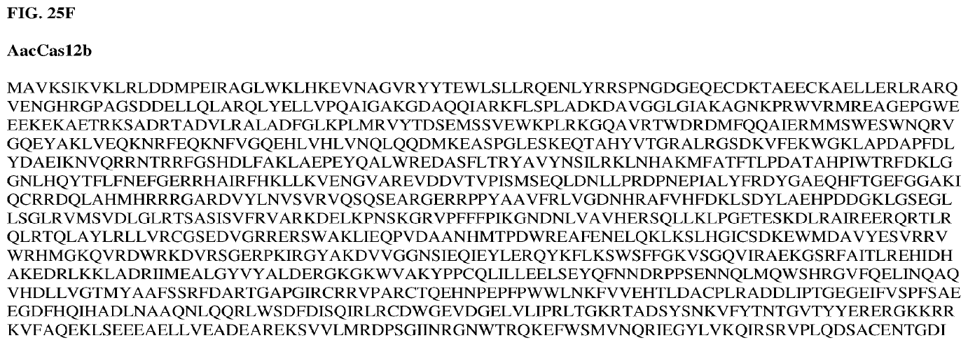

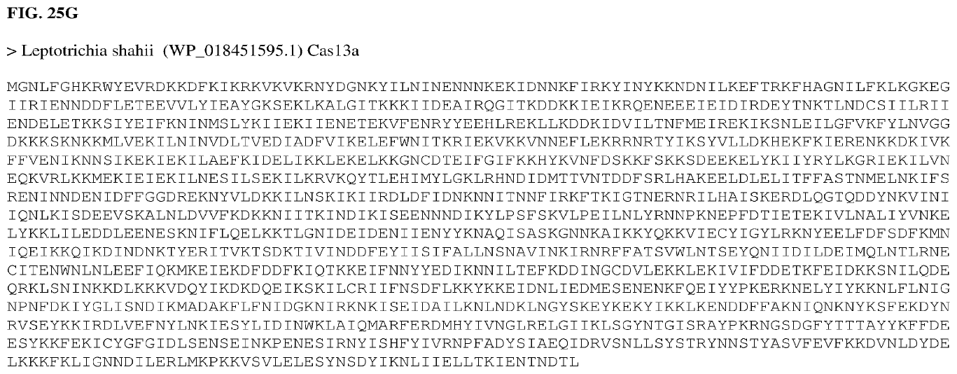

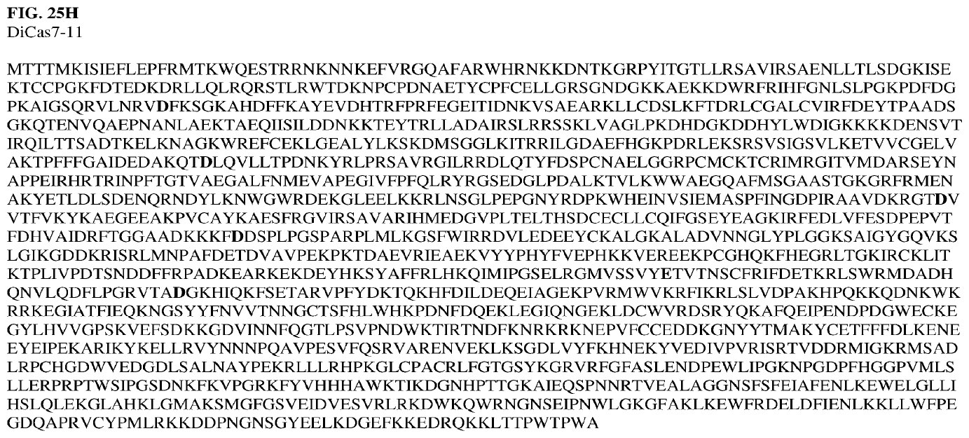

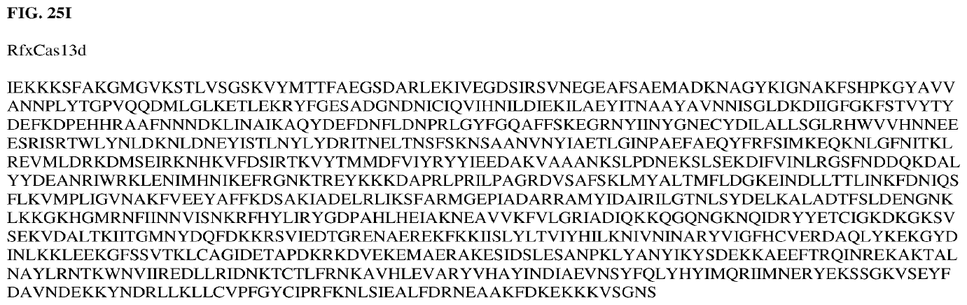

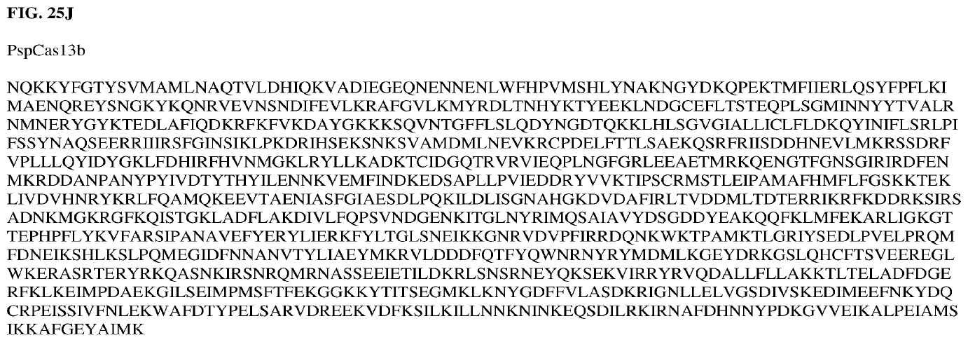

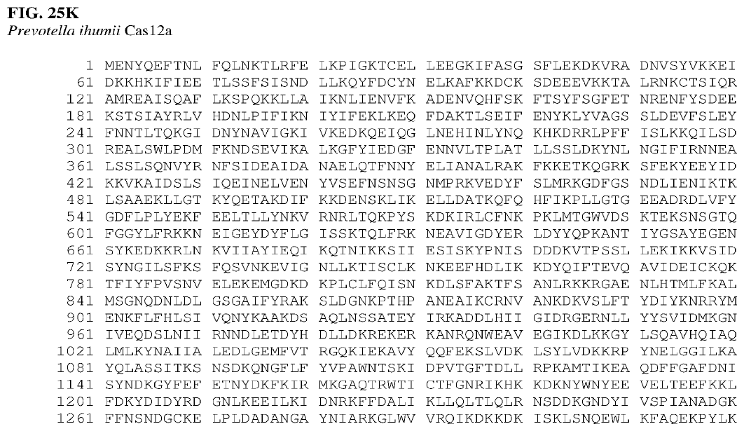

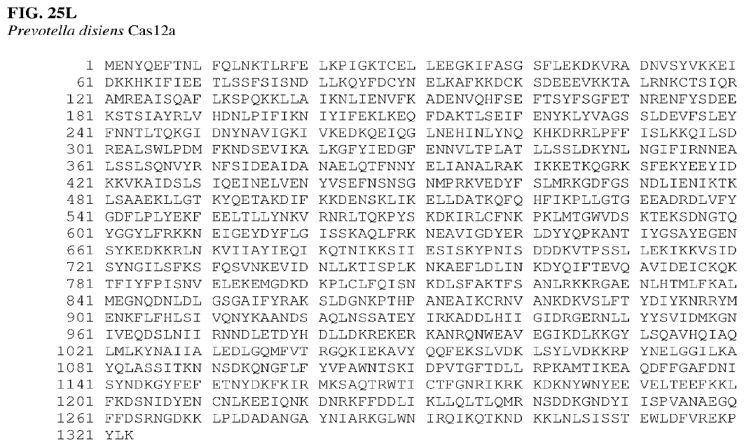

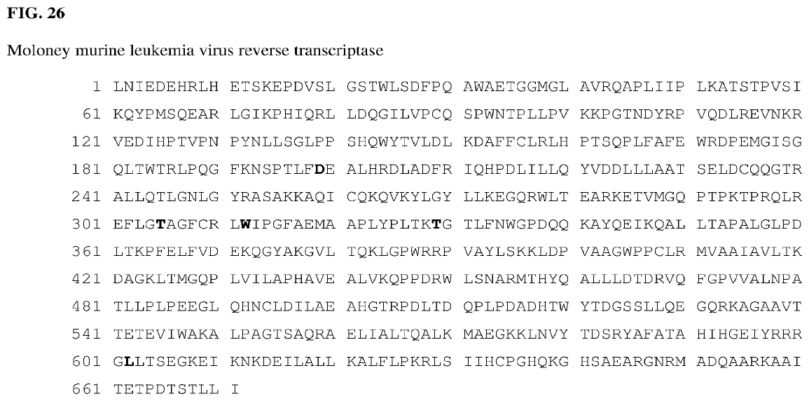

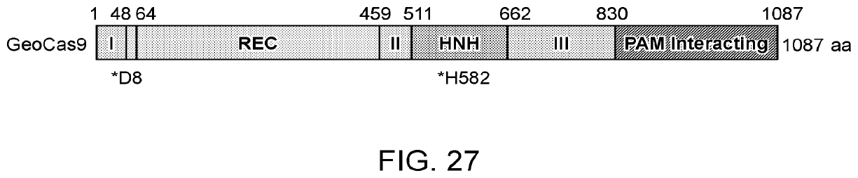

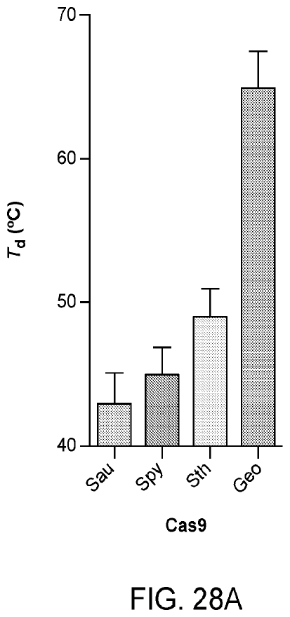

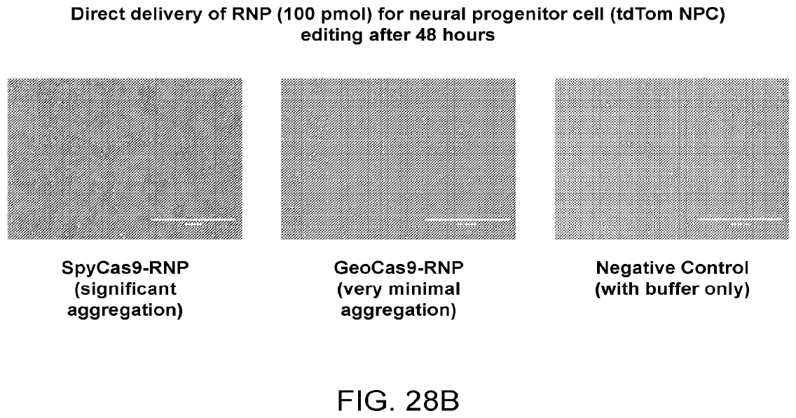

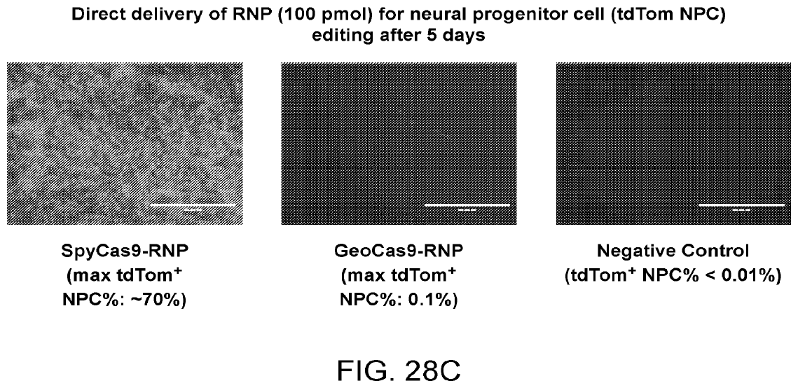

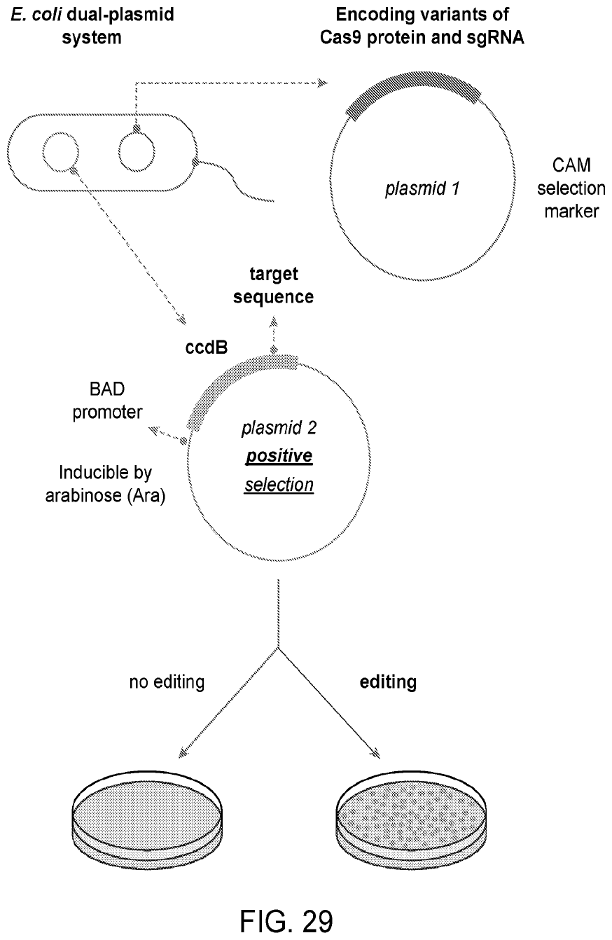

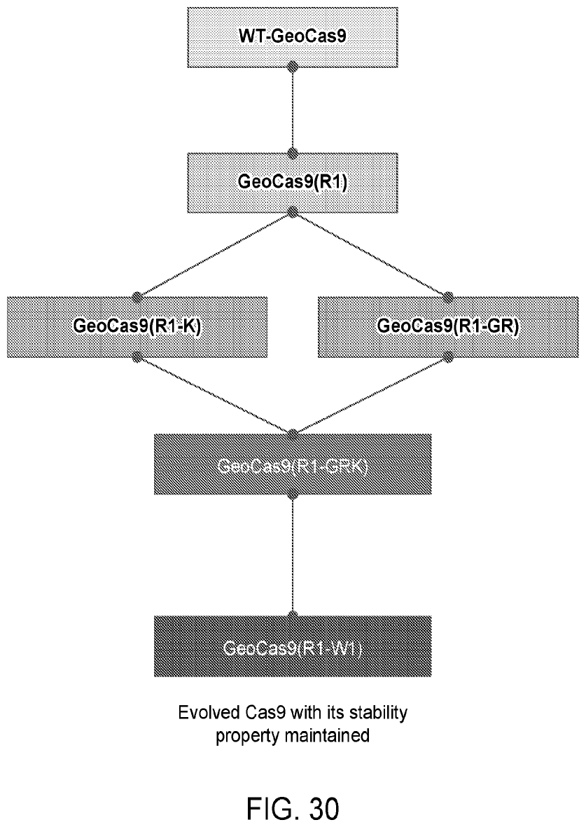

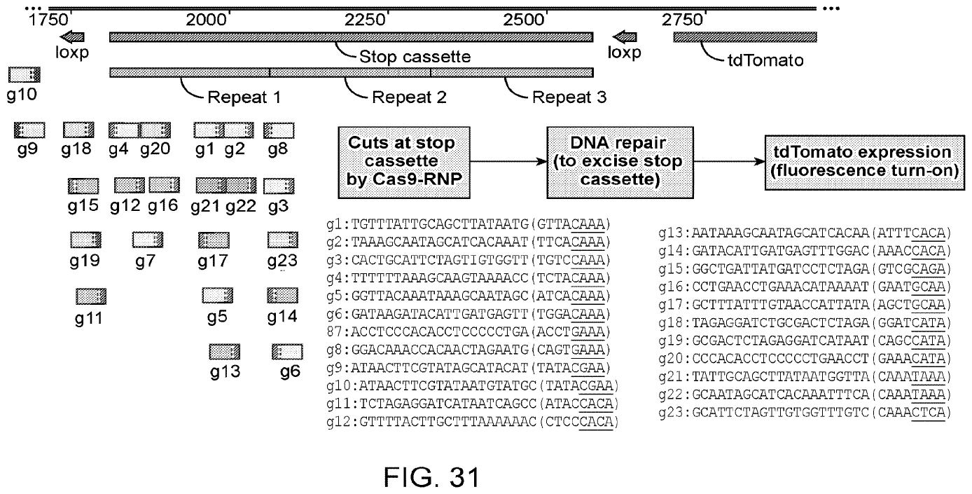

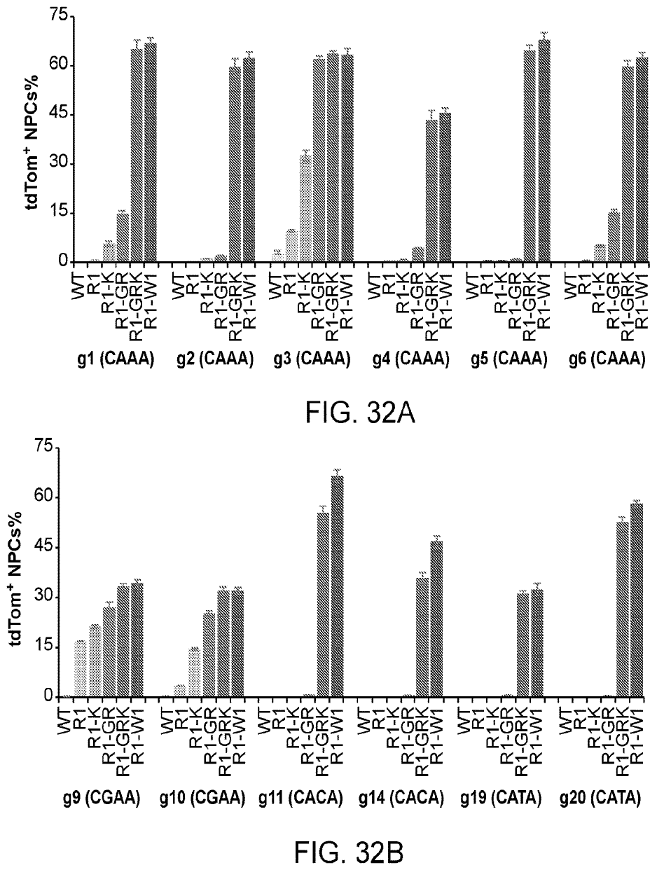

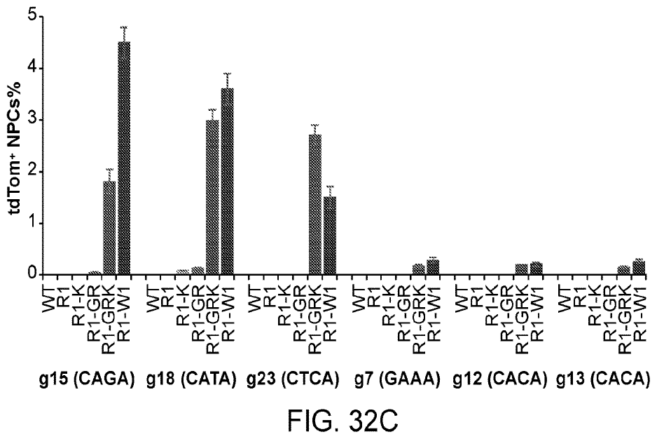

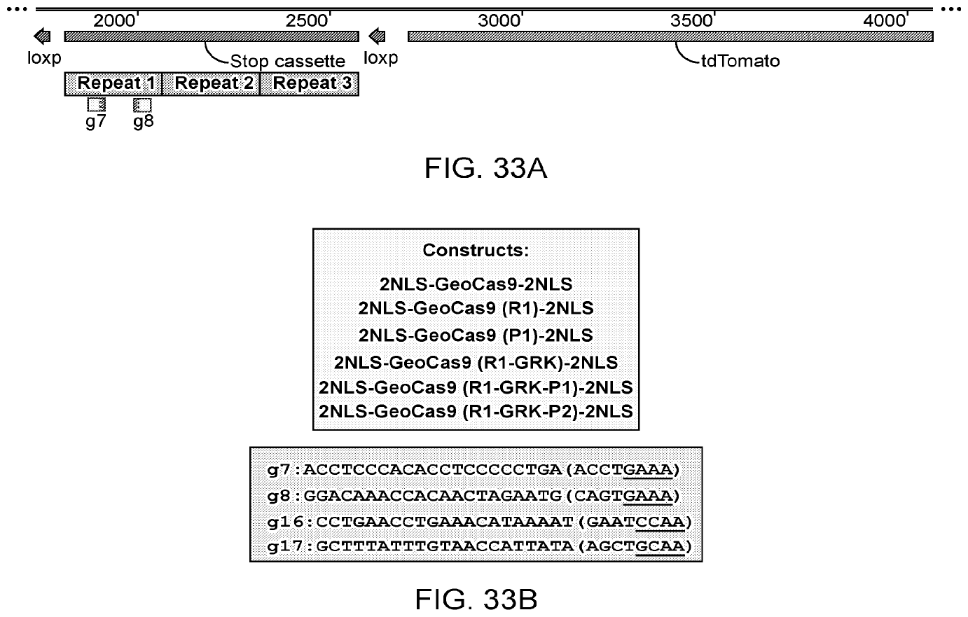

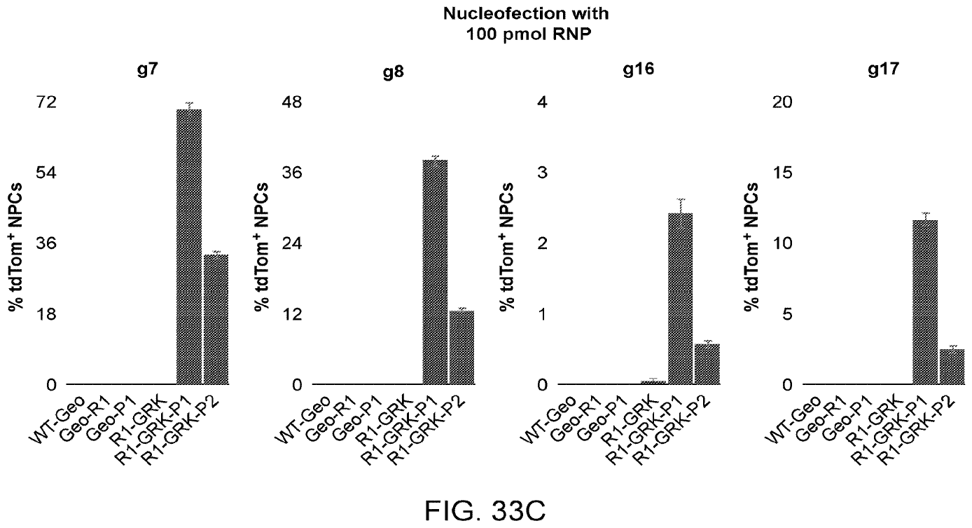

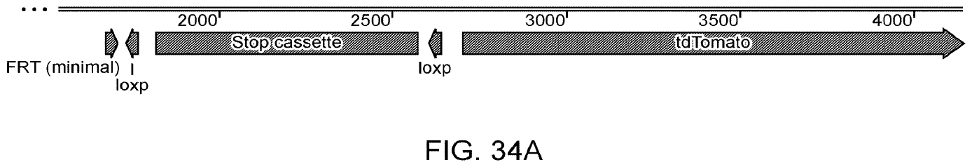

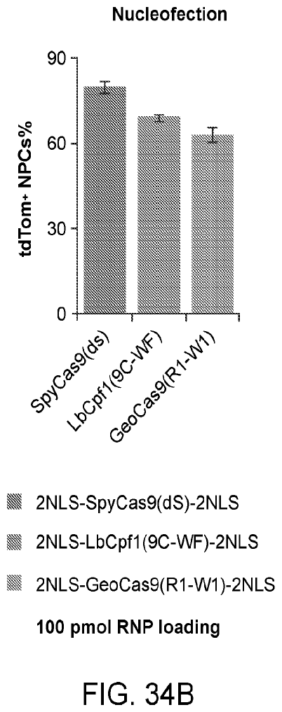

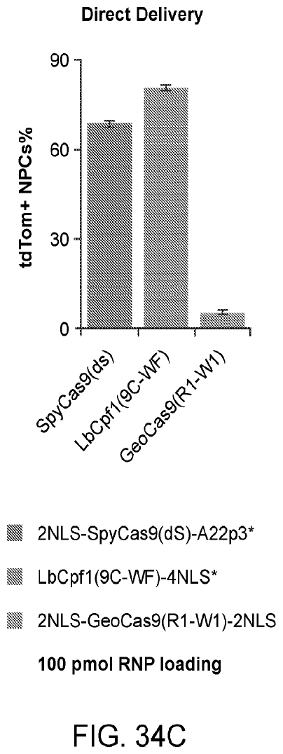

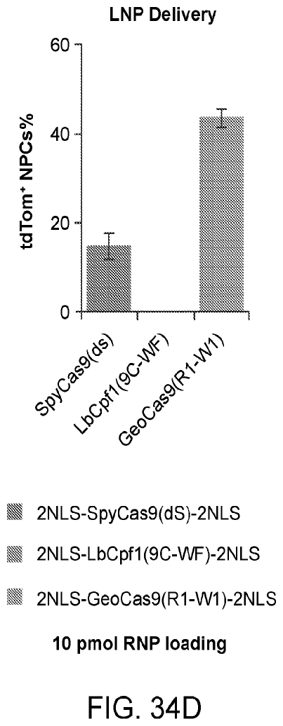

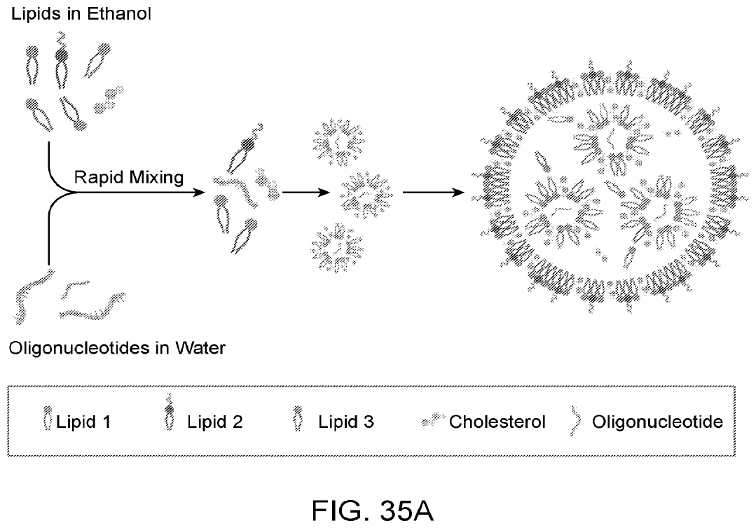

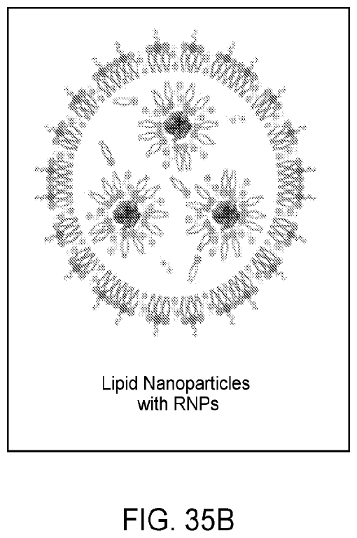

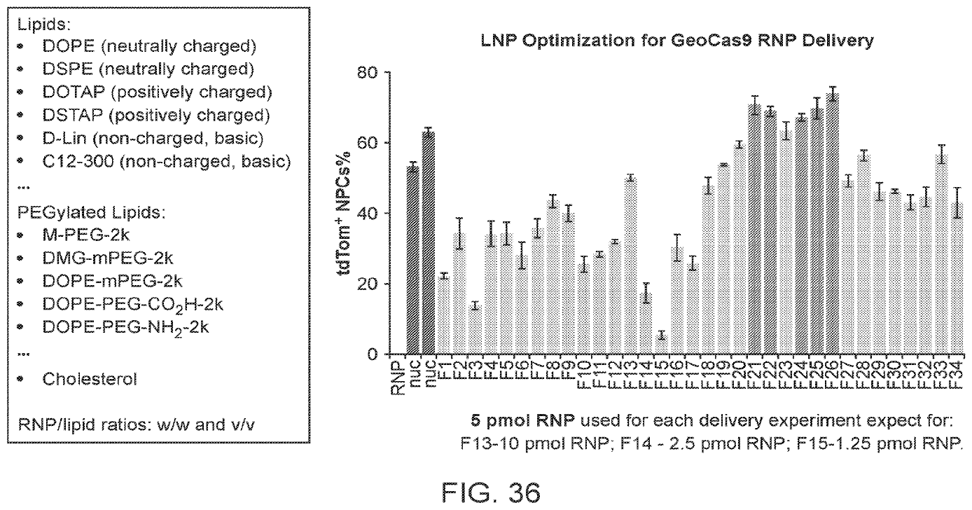

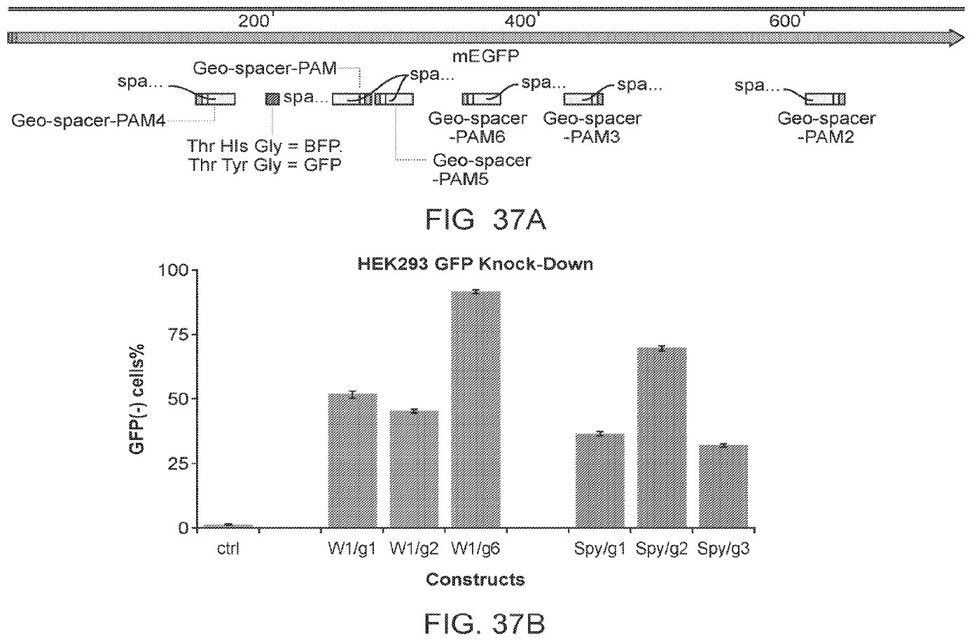

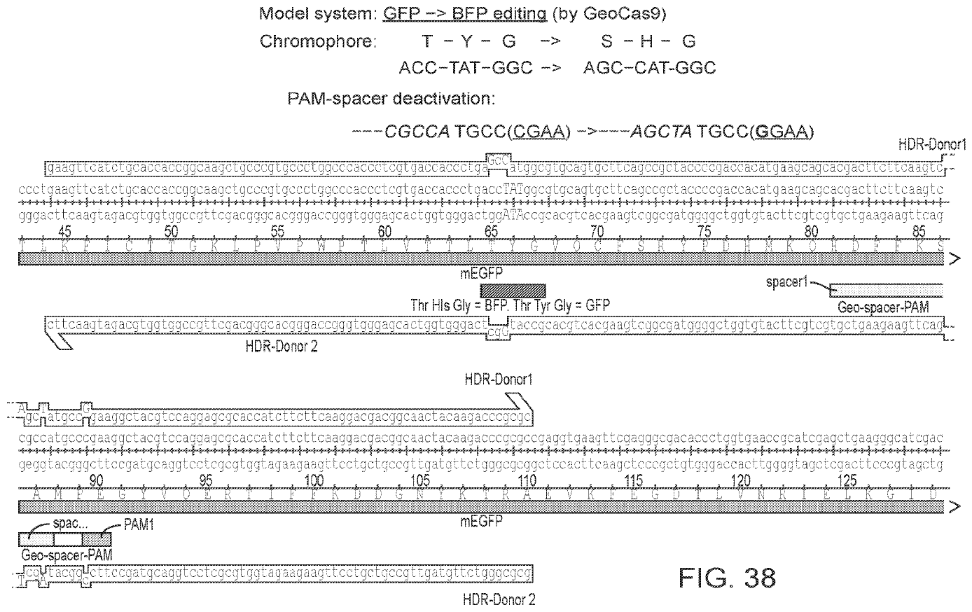

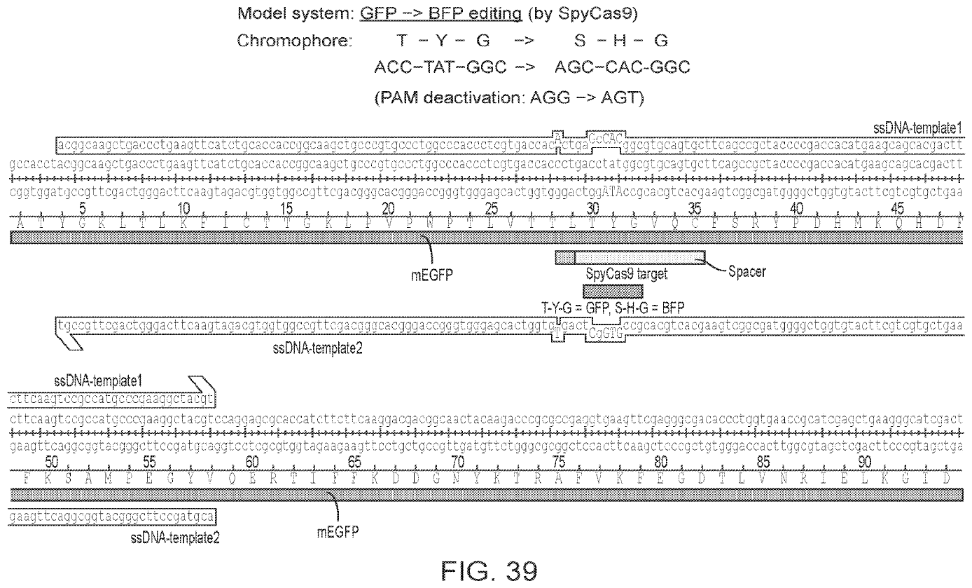

In some cases, the CRISPR-Cas effector polypeptide is a type II CRISPR-Cas effector polypeptide. In some cases, the type II CRISPR-Cas effector polypeptide is a Cas9 polypeptide, e.g., Staphylococcus aureus Cas9, Streptococcus pyogenes Cas9 (SpCas9), etc. In some cases, the CRISPR-Cas effector polypeptide is a variant of a wild-type SpCas9 and comprises one or more of the following substitutions: A61R, L1111R, A1322R, D1135L, S1136W, G1218K, E1219Q, N1317R, R1333P, R1335A, and T1337R. In some cases, the CRISPR-Cas effector polypeptide is an SpG polypeptide or a SpRY polypeptide; see, e.g., Walton et al. (2020) Science 368:290, and WO 2019/051097. For example, a suitable CRISPR-Cas effector polypeptide is an SpCas9 polypeptide includes D1135V, R1135Q, and T1 137R substitutions, relative to wild-type SpCas9. As another example, a suitable CRISPR-Cas effector polypeptide is an SpCas9 polypeptide includes D1 135V, R1335Q, T1337R, and G1218R substitutions, relative to wild-type SpCas9. As another example, a suitable CRISPR-Cas effector polypeptide is an SpCas9 polypeptide includes D1 135L, S1136W, G1218K, E1219Q, R1335A, and T1337R substitutions, relative to wild-type SpCas9. As another example, a suitable CRISPR-Cas effector polypeptide is an SpCas9 polypeptide includes L1111R, A1322R, D1135L, 51136W, G1218K, E1219Q, R1335A, and T1337R substitutions, relative to wild-type SpCas9. As another example, a suitable CRISPR-Cas effector polypeptide is an SpCas9 polypeptide includes A61R, L1111R, A1322R, D1135L, S1136W, G1218K, E1219Q, N1317R, R1333P, R1335A, and T1337R substitutions, relative to wild-type SpCas9. The amino acid sequence of a wild-type SpCas9 polypeptide is provided in FIG. 25A.

In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide. In some cases, the CRISPR-Cas effector polypeptide is a Cas14 polypeptide. In some cases, the CRISPR-Cas effector polypeptide is a Cas14a polypeptide, a Cas14b polypeptide, or a Cas14c polypeptide. For example, a suitable CRISPR-Cas effector polypeptide comprises an amino acid sequence having at least 50%, at least 60%, at least 70%, at least 80%, at least 85%, at least 90%, at least 95%, at least 98%, or at least 99%, amino acid sequence identity to the amino acid sequence depicted in any one of FIG. 25A-25L.

In some cases, the CRISPR-Cas effector polypeptide is a CRISPRi polypeptide; see, e.g., Qi et al. (2013) Cell 152:1173; and Jensen et al. (2021) Genome Research doi:10.1101/gr.275607.121. In some cases, the CRISPR-Cas effector polypeptide is a CRISPRa polypeptide; see, e.g., Jensen et al. (2021) Genome Research doi:10.1101/gr.275607.121; and Breinig et al. (2019) Nature Methods 16:51. In some cases, the CRISPR-Cas effector polypeptide is a CRISPRoff polypeptide. See, e.g., Nunez et al. (2021) Cell 184:2503. In some cases, the CRISPR-Cas effector polypeptide is a nickase. In some cases, the CRISPR-Cas effector polypeptide exhibits reduced catalytic activity compared to a wild-type CRISPR-Cas effector polypeptide.

In some cases, a CRISPR-Cas effector polypeptide present in an LNP is a catalytically inactive CRISPR-Cas effector polypeptide, e.g., the CRISPR-Cas effector polypeptide, when complexed with a guide RNA, binds to a target nucleic acid but does not substantially cleave the target nucleic acid. In some cases, a CRISPR-Cas effector polypeptide present in an LNP is a nickase CRISPR-Cas effector polypeptide, i.e., a CRISPR-Cas effector polypeptide that, when complexed with a guide RNA, binds to a target nucleic acid and cleaves only one strand of the target nucleic acid. Catalytically inactive CRISPR-Cas effector polypeptides are also known as “dead” CRISPR-Cas effector polypeptides. Catalytically inactive CRISPR-Cas effector polypeptides and nickase CRISPR-Cas effector polypeptides are known in the art. See, e.g., Brezgin et al. (2019) Int. J. Mol. Sci. 20:6041. For example, a Streptococcus pyogenes Cas9 polypeptide comprising amino acid substitutions in the RuvC1 and/or the HNH domains can be catalytically inactive; e.g., where the Cas9 polypeptide comprises a D10A and an H84A substitution. Where a CRISPR-Cas polypeptide comprises a HEPN domain, mutations in the HEPN domain can give rise to catalytically inactive CRISPR-Cas effector polypeptides. As another example, mutations in the RuvC domain of Prevotella ihumii Cas12a (“PiCas12a”) and Prevotella disiens Cas12a (“PdCas12a”) (e.g., where the mutations are D946A (D943 of the amino acid sequence depicted in FIG. 25K), E1035A (E1032 of the amino acid sequence depicted in FIG. 25K), and D1279A for PiCas12a (D1277 of the amino acid sequence depicted in FIG. 25K); and D943A for PdCas12a (see FIG. 25L) give rise to a catalytically inactive Cas12a; see, e.g., Jacobsen et al. (2020) Nucl. Acids Res. 48:5624. As another example, a variant Cas7-11 polypeptide comprises a substitution of one or more of D177, D429, D654, D758, E959, and D998 (where the amino acid numbering is as set forth in FIG. 25H). D177, D429, D654, D758, E959, and D998 are in bold in FIG. 25H.

The present disclosure provides methods of delivering an RNP into a eukaryotic cell. The methods comprise contacting the eukaryotic cell with an LNP of the present disclosure, where the LNP comprises a CRISPR-Cas effector polypeptide and a guide nucleic acid. In some cases, the LNP further comprises a donor template nucleic acid. In some cases, the eukaryotic cell is in vitro. In some cases, the eukaryotic cell is in vivo. Target cells of interest include those described above.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects.

This is intended to provide support for all such combinations of aspects and is not limited to combinations of aspects explicitly provided below:
Aspect 1. A variant CRISPR-Cas effector polypeptide comprising an amino acid sequence having at least 50% amino acid sequence identity to the amino acid sequence depicted in FIG. 1, wherein the variant CRISPR-Cas effector polypeptide comprises:
   (a) a substitution of E149, T182, N206. and P466, based on the amino acid numbering of the GeoCas9 amino acid sequence depicted in FIG. 1; or
   (b) a substitution of N206 and I331, based on the amino acid numbering of the GeoCas9 amino acid sequence depicted in FIG. 1; or
   (c) a substitution of D1017, based on the amino acid numbering of the GeoCas9 amino acid sequence depicted in FIG. 1.
Aspect 2. The variant CRISPR-Cas effector polypeptide of aspect 1(a), wherein the variant CRISPR-Cas effector polypeptide further comprises a substitution of one or more of E179, L190. H340, 1379, K455, L601, Q817, K832, E843, K879, E884, K908, S924, K934, T1015. D1017, S1073, and D1087, based on the amino acid numbering of the GeoCas9 amino acid sequence depicted in FIG. 1.
Aspect 3. The variant CRISPR-Cas effector polypeptide of aspect 1, wherein the variant CRISPR-Cas effector polypeptide comprises:
a) a substitution of E149, T182, N206, P466, and E843, based on the amino acid numbering of the GeoCas9 amino acid sequence depicted in FIG. 1; or
b) a substitution of E149, T182, N206, P466, E884, and K908, based on the amino acid numbering of the GeoCas9 amino acid sequence depicted in FIG. 1; or
c) a substitution of E149, T182, N206, P466, E843, E884, and K908, based on the amino acid numbering of the GeoCas9 amino acid sequence depicted in FIG. 1; or
d) a substitution of E149, T182, N206, P466, E843. E884, K908, and Q817, based on the amino acid numbering of the GeoCas9 amino acid sequence depicted in FIG. 1; or
e) a substitution of E149, T182, N206, P466, E843, E884, K908, T1015, and D1017, based on the amino acid numbering of the GeoCas9 amino acid sequence depicted in FIG. 1; or
f) a substitution of E149, T182, N206, P466, E843, E884, K908, and D1017, based on the amino acid numbering of the GeoCas9 amino acid sequence depicted in FIG. 1; or
g) a substitution of E149, T182, N206, P466, E843. E884, K908, L601, and K832, based on the amino acid numbering of the GeoCas9 amino acid sequence depicted in FIG. 1; or
h) a substitution of E149, T182, N206, P466, E843, E884, K908, and E179, based on the amino acid numbering of the GeoCas9 amino acid sequence depicted in FIG. 1; or
i) a substitution of E149, T182, N206, P466, E843, E884, K908, E179, and Q817, based on the amino acid numbering of the GeoCas9 amino acid sequence depicted in FIG. 1; or
j) a substitution of E149, T182, N206, P466, E843, E884, K908, K934, K832, and L601, based on the amino acid numbering of the GeoCas9 amino acid sequence depicted in FIG. 1; or
k) a substitution of E149, T182, N206, P466, E843, E884, K908, K934, and K832, based on the amino acid numbering of the GeoCas9 amino acid sequence depicted in FIG. 1; or
l) a substitution of E149, T182, N206, P466, E843, E884, K908, K832, L601, and T730, based on the amino acid numbering of the GeoCas9 amino acid sequence depicted in FIG. 1; or
m) a substitution of E149, T182, N206, P466, E843, E884, K908, Q817, L190, H340, 1379, K455, K879, and D1087, based on the amino acid numbering of the GeoCas9 amino acid sequence depicted in FIG. 1; or
n) a substitution of E149, T182, N206, P466, E843. E884, K908, Q817, S924, and S1073, based on the amino acid numbering of the GeoCas9 amino acid sequence depicted in FIG. 1.
Aspect 4. The variant CRISPR-Cas effector polypeptide of aspect 3, wherein the variant CRISPR-Cas effector polypeptide comprises:
i) E149G, T182I, N206D, P466Q, and E843K substitutions;
ii) E149G, T182I, N206D, P466Q, E884G, and K908R substitutions;
iii) E149G, T182I, N206D, P466Q, E843K, E884G, and K908R substitutions;
iv) E149G, T182I, N206D, P466Q. E843K, E884G, K908R, and Q817R substitutions;
v) E149G, T182I, N206D, P466Q, E843K, E884G, K908R, T1015A, and D1017N substitutions;
vi) E149G, T182I, N206D, P466Q, E843K, E884G, K908R, and D1017G substitutions;
vii) E149G, T182I, N206D, P466Q, E843K, E884G, K908R, L601A, and K832R substitutions;
viii) E149G, T182I, N206D, P466Q, E843K, E884G, K908R, L601P, and K832R substitutions;
ix) E149G, T182I, N206D, P466Q, E843K, E884G, K908R, and E179G substitutions;
x) E149G, T182I, N206D, P466Q, E843K, E884G. K908R, E179G, and Q817R substitutions;
xi) E149G, T182I, N206D, P466Q, E843K, E884G, K908R, E179R, and Q817R E149G T182I N206D P466Q E843K E884G K908R E179R and Q817R substitutions;
xii) E149G, T182I, N206D, P466Q, E843K, E884G, K908R, K934V, K832R, and L601A substitutions;
xiii) E149G, T182I, N206D, P466Q, E843K, E884G, K908R, K934S, K832R, and L601T substitutions;
xiv) E149G, T182I, N206D, P466Q, E843K, E884G, K908R, K934D, K832R, and L601 P substitutions;
xv) E149G, T182I, N206D, P466Q, E843K, E884G, K908R, K934S, and K832L substitutions;
xvi) E149G, T182I, N206D, P466Q, E843K, E884G, K908R, K832L, L601R, and T730A substitutions;
xvii) E149G, T182I, N206D, P466Q, E843K, E884G, K908R, Q817R, L190P, H340N, I379T, K455R, K879R, and D1087A substitutions; or
xviii) E149G, T182I, N206D, P466Q, E843K, E884G, K908R, Q817R, S924P, and S1073N substitutions.
Aspect 5. The variant CRISPR-Cas effector polypeptide of aspect 1(b), wherein the variant CRISPR-Cas effector polypeptide comprises an N206T substitution and an 1331T substitution.
Aspect 6. The variant CRISPR-Cas effector polypeptide of aspect 1(c), further comprising a substitution of one or more of R829, K888, and T1015, based on the amino acid numbering of the GeoCas9 amino acid sequence depicted in FIG. 1.
Aspect 7. The variant CRISPR-Cas effector polypeptide of aspect 6, wherein the variant CRISPR-Cas effector polypeptide comprises R829H. K888R, T1015A, and D1017N substitutions.
Aspect 8. The variant CRISPR-Cas effector polypeptide of aspect 1(c), wherein the variant CRISPR-Cas effector polypeptide comprises a D1017G substitution.
Aspect 9. The variant CRISPR-Cas effector polypeptide of any one of aspects 1-8, wherein the variant CRISPR-Cas effector polypeptide is enzymatically active in a temperature range of from 15° C. to 75° C.
Aspect 10. The variant CRISPR-Cas effector polypeptide of any one of aspects 1-9, wherein the variant CRISPR-Cas effector polypeptide binds to a target nucleic acid comprising a PAM comprising a GAAA or a GCAA sequence.
Aspect 11. The variant CRISPR-Cas effector polypeptide of any one of aspects 1-10, wherein the variant CRISPR-Cas effector polypeptide has a length of from 1050 amino acids to 1120 amino acids.
Aspect 12. The variant CRISPR-Cas effector polypeptide of any one of aspects 1-10, wherein the variant CRISPR-Cas effector polypeptide has a length of from 1080 amino acids to 1095 amino acids.
Aspect 13. The variant CRISPR-Cas effector polypeptide of any one of aspects 1-12, wherein the variant CRISPR-Cas effector polypeptide comprises an amino acid sequence having at least 60% amino acid sequence identity to the amino acid sequence set forth in SEQ ID NO: 1.
Aspect 14. The variant CRISPR-Cas effector polypeptide of any one of aspects 1-12, wherein the variant CRISPR-Cas effector polypeptide comprises an amino acid sequence having at least 70% amino acid sequence identity to the amino acid sequence set forth in SEQ ID NO: 1.
Aspect 15. The variant CRISPR-Cas effector polypeptide of any one of aspects 1-12, wherein the variant CRISPR-Cas effector polypeptide comprises an amino acid sequence having at least 80% amino acid sequence identity to the amino acid sequence set forth in SEQ ID NO: 1.
Aspect 16. The variant CRISPR-Cas effector polypeptide of any one of aspects 1-12, wherein the variant CRISPR-Cas effector polypeptide comprises an amino acid sequence having at least 90% amino acid sequence identity to the amino acid sequence set forth in SEQ ID NO: 1.
Aspect 17. The variant CRISPR-Cas effector polypeptide of any one of aspects 1-12, wherein the variant CRISPR-Cas effector polypeptide comprises an amino acid sequence having at least 95% amino acid sequence identity to the amino acid sequence set forth in SEQ ID NO: 1.
Aspect 18. The variant CRISPR-Cas effector polypeptide of any one of aspects 1-17, wherein the variant CRISPR-Cas effector polypeptide exhibits at least 25% greater gene editing activity than a CRISPR-Cas effector polypeptide having the amino acid sequence depicted in FIG. 1.
Aspect 19. The variant CRISPR-Cas effector polypeptide of any one of aspects 1-17, wherein the variant CRISPR-Cas effector polypeptide exhibits at least 50% greater gene editing activity than a CRISPR-Cas effector polypeptide having the amino acid sequence depicted in FIG. 1.
Aspect 20. The variant CRISPR-Cas effector polypeptide of any one of aspects 1-17, wherein the variant CRISPR-Cas effector polypeptide exhibits at least 2-fold, at least 3-fold, at least 4-fold, or at least 5-fold, greater gene editing activity than a CRISPR-Cas effector polypeptide having the amino acid sequence depicted in FIG. 1.
Aspect 21. A nucleic acid comprising a nucleotide sequence encoding the variant CRISPR-Cas effector polypeptide of any one of aspects 1-20.
Aspect 22. The nucleic acid of aspect 21, wherein the nucleotide sequence encoding the variant CRISPR-Cas effector polypeptide is codon optimized for expression in a eukaryotic cell.
Aspect 23. The nucleic acid of aspect 22, wherein the nucleotide sequence encoding the variant CRISPR-Cas effector polypeptide is codon optimized for expression in a mammalian cell or a plant cell.
Aspect 24. The nucleic acid of any one of aspects 21-23, wherein the nucleotide sequence encoding the variant CRISPR-Cas effector polypeptide is operably linked to one or more transcriptional control elements.
Aspect 25. The nucleic acid of aspect 24, wherein the one or more transcriptional control elements comprises a promoter.
Aspect 26. The nucleic acid of aspect 25, wherein the promoter is a regulatable promoter or a constitutive promoter.
Aspect 27. The nucleic acid of aspect 25 or aspect 26, wherein the promoter is functional in a eukaryotic cell.
Aspect 28. A recombinant expression vector comprising the nucleic acid of any one of aspects 21-27.
Aspect 29. A composition comprising the variant CRISPR-Cas effector polypeptide of any one of aspects 1-20, the nucleic acid of any one of aspects 21-27, or the recombinant expression vector of aspect 28.
Aspect 30. The composition of aspect 29, comprising one or more of: i) a buffer; ii) a nuclease inhibitor; iii) a protease inhibitor; and iv) a lipid.
Aspect 31. A fusion polypeptide comprising:
a) a variant CRISPR-Cas effector polypeptide of any one of aspects 1-20; and
b) one or more heterologous fusion partner polypeptides.
Aspect 32. The fusion polypeptide of aspect 31, wherein the one or more heterologous fusion partner polypeptides comprises a protein transduction domain that facilitates traversal of the variant CRISPR-Cas effector polypeptide from the cytosol of a cell to within an organelle in the cell.
Aspect 33. The fusion polypeptide of aspect 31, wherein the one or more heterologous fusion partner polypeptides is a reverse transcriptase.
Aspect 34. The fusion polypeptide of aspect 31, wherein the one or more heterologous fusion partner polypeptides has DNA modifying activity, increases transcription, decreases transcription, or modifies a polypeptide associated with a nucleic acid.
Aspect 35. The fusion polypeptide of aspect 31, wherein the one or more heterologous fusion partner polypeptides is a cytosine deaminase or an adenosine deaminase.
Aspect 36. The fusion polypeptide of any one of aspects 31-35, wherein the one or more heterologous fusion partner polypeptides comprises a nuclear localization signal (NLS).
Aspect 37. A nucleic acid comprising a nucleotide sequence encoding the fusion polypeptide of any one of aspects 31-36.
Aspect 38. A recombinant expression vector comprising the nucleic acid of aspect 37.
Aspect 39. A cell comprising:
a) the variant CRISPR-Cas effector polypeptide of any one of aspects 1-20; or
b) the nucleic acid of any one of aspects 21-27; or
c) the recombinant expression vector of aspect 28; or
d) the fusion polypeptide of any one of aspects 31-36; or
e) the nucleic acid of aspect 37; or
f) the expression vector of aspect 38.
Aspect 40. The cell of aspect 39, wherein the cell is a eukaryotic cell.
Aspect 41. The cell of aspect 40, wherein the eukaryotic cell is a mammalian cell, a fish cell, an invertebrate animal cell, a vertebrate cell, a plant cell, an algal cell, a bird cell, an insect cell, an arachnid cell, an ungulate cell, a non-human primate cell, or a human cell.
Aspect 42. The cell of any one of aspects 39-41, wherein the cell is in vitro.
Aspect 43. The cell of any one of aspects 39-41, wherein the cell is in vivo.
Aspect 44. A ribonucleoprotein (RNP) complex comprising:
a1) the variant CRISPR-Cas effector polypeptide of any one of aspects 1-20; and
b1) a guide nucleic acid; or
a2) the fusion polypeptide of any one of aspects 29-51; and
b2) a guide nucleic acid.
Aspect 45. The RNP complex of aspect 44, wherein the guide nucleic acid is a dual-guide RNA.
Aspect 46. The RNP complex of aspect 44, wherein the guide nucleic acid is a single-guide RNA.
Aspect 47. The RNP complex of aspect 44, wherein the guide nucleic acid comprises:
i) a DNA-targeting segment comprising a nucleotide sequence that is complementary to a target nucleotide sequence in a target nucleic acid; and
ii) a protein-binding segment that binds to and activates the variant CRISPR-Cas effector polypeptide, wherein the protein-binding segment comprises a duplex-forming linker segment and a tracrRNA.
Aspect 48. The RNP complex of aspect 44, wherein the guide nucleic acid is a dual-guide RNA comprising:
i) a first RNA comprising the DNA-targeting segment; and
ii) a second RNA comprising the protein-binding segment, wherein the first RNA and the second RNA are not contiguous with one another and are not covalently linked to one another.
Aspect 49. The RNP complex of aspect 44, wherein the guide nucleic acid is a single-molecule guide RNA, wherein the DNA-targeting segment and the protein-binding segment are present in a single RNA molecule.
Aspect 50. The RNP complex of any one of aspects 44-49, wherein the guide nucleic acid comprises one or more of a modified nucleobase, a modified backbone, a non-natural internucleoside linkage, a modified sugar moiety, a Locked Nucleic Acid, and a Peptide Nucleic Acid.
Aspect 51. The RNP complex of any one of aspects 44-50, comprising two or more guide nucleic acids.
Aspect 52. The RNP complex of any one of aspects 44-51, comprising a donor template nucleic acid.
Aspect 53. A multicellular, non-human organism comprising:
a) the variant CRISPR-Cas effector polypeptide of any one of aspects 1-20; or
b) the nucleic acid of any one of aspects 21-27; or
c) the recombinant expression vector of aspect 28; or
d) the fusion polypeptide of any one of aspects 31-36; or
e) the nucleic acid of any one of aspects 373; or
f) the expression vector of aspect 38.
Aspect 54. A method of modifying a target DNA, the method comprising contacting the target DNA with the RNP complex of any one of aspects 44-52.
Aspect 55. The method of aspect 54, wherein said modifying comprises non-homologous end joining.
Aspect 56. The method of aspect 54, wherein said modifying comprises homology-directed repair.
Aspect 57. The method of any one of aspects 54-56, wherein said contacting occurs in a cell in vitro.
Aspect 58. The method of any one of aspects 54-56, wherein said contacting occurs in a cell in vivo.
Aspect 59. The method of any one of aspects 54-58, wherein the target DNA is present in a eukaryotic cell.
Aspect 60. The method of aspect 59, wherein the target DNA is chromosomal DNA, chloroplast DNA, or mitochondrial DNA.
Aspect 61. The method of any one of aspects 54-60, wherein said modifying comprises cleavage of the target DNA.
Aspect 62. A lipid nanoparticle (LNP) comprising:
a) a first lipid, wherein the first lipid comprises a covalently attached poly(ethylene glycol) (PEG) moiety;
b) at least a second and a third lipid, wherein the second lipid and the third lipid do not comprise a covalently attached PEG moiety;
c) a CRISPR-Cas effector polypeptide; and
d) a guide nucleic acid.
Aspect 63. The LNP of aspect 62, wherein the first lipid is selected from the group consisting of M-PEG, DMG-mPEG, DOPE-mPEG, DOPE-PEG-CO2H, DOPE-PEG-NH2, where the PEG has a molecular weight of about 2 kD.
Aspect 64. The LNP of aspect 62 or aspect 63, wherein the second and third lipids are selected from the group consisting of DOPE, DSPE, DOTAP, DSTAP, D-Lin, C12-300, and cholesterol.
Aspect 65. The LNP of any one of aspects 62-64, wherein the LNP comprises DOTAP, D-Lin, DOPE, cholesterol, and M-PEG-2k.
Aspect 66. The LNP of aspect 65, wherein the LNP comprises DOTAP, D-Lin, DOPE, cholesterol, and M-PEG-2k in a ratio of 18-20:36:12-14:40:1.5-2.5 DOTAP:D-Lin:DOPE:cholesterol:M-PEG-2k.
Aspect 67. The LNP of any one of aspects 62-66, wherein the CRISPR-Cas polypeptide is:
a) a variant CRISPR-Cas polypeptide of any one of aspects 1-20; or
b) a fusion polypeptide of any one of aspects 31-36.
Aspect 68. The LNP of any one of aspects 62-66, wherein the CRISPR-Cas effector polypeptide is:
a) a type II CRISPR-Cas effector polypeptide, a type III CRISPR-Cas effector polypeptide, a type IV CRISPR-Cas effector polypeptide, a type V CRISPR-Cas effector polypeptide, or a type VI CRISPR-Cas effector polypeptide; or
b) a fusion polypeptide comprising: i) one or more heterologous fusion polypeptides; and ii) a type II CRISPR-Cas effector polypeptide, a type III CRISPR-Cas effector polypeptide, a type IV CRISPR-Cas effector polypeptide, a type V CRISPR-Cas effector polypeptide, or a type VI CRISPR-Cas effector polypeptide.
Aspect 69. A method of making the LNP of any one of aspects 62-68, the method comprising:
a) combining the CRISPR-Cas polypeptide and the guide nucleic acid with the first lipid in an aqueous solution, to form a first composition; and
b) combining the first composition with a second composition, wherein the second composition comprises, in an organic solvent, the second and third lipids, thereby forming the LNP.
Aspect 70. A method of delivering a ribonucleoprotein (RNP) into a eukaryotic cell, the method comprising contacting the eukaryotic cell with an LNP of any one of aspects 62-68, wherein the RNP comprises a CRISPR-Cas effector polypeptide and a guide nucleic acid, and wherein said contacting results in delivery of the RNP into the eukaryotic cell.
Aspect 71. The method of aspect 70, wherein the eukaryotic cell is in vitro.
Aspect 72. The method of aspect 70, wherein the eukaryotic cell is in vivo.
Aspect 73. A CRISPR-Cas effector polypeptide comprising an amino acid sequence having at least 50% amino acid sequence identity to the amino acid sequence of any one of SEQ ID NOs.: 165-175, wherein the variant CRISPR-Cas effector polypeptide comprises a substitution at an amino acid position corresponding to:
(a) E149, T182, N206, and P466; or
(b) D1017, based on the amino acid numbering of the ThermoCas9 amino acid sequence of SEQ ID NO: 165.
Aspect 74. The CRISPR-Cas effector polypeptide of aspect 73, comprising a substitution at an amino acid position corresponding to:
a) E149, T182, N206, P466, and E843; or
b) E149, T182, N206, P466, E884, and T908; or
c) E149, T182, N206. P466, E843, E884, and T908; or
d) E149, T182, N206, P466, E843, E884, T908, and Q817; or
e) E149, T182, N206, P466, E843, E884, T908, T1015, and D1017; or
f) E149, T182, N206, P466, E843, E884, T908, and D1017, based on the amino acid numbering of the ThermoCas9 amino acid sequence of SEQ ID NO: 165.
Aspect 75. The CRISPR-Cas effector polypeptide of aspect 74, wherein the variant CRISPR-Cas effector polypeptide comprises the substitutions:
i) E149G, T182I, N206D, P466Q, and E843K; or
ii) E149G, T182I, N206D, P466Q, E884G, and T908R; or
iii) E149G, T182I, N206D, P466Q, E843K, E884G, and T908R; or
iv) E149G, T182I, N206D, P466Q, E843K, E884G, T908R, and Q817R; or
v) E149G, T182I, N206D, P466Q, E843K, E884G. T908R, T1015A, and D1017N; or
vi) E149G, T182I, N206D, P466Q, E843K, E884G, T908R, and D1017G.
Aspect 76. The CRISPR-Cas effector polypeptide of any one of aspects 73-75, wherein the CRISPR-Cas effector polypeptide comprises an amino acid sequence having at least 80% amino acid sequence identity to the amino acid sequence of any one of SEQ ID NOs.: 165-175.
Aspect 77. The CRISPR-Cas effector polypeptide of any one of aspects 73-75, wherein the CRISPR-Cas effector polypeptide comprises an amino acid sequence having at least 80% amino acid sequence identity to the amino acid sequence of any one of SEQ ID NOs.: 165.
Aspect 78. The CRISPR-Cas effector polypeptide of aspect 73, wherein the CRISPR-Cas effector polypeptide comprises an amino acid sequence having at least 70% amino acid sequence identity to the amino acid sequence of SEQ ID NOs.: 176.
Aspect 79. A nucleic acid comprising a nucleotide sequence encoding the variant CRISPR-Cas effector polypeptide of any one of aspects 73-78.
Aspect 80. The nucleic acid of aspect 79, wherein the nucleotide sequence encoding the CRISPR-Cas effector polypeptide is codon optimized for expression in a eukaryotic cell.
Aspect 81. The nucleic acid of aspect 79 or aspect 80, wherein the nucleotide sequence encoding the CRISPR-Cas effector polypeptide is operably linked to a promoter functional in a eukaryotic cell.
Aspect 82. A recombinant expression vector comprising the nucleic acid of any one of aspects 79-81.
Aspect 83. A fusion polypeptide comprising:
a) a CRISPR-Cas effector polypeptide of any one of aspects 73-78; and
b) one or more heterologous fusion partner polypeptides.
Aspect 84. The fusion polypeptide of aspect 83, wherein the one or more heterologous fusion partner polypeptides comprises a reverse transcriptase.
Aspect 85. The fusion polypeptide of aspect 83, wherein the one or more heterologous fusion partner polypeptides has DNA modifying activity, increases transcription, decreases transcription, or modifies a polypeptide associated with a nucleic acid.
Aspect 86. The fusion polypeptide of aspect 83, wherein the one or more heterologous fusion partner polypeptides comprises a cytosine deaminase or an adenosine deaminase.
Aspect 87. A nucleic acid comprising a nucleotide sequence encoding the fusion polypeptide of any one of aspects 83-86.
Aspect 88. A cell comprising:
a) the CRISPR-Cas effector polypeptide of any one of aspects 73-78 or a nucleic acid encoding the CRISPR-Cas effector polypeptide; or
b) the fusion polypeptide of any one of aspects 83-86 or a nucleic acid encoding the fusion polypeptide.
Aspect 89. The cell of aspect 88, wherein the cell is a eukaryotic cell.
Aspect 90. A ribonucleoprotein (RNP) complex comprising:
a guide nucleic acid and the CRISPR-Cas effector polypeptide of any one of aspects 73-78; or a guide nucleic acid and the fusion polypeptide of any one of aspects 83-86.
Aspect 91. A multicellular, non-human organism comprising:
a) the CRISPR-Cas effector polypeptide of any one of aspects 73-78 or a nucleic acid encoding the CRISPR-Cas effector polypeptide; or
b) the fusion polypeptide of any one of aspects 83-86 or a nucleic acid encoding the fusion polypeptide.
Aspect 92. A method of modifying a target DNA, the method comprising contacting the target DNA with the RNP complex of aspect 90.
Aspect 93. The method of aspect 92, wherein the target DNA is present in a eukaryotic cell.
Aspect 94. The method of aspect 93, wherein said contacting occurs in the eukaryotic cell in vivo.
Aspect 95. The method of any one of aspects 92-94, wherein said modifying comprises cleavage of the target DNA.
Aspect 96. The LNP of any one of aspects 62-66, wherein the CRISPR-Cas polypeptide is:
a) the CRISPR-Cas polypeptide of any one of aspects 73-78; or
b) the fusion polypeptide of any one of aspects 83-86.
Aspect 97. The LNP of claim 67 or claim 96, wherein the first lipid of the LNP comprises an acid-degradable PEGylated lipid.
Aspect 98. The LNP of aspect 97, wherein the acid-degradable PEGylated lipid is ADP-2k, Pep-1k, or Pep-2k.
Aspect 99. The LNP of any one of aspects 96-98, wherein the LNP comprises ADC as a cationic lipid.
Aspect 100. The method of aspect 58 or aspect 94, wherein the eukaryotic cell is a liver cell and said contacting comprises introducing the RNP to the liver cell as part of a liquid nanoparticle (LNP) that comprises an acid-degradable PEGylated lipid.
Aspect 101. The method of aspect 58 or aspect 94, wherein the eukaryotic cell is a lung cell and said contacting comprises introducing the RNP to the lung cell as part of a liquid nanoparticle (LNP) that comprises ADC as a cationic lipid.
Aspect 102. The method of aspect 101, wherein the LNP further comprises an acid-degradable PEGylated lipid.
Aspect 103. A method of delivering a ribonucleoprotein (RNP) into a eukaryotic cell, the method comprising contacting the eukaryotic cell with an LNP of any one of aspects 96-98, wherein the RNP comprises a CRISPR-Cas effector polypeptide and a guide nucleic acid, and wherein said contacting results in delivery of the RNP into the eukaryotic cell.
Aspect 104. The method of aspect 103, wherein the eukaryotic cell is in vivo.
Aspect 105. The method of aspect 72 or aspect 104, wherein the eukaryotic cell is a liver cell and the LNP comprises an acid-degradable PEGylated lipid.
Aspect 106. The method of aspect 72 or aspect 104, wherein the eukaryotic cell is a lung cell and the LNP comprises ADC as a cationic lipid.
Aspect 107. The method of aspect 106, wherein the LNP further comprises an acid-degradable PEGylated lipid.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example: Thermostable CRISPR Ribonucleoprotein Edits Lung and Liver Tissue after Delivery with Lipid Nanoparticles

The results herein demonstrate engineering of a thermostable genome editor, GeoCas9. via directed evolution. An evolved variant, iGeoCas9, was generated which edited cells with 3 orders of magnitude higher efficiency than wild-type GeoCas9. iGeoCas9 had higher stability than SpyCas9 or LbCas12a in the presence of organic solvents and lipids and performed better genome editing after LNP-mediated delivery to cells. The result show that LNP/iGeoCas9 complexes can edit a variety of cell lines and efficiently induce homology-directed repair (HDR) in cells via co-delivery with ssDNA templates.

Moreover, iGeoCas9 complexed to acid-degradable LNPs edited lung tissue in vivo with an unprecedented efficiency of 35% after intravenous injections. Collectively, these results demonstrate that ultra-stable RNP/LNP complexes are a powerful alternative to mRNA/LNP complexes and can expand the therapeutic applications of genome editing.

For example, the results demonstrate that an evolved GeoCas9, termed iGeoCas9, can edit mammalian cells with >1000-fold higher efficiency than wild-type GeoCas9 and was able to edit cells and animals efficiently, e.g., after LNP-mediated delivery. An LNP-based platform containing pH-sensitive PEGylated and cationic lipids was developed for delivering iGeoCas9 RNP, which edited a variety of cell lines with remarkable efficiency and also triggered homology-directed repair (HDR) after co-delivery with ssDNA templates. iGeoCas9 RNP/LNP formulations were also able to edit the liver and lung tissue with high efficiency after intravenous injections. RNP/LNP formulations containing an acid-degradable PEGylated lipid edited 56% of the liver tissue in the Ai9 mouse model, and formulations containing acid-degradable PEGylated and cationic lipids edited 35% of the entire lung tissue. Developing CRISPR therapeutics that can efficiently edit lung tissue after an intravenous injection is a major challenge in the field of genome editing and the lung editing observed with iGeoCas9 RNP/LNP formulations represents the most efficient lung editing observed to date after an intravenous injection of CRISPR reagents using either viral or non-viral delivery strategies. Collectively, the results presented herein demonstrate that ultra-stable genome editors (e.g., GeoCas9, iGeo Cas9) coupled with LNPs can efficiently edit cells in vitro and in vivo.

### Results

### Directed Evolution of GeoCas9 Improves its Editing Efficiency and Broadens its PAM Compatibility

GeoCas9 is a compact, thermostable type II-C CRISPR-Cas protein identified from the thermophilic bacterium Geobacillus stearothermophilus. Its superior thermal stability allows it to function as an RNA-guided endonuclease under complex conditions, such as at elevated temperatures or in the presence of human plasma. These properties make GeoCas9 an attractive editor for delivery in vivo, particularly in the RNP format. However, GeoCas9 is less effective than SpyCas9 at genome editing in mammalian cells and has a more restricted PAM. Wild-type GeoCas9 recognizes a PAM sequence of 5′-NNNNCRAA-3′ (where R is A/G) and can consequently target a much smaller fraction of the genome than canonical SpyCas9, which has a PAM sequence of 5′-NGG-3′.

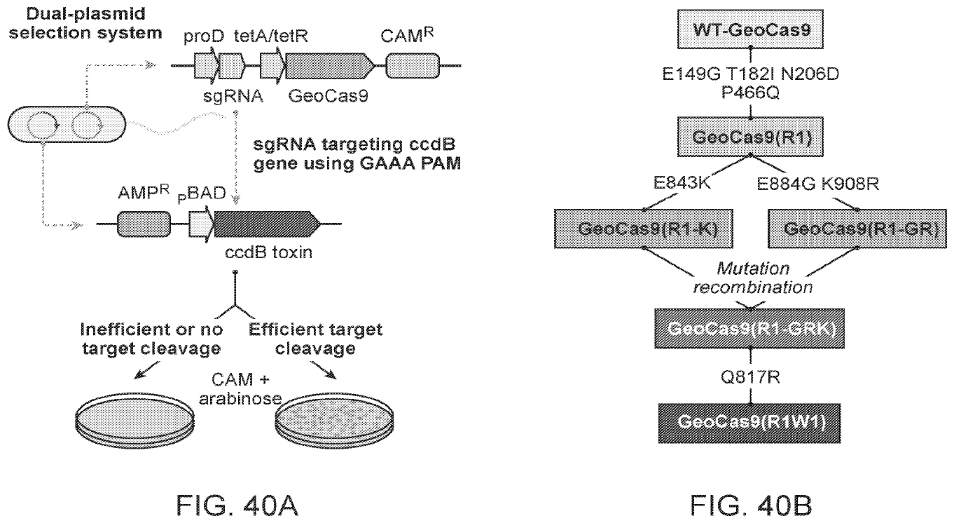

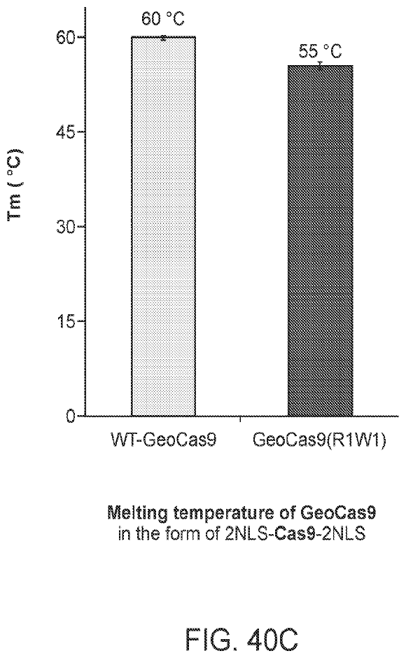

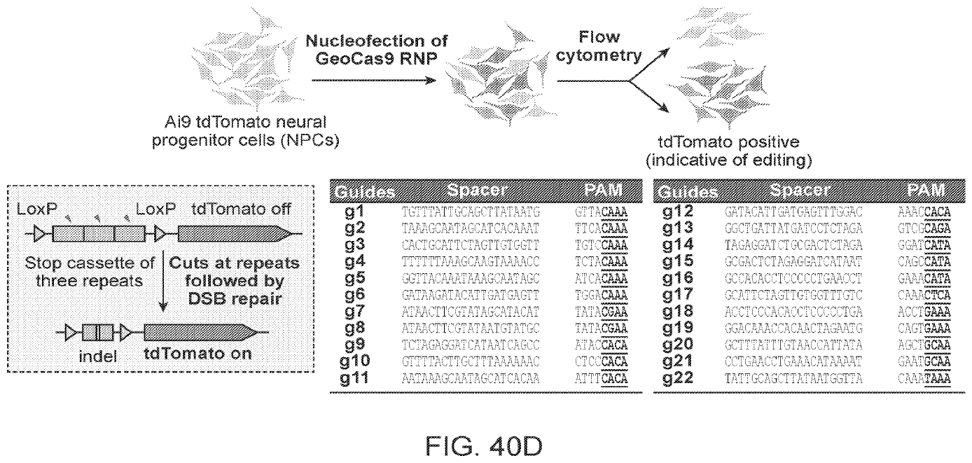

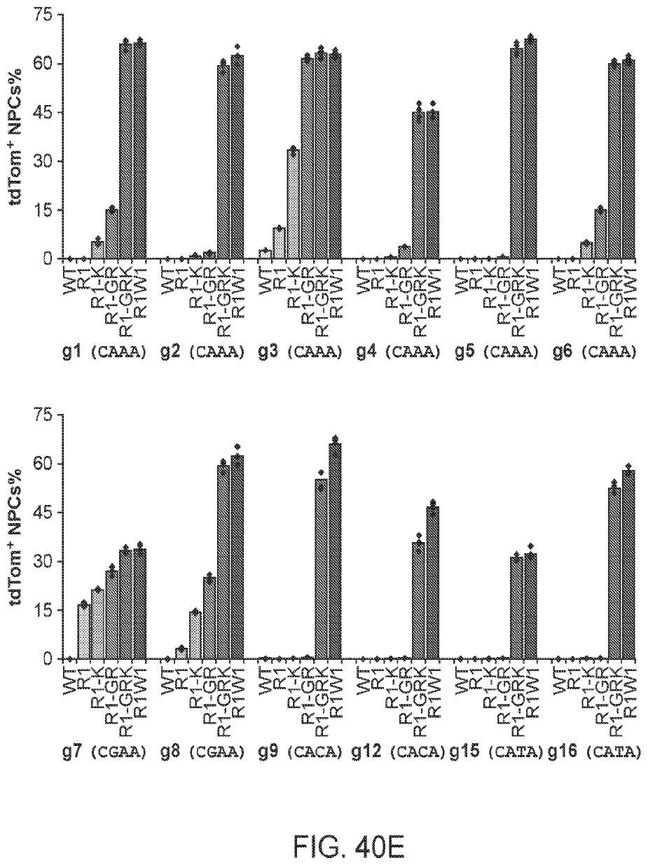

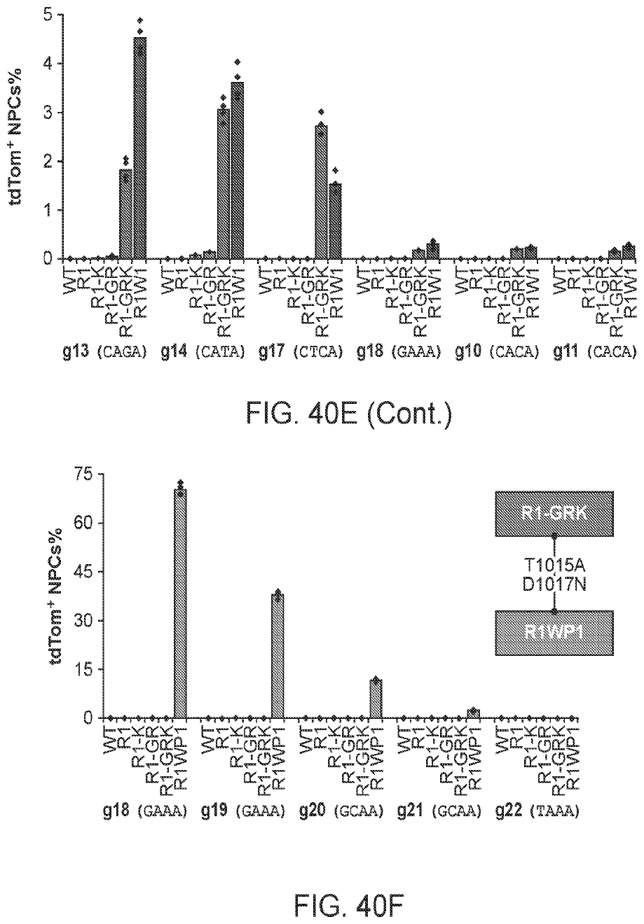

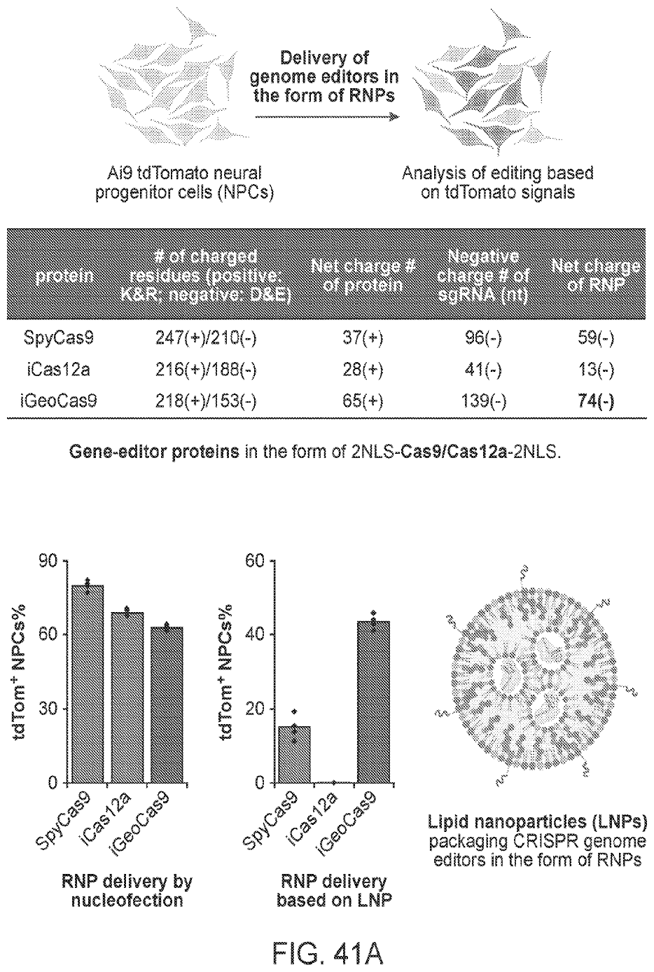

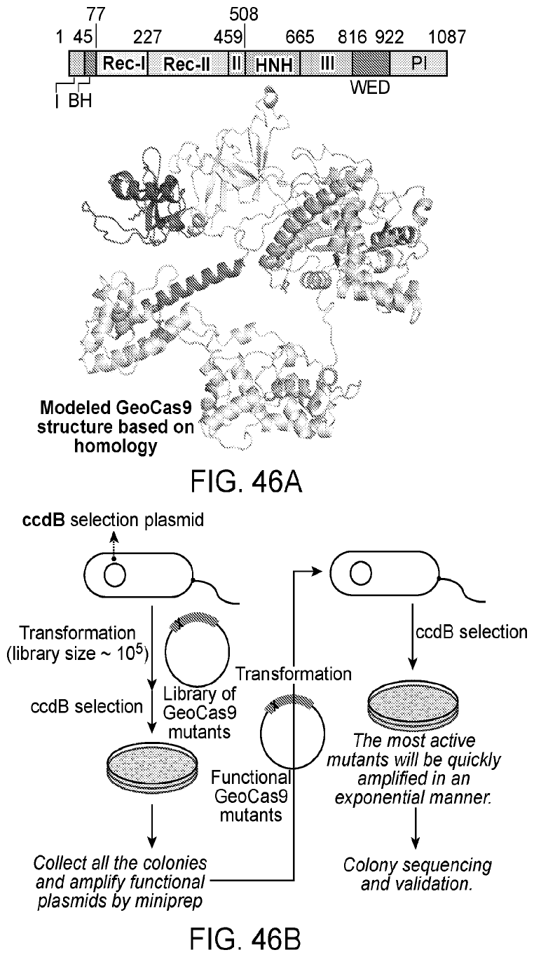

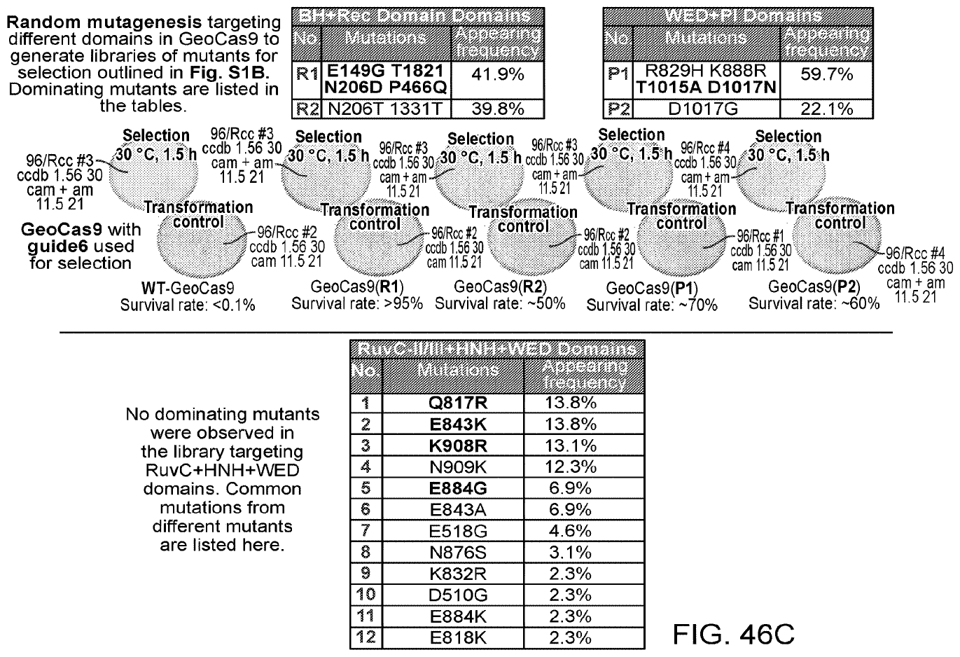

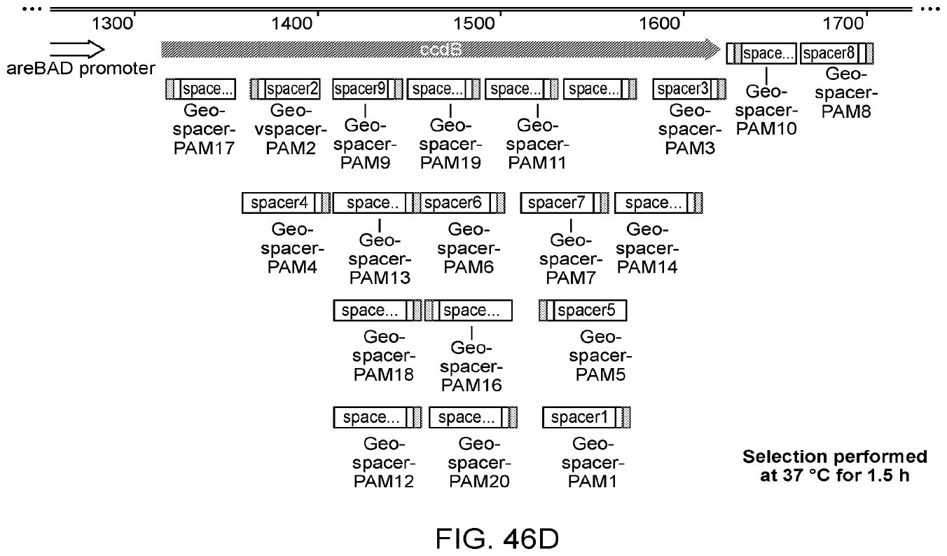

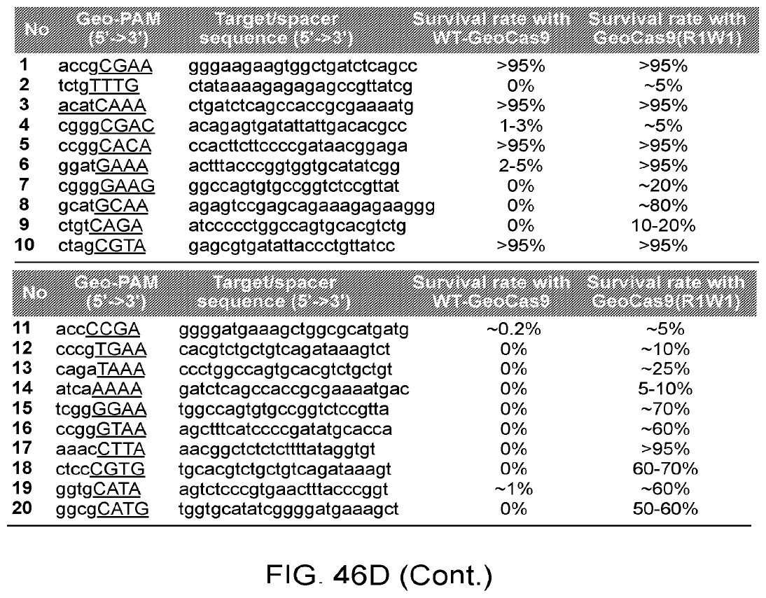

Directed evolution was harnessed to improve the editing efficiency of GeoCas9 and to expand the PAM sequences it could recognize. A bacterial dual-plasmid selection system was employed for the directed evolution of GeoCas9, which has been successfully employed for engineering other CRISPR Cas effectors. In this approach, the selection of active GeoCas9 variants relies on the Cas9-mediated cleavage of a plasmid encoding the ccdB toxin gene under the control of an inducible pBAD promoter (FIG. 40a). To search for a reliable evolution starting point with minimal activity in the E. coli assay, 20 different sgRNAs that target the ccdB gene at the protospacers associated with different PAM sequences were screened (FIG. 46d) and selection under two sets of conditions was performed (37 C or 30 C for 1.5 hours). The target sequence #6 with a disfavored PAM sequence (ggatGAAA) gave a minimal survival rate under either condition (<0.1% for 30° C. and 2-5% for 37 C) and was then chosen for engineering. Libraries of GeoCas9 mutants were generated by targeting different domains of the protein for random mutagenesis (FIG. 46a) and then subjected to the selection system under the conditions at 30° C. To amplify the most active mutants in the libraries, the selected mutants were collected and subjected to another round of selection (FIG. 46b). Sequencing of the selected colonies identified top mutants or frequently appearing beneficial mutations from each library (FIG. 46c). For instance, the library targeting BH+Rec domains for random mutagenesis afforded mutant GeoCas9(R1) bearing four mutations, E149G, T182I, N206D, and P466Q, which gave >95% survival (vs. <5% with the wild-type protein) in the bacterial assay. Further recombination of beneficial mutations identified in the library targeting RuvC+HNH+WED domains, including E843K, K908R, E884G, and Q817R, to the mutant R1, furnished a lineage of mutant proteins (FIG. 1b). Accumulating all the beneficial mutations yielded a final mutant, GeoCas9(R1W1) (hereafter referred to as iGeoCas9 for improved GeoCas9), which possesses greatly improved target dsDNA cleavage activity (FIG. 46d) and well-preserved thermostability (Tₘ: 55 C vs. 60° C., R1W1 mutant vs. wild-type protein, FIG. 1e; GeoCas9 RNP vs. SpyCas9 RNP, FIG. 47a).

The genome editing ability of the engineered GeoCas9 mutants was assessed in neural progenitor cells (NPCs) isolated from the Ai9 tdTomato mouse. In these cells, successful editing of the stop cassette sequence turns on the tdTomato gene, as illustrated in FIG. 1d. Twenty-two sgRNAs were designed to target the SV40-derived poly(A) region using various PAM sequences. RNPs, assembled from GeoCas9 mutants and sgRNAs, were electroporated into NPCs, and the percentage of tdTomato-positive cells was determined by flow cytometry. The evolved mutant, R1W1, exhibited substantially improved editing levels and edited cells with >1,000-fold efficiency relative to the wild-type GeoCas9 with most sgRNAs investigated (FIG. 1e). In addition to editing NPCs, the evolved R1W1 mutant also exhibited robust genome editing in immortalized human embryonic kidney (HEK293T) cells and was able to knock down the enhanced green fluorescent protein (EGFP) with up to >99% editing efficiency (FIG. 47b). These experiments demonstrate that the engineered GeoCas9 can accept a broader range of PAM sequences, including but not limited to 5′-NNNNCNNA-3′ (vs. wild-type PAM sequences: 5′-NNNNCRAA-3′).

To further expand the PAM compatibility of GeoCas9, the mutations T1015A and D1017N identified from the library targeting WED+PI domains (FIG. 46c) were incorporated into a later variant in the engineering lineage, GeoCas9(RJ-GRK), to furnish a new mutant, GeoCas9(R1WP1), which alters the preference of the first base in the essential 4-nt PAM sequence from C to G (Figure if). In addition, the beneficial mutations identified from GeoCas9 engineering were investigated for their ability to improve the performance of the homolog protein ThermoCas9. Mutations beneficial for GeoCas9 can also improve the genome editing efficiency of ThermoCas9 (FIG. 47c). Taken together, these results showed that directed evolution can be used to engineer GeoCas9 for improved genome editing activity and broadened PAM compatibility and that these engineering efforts can guide rational optimization/engineering of other CRISPR genome editors.

iGeoCas9 RNP Complexed to LNPs Efficiently Edits Cells In Vitro

The engineered iGeoCas9 has great potential for highly efficient editing cells and tissues in vivo in the RNP format, given its higher thermostability and efficient genome editing ability. LNPs are a powerful delivery tool to package and transport therapeutic agents to cells or tissues, including small-molecule drugs, oligonucleotides (e.g., siRNA, mRNA, etc.), and proteins. Previous studies demonstrated that SpyCas9 RNP could be packaged into LNPs and delivered into cell lines and in the liver and lungs of mice by recruiting the charge interaction between the sgRNA and cationic lipids. However, the delivery of SpyCas9 RNP/LNP complexes is less efficient than CRISPR editing with SpyCas9 mRNA/LNP complexes, and strategies for improving the delivery of RNP/LNP complexes are needed before they are a viable alternative to mRNA/LNP complexes.

The ability of LNPs to deliver GeoCas9 RNPs was investigated, using a variety of commercially available and in-house synthesized lipids. To have a comprehensive comparison, three genome editors based on SpyCas9, iCas12a, an engineered version of LbCas12a, and iGeoCas9 were initially investigated (FIGS. 41a and 48a). Delivery by nucleofection shows that all three genome editors gave robust and similar levels of genome editing in NPCs from Ai9 mice. However, the LNP-based delivery strategy gave totally different results: iGeoCas9 RNP was most effectively delivered to NPCs by LNPs, giving >2-fold higher editing efficiency compared to SpyCas9 RNP delivered by LNPs. The Cas12a RNP could not be packaged into LNPs. The improved performance of iGeoCas9 RNP, in comparison to SpyCas9 RNP, is likely due to its higher thermal stability and higher negative charge density. The high RNP stability of iGeoCas9 is beneficial for the LNP packaging process and should allow it to remain folded during exposure to organic solvents and lipids. In addition, iGeoCas9 RNP has a large number of negative charges, due to its long sgRNA, and this should facilitate efficient encapsulation into LNPs (FIG. 41a).

To set up a robust LNP-based system for iGeoCas9 RNP delivery, the lipid formulation for RNP encapsulation and LNP assembly were further optimized. Four commercial lipids were used, including DOTAP, D-Lin, DOPE, and cholesterol, and two synthetic lipids derived from cholesterol, ADP-2k and ADC, which are newly developed for mRNA delivery, as the basis for the optimization of LNP formulation (FIG. 41b). The pegylated lipid, ADP-2k, is the key to the successful encapsulation of RNP into LNPs and delivery to NPCs (FIG. 48b). Low percentages (<1%) of ADP-2k led to relatively large particle sizes, which is not beneficial for LNP delivery; on the other hand, high percentages (>5%) of ADP-2k had better control in particle size but showed lower efficacy for RNP packaging, which resulted in reduced editing in NPCs. These observations correspond to the known functions of pegylated lipids in enhancing LNP stability, controlling particle size, and regulating circulation time.

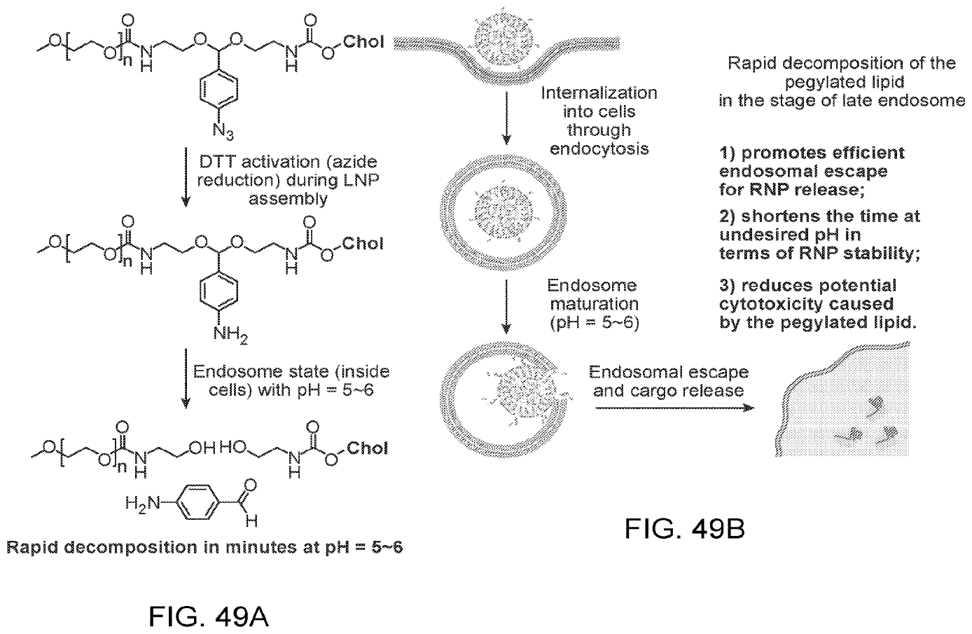

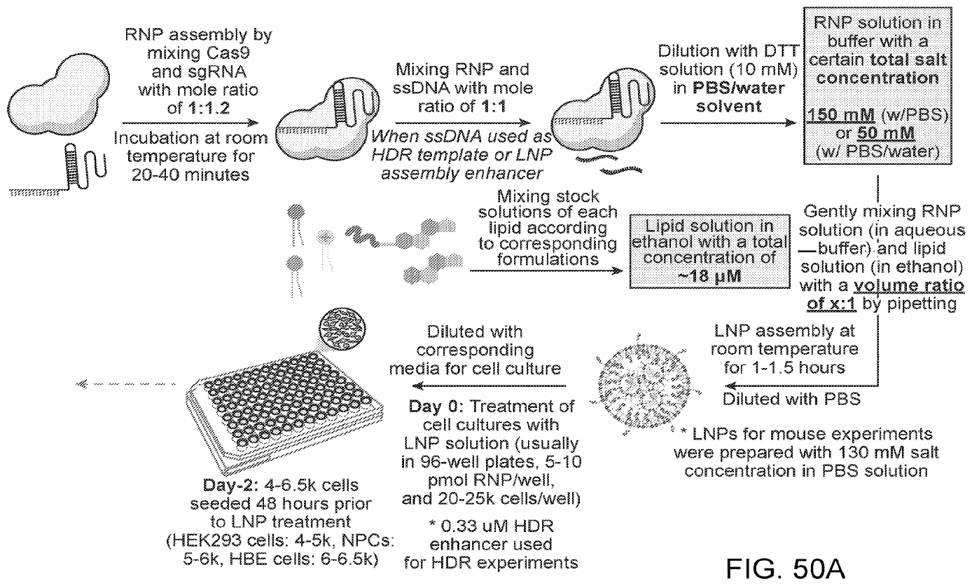

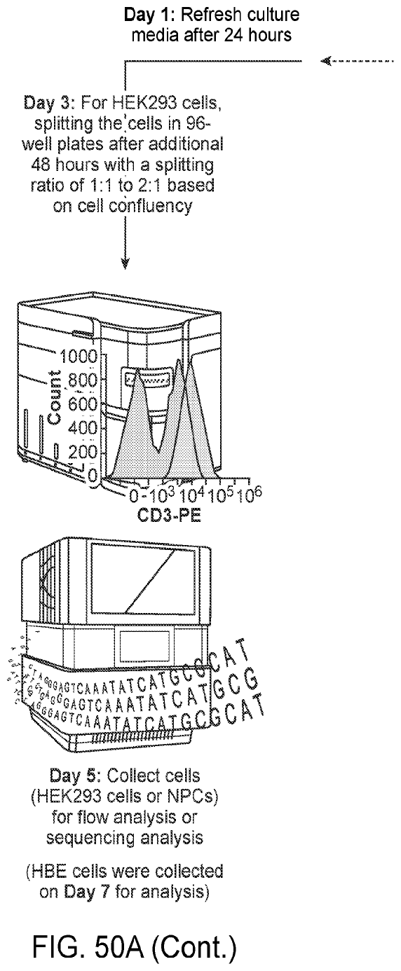

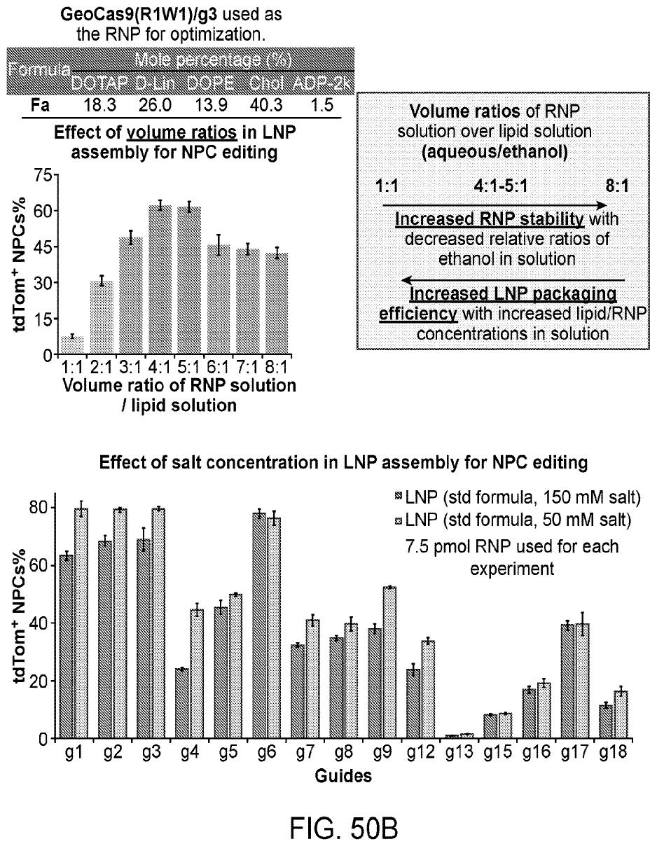

Several pegylated lipids, commercial and synthetic, were examiner for their ability to encapsulate and deliver iGeoCas9 RNPs in LNPs (FIG. 48c). However, the commonly used DMG-PEG and other PEG lipids derived from DOPE were detrimental to NPC viability, which is presumably caused by the surfactant properties of PEG-lipids. Interestingly, the synthetic pegylated lipid, ADP-2k, exhibited minimal toxicity with NPCs and had >90% cell viability; another two dipeptide-fused PEG lipids, Pep-1k and Pep-2k, also showed decent biocompatibility and high editing levels with NPCs. The reduced toxicity and enhanced delivery efficiency of these 3 PEGylated lipids stems from the pH-sensitive, acid-degradable acetal linker used in their synthesis (FIG. 49): the labile acetal linker could be cleaved in the late endosome stage of LNP delivery with a pH of 5-6, which frees the PEG moiety from the lipid molecules to reduce cytotoxicity and meanwhile destabilizes the endosome to promote the release of RNP into the cytosol. Further optimization of other parameters (e.g., mole and volume ratios of lipids to RNP, and salt concentration in the buffer, FIG. 50b) in LNP assembly established two sets of lipid formulations, i.e., standard formula (with DOTAP as the cationic lipid) and cationic formula (with ADC as the cationic lipid), which encapsulate GeoCas9 RNPs with good particle uniformity (diameter: 170-180 nm, PDI: 0.13-0.17) (FIG. 41b).

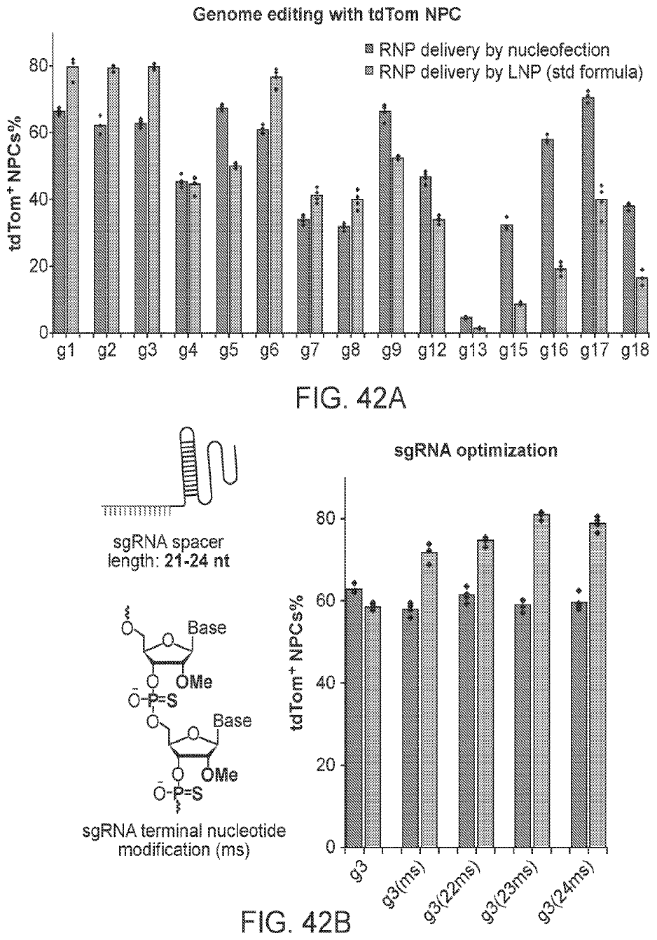

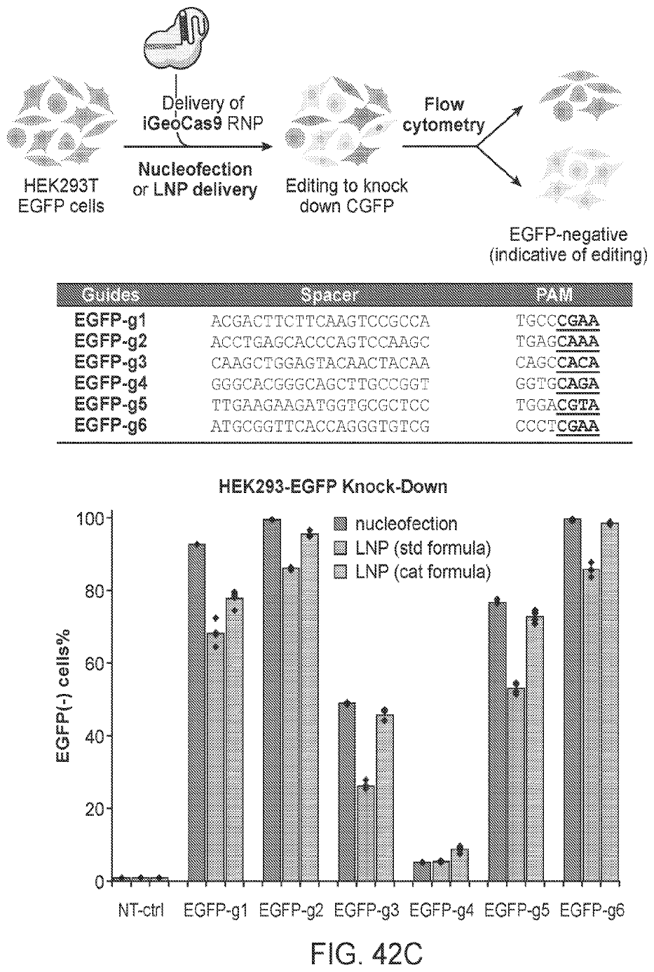

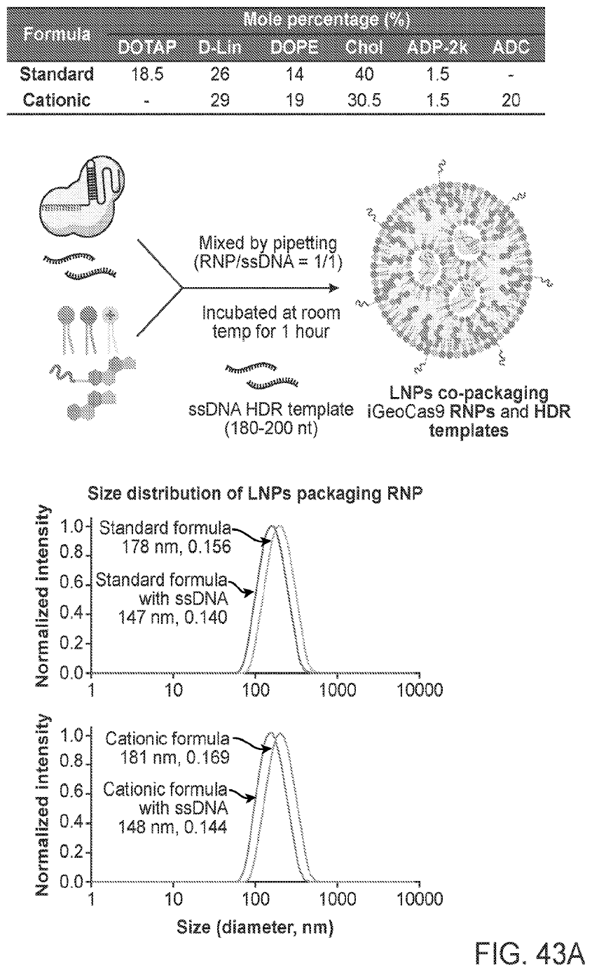

The genome editing efficacy of the standard formula and the cationic formula were evaluated in NPCs from the Ai9 mouse. RNPs were assembled using the engineered GeoCas9 mutants, R1W1 or R1WP1, with corresponding sgRNAs, and then encapsulated into LNPs following the procedure in FIG. 50a. Quantification of genome editing by flow cytometry after LNP treatment established that LNP-based delivery had comparable delivery efficacy to nucleofection (FIG. 42a). Simple changes to the sgRNA could further enhance the editing efficiency using the LNP delivery strategy. The protospacer region was extended from 21 nt to 23 or 24 nt and 2′-O methylation and phosphorothioate linkages were introduced to the last three nucleotides at both the 5′- and 3′-ends. These chemical modifications enhance the chemical stability of the sgRNA and are beneficial to RNP delivery with LNPs (FIG. 42b). The LNP strategy was also capable of delivering GeoCas9 RNPs to HEK293T cells and knocking down the EGFP transgene with comparable editing levels to nucleofection (FIG. 42c). The cationic lipid formulation for LNP assembly was found to be slightly more effective in RNP delivery to HEK cells. Altogether these experiments establish a robust LNP-based system for delivering GeoCas9 RNP to different cell lines to perform effective genome editing.

iGeoCas9 RNPs Delivered with ssDNA Templates Efficiently Induce HDR in Cells

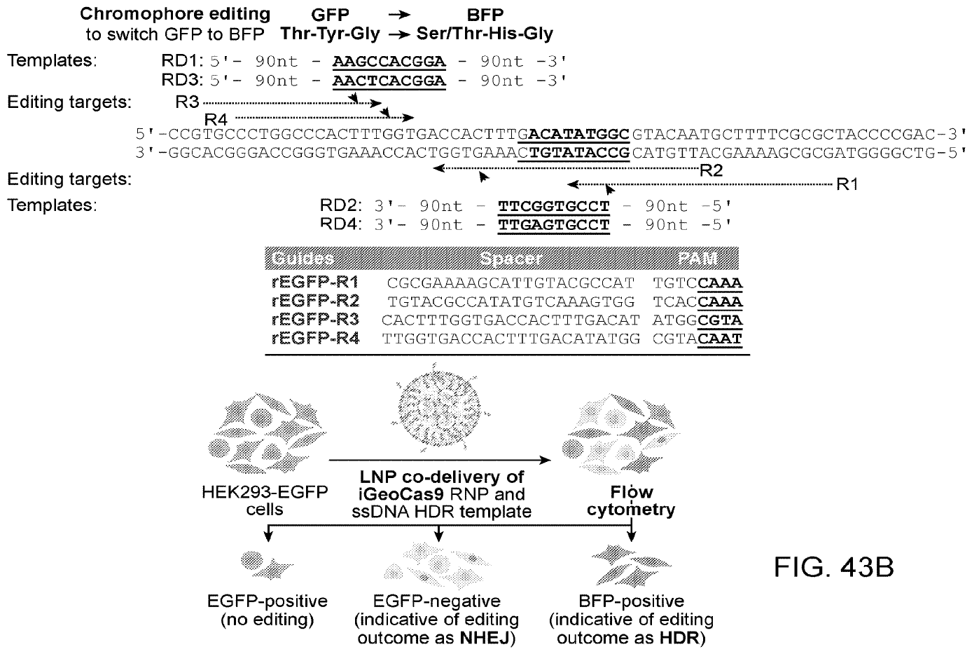

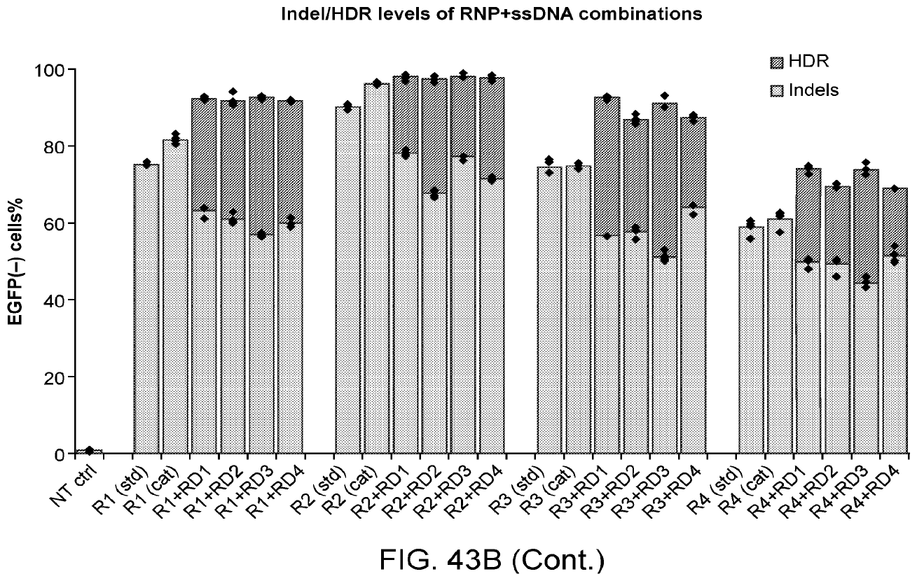

LNPs can efficiently deliver DNA into cells, and it was, therefore, hypothesized that LNPs would co-deliver iGeoCas9 RNP with a ssDNA template and induce specific genomic changes through homology-directed repair (HDR). The physical features of LNPs co-packaging of iGeoCas9 RNP and ssDNA templates 180-200 nt in size were first characterized (FIG. 43a). Interestingly, in the presence of ssDNA (with a mole ratio of 1:1 for RNP:ssDNA), the nanoparticle size was reduced from ˜180 nm to 140-150 nm. This phenomenon is consistent with a recent study showing that ssDNA could help the encapsulation of RNPs into LNPs and prevent LNP aggregation by transient binding to Cas9 RNPs.

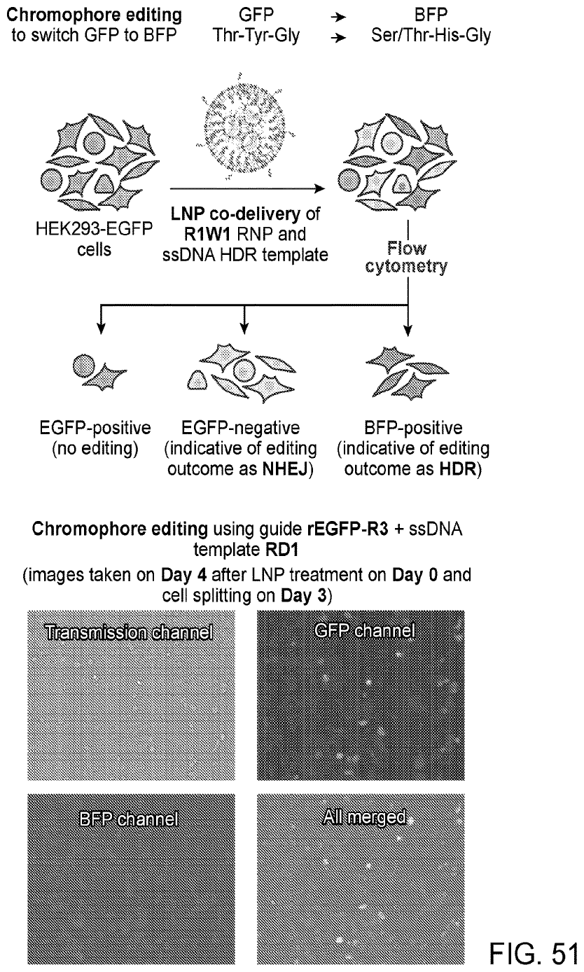

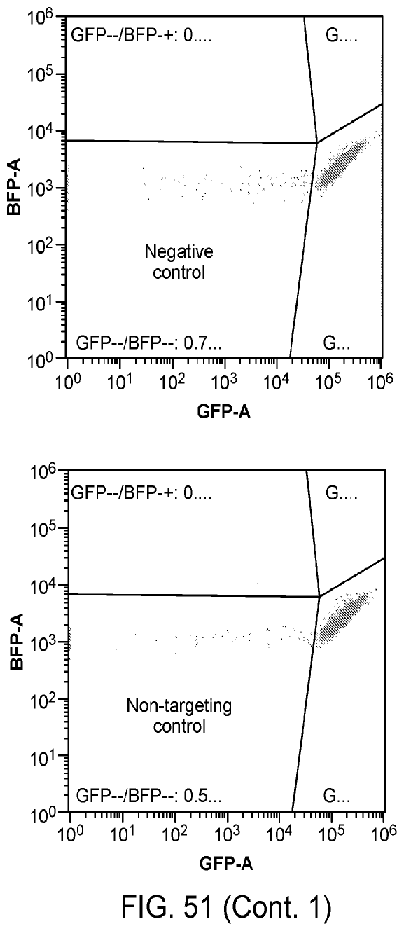

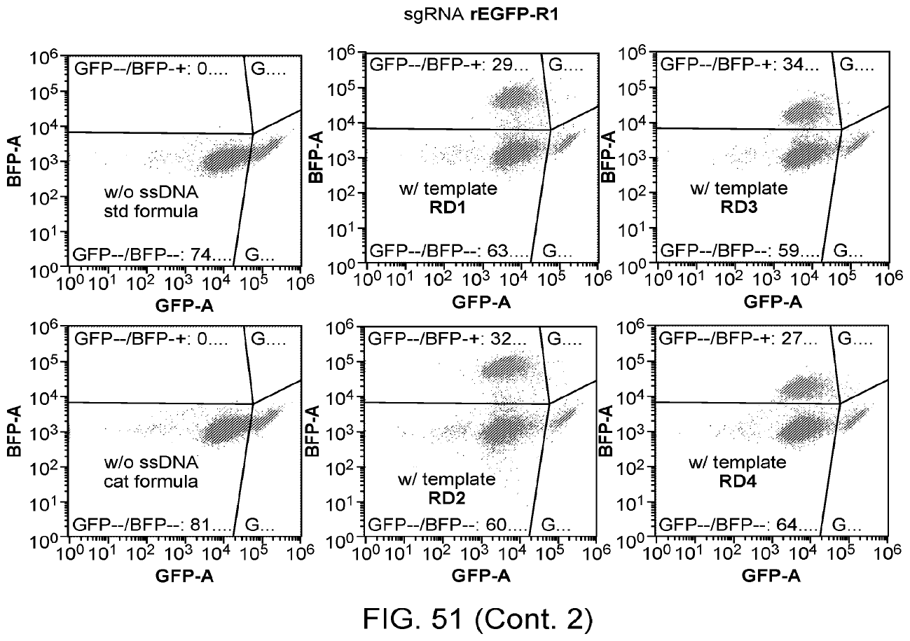

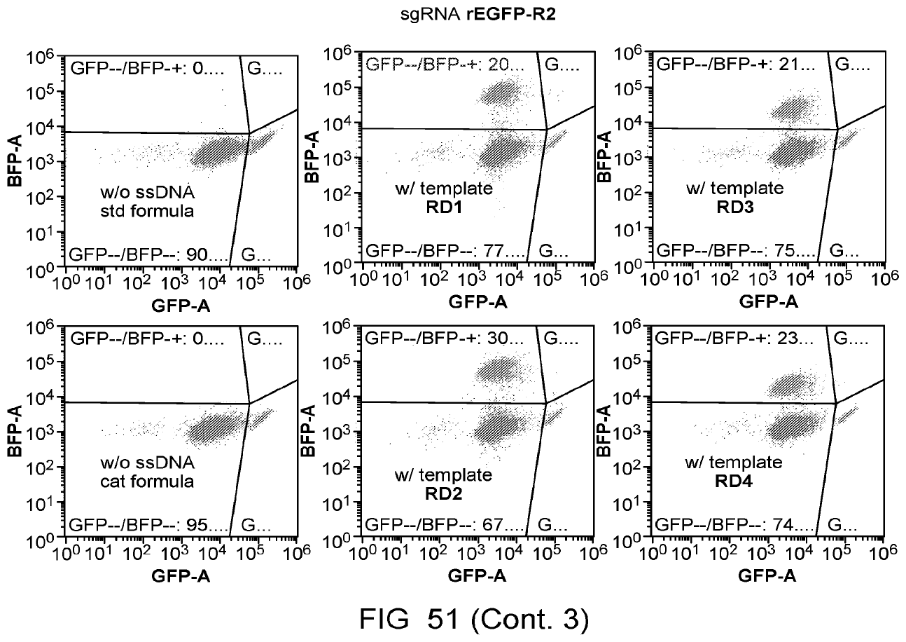

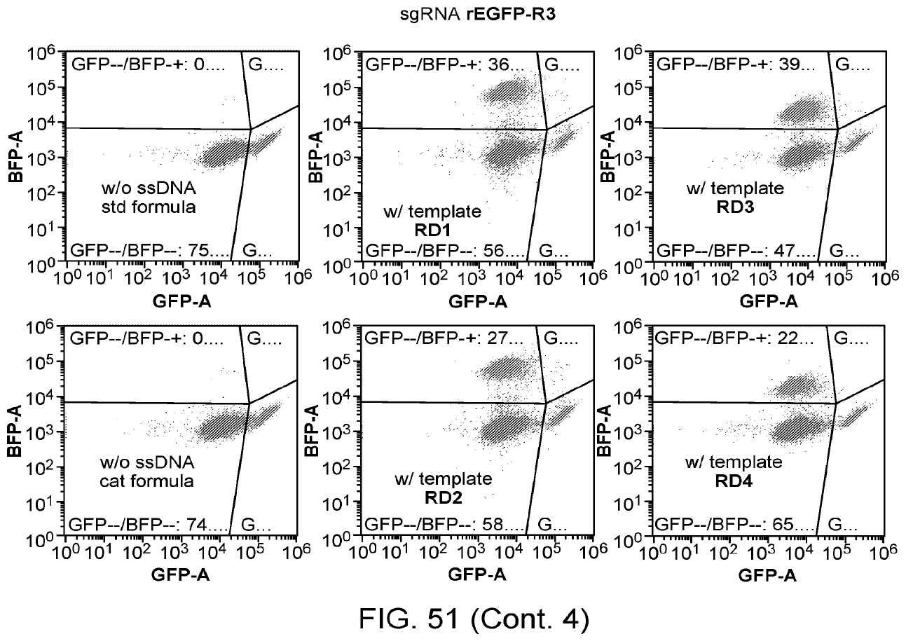

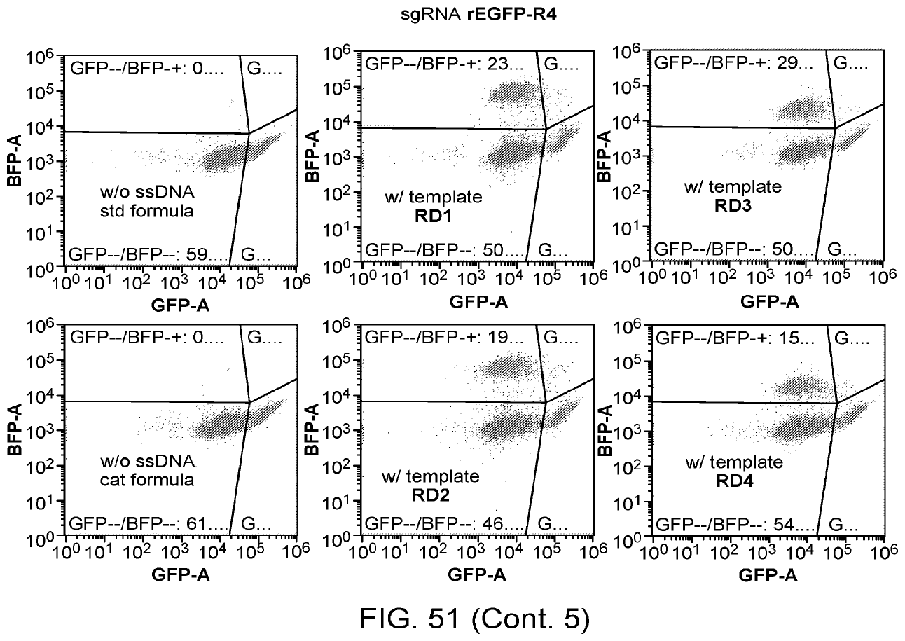

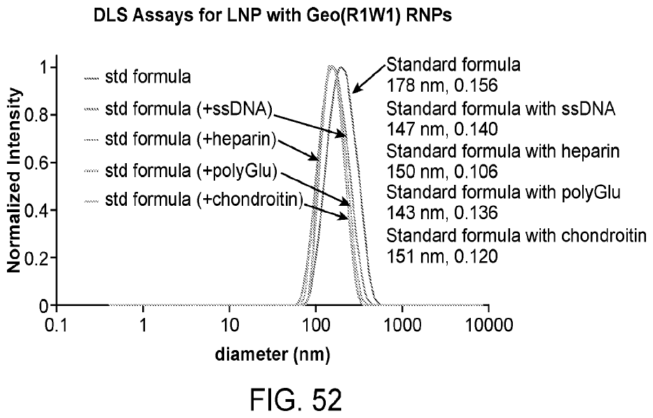

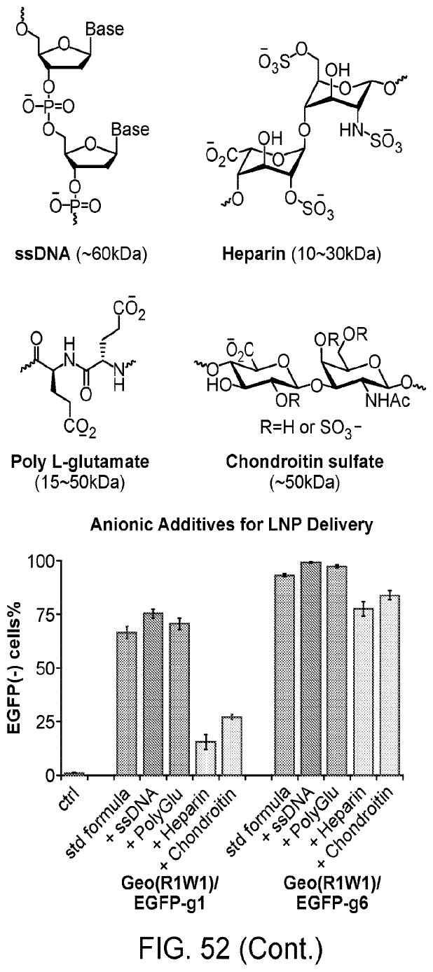

It was investigated if the co-delivery of iGeoCas9 RNPs and ssDNA templates in LNPs could switch EGFP to the blue fluorescent protein (BFP) in a model HEK293T cell line (FIG. 43b). In this cell assay, editing of the chromophore Thr-Tyr-Gly in the EGFP transgene followed by HDR to install a Ser/Thr-His-Gly chromophore turns EGFP into BFP. Four sgRNAs, rEGFP-R1 to R4, were designed to target the coding and non-coding strands in the chromophore region for editing. To avoid possible recutting events after the incorporation of the desired edits, four ssDNA HDR templates were designed to introduce GFP-to-BFP edits together with additional silent mutations in the DNA sequence. BFP signals were observed with the co-delivery tests based on all 16 combinations of RNPs and ssDNA templates using the standard lipid formulation for LNP assembly. The HDR level was quantified to be between 20-40% by flow cytometry with 50-75% of non-homologous end-joining (NHEJ) indicated as EGFP knockdown (FIGS. 4b and 51). Interestingly, the HDR experiments gave overall higher editing (HDR+NHEJ) levels compared to EGFP knockdown by RNP only, which speaks to the collateral role of ssDNA in promoting RNP encapsulation into LNP. It was then tested whether other anionic polymers, such as poly-L-glutamate (MW 15-50 kDa) and heparin (MW 10-30 kDa), could have similar effects. As expected, the anionic polymer, poly-L-glutamate, could also help shrink the LNP size and mildly improve the editing levels; however, the addition of heparin resulted in reduced editing, probably due to its inhibitive effect on Cas9 function (FIG. 52). These results suggest that anionic polymers could promote RNP packaging into LNPs through the charge interaction between the polymer additives and cationic lipids.

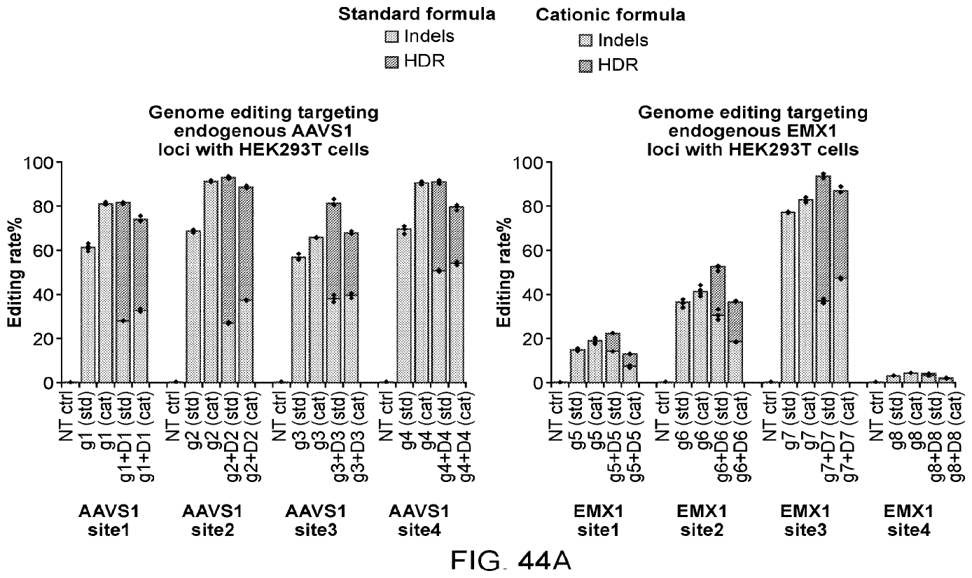

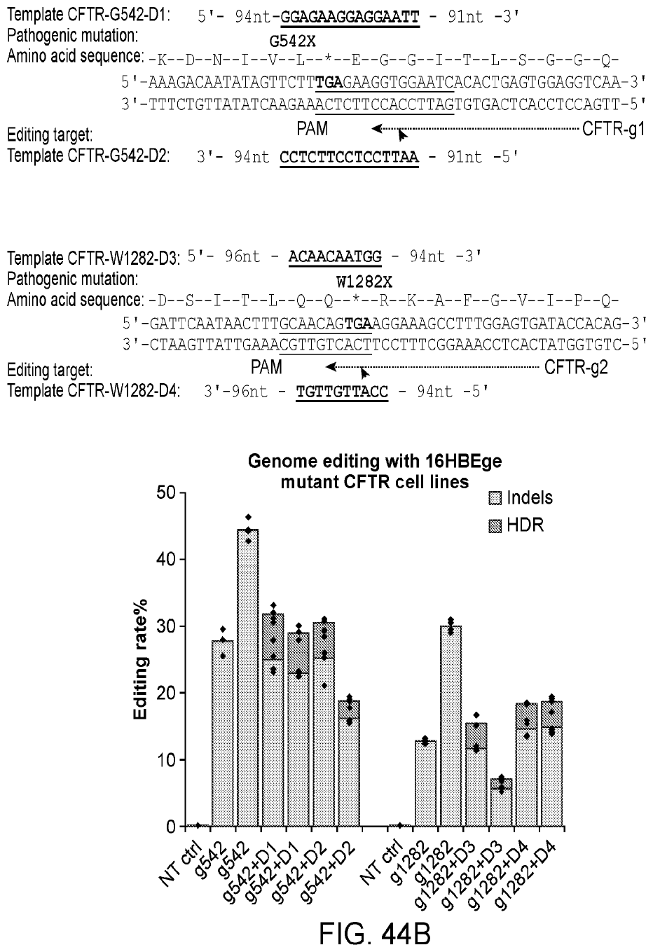

LNP-based co-delivery of iGeoCas9 RNPs and ssDNA templates was further used to induce HDR at endogenous genomic sites in human cells. Four sets of guide RNAs and corresponding donor ssDNAs were designed to target different loci in the EMX1 and AAVS1 genes, respectively, for genome editing based on HDR (FIG. 44a). Both the standard and cationic LNP formulations were evaluated for their ability to deliver editing materials to HEK293T cells. HDR levels were quantified using next-generation sequencing (NGS), and LNP/RNP complexes generated HDR up to 66%, with total editing levels up to 95%. This co-delivery system was then applied to cell lines of disease models and investigated if LNP/RNP complexes can correct pathogenic mutations. Cystic fibrosis is a genetic disease caused by pathogenic mutations in the CFTR gene, which encodes the ion channel protein, cystic fibrosis transmembrane conductance regulator (CFTR). Two human bronchial epithelial cell (16HBEge) lines with non-sense mutations in the CFTR gene, G542X and W1282X, respectively, were employed for the HDR tests (FIG. 44b). iGeoCas9 RNPs and HDR donors were co-delivered to the HBE cells to revert the pathogenic mutations G542X and W1282X, and gave appreciable HDR levels, generating up to 7% HDR, as quantified by NGS. Based on these results, this LNP-based platform should be useful for therapeutic genome engineering.

LNP-Based Delivery of iGeoCas9 RNP Edits Multiple Organs Efficiently In Vivo

Having demonstrated that iGeoCas9 RNPs could be delivered by LNPs to a variety of cell lines for different editing purposes, it was then asked if the RNP-LNP strategy could be applied to in vivo genome editing in mice after a retro-orbital injection. It was tested whether the iGeoCas9 RNPs can be delivered to organs beyond the liver, which represents a challenge for LNP-mediated delivery of CRISPR genome editors.

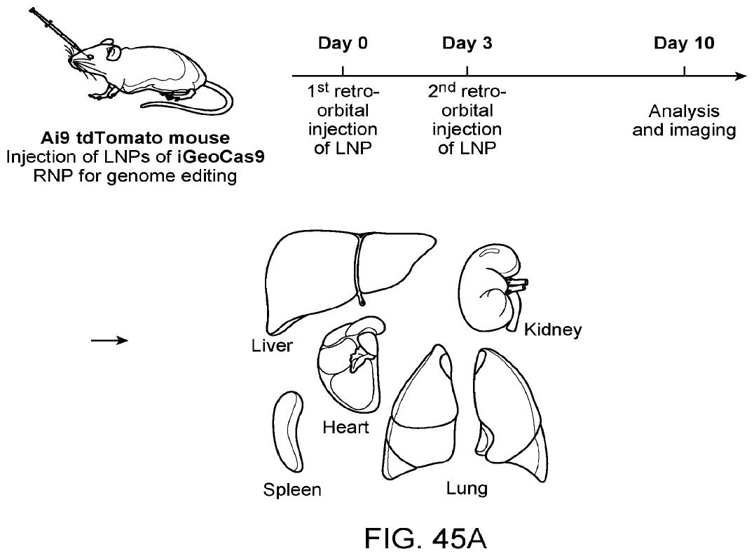

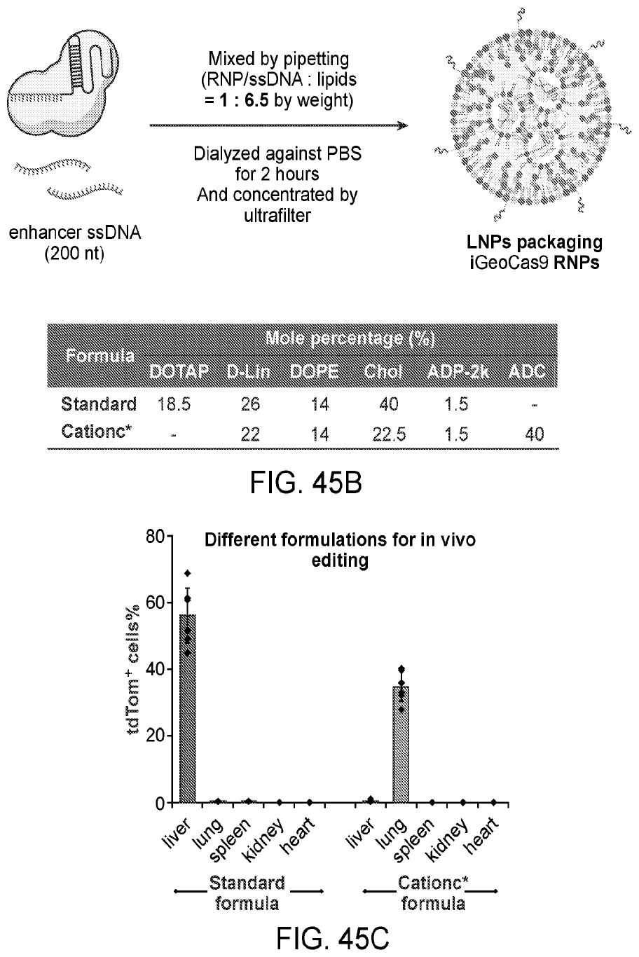

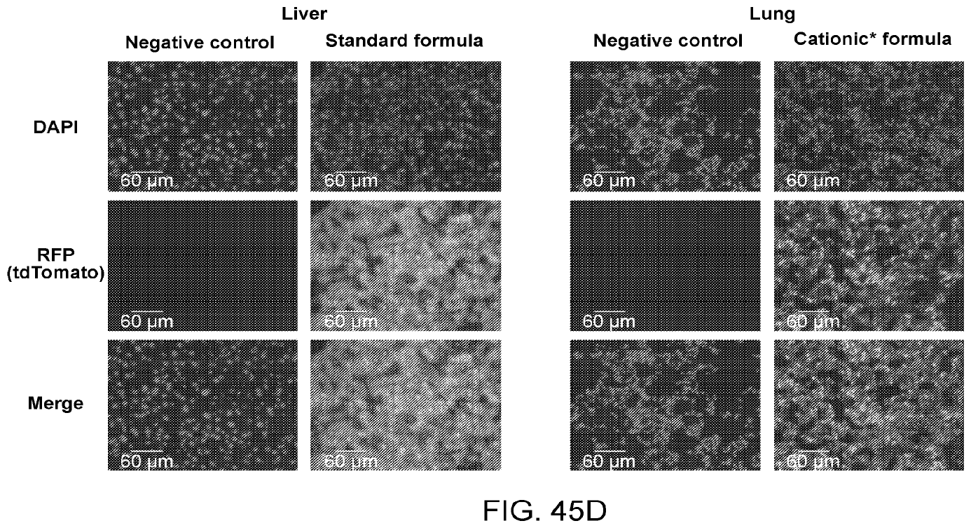

tdTomato Ai9 mouse models were employed to assess the delivery and editing efficacy in vivo of iGeoCas9 using the LNP-based RNP delivery system disclosed herein (FIG. 45a). Previous demonstration on SORT LNPs to deliver mRNA in an organ-specific manner by the Siegwart lab prompted testing of different lipid formulations to see whether the genome editor can be efficiently delivered to organs beyond the liver. In addition, with the beneficial effect of ssDNA in RNP encapsulation and LNP delivery as disclosed in the present results, a 200-nt ssDNA was used as an enhancer for encapsulating iGeoCas9 RNPs in LNPs based on different lipid formulations. Two retro-orbital injections of LNPs were performed at a dose of 3.0 mg/kg based on sgRNA (-˜1.0 nmol RNP/injection based on mouse weight of 18-20 grams) at 3-day intervals. Mice were sacrificed a week after the second injection, and organs, including the liver, lung, spleen, heart, and kidney, were collected to analyze the tdTomato signal as the genome-editing outcome (FIG. 45b). Imaging of the organ slices together with flow quantification of tdTomato-positive cells revealed that iGeoCas9 RNP can be successfully delivered and execute robust genome editing in vivo with up to 56% editing in the liver and 35% editing in the lungs (FIG. 45c). Notably, the standard LNP formulation drove the delivery of RNPs primarily to the liver, while a modified cationic formulation using 40% ADC as the cationic lipid shifted the delivery specificity to the lungs with unprecedented genome editing levels. In addition, genome editing was also observed in other tissues known as challenging delivery targets, such as the heart (0.2% genome editing indicated by the tdTomato signals), which facilitates the development of therapeutics for those targets.

### Discussion:

Here is described a generalizable approach for CRISPR genome editing via the efficient delivery of a thermostable genome editor, e.g., in the RNP format, both in vitro and in vivo. The direct delivery of RNPs has several advantages over viral-based or mRNA-based delivery strategies, however, efficient RNP delivery can be challenging for in vivo genome editing. Different packaging systems have been shown to deliver RNPs in vitro, but the in vivo applications of these systems have been limited due to issues regarding particle uniformity, stability, and biocompatibility. Therefore, the LNP strategy was used to develop a robust vehicle system to encapsulate and deliver genome editors based on a thermostable GeoCas9.

GeoCas9 exhibits superior stability to the commonly used SpyCas9 under a variety of different conditions relevant to in vivo delivery, but it is a much less efficient genome editor. Nevertheless, the extra stability endows GeoCas9 the ability to accept a wide range of mutations beneficial to its function while maintaining its native structure, and this would promote its evolvability in nature or in the laboratory, as indicated by the large set of natural homologous proteins of GeoCas9 (with >75% sequence identity). The successful directed evolution of GeoCas9 reported here further confirms its high tunability. The evolved mutant, iGeoCas9, can robustly edit different cell lines with efficiency orders of magnitude higher than the wild-type GeoCas9 and is an alternative choice to SpyCas9 for genome editing. Beyond that, the engineered GeoCas9 also showed well-preserved protein stability, which facilitates using LNPs to deliver GeoCas9 RNPs in vitro and in vivo.

LNPs are a powerful non-viral delivery platform for many therapeutic agents, ranging from small molecule drugs to nucleic acids to proteins, as exemplified by the siRNA drug, patisiran (ONPATTRO®), and the mRNA-based COVID-19 vaccines. The stability and charge properties of GeoCas9 RNP allow for its effective interaction with positively charged lipids and encapsulation into LNPs, rendering it a desirable genome editor for LNP delivery. With optimized LNP formulations described herein, iGeoCas9 RNPs were delivered to different cell lines with genome editing efficiency comparable to or even higher than nucleofection while showing minimal toxicity. A synthetic pegylated lipid, ADP-2k, aided development of the RNP/LNP delivery strategy, where a designed acid-degradable linker allowed for rapid decomposition of the lipid in the late endosome stage of delivery, thus promoting RNP release into the cytosol and reducing cytotoxicity associated with the pegylated lipid. The LNP-based delivery vehicle was also utilized for the co-delivery of RNPs and ssDNA templates to incorporate specific genomic changes through homology-directed repair. Interestingly, ssDNA templates were found to simultaneously promote the encapsulation of RNPs into LNPs, presumably through transient binding of ssDNA to RNPs, and shrunk the nanoparticle size. Successful HDR was observed with HEK293T cells and cell lines of disease models to correct pathogenic mutations, highlighting the capability of the LNP-based delivery platform disclosed herein for therapeutic applications. With these results, the LNP strategy can be extended to other related genome-editing tools, including prime editors and base editors.

Genome editing in vivo was also demonstrated using the LNP-assisted RNP delivery system. Two established formulations, standard and cationic formulas, were found to target the liver and the lungs for RNP delivery, and the editing levels were quantified to be >35% in the targeted tissues in Ai9 mouse models. The delivery specificity can be regulated by the charge properties of the formulations; e.g., the use of the cationic lipid, ADC in 40%, shifted the specificity from the liver to the lung. Previous delivery methods based on viral vectors or LNPs have been successfully demonstrated to transport genome editors to the liver, oftentimes as the result of liver hepatocyte accumulation, while the LNP-based delivery strategy used herein could switch the destination of the genome editors to the lungs and effect high levels of genome editing. As illustrated by Siegwart, plasma proteins can be recruited by the LNPs with different chemical properties, which leads to different cellular internalization specificity based on a receptor-mediated uptake mechanism. Together with the genome-editing activities observed in other organs, such as the heart, it is now possible to screen and optimize new LNP formulations to further switch the delivery specificity to these other targets. These developments allow researchers to carry out in vivo genome editing in tissues other than the liver and will facilitate expanding the therapeutic applications of CRISPR-Cas9 gene therapy.

### Materials and Methods

### Plasmid Construction

Plasmids used for the expression of different Cas proteins in this study were built based on a pCold vector. The inserts encoding Cas proteins contain an N-terminal CL7 tag followed by an HRV-3C protease cleavage site, and a C-terminal His₆ tag following another HRV-3C protease cleavage sequence. The insert for the final NLS-GeoCas9(R1W1)-2NLS protein contains an N-terminal sequence consisting of different tags, His₆-CL7-MBP (MBP: maltose-binding protein) followed by an HRV-3C protease cleavage site. The cloning reactions were carried out in a 50-μl reaction containing 1 ng of template plasmid, 1.25 μl of 10 mM dNTP, and 1.25 μl of 10 μM each primer using Phusion™ high-fidelity DNA polymerase (New England BioLabs). After PCR, the reactions were treated with 1 μl of DpnI (New England BioLabs) for 1 hour at 37° C. before gel purification. The plasmids were ligated based on Gibson assembly (New England BioLabs master mix) of plasmid backbone and insert sequences. The sequences of all the plasmid constructs were confirmed via full plasmid sequencing (Primordium).

### Nucleic Acid Preparation

All of the DNA and RNA oligos used in this study were purchased from Integrated DNA Technologies, Inc. (IDT) and HPLC or PAGE-purified. Some of the sgRNAs purchased from IDT possess chemical modifications at 3′- or 5′-ends.

### Directed Evolution of GeoCas9

A chloramphenicol-resistant (CAM+) bacterial expression plasmid was built to have the insert gene of GeoCas9 together with its corresponding sgRNA that targets the ccdB gene in the selection plasmid with a PAM of GAAA (g6). Libraries of GeoCas9 mutants were generated by error-prone PCR to introduce random mutagenesis in three different regions (BH-Rec, RuvC-HNH-WED, and WED-PI). The error-prone PCR (with an error rate of 3- to 5-nucleotide mutations per kilobase) was carried out with the Taq DNA polymerase (New England BioLabs) in a reaction containing 2 μl of 10 mM primers, 1.5 μl of 10 mM MnCl2, 2 ng of template plasmid. The plasmid libraries were generated by ligating the mutated fragments with the remaining part of the plasmid through Gibson assembly. The plasmid libraries (-100 ng DNA after clean-up) were electroporated into 50 μl of electronically competent cells made from E. coli strain BW25141(DE3) that contains the selection plasmid encoding the arabinose-inducible ccdB toxin gene. After recovery of the electroporated bacteria in 750 μl of SOB for 1.5 hours at 30° C., the bacteria culture was concentrated; 1% of the total culture was plated onto a Petri agar dish containing only CAM (as control), and the remainder culture was plated on another Petri agar-dish containing both arabinose and CAM. Positive colonies that grew on the plates containing both arabinose and CAM were collected in a pool, retransformed (with ˜2 ng plasmid), and replated (100 μl of transformed culture on both control and selection plates). Plasmids of individual colonies from the replated plate were sequenced to obtain mutational information. Validation of the positive clones in the bacterial assay followed the same procedure.

### Protein Expression

All the proteins in this study were expressed in E. coli BL21 (DE3) cells (Sigma-Aldrich) cultured in 2×YT medium supplemented with the antibiotics of ampicillin. The cultivation was carried out at 37° C. with a shaking speed of 160 rpm after inoculation with an overnight starter culture in LB medium containing ampicillin at a ratio of 1:40. When the optical density (OD₆₀₀) of the culture reached 0.8-0.9, the culture was cooled down to 4° C. on ice. The expression of Cas proteins was induced by the addition of isopropyl P-D-1-thioglalacctopyranoside (IPTG) to a final concentration of 0.1 mM and incubated at 15.8-16° C. with a shaking speed of 120 rpm for 14-16 hours.

To purify the Cas proteins, the cultured cells were harvested and resuspended in lysis buffer (50 mM Tris-HCl, 20 mM imidazole, 1.2 M NaCl, 10% (v/v) glycerol, 1 mM TCEP, 0.5 mM, and cOmplete™ protease inhibitor cocktail tablets (Millipore Sigma, 1 tablet per 50 ml) at pH 7.5), disrupted by sonication and centrifuged at 35,000×g for 45 min. Ni-NTA resin was treated with the supernatant at 4° C. for 60 min, washed with wash buffer-1 (lysis buffer without protease inhibitor cocktail tablet), and eluted with elution buffer (50 mM Tris-HCl, 300 mM imidazole, 1.2 M NaCl, 10% (v/v) glycerol, and 1 mM TCEP at pH 7.5) to give crude His-tagged Cas proteins. The nickel elution was then subjected to Im7-6B resin in a slow gravity column repeatedly (3-4 times). The Im7-6B resin was washed with wash buffer-2 (50 mM Tris-HCl, 1.2 M NaCl, 10% (v/v) glycerol, and 1 mM TCEP at pH 7.5) before being treated with HRV-3C protease (1% weight to crude Cas protein) for 2-2.5 hours to release the Cas proteins from the CL7 and His₆ tags. Heparin affinity column was used to further purify the desired proteins. The protein fractions were collected, concentrated, and stored in storage buffer (25 mM NaPi, 150 mM NaCl, and 200 mM trehalose at pH 7.50) after buffer exchange. The final yields of different Cas proteins (all with two copies of NLS at both N- and C-termini): wild-type GeoCas9, −10 mg per 1 L culture; GeoCas9 mutants, in a range of 2-10 mg per 1 L culture; SpyCas9, −4 mg per 1 L culture; iCas12a, −30 mg per 1 L culture.

The purification of the final NLS-GeoCas9(R1W1)-2NLS protein is slightly different after Ni-NTA resin purification. The nickel elution was subjected to dialysis against dialysis buffer (50 mM Tris-HCl, 10 mM imidazole, 1.2 M NaCl, 10% (v/v) glycerol, and 1 mM TCEP at pH 7.5) containing HRV-3C protease (1% weight to crude Cas protein) for 12-15 hours. The tag-cleaved protein was then loaded to a heparin column and washed with 80 column volumes of buffer containing 0.1% Triton X-114 at 4° C. to minimize endotoxin impurities. The protein fractions were collected, concentrated, and subjected to further purification using a size-exclusion column in an endotoxin-free manner. The purified protein was stored in an endotoxin-free storage buffer (25 mM NaPi, 150 mM NaCl, and 200 mM trehalose at pH 7.50). The final yield of the desired GeoCas9 mutant: 5-8 mg per 1 L culture.

### Measurement of Protein Melting Temperatures

Protein melting temperatures were measured using the thermal shift assay (GloMelt™, CAT #33021). The assay was performed on a quantitative PCR system with a temperature increase rate of 2° C./min. The protein melting temperatures were determined as the peak values in the derivative curves of the melting curves.

### Cell Lines and Culture Conditions

NPCs were isolated from embryonic day 13.5 Ai9-tdTomato homozygous mouse brains. Cells were cultured as neurospheres at 37° C. with 5% CO2 in NPC medium: DMEM/F12 (Gibco, CAT #10565018) with GlutaMAX™ supplement, sodium pyruvate, 10 mM HEPES, nonessential amino acid (Gibco, CAT #11140076), penicillin and streptomycin (Gibco, CAT #10378016), 2-mercaptoethanol (Gibco, CAT #21985023), B-27 without vitamin A (Gibco, CAT #12587010), N2 supplement (Gibco, CAT #17502048), and growth factors, bFGF (BioLegand, CAT #579606) and EGF (Gibco, CAT #PHG0311) (both 20 ng/ml as final concentration). NPCs were passaged using MACS Neural Dissociation Kit (Papain, CAT #130-092-628) following manufacturer's protocol. bFGF and EGF were refreshed every three days and cells were passaged every 5 days. Pre-coating with a coating solution containing poly-DL-ornithine hydrobromide (Sigma-Aldrich, CAT #P8638), laminin (Sigma-Aldrich, CAT #11243217001), fibronectin bovine plasma (Sigma-Aldrich, CAT #F4759) was required for culturing cells in 96-well plates.

HEK293T and HEK293T-EGFP cells were grown in medium containing DMEM (Gibco, CAT #10569010), high glucose, GlutaMAX™ supplement, sodium pyruvate, 10% FBS, and penicillin and streptomycin (Gibco, CAT #10378016) at 37° C. with 5% CO2. Cells were passaged every 3 days.

16HBEge cells were grown in medium containing MEM (Gibco, CAT #11090099), 10% FBS, and penicillin and streptomycin (Gibco, CAT #10378016) at 37° C. with 5% CO2. T75 flasks pre-coated with a coating solution containing LHC-8 basal medium (Gibco, CAT #12677-027), bovine serum albumin 7.5% (Gibco, CAT #15260-037), bovine collagen solution, Type 1 (Advanced BioMatrix, CAT #5005), fibronectin from human plasma (ThermoFisher, CAT #33016-015) were used for culturing 16HBEge cells. Cells were passaged every 4-5 days. Pre-coating was required for culturing cells in 96-well plates.

Genome Editing with Different Cell Lines

Nucleofection: 250k NPCs or 200k HEK293T cells were nucleofected with 100 pmol pre-assembled RNP (with 100 pmol ssDNA enhancer) with program codes of EH-100 and CM-130, respectively, according to the manufacturer's instructions. Lonza SF (for HEK293T cells) and P3 (for tdTomato NPCs) buffers were used for the preparation of nucleofection mixtures (with a total volume of 20 μl). 10% of the nucleofected cells were transferred to 96-well plates. The culture media for NPCs was refreshed after 3 days; HEK293T cells were split with a ratio of 5:1 after 3 days. Cells were harvested for analysis after further incubation at 37° C. for 2 days.

LNP delivery: 4-6.5k cells/well were seeded in 96-well plates 48 hours prior to LNP treatment (HEK293 cells: 4-5k. NPCs: 5-6k, and 16HBEge cells: 6-6.5k). The culture media was refreshed 24 hours after LNP treatment. HEK293T cells were split after 2 additional days with a ratio of 1:1 to 2:1 based on cell confluency. Cells were harvested for analysis after a total incubation time of 5 days.

### Flow Cytometry

Cell fluorescence was assayed on an Attune NxT acoustic focusing cytometer (Thermo Fisher Scientific) equipped with 554 nm excitation laser and 585/16 emission filter (tdTomato). 488 nm excitation laser and 530/30 emission filter (EGFP), and 400 nm excitation laser and 440/50 emission filter (BFP). Data were analyzed using Attune Cytometric Software v5.1.1.

### Next-Generation Sequencing

Edited cells were harvested and treated with Quick Extraction solution (Epicentre, Madison, WI) to lyse the cells (65° C. for 20 min and then 95° C. for 20 min). Amplicons of genomic targets were PCR-amplified in the presence of corresponding primers which were designed to have no overlap with their corresponding donor ssDNA sequence in the case of HDR. The PCR products were purified with magnetic beads (Berkeley Sequencing Core Facility) before being subjected to next-generation sequencing (NGS) with MiSeq (Illumina) at 2×300 bp with a depth of at least 20,000 reads per sample. The sequencing reads were subjected to CRISPResso2 (https://github.com/pinellolab/CRISPResso2) to quantify the levels of indels and HDR.

### LNP Formulations and Characterization

An aqueous solution (PBS/water 1:1, with 5 mM DTT, pH 7.3-7.5) of GeoCas9/sgRNA RNPs (±ssDNA) was prepared and pipette-mixed rapidly with the lipid solution in ethanol at a volume ratio of 4:1 and mole ratio of 0.5-1.1:1000, and then incubated for 1-1.5 hours at room temperature. The LNP mixture was diluted with PBS before the treatment with cell cultures. Under this procedure, RNPs were largely in excess. The size distribution of nano-formulations was measured using Zetasizer (version 7.13, Malvern Panalytical; He—Ne Laser, λ=632 nm; detection angle=173°).

The preparation of LNPs for animal experiments was slightly different. An aqueous solution (PBS/water 1:1, with 5 mM DTT, pH 7.3-7.5) of GeoCas9/sgRNA/ssDNA complexes was pipette-mixed rapidly with the lipid solution in ethanol at a volume ratio of 4:1 and weight ratio of 1:6.5 (GeoCas9/sgRNA/ssDNA: lipids), and then incubated for 1 hour at room temperature. The LNP mixture was dialyzed against PBS using a dialysis membrane with a molecular weight cut-off of 10 kDa (ThermoFisher) at 4° C. for 2 hours and then concentrated by ultrafiltration using Amicon Ultra-15 with a molecular weight cut-off of 100 kDa (Millipore).

### In Vivo Genome Editing

Two retro-orbital injections of LNPs consisting of different lipid formulations were performed with Ai9 tdTomato mice with a time window of three days. A week after the second injection, the mice were sacrificed, and all tissues were collected for further analysis. For flow analysis, isolated tissues were minced using a sterile blade and then subjected to collagenase digestion at 37° C. for 1 hour with shaking. Next, the digested solution was filtered using a 70-μm filter and quenched with PBS containing 2% FBS. A cell pellet was obtained by centrifuging for 5 min at a speed of 1500×g at 4° C. The supernatant was removed, and the cell pellet was resuspended in 1 ml of PBS containing 2% FBS, which could be used for flow analysis. For analysis by imaging, tissue blocks were embedded into optimal cutting temperature compounds (Sakura Finetek) and co-sectioned (8 μm) on a Cryostat instrument (Leica Biosystems) to prepare tissue sections. The mounted tissue slices were stained with DAPI before microscopy imaging.

What is claimed is:

1. A variant CRISPR-Cas effector polypeptide comprising an amino acid sequence having at least 50% amino acid sequence identity to the amino acid sequence depicted in FIG. 1 (SEQ ID NO: 1), wherein the variant CRISPR-Cas effector polypeptide comprises:
(a) a substitution of E149, T182, N206, and P466, based on the amino acid numbering of the GeoCas9 amino acid sequence depicted in FIG. 1; or
(b) a substitution of N206 and I331, based on the amino acid numbering of the GeoCas9 amino acid sequence depicted in FIG. 1; or
(c) a substitution of D1017, based on the amino acid numbering of the GeoCas9 amino acid sequence depicted in FIG. 1.

2. The variant CRISPR-Cas effector polypeptide of claim 1(a), wherein the variant CRISPR-Cas effector polypeptide further comprises a substitution of one or more of E179, L190, H340, 1379, K455, L601, Q817, K832, E843, K879, E884, K908, S924, K934, T1015, D1017, S1073, and D1087, based on the amino acid numbering of the GeoCas9 amino acid sequence depicted in FIG. 1 (SEQ ID NO: 1).

3. The variant CRISPR-Cas effector polypeptide of claim 1, wherein the variant CRISPR-Cas effector polypeptide comprises:
a) a substitution of E149, T182, N206, P466, and E843, based on the amino acid numbering of the GeoCas9 amino acid sequence depicted in FIG. 1; or
b) a substitution of E149, T182, N206, P466, E884, and K908, based on the amino acid numbering of the GeoCas9 amino acid sequence depicted in FIG. 1; or
c) a substitution of E149, T182, N206, P466, E843, E884, and K908, based on the amino acid numbering of the GeoCas9 amino acid sequence depicted in FIG. 1; or
d) a substitution of E149, T182, N206, P466, E843, E884, K908, and Q817, based on the amino acid numbering of the GeoCas9 amino acid sequence depicted in FIG. 1; or
e) a substitution of E149, T182, N206, P466, E843, E884, K908, T1015, and D1017, based on the amino acid numbering of the GeoCas9 amino acid sequence depicted in FIG. 1; or
f) a substitution of E149, T182, N206, P466, E843, E884, K908, and D1017, based on the amino acid numbering of the GeoCas9 amino acid sequence depicted in FIG. 1; or
g) a substitution of E149, T182, N206, P466, E843, E884, K908, L601, and K832, based on the amino acid numbering of the GeoCas9 amino acid sequence depicted in FIG. 1; or
h) a substitution of E149, T182, N206, P466, E843, E884, K908, and E179, based on the amino acid numbering of the GeoCas9 amino acid sequence depicted in FIG. 1; or
i) a substitution of E149, T182, N206, P466, E843, E884, K908, E179, and Q817, based on the amino acid numbering of the GeoCas9 amino acid sequence depicted in FIG. 1; or
j) a substitution of E149, T182, N206, P466, E843, E884, K908, K934, K832, and L601, based on the amino acid numbering of the GeoCas9 amino acid sequence depicted in FIG. 1; or
k) a substitution of E149, T182, N206, P466, E843, E884, K908, K934, and K832, based on the amino acid numbering of the GeoCas9 amino acid sequence depicted in FIG. 1; or
l) a substitution of E149, T182, N206, P466, E843, E884, K908, K832, L601, and T730, based on the amino acid numbering of the GeoCas9 amino acid sequence depicted in FIG. 1; or
m) a substitution of E149, T182, N206, P466, E843, E884, K908, Q817, L190, H340, 1379, K455, K879, and D1087, based on the amino acid numbering of the GeoCas9 amino acid sequence depicted in FIG. 1; or
n) a substitution of E149, T182, N206, P466, E843, E884, K908, Q817, 8924, and S1073, based on the amino acid numbering of the GeoCas9 amino acid sequence depicted in FIG. 1.

4. The variant CRISPR-Cas effector polypeptide of claim 3, wherein the variant CRISPR-Cas effector polypeptide comprises:
i) E149G, T182I, N206D, P466Q, and E843K substitutions;
ii) E149G, T182I, N206D, P466Q, E884G, and K908R substitutions;
iii) E149G, T182I, N206D, P466Q, E843K, E884G, and K908R substitutions;
iv) E149G, T182I, N206D, P466Q, E843K, E884G, K908R, and Q817R substitutions;
v) E149G, T182I, N206D, P466Q, E843K, E884G, K908R, T1015A, and D1017N substitutions;
vi) E149G, T182I, N206D, P466Q, E843K, E884G, K908R, and D1017G substitutions;
vii) E149G, T182I, N206D, P466Q, E843K, E884G, K908R, L601A, and K832R substitutions;
viii) E149G, T182I, N206D, P466Q, E843K, E884G, K908R, L601P, and K832R substitutions;
ix) E149G, T182I, N206D, P466Q, E843K, E884G, K908R, and E179G substitutions;
x) E149G, T182I, N206D, P466Q, E843K, E884G, K908R, E179G, and Q817R substitutions;
xi) E149G, T182I, N206D, P466Q, E843K, E884G, K908R, E179R, and Q817R E149G T182I N206D P466Q E843K E884G K908R E179R and Q817R substitutions;
xii) E149G, T182I, N206D, P466Q, E843K, E884G, K908R, K934V, K832R, and L601A substitutions;
xiii) E149G, T182I, N206D, P466Q, E843K, E884G, K908R, K934S, K832R, and L601T substitutions;
xiv) E149G, T182I, N206D, P466Q, E843K, E884G, K908R, K934D, K832R, and L601P substitutions;
xv) E149G, T182I, N206D, P466Q, E843K, E884G, K908R, K934S, and K832L substitutions;
xvi) E149G, T182I, N206D, P466Q, E843K, E884G, K908R, K832L, L601R, and T730A substitutions;
xvii) E149G. T182I, N206D, P466Q, E843K, E884G, K908R, Q817R, L190P, H340N, 1379T, K455R, K879R, and D1087A substitutions; or
xviii) E149G, T182I, N206D, P466Q, E843K, E884G, K908R, Q817R, S924P, and S1073N substitutions.

5. The variant CRISPR-Cas effector polypeptide of claim 1(b), wherein the variant CRISPR-Cas effector polypeptide comprises an N206T substitution and an 1331T substitution.

6. The variant CRISPR-Cas effector polypeptide of claim 1(c), further comprising a substitution of one or more of R829, K888, and T1015, based on the amino acid numbering of the GeoCas9 amino acid sequence depicted in FIG. 1.

7. The variant CRISPR-Cas effector polypeptide of claim 6, wherein the variant CRISPR-Cas effector polypeptide comprises R829H, K888R, T1015A, and D1017N substitutions.

8. The variant CRISPR-Cas effector polypeptide of claim 1(c), wherein the variant CRISPR-Cas effector polypeptide comprises a D1017G substitution.

9. The variant CRISPR-Cas effector polypeptide of any one of claims 1-8, wherein the variant CRISPR-Cas effector polypeptide is enzymatically active in a temperature range of from 15° C. to 75° C.

10. The variant CRISPR-Cas effector polypeptide of any one of claims 1-9, wherein the variant CRISPR-Cas effector polypeptide binds to a target nucleic acid comprising a PAM comprising a GAAA or a GCAA sequence.

11. The variant CRISPR-Cas effector polypeptide of any one of claims 1-10, wherein the variant CRISPR-Cas effector polypeptide has a length of from 1050 amino acids to 1120 amino acids.

12. The variant CRISPR-Cas effector polypeptide of any one of claims 1-10, wherein the variant CRISPR-Cas effector polypeptide has a length of from 1080 amino acids to 1095 amino acids.

13. The variant CRISPR-Cas effector polypeptide of any one of claims 1-12, wherein the variant CRISPR-Cas effector polypeptide comprises an amino acid sequence having at least 60% amino acid sequence identity to the amino acid sequence set forth in SEQ ID NO: 1.

14. The variant CRISPR-Cas effector polypeptide of any one of claims 1-12, wherein the variant CRISPR-Cas effector polypeptide comprises an amino acid sequence having at least 70% amino acid sequence identity to the amino acid sequence set forth in SEQ ID NO: 1.

15. The variant CRISPR-Cas effector polypeptide of any one of claims 1-12, wherein the variant CRISPR-Cas effector polypeptide comprises an amino acid sequence having at least 80% amino acid sequence identity to the amino acid sequence set forth in SEQ ID NO: 1.

16. The variant CRISPR-Cas effector polypeptide of any one of claims 1-12, wherein the variant CRISPR-Cas effector polypeptide comprises an amino acid sequence having at least 90% amino acid sequence identity to the amino acid sequence set forth in SEQ ID NO: 1.

17. The variant CRISPR-Cas effector polypeptide of any one of claims 1-12, wherein the variant CRISPR-Cas effector polypeptide comprises an amino acid sequence having at least 95% amino acid sequence identity to the amino acid sequence set forth in SEQ ID NO: 1.

18. The variant CRISPR-Cas effector polypeptide of any one of claims 1-17, wherein the variant CRISPR-Cas effector polypeptide exhibits at least 25% greater gene editing activity than a CRISPR-Cas effector polypeptide having the amino acid sequence depicted in FIG. 1.

19. The variant CRISPR-Cas effector polypeptide of any one of claims 1-17, wherein the variant CRISPR-Cas effector polypeptide exhibits at least 50% greater gene editing activity than a CRISPR-Cas effector polypeptide having the amino acid sequence depicted in FIG. 1.

20. The variant CRISPR-Cas effector polypeptide of any one of claims 1-17, wherein the variant CRISPR-Cas effector polypeptide exhibits at least 2-fold, at least 3-fold, at least 4-fold, or at least 5-fold, greater gene editing activity than a CRISPR-Cas effector polypeptide having the amino acid sequence depicted in FIG. 1.

21. A nucleic acid comprising a nucleotide sequence encoding the variant CRISPR-Cas effector polypeptide of any one of claims 1-20.

22. The nucleic acid of claim 21, wherein the nucleotide sequence encoding the variant CRISPR-Cas effector polypeptide is codon optimized for expression in a eukaryotic cell.

23. The nucleic acid of claim 22, wherein the nucleotide sequence encoding the variant CRISPR-Cas effector polypeptide is codon optimized for expression in a mammalian cell or a plant cell.

24. The nucleic acid of any one of claims 21-23, wherein the nucleotide sequence encoding the variant CRISPR-Cas effector polypeptide is operably linked to one or more transcriptional control elements.

25. The nucleic acid of claim 24, wherein the one or more transcriptional control elements comprises a promoter.

26. The nucleic acid of claim 25, wherein the promoter is a regulatable promoter or a constitutive promoter.

27. The nucleic acid of claim 25 or claim 26, wherein the promoter is functional in a eukaryotic cell.

28. A recombinant expression vector comprising the nucleic acid of any one of claims 21-27.

29. A composition comprising the variant CRISPR-Cas effector polypeptide of any one of claims 1-20, the nucleic acid of any one of claims 21-27, or the recombinant expression vector of claim 28.

30. The composition of claim 29, comprising one or more of: i) a buffer; ii) a nuclease inhibitor; iii) a protease inhibitor; and iv) a lipid.

31. A fusion polypeptide comprising:
a) a variant CRISPR-Cas effector polypeptide of any one of claims 1-20; and
b) one or more heterologous fusion partner polypeptides.

32. The fusion polypeptide of claim 31, wherein the one or more heterologous fusion partner polypeptides comprises a protein transduction domain that facilitates traversal of the variant CRISPR-Cas effector polypeptide from the cytosol of a cell to within an organelle in the cell.

33. The fusion polypeptide of claim 31, wherein the one or more heterologous fusion partner polypeptides is a reverse transcriptase.

34. The fusion polypeptide of claim 31, wherein the one or more heterologous fusion partner polypeptides has DNA modifying activity, increases transcription, decreases transcription, or modifies a polypeptide associated with a nucleic acid.

35. The fusion polypeptide of claim 31, wherein the one or more heterologous fusion partner polypeptides is a cytosine deaminase or an adenosine deaminase.

36. The fusion polypeptide of any one of claims 31-35, wherein the one or more heterologous fusion partner polypeptides comprises a nuclear localization signal (NLS).

37. A nucleic acid comprising a nucleotide sequence encoding the fusion polypeptide of any one of claims 31-36.

38. A recombinant expression vector comprising the nucleic acid of claim 37.

39. A cell comprising:
a) the variant CRISPR-Cas effector polypeptide of any one of claims 1-20; or
b) the nucleic acid of any one of claims 21-27; or
c) the recombinant expression vector of claim 28; or
d) the fusion polypeptide of any one of claims 31-36; or
e) the nucleic acid of claim 37; or
f) the expression vector of claim 38.

40. The cell of claim 39, wherein the cell is a eukaryotic cell.

41. The cell of claim 40, wherein the eukaryotic cell is a mammalian cell, a fish cell, an invertebrate animal cell, a vertebrate cell, a plant cell, an algal cell, a bird cell, an insect cell, an arachnid cell, an ungulate cell, a non-human primate cell, or a human cell.

42. The cell of any one of claims 39-41, wherein the cell is in vitro.

43. The cell of any one of claims 39-41, wherein the cell is in vivo.

44. A ribonucleoprotein (RNP) complex comprising:
a1) the variant CRISPR-Cas effector polypeptide of any one of claims 1-20; and
b1) a guide nucleic acid; or
a2) the fusion polypeptide of any one of claims 29-51; and
b2) a guide nucleic acid.

45. The RNP complex of claim 44, wherein the guide nucleic acid is a dual-guide RNA.

46. The RNP complex of claim 44, wherein the guide nucleic acid is a single-guide RNA.

47. The RNP complex of claim 44, wherein the guide nucleic acid comprises:
i) a DNA-targeting segment comprising a nucleotide sequence that is complementary to a target nucleotide sequence in a target nucleic acid; and
ii) a protein-binding segment that binds to and activates the variant CRISPR-Cas effector polypeptide, wherein the protein-binding segment comprises a duplex-forming linker segment and a tracrRNA.

48. The RNP complex of claim 44, wherein the guide nucleic acid is a dual-guide RNA comprising:
i) a first RNA comprising the DNA-targeting segment; and
ii) a second RNA comprising the protein-binding segment, wherein the first RNA and the second RNA are not contiguous with one another and are not covalently linked to one another.

49. The RNP complex of claim 44, wherein the guide nucleic acid is a single-molecule guide RNA, wherein the DNA-targeting segment and the protein-binding segment are present in a single RNA molecule.

50. The RNP complex of any one of claims 44-49, wherein the guide nucleic acid comprises one or more of a modified nucleobase, a modified backbone, a non-natural internucleoside linkage, a modified sugar moiety, a Locked Nucleic Acid, and a Peptide Nucleic Acid.

51. The RNP complex of any one of claims 44-50, comprising two or more guide nucleic acids.

52. The RNP complex of any one of claims 44-51, comprising a donor template nucleic acid.

53. A multicellular, non-human organism comprising:
a) the variant CRISPR-Cas effector polypeptide of any one of claims 1-20; or
b) the nucleic acid of any one of claims 21-27; or
c) the recombinant expression vector of claim 28; or
d) the fusion polypeptide of any one of claims 31-36; or
c) the nucleic acid of any one of claim 373; or
f) the expression vector of claim 38.

54. A method of modifying a target DNA, the method comprising contacting the target DNA with the RNP complex of any one of claims 44-52.

55. The method of claim 54, wherein said modifying comprises non-homologous end joining.

56. The method of claim 54, wherein said modifying comprises homology-directed repair.

57. The method of any one of claims 54-56, wherein said contacting occurs in a cell in vitro.

58. The method of any one of claims 54-56, wherein said contacting occurs in a cell in vivo.

59. The method of any one of claims 54-58, wherein the target DNA is present in a eukaryotic cell.

60. The method of claim 115, wherein the target DNA is chromosomal DNA, chloroplast DNA, or mitochondrial DNA.

61. The method of any one of claims 54-60, wherein said modifying comprises cleavage of the target DNA.

62. A lipid nanoparticle (LNP) comprising:
a) a first lipid, wherein the first lipid comprises a covalently attached poly(ethylene glycol) (PEG) moiety;
b) at least a second and a third lipid, wherein the second lipid and the third lipid do not comprise a covalently attached PEG moiety;
c) a CRISPR-Cas effector polypeptide; and
d) a guide nucleic acid.

63. The LNP of claim 62, wherein the first lipid is selected from the group consisting of M-PEG, DMG-mPEG, DOPE-mPEG, DOPE-PEG-CO₂H, DOPE-PEG-NH₂, where the PEG has a molecular weight of about 2 kD.

64. The LNP of claim 62 or claim 63, wherein the second and third lipids are selected from the group consisting of DOPE, DSPE, DOTAP, DSTAP, D-Lin, C12-300, and cholesterol.

65. The LNP of any one of claims 62-64, wherein the LNP comprises DOTAP, D-Lin, DOPE, cholesterol, and M-PEG-2k.

66. The LNP of claim 65, wherein the LNP comprises DOTAP, D-Lin, DOPE, cholesterol, and M-PEG-2k in a ratio of 18-20:36:12-14:40:1.5-2.5 DOTAP:D-Lin:DOPE:cholesterol:M-PEG-2k.

67. The LNP of any one of claims 62-66, wherein the CRISPR-Cas polypeptide is:
a) a variant CRISPR-Cas polypeptide of any one of claims 1-20; or
b) a fusion polypeptide of any one of claims 31-36.

68. The LNP of any one of claims 62-66, wherein the CRISPR-Cas effector polypeptide is:
a) a type II CRISPR-Cas effector polypeptide, a type III CRISPR-Cas effector polypeptide, a type IV CRISPR-Cas effector polypeptide, a type V CRISPR-Cas effector polypeptide, or a type VI CRISPR-Cas effector polypeptide; or
b) a fusion polypeptide comprising: i) one or more heterologous fusion polypeptides; and ii) a type II CRISPR-Cas effector polypeptide, a type III CRISPR-Cas effector polypeptide, a type IV CRISPR-Cas effector polypeptide, a type V CRISPR-Cas effector polypeptide, or a type VI CRISPR-Cas effector polypeptide.

69. A method of making the LNP of any one of claims 62-68, the method comprising:
a) combining the CRISPR-Cas polypeptide and the guide nucleic acid with the first lipid in an aqueous solution, to form a first composition; and
b) combining the first composition with a second composition, wherein the second composition comprises, in an organic solvent, the second and third lipids,
thereby forming the LNP.

70. A method of delivering a ribonucleoprotein (RNP) into a eukaryotic cell, the method comprising contacting the eukaryotic cell with an LNP of any one of claims 62-68, wherein the RNP comprises a CRISPR-Cas effector polypeptide and a guide nucleic acid, and wherein said contacting results in delivery of the RNP into the eukaryotic cell.

71. The method of claim 70, wherein the eukaryotic cell is in vitro.

72. The method of claim 70, wherein the eukaryotic cell is in vivo.

73. A CRISPR-Cas effector polypeptide comprising an amino acid sequence having at least 50% amino acid sequence identity to the amino acid sequence of any one of SEQ ID NOs.: 165-175, wherein the variant CRISPR-Cas effector polypeptide comprises a substitution at an amino acid position corresponding to:
(a) E149, T182, N206, and P466; or
(b) D1017,
based on the amino acid numbering of the ThermoCas9 amino acid sequence of SEQ ID NO: 165.

74. The CRISPR-Cas effector polypeptide of claim 73, comprising a substitution at an amino acid position corresponding to:
a) E149, T182, N206, P466, and E843; or
b) E149, T182, N206, P466, E884, and T908; or
c) E149, T182, N206, P466, E843, E884, and T908; or
d) E149, T182, N206, P466, E843, E884, T908, and Q817; or
e) E149, T182, N206, P466, E843, E884, T908, T1015, and D1017; or
f) E149, T182, N206, P466, E843, E884, T908, and D1017,
based on the amino acid numbering of the ThermoCas9 amino acid sequence of SEQ ID NO: 165.

75. The CRISPR-Cas effector polypeptide of claim 74, wherein the variant CRISPR-Cas effector polypeptide comprises the substitutions:
i) E149G, T182I, N206D, P466Q, and E843K; or
ii) E149G, T182I, N206D, P466Q, E884G, and T908R; or
iii) E149G, T182I, N206D, P466Q, E843K, E884G, and T908R; or
iv) E149G, T182I, N206D, P466Q, E843K, E884G, T908R, and Q817R; or
v) E149G, T182I, N206D, P466Q, E843K, E884G, T908R, T1105A, and D1017N; or
vi) E149G, T182I, N206D, P466Q, E843K, E884G, T908R, and D1017G.

76. The CRISPR-Cas effector polypeptide of any one of claims 73-75, wherein the CRISPR-Cas effector polypeptide comprises an amino acid sequence having at least 80% amino acid sequence identity to the amino acid sequence of any one of SEQ ID NOs.: 165-175.

77. The CRISPR-Cas effector polypeptide of any one of claims 73-75, wherein the CRISPR-Cas effector polypeptide comprises an amino acid sequence having at least 80% amino acid sequence identity to the amino acid sequence of any one of SEQ ID NOs.: 165.

78. The CRISPR-Cas effector polypeptide of claim 73, wherein the CRISPR-Cas effector polypeptide comprises an amino acid sequence having at least 70% amino acid sequence identity to the amino acid sequence of SEQ ID NOs.: 176.

79. A nucleic acid comprising a nucleotide sequence encoding the variant CRISPR-Cas effector polypeptide of any one of claims 73-78.

80. The nucleic acid of claim 79, wherein the nucleotide sequence encoding the CRISPR-Cas effector polypeptide is codon optimized for expression in a eukaryotic cell.

81. The nucleic acid of claim 79 or claim 80, wherein the nucleotide sequence encoding the CRISPR-Cas effector polypeptide is operably linked to a promoter functional in a eukaryotic cell.

82. A recombinant expression vector comprising the nucleic acid of any one of claims 79-81.

83. A fusion polypeptide comprising:
a) a CRISPR-Cas effector polypeptide of any one of claims 73-78; and
b) one or more heterologous fusion partner polypeptides.

84. The fusion polypeptide of claim 83, wherein the one or more heterologous fusion partner polypeptides comprises a reverse transcriptase.

85. The fusion polypeptide of claim 83, wherein the one or more heterologous fusion partner polypeptides has DNA modifying activity, increases transcription, decreases transcription, or modifies a polypeptide associated with a nucleic acid.

86. The fusion polypeptide of claim 83, wherein the one or more heterologous fusion partner polypeptides comprises a cytosine deaminase or an adenosine deaminase.

87. A nucleic acid comprising a nucleotide sequence encoding the fusion polypeptide of any one of claims 83-86.

88. A cell comprising:
a) the CRISPR-Cas effector polypeptide of any one of claims 73-78 or a nucleic acid encoding the CRISPR-Cas effector polypeptide; or
b) the fusion polypeptide of any one of claims 83-86 or a nucleic acid encoding the fusion polypeptide.

89. The cell of claim 88, wherein the cell is a eukaryotic cell.

90. A ribonucleoprotein (RNP) complex comprising:
a guide nucleic acid and the CRISPR-Cas effector polypeptide of any one of claims 73-78; or
a guide nucleic acid and the fusion polypeptide of any one of claims 83-86.

91. A multicellular, non-human organism comprising:
a) the CRISPR-Cas effector polypeptide of any one of claims 73-78 or a nucleic acid encoding the CRISPR-Cas effector polypeptide; or
b) the fusion polypeptide of any one of claims 83-86 or a nucleic acid encoding the fusion polypeptide.

92. A method of modifying a target DNA, the method comprising contacting the target DNA with the RNP complex of claim 90.

93. The method of claim 92, wherein the target DNA is present in a eukaryotic cell.

94. The method of claim 93, wherein said contacting occurs in the eukaryotic cell in vivo.

95. The method of any one of claims 92-94, wherein said modifying comprises cleavage of the target DNA.

96. The LNP of any one of claims 62-66, wherein the CRISPR-Cas polypeptide is:
a) the CRISPR-Cas polypeptide of any one of claims 73-78; or
b) the fusion polypeptide of any one of claims 83-86.

97. The LNP of claim 67 or claim 96, wherein the first lipid of the LNP comprises an acid-degradable PEGylated lipid.

98. The LNP of claim 97, wherein the acid-degradable PEGylated lipid is ADP-2k, Pep-1k, or Pep-2k.

99. The LNP of any one of claims 96-98, wherein the LNP comprises ADC as a cationic lipid.

100. The method of claim 58 or claim 94, wherein the eukaryotic cell is a liver cell and said contacting comprises introducing the RNP to the liver cell as part of a liquid nanoparticle (LNP) that comprises an acid-degradable PEGylated lipid.

101. The method of claim 58 or claim 94, wherein the eukaryotic cell is a lung cell and said contacting comprises introducing the RNP to the lung cell as part of a liquid nanoparticle (LNP) that comprises ADC as a cationic lipid.

102. The method of claim 101, wherein the LNP further comprises an acid-degradable PEGylated lipid.

103. A method of delivering a ribonucleoprotein (RNP) into a eukaryotic cell, the method comprising contacting the eukaryotic cell with an LNP of any one of claims 96-98, wherein the RNP comprises a CRISPR-Cas effector polypeptide and a guide nucleic acid, and wherein said contacting results in delivery of the RNP into the eukaryotic cell.

104. The method of claim 103, wherein the eukaryotic cell is in vivo.

105. The method of claim 72 or claim 104, wherein the eukaryotic cell is a liver cell and the LNP comprises an acid-degradable PEGylated lipid.

106. The method of claim 72 or claim 104, wherein the eukaryotic cell is a lung cell and the LNP comprises ADC as a cationic lipid.

107. The method of claim 106, wherein the LNP further comprises an acid-degradable PEGylated lipid.
